(19) United States
(12) Patent Application Publication
(10) Pub. No.: US 2009/0203692 A1
(43) Pub. Date: Aug. 13, 2009

(54) NOVEL CHEMICAL COMPOUNDS

(75) Inventors: Kevin Duffy, Collegeville, PA (US); Deping Chai, Collegeville, PA (US); Mirela Colon, Collegeville, PA (US); Duke M. Fitch, Collegeville, PA (US); Sarah Rae King, Rural Retreat, VA (US); Antony N. Shaw, Collegeville, PA (US); Rosanna Tedesco, Collegeville, PA (US); Kenneth Wiggall, Collegeville, PA (US); Neil W. Johnson, Collegeville, PA (US); Michael N. Zimmerman, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION

(21) Appl. No.: 11/912,965

(22) PCT Filed: May 18, 2006
(86) PCT No.: PCT/US06/19447

§ 371 Date: Jul. 12, 2008

(30) Foreign Application Priority Data
May 23, 2005 (US) 60/683752

(51) Int. Cl. A61K 31/4709; C07D 417/14; C07D 417/06; A61K 31/5377; A61K 31/498; A61P 7/06; A61P 7/00

(52) U.S. Cl. 514/235.2; 546/175; 544/353; 544/331; 544/128; 514/249; 514/314

(57) ABSTRACT

This invention relates to newly identified compounds for inhibiting hYAK3 proteins and methods for treating diseases associated with hYAK3 activity.

### FIELD OF THE INVENTION

This invention relates to newly identified compounds for inhibiting hYAK3 proteins and methods for treating diseases associated with hYAK3 activity.

### BACKGROUND OF THE INVENTION

A number of polypeptide growth factors and hormones mediate their cellular effects through a signal transduction pathway. Transduction of signals from the cell surface receptors for these ligands to intracellular effectors frequently involves phosphorylation or dephosphorylation of specific protein substrates by regulatory protein serine/threonine kinases (PSTK) and phosphatases. Serine/threonine phosphorylation is a major mediator of signal transduction in multicellular organisms. Receptor-bound, membrane-bound and intracellular PSTKs regulate cell proliferation, cell differentiation and signalling processes in many cell types.

Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are potential targets for drug design.

A subset of PSTKs are involved in regulation of cell cycling. These are the cyclin-dependent kinases or CDKs (Peter and Herskowitz, Cell 1994: 79, 181-184). CDKs are activated by binding to regulatory proteins called cyclins and control passage of the cell through specific cell cycle checkpoints. For example, CDK2 complexed with cyclin E allows cells to progress through the G1 to S phase transition. The complexes of CDKs and cyclins are subject to inhibition by low molecular weight proteins such as p16 (Serrano et al, Nature 1993: 366, 704), which binds to and inhibits CDK4. Deletions or mutations in p16 have been implicated in a variety of tumors (Kamb et al, Science 1994: 264, 436-440). Therefore, the proliferative state of cells and diseases associated with this state are dependent on the activity of CDKs and their associated regulatory molecules. In diseases such as cancer where inhibition of proliferation is desired, compounds that inhibit CDKs may be useful therapeutic agents. Conversely, activators of CDKs may be useful where enhancement of proliferation is needed, such as in the treatment of immunodeficiency.

YAK1, a PSTK with sequence homology to CDKs, was originally identified in yeast as a mediator of cell cycle arrest caused by inactivation of the cAMP-dependent protein kinase PKA (Garrett et al, Mol Cell Biol. 1991: 11-6045-4052). YAK1 kinase activity is low in cycling yeast but increases dramatically when the cells are arrested prior to the S-G2 transition. Increased expression of YAK1 causes growth arrest in yeast cells deficient in PKA. Therefore, YAK1 can act as a cell cycle suppressor in yeast.

U.S. Pat. No. 6,323,318 describes two novel human homologs of yeast YAK1 termed hYAK3-2, one protein longer than the other by 20 amino acids. hYAK3-2 proteins (otherwise reported as REDK-L and REDK-S in Blood, 1 May 2000, Vol 95, No. 9, pp 2838) are primarily localized in the nucleus. hYAK-2 proteins (hereinafter simply referred as hYAK3 or hYAK3 proteins) are present in hematopoietic tissues, such as bone marrow and fetal liver, but the RNA is expressed at significant levels only in erythroid or erthropoietin (EPO)-responsive cells. Two forms of REDK cDNAs appear to be alternative splice products. Antisense REDK oligonucleotides promote erythroid colony formation by human bone marrow cells, without affecting colony-forming unit (CFU)-GM, CFU-G, or CFU-GEMM numbers. Maximal numbers of CFU-E and burst-forming unit-erythroid were increased, and CFU-E displayed increased sensitivity to suboptimal EPO concentrations. The data indicate that REDK acts as a brake to retard erythropoiesis. Thus inhibitors of hYAK3 proteins are expected to stimulate proliferation of cells in which it is expressed. More particularly, inhibitors of hYAK3 proteins are useful to treat or prevent diseases of the erythroid and hematopoietic systems associated with hYAK3 activity, including but not limited to, anemia, anemias due to renal insufficiency or to chronic disease, such as autoimmunity, HIV, or cancer, and drug-induced anemias, myelodysplastic syndrome, aplastic anemia and myelosuppression, and cytopenia.

### SUMMARY OF THE INVENTION

This invention relates to novel compounds of Formula (I):

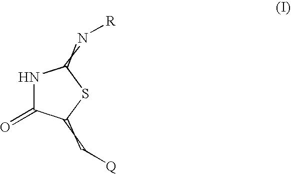

in which

R is selected form: aryl and substituted aryl; and

### Q is

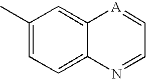

wherein

A is selected from CH and N;

and/or pharmaceutically acceptable salts, hydrates, solvates and pro-drugs thereof, provided that when R is

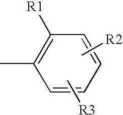

R¹ is not hydrogen, halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂C₁₋₆alkyl,
when R² and R³ are independently selected from: hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —CONH₂, —NH₂, —OCH₂(C═O)OH, —OCH₂CH₂OCH₃, —SO₂NH₂, —CH₂SO₂CH₃, and —NH(C═NH)CH₃, and
   further provided that R is not naphthyl.

This invention relates to a compound of Formula I, as described above, further provided that R is not t-butylthiazol.

This invention relates to a compound of Formula I, as described above, further provided that R is not t-butylthiazol and further provided that R¹ is not hydrogen, halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂C₁₋₆alkyl when R² and R³ are independently selected from:

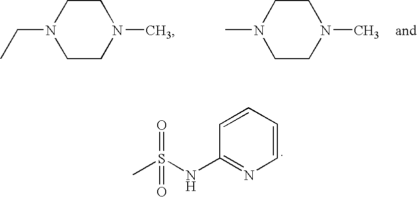

This invention relates a method of inhibiting hYAK3 in a mammal; comprising, administering to the mammal a therapeutically effective amount of a compound of the formula (I).

This invention relates to a method of treating or preventing diseases of the erythroid and hematopoietic systems, caused by hYAK3 activity including, but not limited to, anemias due to renal insufficiency or to chronic disease, such as autoimmunity, HIV, or cancer, and drug-induced anemias, myelodysplastic syndrome, aplastic anemia and myelosuppression, and cytopenia; comprising administering to a mammal a therapeutically effective amount of a compound of formula (I).

In a further aspect of the invention there is provided novel processes and novel intermediates useful in preparing the presently invented hYAK3 inhibiting compounds.

Included in the present invention are pharmaceutical compositions that comprise a pharmaceutical carrier and compounds useful in the methods of the invention.

Also included in the present invention are methods of co-administering the presently invented hYAK3 inhibiting compounds with further active ingredients.

### DETAILED DESCRIPTION

This invention relates to compounds of Formula (I) as described above.

The presently invented compounds of Formula (I) inhibit hYAK3 activity.

Included among the presently invented compounds of Formula I are those in which A is nitrogen.

Included among the presently invented compounds of Formula (I) are those having Formula (II):

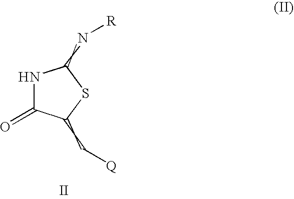

in which

R is selected form: C₁-C₁₂aryl and substituted C₁-C₁₂aryl; and

### Q is

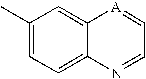

wherein

A is selected from CH and N;

and/or pharmaceutically acceptable salts, hydrates, solvates and pro-drugs thereof, provided that when R is

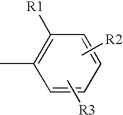

R¹ is not hydrogen, halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂C₁₋₆alkyl,
when R² and R³ are independently selected from: hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —CONH₂, —NH₂, —OCH₂(C═O)OH, —OCH₂CH₂OCH₃, —SO₂NH₂, —CH₂SO₂CH₃, and —NH(C═NH)CH₃, and
   further provided that R is not naphthyl.

Included among the presently invented compounds of Formula II are those where it is further provided that R is not t-butylthiazol.

Included among the presently invented compounds of Formula II are those where it is further provided that R is not t-butylthiazol and further provided that R¹ is not hydrogen, halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂C₁₋₆alkyl when R² and R³ are independently selected from:

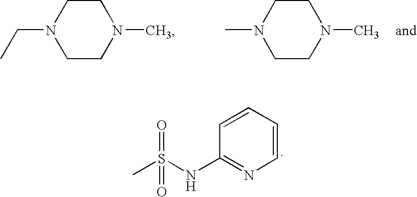

Included among the presently invented compounds of Formula II are those in which A is nitrogen.

Included among the presently invented compounds of Formulas (I) and (II) are those in which:

R is substituted phenyl; and

### Q is

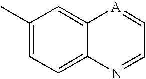

wherein

A is selected from CH and N;

and/or pharmaceutically acceptable salts, hydrates, solvates and pro-drugs thereof, provided that when R is

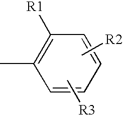

R¹ is not hydrogen, halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂C₁₋₆alkyl,
when R² and R³ are independently selected from: hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —CONH₂, —NH₂, —OCH₂(C═O)OH, —OCH₂CH₂OCH₃, —SO₂NH₂, —CH₂SO₂CH₃, and —NH(C═NH)CH₃.

Included among the presently invented compounds of Formula (I) and Formula (II), described immediately above, are those where it is further provided that that R¹ is not hydrogen, halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂C₁₋₆alkyl when R² and R³ are independently selected from:

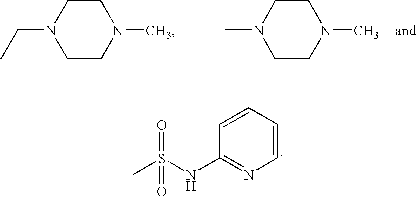

Included among the presently invented compounds of Formula I and Formula II, described immediately above, are those in which A is nitrogen.

Included among the presently invented compounds of Formulas (I) and (II) are those in which:

### R is

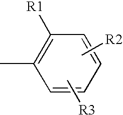 [0042] in which R¹ is selected form: hydrogen, halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —OH, and —CN; and
R² and R³ are independently selected from: hydrogen, halogen, —C₁₋₆ alkyl, substituted —C₁₋₆alkyl, C₁-C₁₂aryl, cycloalkyl, cycloalkyl containing from 1 to 4 heteroatoms, —SC₁₋₆alkyl, substituted —SC₁₋₆alkyl, —OC₁₋₆alkyl, substituted —OC₁₋₆alkyl, —NO₂, —OH, —CN, —NH₂, alkylamino, dialkylamino, —SO₂NH₂,
—S(O)₂NR⁴⁰R³⁰, where R³⁰ is selected from alkyl, cycloalkyl, substituted cycloalkyl, cycloalkyl containing 1 to 4 heteroatoms, substituted cycloalkyl containing 1 to 4 heteroatoms and aryl, and R⁴⁰ is selected from hydrogen and C₁-C₆alkyl,
—NR⁴¹C(O)R³¹, where R³¹ is selected from aryl, -Oalkyl, -Oaryl, cycloalkyl, substituted cycloalkyl, cycloalkyl containing 1 to 4 heteroatoms, substituted cycloalkyl containing 1 to 4 heteroatoms, optionally substituted alkyl, and —NR³²R³³, where R³² and R³³ are selected from alkyl and aryl, and R⁴¹ is selected from hydrogen and C₁-C₆alkyl,
—NR⁴⁴S(O)₂R³⁴, where R³⁴ is selected from hydrogen, alkyl, cycloalkyl, substituted cycloalkyl, cycloalkyl containing 1 to 4 heteroatoms, substituted cycloalkyl containing 1 to 4 heteroatoms and aryl, and R⁴⁴ is selected from hydrogen and C₁-C₆alkyl,
—CONR⁴⁵R³⁵, where R³⁵ is selected from alkyl, cycloalkyl, substituted cycloalkyl, cycloalkyl containing 1 to 4 heteroatoms, substituted cycloalkyl containing 1 to 4 heteroatoms and aryl, and R⁴⁵ is selected from hydrogen and C₁-C₆alkyl, and
—NH(C═NH)CH₃;
and

### Q is

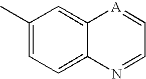

wherein

A is selected from CH and N;

and/or pharmaceutically acceptable salts, hydrates, solvates and pro-drugs thereof, provided that
R¹ is not hydrogen, halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂C₁₋₆alkyl,
when R² and R³ are independently selected from: hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —CONH₂, —NH₂, —OCH₂(C═O)OH, —OCH₂CH₂OCH₃, —SO₂NH₂, —CH₂SO₂CH₃, and —NH(C═NH)CH₃.

Included among the presently invented compounds of Formula (I) and Formula (II), described immediately above, are those where it is further provided that that R¹ is not hydrogen, halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂C₁₋₆alkyl when R² and R³ are independently selected from:

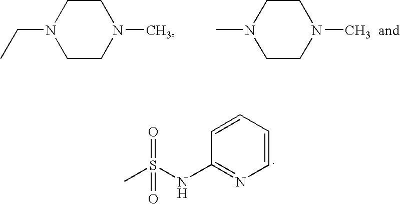

Included among the presently invented compounds of Formula I and Formula II, described immediately above, are those in which A is nitrogen.

Included among the presently invented compounds of Formulas (I) and (II) are those in which:

### R is

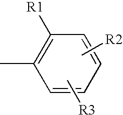 [0058] in which R1 is selected from: halogen, —C₁₋₆alkyl, substituted —C₁₋₆alkyl, —SC₁₋₆alkyl, substituted —SC₁₋₆alkyl, —OC₁₋₆alkyl, substituted —OC₁₋₆alkyl, —NO₂, —OH, and —CN; and
R2 and R3 are independently selected from: hydrogen, halogen, —C₁₋₆ alkyl, substituted —C₁₋₆ alkyl, C₁-C₁₂aryl, cycloalkyl, cycloalkyl containing from 1 to 4 heteroatoms, —SC₁₋₆alkyl, substituted —SC₁₋₆alkyl, —OC₁₋₆alkyl, substituted —OC₁₋₆alkyl, —NO₂, —OH, —CN,
—S(O)₂NR⁴⁰R³⁰, where R³⁰ is selected from alkyl, cycloalkyl, substituted cycloalkyl, cycloalkyl containing 1 to 4 heteroatoms, substituted cycloalkyl containing 1 to 4 heteroatoms and aryl, and R⁴⁰ is selected from hydrogen and C₁-C₆alkyl,
—NR⁴¹C(O)R³¹, where R³¹ is selected from aryl, -Oalkyl, -Oaryl, cycloalkyl, substituted cycloalkyl, cycloalkyl containing 1 to 4 heteroatoms, substituted cycloalkyl containing 1 to 4 heteroatoms, optionally substituted alkyl, and —NR³²R³³, where R³² and R³³ are selected from alkyl and aryl, and R⁴¹ is selected from hydrogen and C₁-C₆alkyl,
—NR⁴⁴S(O)₂R³⁴, where R³⁴ is selected from hydrogen, alkyl, cycloalkyl, substituted cycloalkyl, cycloalkyl containing 1 to 4 heteroatoms, substituted cycloalkyl containing 1 to 4 heteroatoms and aryl, and R⁴⁴ is selected from hydrogen and C₁-C₆alkyl,
—CONR⁴⁵R³⁵, where R³⁵ is selected from alkyl, cycloalkyl, substituted cycloalkyl, cycloalkyl containing 1 to 4 heteroatoms, substituted cycloalkyl containing 1 to 4 heteroatoms and aryl, and R⁴⁵ is selected from hydrogen and C₁-C₆alkyl,
—NH₂, alkylamino, dialkylamino, and —NH(C═NH)CH₃;
and

### Q is

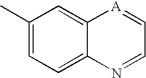

wherein

A is selected from CH and N;

and/or pharmaceutically acceptable salts, hydrates, solvates and pro-drugs thereof, provided that
R¹ is not halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂C₁₋₆alkyl,
when R² and R³ are independently selected from: hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —CONH₂, —NH₂, —OCH₂(C═O)OH, —OCH₂CH₂OCH₃, —CH₂SO₂CH₃, and —NH(C═NH)CH₃.

Included among the presently invented compounds of Formula (I) and Formula (II), described immediately above, are those where it is further provided that that R¹ is not hydrogen, halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂C₁₋₆alkyl when R² and R³ are independently selected from:

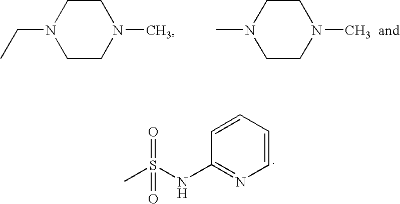

Included among the presently invented compounds of Formula I and Formula II, described immediately above, are those in which A is nitrogen.

Included among the presently invented compounds of Formulas (I) and (II) are those in which:

### R is

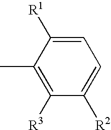 [0074] in which R¹ is selected from: halogen, C₁₋₆alkyl, substituted C₁₋₆alkyl, amine, C₁₋₆alkylamine and C₁₋₆dialkylamine;
R³ is selected from: hydrogen, halogen, C₁₋₆alkyl, substituted C₁₋₆alkyl, amine, C₁₋₆alkylamine and C₁₋₆dialkylamine; and
R² is selected from: —NR⁶⁰S(O)₂R⁷⁰ and —N(R⁷⁰)C(O)R⁷⁰,
where,
R⁶⁰ is selected from: hydrogen, C₁₋₆alkyl, C₁-C₁₂aryl and C₁-C₁₂arylC₁-C₆alkyl, and
each R⁷⁰ is independently selected from: hydrogen, C₁₋₆alkyl, C₁-C₆alkyloxyC₁-C₆alkyl, C₁-C₆alkyloxyC₁-C₆alkylamine, C₁-C₄alkylC(O)OC₁-C₄alkyl, —C₁-C₆alkylC(O)OH, amino, alkylamino where the alkyl is optionally substituted with one or more substituents selected from hydroxy, oxo, cycloalkyl containing from 1 to 4 heteroatoms where cycloalkyl containing from 1 to 4 heteroatoms is optionally substituted by one or more substituents selected form C₁-C₆alkyl, halogen and oxo, and C₁-C₆alkyl, dialkylamino where each alkyl is independently and optionally substituted with one or more substituents selected from hydroxyl, oxo, C₁-C₆alkyl, amino, alkylamino and dialkylamino, aminoC₁-C₆alkyl, alkylaminoC₁-C₆alkyl where the alkyl is optionally substituted by one or more substituents selected from oxo, alkoxy and halogen, dialkylaminoC₁-C₆alkyl where each alkyl is independently and optionally substituted with one or more substituents selected from hydroxy, oxo, C₁-C₆alkyl, amino, alkylamino and dialkylamino, —C(O)OH, alkoxy, aryloxy, arylamino, diarylamino, arylalkylamino where the aryl is optionally substituted with one or more substituents selected from hydroxyl, alkoxy, hydroxyalkyl, oxo, C₁-C₆alkyl, amino, alkylamino and dialkylamino, cycloalkylalkylamino, aryl, aryl substituted with one or more substituents selected from oxo, hydroxyl, —N(H)C(O)C₁-C₆alkyl, alkoxy, nitro, amine and alkyl, arylC₁-C₄alkyl optionally substituted with one or more substituents selected from oxo, amino, alkylamino, alkylamino substituted by oxo, dialkylamino, dialkylamino substituted by one or more oxo groups, hydroxy, halogen, alkoxy and alkyl, —CH₂C(O)cycloalkyl containing from 1 to 4 heteroatoms, cycloalkylC₁-C₆alkyl where the cycloalkyl is optionally substituted with one or more substituents selected from C₁-C₆alkyl, halogen, —C(O)OC₁-C₆alkyl and —C₁-C₆alkylC(O)OH, C₁-C₄alkyl substituted with cycloalkyl containing from 1 to 4 heteroatoms where the cycloalkyl containing from 1 to 4 heteroatoms is optionally substituted with one or more substituents selected from C₁-C₆alkyl, oxo, halogen, —C(O)OC₁-C₆alkyl and —C₁-C₆alkylC(O)OH, cycloalkyl, —N(H)cycloalkyl, cycloalkyl substituted with one or more substituents selected from oxo, hydroxy, halogen, amino, alkylamino, dialkylamino, —C(O)OH, —C(O)NR⁸⁰R⁹⁰ where R⁸⁰ and R⁹⁰ are each independently selected form hydrogen and C₁-C₈alkyl, and alkyl, —N(H)cycloalkyl substituted with one or more substituents selected from oxo, hydroxy and alkyl, cycloalkyl containing from 1 to 4 heteroatoms, cycloalkyl containing from 1 to 4 heteroatoms substituted with one or more substituents selected from oxo, alkoxy, hydroxyl and alkyl where alkyl is optionally substituted with one or more substituents selected from halogen, hydroxy, alkoxy, oxo and cycloalkyl containing from 1 to 4 heteroatoms, —N(H)cycloalkyl containing from 1 to 4 heteroatoms substituted with one or more substituents selected from oxo, hydroxy and alkyl, and trifluoromethyl;
and

### Q is

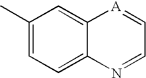

wherein

A is N;

and/or pharmaceutically acceptable salts, hydrates, solvates and pro-drugs thereof.

Included among the presently invented compounds of Formulas (I) and (II) are those in which:

### R is

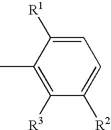 [0085] in which R¹ is selected from: halogen and C₁₋₆alkyl;
R³ is selected from: hydrogen, halogen and C₁₋₆alkyl; and
R² is selected from: —NR⁶¹S(O)₂R⁷¹ and —N(R⁷¹)C(O)R⁷¹,
where,
R⁶¹ is selected from: hydrogen and C₁₋₆alkyl, and
each R⁷¹ is independently selected from: hydrogen, C₁₋₆alkyl, C₁-C₆alkyloxyC₁-C₆alkyl, C₁-C₆alkyloxyC₁-C₆alkylamine, C₁-C₄alkylC(O)OC₁-C₄alkyl, —C₁-C₆alkylC(O)OH, amino, alkylamino where the alkyl is optionally substituted with one or more substituents selected from hydroxy, cycloalkyl containing from 1 to 4 heteroatoms where cycloalkyl containing from 1 to 4 heteroatoms is optionally substituted by one or more substituents selected form C₁-C₆alkyl, halogen and oxo, and C₁-C₆alkyl, dialkylamino where each alkyl is independently and optionally substituted with one or more substituents selected from hydroxy, oxo, C₁-C₆alkyl, amino, alkylamino and dialkylamino, aminoC₁-C₆alkyl, alkylaminoC₁-C₆alkyl where the alkyl is optionally substituted by one or more substituents selected from oxo, alkoxy and halogen, dialkylaminoC₁-C₆alkyl where each alkyl is independently and optionally substituted with one or more substituents selected from hydroxy, oxo, C₁-C₆alkyl, amino, alkylamino and dialkylamino, alkoxy, aryloxy, arylamino, diarylamino, arylalkylamino where the aryl is optionally substituted with one or more substituents selected from hydroxyl, alkoxy, hydroxyalkyl, oxo, C₁-C₆alkyl, amino, alkylamino and dialkylamino, cycloalkylalkylamino, aryl, aryl substituted with one or more substituents selected from oxo, hydroxyl, —N(H)C(O)C₁-C₆alkyl, alkoxy, nitro, amine and alkyl, arylC₁-C₄alkyl optionally substituted with one or more substituents selected from oxo, amino, alkylamino, alkylamino substituted by oxo, dialkylamino, dialkylamino substituted by one or more oxo groups, hydroxy, halogen, alkoxy and alkyl, —CH₂C(O)cycloalkyl containing from 1 to 4 heteroatoms, cycloalkylC₁-C₆alkyl where the cycloalkyl is optionally substituted with one or more substituents selected from C₁-C₆alkyl, halogen, —C(O)OC₁-C₆alkyl and —C₁-C₆alkylC(O)OH, C₁-C₄alkyl substituted with cycloalkyl containing from 1 to 4 heteroatoms where the cycloalkyl containing from 1 to 4 heteroatoms is optionally substituted with one or more substituents selected from C₁-C₆alkyl, oxo, halogen, —C(O)OC₁-C₆alkyl and —C₁-C₆alkylC(O)OH, cycloalkyl, —N(H)cycloalkyl, cycloalkyl substituted with one or more substituents selected from oxo, hydroxy, halogen, amino, alkylamino, dialkylamino, —C(O)OH, —C(O)NR⁸⁰R⁹⁰ where R⁸⁰ and R⁹⁰ are each independently selected form hydrogen and C₁-C₈alkyl, and alkyl, —N(H)cycloalkyl substituted with one or more substituents selected from oxo, hydroxy and alkyl, cycloalkyl containing from 1 to 4 heteroatoms, cycloalkyl containing from 1 to 4 heteroatoms substituted with one or more substituents selected from oxo, alkoxy, hydroxyl and alkyl where alkyl is optionally substituted with one or more substituents selected from halogen, hydroxy, alkoxy, oxo and cycloalkyl containing from 1 to 4 heteroatoms, —N(H)cycloalkyl containing from 1 to 4 heteroatoms substituted with one or more substituents selected from oxo, hydroxy and alkyl, and trifluoromethyl;
and

### Q is

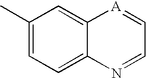

wherein

A is N;

and/or pharmaceutically acceptable salts, hydrates, solvates and pro-drugs thereof.

Included among the presently invented compounds of Formulas (I) and (II) are those in which:

### R is

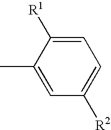 [0096] in which R¹ is halogen; and
R² is selected from: —NR⁶²S(O)₂R⁷² and —N(R⁷²)C(O)R⁷²,
where,
R⁶² is hydrogen, and
each R⁷² is independently selected from: hydrogen, C₁₋₆alkyl, C₁-C₆alkyloxyC₁-C₆alkyl, C₁-C₆alkyloxyC₁-C₆alkylamine, C₁-C₄alkylC(O)OC₁-C₄alkyl, —C₁-C₆alkylC(O)OH, amino, alkylamino where the alkyl is optionally substituted with one or more substituents selected from hydroxy, cycloalkyl containing from 1 to 4 heteroatoms where cycloalkyl containing from 1 to 4 heteroatoms is optionally substituted by one or more substituents selected form C₁-C₆alkyl, halogen and oxo, and C₁-C₆alkyl, dialkylamino where each alkyl is independently and optionally substituted with one or more substituents selected from hydroxy, oxo, C₁-C₆alkyl, amino, alkylamino and dialkylamino, aminoC₁-C₆alkyl, alkylaminoC₁-C₆alkyl where the alkyl is optionally substituted by one or more substituents selected from oxo, alkoxy and halogen, dialkylaminoC₁-C₆alkyl where each alkyl is independently and optionally substituted with one or more substituents selected from hydroxy, oxo, C₁-C₆alkyl, amino, alkylamino and dialkylamino, alkoxy, aryloxy, arylamino, diarylamino, arylalkylamino where the aryl is optionally substituted with one or more substituents selected from hydroxyl, alkoxy, hydroxyalkyl, oxo, C₁-C₆alkyl, amino, alkylamino and dialkylamino, cycloalkylalkylamino, aryl, aryl substituted with one or more substituents selected from oxo, hydroxyl, —N(H)C(O)C₁-C₆alkyl, alkoxy, nitro, amine and alkyl, arylC₁-C₄alkyl optionally substituted with one or more substituents selected from oxo, amino, alkylamino, alkylamino substituted by oxo, dialkylamino, dialkylamino substituted by one or more oxo groups, hydroxy, halogen, alkoxy and alkyl, —CH₂C(O)cycloalkyl containing from 1 to 4 heteroatoms, cycloalkylC₁-C₆alkyl where the cycloalkyl is optionally substituted with one or more substituents selected from C₁-C₆alkyl, halogen, —C(O)OC₁-C₆alkyl and —C₁-C₆alkylC(O)OH, C₁-C₄alkyl substituted with cycloalkyl containing from 1 to 4 heteroatoms where the cycloalkyl containing from 1 to 4 heteroatoms is optionally substituted with one or more substituents selected from C₁-C₆alkyl, oxo, halogen, —C(O)OC₁-C₆alkyl and —C₁-C₆alkylC(O)OH, cycloalkyl, —N(H)cycloalkyl, cycloalkyl substituted with one or more substituents selected from oxo, hydroxyl and alkyl, —N(H)cycloalkyl substituted with one or more substituents selected from oxo, hydroxy and alkyl, cycloalkyl containing from 1 to 4 heteroatoms, cycloalkyl containing from 1 to 4 heteroatoms substituted with one or more substituents selected from oxo, alkoxy, hydroxyl and alkyl where alkyl is optionally substituted with one or more substituents selected from halogen, hydroxy, alkoxy, oxo and cycloalkyl containing from 1 to 4 heteroatoms, —N(H)cycloalkyl containing from 1 to 4 heteroatoms substituted with one or more substituents selected from oxo, hydroxy and alkyl, and trifluoromethyl;
and

### Q is

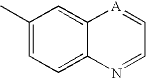

wherein

A is N;

and/or pharmaceutically acceptable salts, hydrates, solvates and pro-drugs thereof.

Included among the presently invented compounds of Formulas (I) and (II) are those in which:

### R is

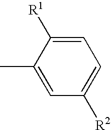 [0106] in which R¹ is chlorine; and
R² is —N(R⁷³)C(O)R⁷³,
where,
each R⁷³ is independently selected from: hydrogen, C₁₋₆alkyl, C₁-C₆alkyloxyC₁-C₆alkyl, C₁-C₆alkyloxyC₁-C₆alkylamine, C₁-C₄alkylC(O)OC₁-C₄alkyl, —C₁-C₆alkylC(O)OH, amino, alkylamino dialkylamino, aminoC₁-C₆alkyl, alkylaminoC₁-C₆alkyl, dialkylaminoC₁-C₆alkyl, alkoxy, aryloxy, arylamino, diarylamino, arylalkylamino, cycloalkylalkylamino, aryl, arylC₁-C₄alkyl, cycloalkylC₁-C₆alkyl, C₁-C₄alkyl substituted with cycloalkyl containing from 1 to 4 heteroatoms, cycloalkyl, —N(H)cycloalkyl, cycloalkyl substituted with one or more substituents selected from oxo, hydroxy and alkyl, —N(H)cycloalkyl substituted with one or more substituents selected from oxo, hydroxy and alkyl, cycloalkyl containing from 1 to 4 heteroatoms, cycloalkyl containing from 1 to 4 heteroatoms substituted with one or more substituents selected from oxo, alkoxy, hydroxyl and alkyl, —N(H)cycloalkyl containing from 1 to 4 heteroatoms substituted with one or more substituents selected from oxo, hydroxy and alkyl, and trifluoromethyl;
and

### Q is

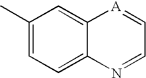

wherein

A is N;

and/or pharmaceutically acceptable salts, hydrates, solvates and pro-drugs thereof.

Included among the presently invented compounds of Formulas (I) and (II) are those in which:

### R is

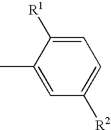 [0115] in which R¹ is chlorine; and
R² is —N(R⁷⁴)C(O)R⁷⁴,
where,
each R⁷⁴ is independently selected from: hydrogen, C₁₋₆alkyl, C₁-C₆alkyloxyC₁-C₆alkyl, C₁-C₆alkyloxyC₁-C₆alkylamine, C₁-C₄alkylC(O)OC₁-C₄alkyl, —C₁-C₆alkylC(O)OH, amino, alkylamino dialkylamino, aminoC₁-C₆alkyl, alkylaminoC₁-C₆alkyl, dialkylaminoC₁-C₆alkyl, alkoxy, C₁-C₁₂aryl, C₁-C₁₂arylC₁-C₄alkyl, cycloalkylC₁-C₆alkyl, C₁-C₄alkyl substituted with cycloalkyl containing from 1 to 4 heteroatoms, cycloalkyl, —N(H)cycloalkyl, cycloalkyl containing from 1 to 4 heteroatoms, —N(H)cycloalkyl containing from 1 to 4 heteroatoms, and trifluoromethyl;
and

### Q is

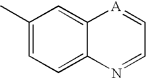

wherein

A is N;

and/or pharmaceutically acceptable salts, hydrates, solvates and pro-drugs thereof.

Included among the novel compounds useful in the present invention are:
(5Z)-2-[(2-Chloro-3-pyridinyl)amino]-5-(6-quinolinylmethylidene)-1,3-thiazol-4(5H)-one;
(5Z)-2-{[2-Chloro-5-(2-pyridinyl)phenyl]amino}-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)cyclobutanecarboxamide;
N-(4-chloro-3-{[(5Z)-4-oxo-5-(6-quinoxalinylmethylidene)-4,5-dihydro-1,3-thiazol-2-yl]amino}phenyl)-2-methylpropanamide;
N-(4-chloro-3-{[(5Z)-4-oxo-5-(6-quinolinylmethylidene)-4,5-dihydro-1,3-thiazol-2-yl]amino}phenyl)methanesulfonamide;
N-[(4-Chloro-3-{[(5Z)-4-oxo-5-(6-quinolinylmethylidene)-4,5-dihydro-1,3-thiazol-2-yl]amino}phenyl)methyl]-2-(methyloxy)acetamide;
(5Z)-2-[(2-Chloro-5-{[(1-methylethyl)amino]methyl}phenyl)amino]-5-(6-quinolinylmethylidene)-1,3-thiazol-4(5H)-one;
(2Z,5Z)-2-[(5-Chloro-1H-benzimidazol-6-yl)imino]-5-(quinolin-6-ylmethylene)-1,3-thiazolidin-4-one;
4-Chloro-N-cyclobutyl-3-{[(5Z)-4-oxo-5-(6-quinolinylmethylidene)-4,5-dihydro-1,3-thiazol-2-yl]amino}benzenesulfonamide;
(5Z)-2-(4-Pyrimidinylamino)-5-(6-quinolinylmethylidene)-1,3-thiazol-4(5H)-one;
(5Z)-2-(1H-Pyrazol-3-ylamino)-5-(6-quinolinylmethylidene)-1,3-thiazol-4(5H)-one;
(5Z)-2-(4-Pyridinylamino)-5-(6-quinolinylmethylidene)-1,3-thiazol-4(5H)-one;
(5Z)-2-(2-Quinolinylamino)-5-(6-quinolinylmethylidene)-1,3-thiazol-4(5H)-one;
(5Z)-2-[(4-Methyl-2-pyridinyl)amino]-5-(6-quinolinylmethylidene)-1,3-thiazol-4(5H)-one;
(5Z)-2-[(3-Methyl-2-pyridinyl)amino]-5-(6-quinolinylmethylidene)-1,3-thiazol-4(5H)-one;
(5Z)-2-[(6-Methyl-2-pyridinyl)amino]-5-(6-quinolinylmethylidene)-1,3-thiazol-4(5H)-one;
(5Z)-2-[(5-Iodo-2-pyridinyl)amino]-5-(6-quinolinylmethylidene)-1,3-thiazol-4(5H)-one;
(5Z)-2-(1H-Benzimidazol-2-ylamino)-5-(6-quinolinylmethylidene)-1,3-thiazol-4(5H)-one;
N-(3-{[(5Z)-4-Oxo-5-(6-quinolinylmethylidene)-4,5-dihydro-1,3-thiazol-2-yl]amino}phenyl)acetamide;
1,1-Dimethylethyl (2-{[(5Z)-4-oxo-5-(6-quinolinylmethylidene)-4,5-dihydro-1,3-thiazol-2-yl]amino}phenyl) carbamate;
N-(2-{[(5Z)-4-Oxo-5-(6-quinolinylmethylidene)-4,5-dihydro-1,3-thiazol-2-yl]amino}phenyl)acetamide;
(5Z)-2-(2-Pyrimidinylamino)-5-(6-quinolinylmethylidene)-1,3-thiazol-4(5H)-one;
(5Z)-2-(3-Quinolinylamino)-5-(6-quinolinylmethylidene)-1,3-thiazol-4(5H)-one;
(5Z)-2-[(1-Methyl-1H-indol-2-yl)amino]-5-(6-quinolinylmethylidene)-1,3-thiazol-4(5H)-one;
(5Z)-2-[(5-Chloro-2-pyridinyl)amino]-5-(6-quinolinylmethylidene)-1,3-thiazol-4(5H)-one;
1,1-Dimethylethyl [(2-{[(5Z)-4-oxo-5-(6-quinolinylmethylidene)-4,5-dihydro-1,3-thiazol-2-yl]amino}phenyl)methyl]carbamate;
(5Z)-2-{[2-Chloro-6-(trifluoromethyl)-3-pyridinyl]amino}-5-(6-quinolinylmethylidene)-1,3-thiazol-4(5H)-one;
N-(4-{[(5Z)-4-Oxo-5-(6-quinolinylmethylidene)-4,5-dihydro-1,3-thiazol-2-yl]amino}phenyl)acetamide;
(5Z)-2-{[6-(Methyloxy)-4-pyrimidinyl]amino}-5-(6-quinolinylmethylidene)-1,3-thiazol-4(5H)-one;
3-Methyl-N-(2-{[(5Z)-4-oxo-5-(6-quinolinylmethylidene)-4,5-dihydro-1,3-thiazol-2-yl]amino}phenyl)butanamide;
N-(4-Chloro-3-{[(5Z)-4-oxo-5-(6-quinolinylmethylidene)-4,5-dihydro-1,3-thiazol-2-yl]amino}phenyl)-3-methylbutanamide;
(5Z)-2-[(4-Methyl-1,3-thiazol-2-yl)amino]-5-(6-quinolinylmethylidene)-1,3-thiazol-4(5H)-one;
Ethyl 2-{[(5Z)-4-oxo-5-(6-quinolinylmethylidene)-4,5-dihydro-1,3-thiazol-2-yl]amino}-4,5,6,7-tetrahydro-1-benzothiophene-3-carboxylate;
(5Z)-2-(2-Biphenylylamino)-5-(6-quinolinylmethylidene)-1,3-thiazol-4(5H)-one;
(5Z)-2-{[4′-(Methyloxy)-2-biphenylyl]amino}-5-(6-quinolinylmethylidene)-1,3-thiazol-4(5H)-one;
(5Z)-2-{[4′-(Dimethylamino)-2-biphenylyl]amino}-5-(6-quinolinylmethylidene)-1,3-thiazol-4(5H)-one;
2′-{[(5Z)-4-Oxo-5-(6-quinolinylmethylidene)-4,5-dihydro-1,3-thiazol-2-yl]amino}-3-biphenylcarbonitrile;
(5Z)-2-{[2-(1,3-Benzodioxol-5-yl)phenyl]amino}-5-(6-quinolinylmethylidene)-1,3-thiazol-4(5H)-one;
Ethyl 1-methyl-5-{[(5Z)-4-oxo-5-(6-quinolinylmethylidene)-4,5-dihydro-1,3-thiazol-2-yl]amino}-1H-pyrazole-4-carboxylate;
(5Z)-2-{[2-(4-Pyridinyl)phenyl]amino}-5-(6-quinolinylmethylidene)-1,3-thiazol-4(5H)-one;
(5Z)-2-{[2-Chloro-5-(hydroxymethyl)phenyl]amino}-5-(6-quinolinylmethylidene)-1,3-thiazol-4(5H)-one;
1,1-Dimethylethyl [(4-chloro-3-{[(5Z)-4-oxo-5-(6-quinolinylmethylidene)-4,5-dihydro-1,3-thiazol-2-yl]amino}phenyl)methyl]carbamate;
(5Z)-2-{[5-(Aminomethyl)-2-chlorophenyl]amino}-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)propanamide;
N-(4-Chloro-3-{[(5Z)-4-oxo-5-(6-quinolinylmethylidene)-4,5-dihydro-1,3-thiazol-2-yl]amino}phenyl)-2-(methyloxy)acetamide;
N-(4-Chloro-3-{[(5Z)-4-oxo-5-(6-quinolinylmethylidene)-4,5-dihydro-1,3-thiazol-2-yl]amino}phenyl)-2-(2-thienyl)acetamide;
N-[(4-Chloro-3-{[(5Z)-4-oxo-5-(6-quinolinylmethylidene)-4,5-dihydro-1,3-thiazol-2-yl]amino}phenyl)methyl]acetamide;
N-[(4-Chloro-3-{[(5Z)-4-oxo-5-(6-quinolinylmethylidene)-4,5-dihydro-1,3-thiazol-2-yl]amino}phenyl)methyl]-2-methylpropanamide;
N˜1˜-(4-Chloro-3-{[(5Z)-4-oxo-5-(6-quinolinylmethylidene)-4,5-dihydro-1,3-thiazol-2-yl]amino}phenyl)-N˜2˜,N˜2˜-dimethylglycinamide;
N-[(4-Chloro-3-{[(5Z)-4-oxo-5-(6-quinolinylmethylidene)-4,5-dihydro-1,3-thiazol-2-yl]amino}phenyl)methyl]urea;
N-[(4-Chloro-3-{[(5Z)-4-oxo-5-(6-quinolinylmethylidene)-4,5-dihydro-1,3-thiazol-2-yl]amino}phenyl)methyl]-2-hydroxyacetamide;
N˜1˜-[(4-Chloro-3-{[(5Z)-4-oxo-5-(6-quinolinylmethylidene)-4,5-dihydro-1,3-thiazol-2-yl]amino}phenyl)methyl]-N˜2˜,N˜2˜-dimethylglycinamide;
1,1-Dimethylethyl (2-{[(4-chloro-3-{[(5Z)-4-oxo-5-(6-quinolinylmethylidene)-4,5-dihydro-1,3-thiazol-2-yl]amino}phenyl)methyl]amino}-2-oxoethyl)carbamate;
4-{[(4-Chloro-3-{[(5Z)-4-oxo-5-(6-quinolinylmethylidene)-4,5-dihydro-1,3-thiazol-2-yl]amino}phenyl)methyl]amino}-4-oxobutanoic acid;
(5Z)-2-{[3-(1,3-Oxazol-4-yl)phenyl]amino}-5-(6-quinolinylmethylidene)-1,3-thiazol-4(5H)-one;
(5Z)-2-[(1-Ethyl-1H-pyrazol-5-yl)amino]-5-(6-quinolinylmethylidene)-1,3-thiazol-4(5H)-one;
N˜1˜-[(4-Chloro-3-{[(5Z)-4-oxo-5-(6-quinolinylmethylidene)-4,5-dihydro-1,3-thiazol-2-yl]amino}phenyl)methyl]glycinamide;
Ethyl (4-chloro-3-{[(5Z)-4-oxo-5-(6-quinolinylmethylidene)-4,5-dihydro-1,3-thiazol-2-yl]amino}phenyl)carbamate;
N-(4-Chloro-3-{[(5Z)-4-oxo-5-(6-quinolinylmethylidene)-4,5-dihydro-1,3-thiazol-2-yl]amino}phenyl)-2-cyclopropylacetamide;
N-(4-Chloro-3-{[(5Z)-4-oxo-5-(6-quinolinylmethylidene)-4,5-dihydro-1,3-thiazol-2-yl]amino}phenyl)-N′-(phenylmethyl)urea;
Ethyl 4-[(4-chloro-3-{[(5Z)-4-oxo-5-(6-quinolinylmethylidene)-4,5-dihydro-1,3-thiazol-2-yl]amino}phenyl)amino]-4-oxobutanoate;
N-(4-Chloro-3-{[(5Z)-4-oxo-5-(6-quinolinymethylidene)-4,5-dihydro-1,3-thiazol-2-yl]amino}phenyl)-N′-(1-methylethyl)urea;
N-(4-Chloro-3-{[(5Z)-4-oxo-5-(6-quinolinylmethylidene)-4,5-dihydro-1,3-thiazol-2-yl]amino}phenyl)-1-piperidinecarboxamide;
(5Z)-2-{[2-Chloro-5-(1H-imidazol-4-yl)phenyl]amino}-5-(6-quinolinylmethylidene)-1,3-thiazol-4(5H)-one;
(5Z)-2-{[2-Chloro-5-(2-methyl-1,3-thiazol-4-yl)phenyl]amino}-5-(6-quinolinylmethylidene)-1,3-thiazol-4(5H)-one;
5-Chloro-6-{[(5Z)-4-oxo-5-(6-quinolinylmethylidene)-4,5-dihydro-1,3-thiazol-2-yl]amino}-1,3-dihydro-2H-benzimidazol-2-one;
Methyl [(4-chloro-3-{[(5Z)-4-oxo-5-(6-quinolinylmethylidene)-4,5-dihydro-1,3-thiazol-2-yl]amino}phenyl)methyl]carbamate;
N-[(4-Chloro-3-{[(5Z)-4-oxo-5-(6-quinolinylmethylidene)-4,5-dihydro-1,3-thiazol-2-yl]amino}phenyl)methyl]-N′-(1-methylethyl)urea;
2-[3,4-Bis(methyloxy)phenyl]-N-(4-chloro-3-{[(5Z)-4-oxo-5-(6-quinolinylmethylidene)-4,5-dihydro-1,3-thiazol-2-yl]amino}phenyl)acetamide;
(5Z)-2-{[2-Chloro-5-(1,3-oxazol-4-yl)phenyl]amino}-5-(6-quinolinylmethylidene)-1,3-thiazol-4(5H)-one;
(5Z)-2-{[5-(1,3-Benzothiazol-2-yl)-2-chlorophenyl]amino}-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)-3-phenylpropanamide;
N-(4-Chloro-3-{[(5Z)-4-oxo-5-(6-quinolinylmethylidene)-4,5-dihydro-1,3-thiazol-2-yl]amino}phenyl)-2-(4-fluorophenyl)acetamide;
(5Z)-2-[(2-Amino-5-chloro-1H-benzimidazol-6-yl)amino]-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)-1-phenylmethanesulfonamide;
N′-(4-Chloro-3-{[(5Z)-4-oxo-5-(6-quinolinylmethylidene)-4,5-dihydro-1,3-thiazol-2-yl]amino}phenyl)-N,N-dimethylsulfamide;
(5Z)-2-[(7-Chloro-6-quinoxalinyl)amino]-5-(6-quinolinylmethylidene)-1,3-thiazol-4(5H)-one;
N-(4-{[(4-Chloro-3-{[(5Z)-4-oxo-5-(6-quinolinylmethylidene)-4,5-dihydro-1,3-thiazol-2-yl]amino}phenyl)amino]sulfonyl}-5-methyl-1,3-thiazol-2-yl)acetamide;
N-(4-Chloro-3-{[(5Z)-4-oxo-5-(6-quinolinylmethylidene)-4,5-dihydro-1,3-thiazol-2-yl]amino}phenyl)-2-thiophenesulfonamide;
(5Z)-2-{[2-Chloro-5-(1H-tetrazol-5-yl)phenyl]amino}-5-(6-quinolinylmethylidene)-1,3-thiazol-4(5H)-one;
N˜1˜-(4-Chloro-3-{[(5Z)-4-oxo-5-(6-quinolinylmethylidene)-4,5-dihydro-1,3-thiazol-2-yl]amino}phenyl)glycinamide;
N-(4-Chloro-3-{[(5Z)-4-oxo-5-(6-quinolinylmethylidene)-4,5-dihydro-1,3-thiazol-2-yl]amino}phenyl)-2-(1-piperidinyl)acetamide;
((5Z)-2-{[2-Chloro-5-(2-pyrimidinyl)phenyl]amino}-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)-2-cyclopentylacetamide;
N-(4-Chloro-3-{[(5Z)-4-oxo-5-(6-quinolinylmethylidene)-4,5-dihydro-1,3-thiazol-2-yl]amino}phenyl)-2-(1-pyrrolidinyl)acetamide;
N˜1˜-(4-Chloro-3-{[(5Z)-4-oxo-5-(6-quinolinylmethylidene)-4,5-dihydro-1,3-thiazol-2-yl]amino}phenyl)-N˜2˜-ethyl-N˜2˜-methylglycinamide;
N-(4-Chloro-3-{[(5Z)-4-oxo-5-(6-quinolinylmethylidene)-4,5-dihydro-1,3-thiazol-2-yl]amino}phenyl)-2-(4-morpholinyl)acetamide;
1,1-Dimethylethyl 4-{2-[(4-chloro-3-{[(5Z)-4-oxo-5-(6-quinolinylmethylidene)-4,5-dihydro-1,3-thiazol-2-yl]amino}phenyl)amino]-2-oxoethyl}-1-piperazinecarboxylate;
N′-[(4-Chloro-3-{[(5Z)-4-oxo-5-(6-quinolinylmethylidene)-4,5-dihydro-1,3-thiazol-2-yl]amino}phenyl)methyl]-N,N-dimethylimidoformamide;
N-(4-Chloro-3-{[(5Z)-4-oxo-5-(6-quinolinylmethylidene)-4,5-dihydro-1,3-thiazol-2-yl]amino}phenyl)-2-(1-piperazinyl)acetamide;
N-(4-Chloro-3-{[(5Z)-4-oxo-5-(6-quinolinylmethylidene)-4,5-dihydro-1,3-thiazol-2-yl]amino}phenyl)-2-pyridinecarboxamide;
N-(4-Chloro-3-{[(5Z)-4-oxo-5-(6-quinolinylmethylidene)-4,5-dihydro-1,3-thiazol-2-yl]amino}phenyl)cyclohexanecarboxamide;
N-(4-Chloro-3-{[(5Z)-4-oxo-5-(6-quinolinylmethylidene)-4,5-dihydro-1,3-thiazol-2-yl]amino}phenyl)benzamide;
N-(4-Chloro-3-{[(5Z)-4-oxo-5-(6-quinolinylmethylidene)-4,5-dihydro-1,3-thiazol-2-yl]amino}phenyl)cyclopentanecarboxamide;
(5Z)-2-{[2-Chloro-5-(3-thienyl)phenyl]amino}-5-(6-quinolinylmethylidene)-1,3-thiazol-4(5H)-one;
(5Z)-2-({2-Chloro-5-[6-(methyloxy)-2-pyridinyl]phenyl}amino)-5-(6-quinolinylmethylidene)-1,3-thiazol-4(5H)-one;
(5Z)-2-[(3,5-Dichloro-2,6-dimethyl-4-pyridinyl)amino]-5-(6-quinoxalinylmethylidene)-1,3-thiazol-4(5H)-one;
(5Z)-2-{[2-Chloro-5-(4-morpholinyl)phenyl]amino}-5-(6-quinolinylmethylidene)-1,3-thiazol-4(5H)-one;
N˜1˜-(4-Chloro-3-{[(5Z)-4-oxo-5-(6-quinolinylmethylidene)-4,5-dihydro-1,3-thiazol-2-yl]amino}phenyl)leucinamide;
N-(4-Chloro-3-{[(5Z)-4-oxo-5-(6-quinolinylmethylidene)-4,5-dihydro-1,3-thiazol-2-yl]amino}phenyl)-2-methylpropanamide;
(5Z)-2-{[5-(2-Amino-5-pyrimidinyl)-2-chlorophenyl]amino}-5-(6-quinolinylmethylidene)-1,3-thiazol-4(5H)-one;
(5Z)-2-({2-Chloro-5-[(dimethylamino)methyl]phenyl}amino)-5-(6-quinolinylmethylidene)-1,3-thiazol-4(5H)-one;
N-[(4-Chloro-3-{[(5Z)-4-oxo-5-(6-quinolinylmethylidene)-4,5-dihydro-1,3-thiazol-2-yl]amino}phenyl)methyl]methanesulfonamide;
4-Chloro-N,N-dimethyl-3-{[(5Z)-4-oxo-5-(6-quinolinylmethylidene)-4,5-dihydro-1,3-thiazol-2-yl]amino}benzenesulfonamide;
N-(2,4-Dichloro-5-{[(5Z)-4-oxo-5-(6-quinolinylmethylidene)-4,5-dihydro-1,3-thiazol-2-yl]amino}phenyl)cyclobutanecarboxamide;
(5Z)-2-{[4-Chloro-3′-(methyloxy)-3-biphenylyl]amino}-5-(6-quinolinylmethylidene)-1,3-thiazol-4(5H)-one;
(5Z)-2-({2-Chloro-5-[(cyclopentylamino)methyl]phenyl}amino)-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)-N-methylcyclobutanecarboxamide;
N-(4-Chloro-3-{[(5Z)-4-oxo-5-(6-quinolinylmethylidene)-4,5-dihydro-1,3-thiazol-2-yl]amino}phenyl)-N-(phenylsulfonyl)benzenesulfonamide;
(5Z)-2-({2-Chloro-5-[(phenylmethyl)amino]phenyl}amino)-5-(6-quinolinylmethylidene)-1,3-thiazol-4(5H)-one;
(5Z)-2-({2-Chloro-5-[(1-methylethyl)amino]phenyl}amino)-5-(6-quinolinylmethylidene)-1,3-thiazol-4(5H)-one;
1,1-Dimethylethyl (4-chloro-3-{[(5Z)-4-oxo-5-(6-quinolinylmethylidene)-4,5-dihydro-1,3-thiazol-2-yl]amino}phenyl)carbamate;
N-(4-Chloro-3-{[(5Z)-4-oxo-5-(6-quinolinylmethylidene)-4,5-dihydro-1,3-thiazol-2-yl]amino}phenyl)-L-prolinamide;

N˜1˜-(4-Chloro-3-{[(5Z)-4-oxo-5-(6-quinolinylmethylidene)-4,5-dihydro-1,3-thiazol-2-yl]amino}phenyl)-L-alaninamide;
1,1-Dimethylethyl 4-{[(4-chloro-3-{[(5Z)-4-oxo-5-(6-quinolinylmethylidene)-4,5-dihydro-1,3-thiazol-2-yl]amino}phenyl)amino]carbonyl}-1-piperidinecarboxylate;
(5Z)-2-({2-Chloro-5-[6-(methylamino)-2-pyridinyl]phenyl}amino)-5-(6-quinolinylmethylidene)-1,3-thiazol-4(5H)-one;
(5Z)-2-[(3,5-Dichloro-4-pyridinyl)amino]-5-(6-quinolinylmethylidene)-1,3-thiazol-4(5H)-one;
1,1-Dimethylethyl (3-chloro-4-{[(5Z)-4-oxo-5-(6-quinolinylmethylidene)-4,5-dihydro-1,3-thiazol-2-yl]amino}phenyl)carbamate;
N-(4-Chloro-3-{[(5Z)-4-oxo-5-(6-quinolinylmethylidene)-4,5-dihydro-1,3-thiazol-2-yl]amino}phenyl)-4-piperidinecarboxamide;
N-(4-Chloro-3-{[(5Z)-4-oxo-5-(6-quinolinylmethylidene)-4,5-dihydro-1,3-thiazol-2-yl]amino}phenyl)ethanesulfonamide;
N-(4-Chloro-3-{[(5Z)-4-oxo-5-(6-quinolinylmethylidene)-4,5-dihydro-1,3-thiazol-2-yl]amino}phenyl)benzenesulfonamide;
N-(4-Chloro-3-{[(5Z)-4-oxo-5-(6-quinolinylmethylidene)-4,5-dihydro-1,3-thiazol-2-yl]amino}phenyl)-1-propanesulfonamide;
N-(3-Chloro-4-{[(5Z)-4-oxo-5-(6-quinolinylmethylidene)-4,5-dihydro-1,3-thiazol-2-yl]amino}phenyl)acetamide;
N-(3-Chloro-4-{[(5Z)-4-oxo-5-(6-quinolinylmethylidene)-4,5-dihydro-1,3-thiazol-2-yl]amino}phenyl)methanesulfonamide;
N-(3-Chloro-4-{[(5Z)-4-oxo-5-(6-quinolinylmethylidene)-4,5-dihydro-1,3-thiazol-2-yl]amino}phenyl)benzamide;
N-(3-Chloro-4-{[(5Z)-4-oxo-5-(6-quinolinylmethylidene)-4,5-dihydro-1,3-thiazol-2-yl]amino}phenyl)-2-methylpropanamide;
N-(4-Chloro-3-{[(5Z)-4-oxo-5-(6-quinolinylmethylidene)-4,5-dihydro-1,3-thiazol-2-yl]amino}phenyl)-4-methylbenzenesulfonamide;
N-(3-Chloro-4-{[(5Z)-4-oxo-5-(6-quinolinylmethylidene)-4,5-dihydro-1,3-thiazol-2-yl]amino}phenyl)-2,2,2-trifluoroacetamide;
(3S)-3-Amino-N-(4-chloro-3-{[(5Z)-4-oxo-5-(6-quinolinylmethylidene)-4,5-dihydro-1,3-thiazol-2-yl]amino}phenyl)butanamide;
N-(4-Chloro-3-{[(5Z)-4-oxo-5-(6-quinolinylmethylidene)-4,5-dihydro-1,3-thiazol-2-yl]amino}phenyl)-L-phenylalaninamide;
N-(4-Chloro-3-{[(5Z)-4-oxo-5-(6-quinolinylmethylidene)-4,5-dihydro-1,3-thiazol-2-yl]amino}phenyl)-4-nitrobenzenesulfonamide;
N-(4-Chloro-3-{[(5Z)-4-oxo-5-(6-quinolinylmethylidene)-4,5-dihydro-1,3-thiazol-2-yl]amino}phenyl)-2,2-dimethylpropanamide;
4-Chloro-N-(2-methylpropyl)-3-{[(5Z)-4-oxo-5-(6-quinoxalinylmethylidene)-4,5-dihydro-1,3-thiazol-2-yl]amino}benzamide;
N-(4-Chloro-3-{[(5Z)-4-oxo-5-(6-quinoxalinylmethylidene)-4,5-dihydro-1,3-thiazol-2-yl]amino}phenyl)propanamide;
N-(4-Chloro-3-{[(5Z)-4-oxo-5-(6-quinoxalinylmethylidene)-4,5-dihydro-1,3-thiazol-2-yl]amino}phenyl)-2-(methyloxy)acetamide;
N-(4-Chloro-3-{[(5Z)-4-oxo-5-(6-quinoxalinylmethylidene)-4,5-dihydro-1,3-thiazol-2-yl]amino}phenyl)-2-(2-thienyl)acetamide;
N-(4-Chloro-3-{[(5Z)-4-oxo-5-(6-quinoxalinylmethylidene)-4,5-dihydro-1,3-thiazol-2-yl]amino}phenyl)-2-methylpropanamide;
N-(4-Chloro-3-{[(5Z)-4-oxo-5-(6-quinoxalinylmethylidene)-4,5-dihydro-1,3-thiazol-2-yl]amino}phenyl)cyclobutanecarboxamide;
N-(4-Chloro-3-{[(5Z)-4-oxo-5-(6-quinoxalinylmethylidene)-4,5-dihydro-1,3-thiazol-2-yl]amino}phenyl)cyclopentanecarboxamide;
(5Z)-2-[(3,5-Dichloro-4-pyridinyl)amino]-5-(6-quinoxalinylmethylidene)-1,3-thiazol-4(5H)-one;
N-(4-Chloro-3-{[(5Z)-4-oxo-5-(6-quinoxalinylmethylidene)-4,5-dihydro-1,3-thiazol-2-yl]amino}phenyl)-4-piperidinecarboxamide;
N-(2-Chloro-3-{[(5Z)-4-oxo-5-(6-quinoxalinylmethylidene)-4,5-dihydro-1,3-thiazol-2-yl]amino}phenyl)-2-methylpropanamide;
N-(4-Chloro-3-{[(5Z)-4-oxo-5-(6-quinoxalinylmethylidene)-4,5-dihydro-1,3-thiazol-2-yl]amino}phenyl)-2,2-dimethylpropanamide;
1,1-Dimethylethyl {2-[(4-chloro-3-{[(5Z)-4-oxo-5-(6-quinoxalinylmethylidene)-4,5-dihydro-1,3-thiazol-2-yl]amino}phenyl)amino]-1,1-dimethyl-2-oxoethyl}carbamate;
N-(2-Chloro-3-{[(5Z)-4-oxo-5-(6-quinoxalinylmethylidene)-4,5-dihydro-1,3-thiazol-2-yl]amino}phenyl)cyclopropanecarboxamide;
N-(2-Chloro-3-{[(5Z)-4-oxo-5-(6-quinoxalinylmethylidene)-4,5-dihydro-1,3-thiazol-2-yl]amino}phenyl)cyclobutanecarboxamide;
N˜1˜-(4-Chloro-3-{[(5Z)-4-oxo-5-(6-quinoxalinylmethylidene)-4,5-dihydro-1,3-thiazol-2-yl]amino}phenyl)-2-methylalaninamide;
N-(4-Chloro-3-{[(5Z)-4-oxo-5-(6-quinoxalinylmethylidene)-4,5-dihydro-1,3-thiazol-2-yl]amino}phenyl)ethanesulfonamide;
N-(4-Chloro-3-{[(5Z)-4-oxo-5-(6-quinoxalinylmethylidene)-4,5-dihydro-1,3-thiazol-2-yl]amino}phenyl)-2-propanesulfonamide;
N-(4-Chloro-3-{[(5Z)-4-oxo-5-(6-quinolinylmethylidene)-4,5-dihydro-1,3-thiazol-2-yl]amino}phenyl)-N-(1-methylethyl)acetamide;
N-(4-Chloro-3-{[(5Z)-4-oxo-5-(6-quinoxalinylmethylidene)-4,5-dihydro-1,3-thiazol-2-yl]amino}phenyl)-N-(1-methylethyl)acetamide;
N-(4-Chloro-3-{[(5Z)-4-oxo-5-(6-quinolinylmethylidene)-4,5-dihydro-1,3-thiazol-2-yl]amino}phenyl)-N-(1-methylethyl)methanesulfonamide;
N-(4-Chloro-3-{[(5Z)-4-oxo-5-(6-quinoxalinylmethylidene)-4,5-dihydro-1,3-thiazol-2-yl]amino}phenyl)-N-(1-methylethyl)methanesulfonamide;
(5Z)-2-({2-Chloro-5-[(1-methylethyl)amino]phenyl}amino)-5-(6-quinoxalinylmethylidene)-1,3-thiazol-4(5H)-one;
N-(2-Chloro-3-{[(5Z)-4-oxo-5-(6-quinoxalinylmethylidene)-4,5-dihydro-1,3-thiazol-2-yl]amino}phenyl)methanesulfonamide;
N-(2-Chloro-3-{[(5Z)-4-oxo-5-(6-quinoxalinylmethylidene)-4,5-dihydro-1,3-thiazol-2-yl]amino}phenyl)benzamide;
1,1-Dimethylethyl {2-[(4-chloro-3-{[(5Z)-4-oxo-5-(6-quinolinylmethylidene)-4,5-dihydro-1,3-thiazol-2-yl]amino}phenyl)amino]-1,1-dimethyl-2-oxoethyl}carbamate;
N¹-(4-Chloro-3-{[(5Z)-4-oxo-5-(6-quinolinylmethylidene)-4,5-dihydro-1,3-thiazol-2-yl]amino}phenyl)-2-methylalaninamide;
N-(4-Chloro-3-{[(5Z)-4-oxo-5-(6-quinolinylmethylidene)-4,5-dihydro-1,3-thiazol-2-yl]amino}phenyl)-2-propanesulfonamide;
4-Chloro-N-[2-(methyloxy)ethyl]-3-{[(5Z)-4-oxo-5-(6-quinolinylmethylidene)-4,5-dihydro-1,3-thiazol-2-yl]amino}benzamide;
4-Chloro-3-{[(5Z)-4-oxo-5-(6-quinolinylmethylidene)-4,5-dihydro-1,3-thiazol-2-yl ]amino}-N-propylbenzamide;
4-Chloro-N-(2-hydroxyethyl)-3-{[(5Z)-4-oxo-5-(6-quinolinylmethylidene)-4,5-dihydro-1,3-thiazol-2-yl]amino}benzamide;
4-Chloro-N-[3-(2-oxo-1-pyrrolidinyl)propyl]-3-{[(5Z)-4-oxo-5-(6-quinolinylmethylidene)-4,5-dihydro-1,3-thiazol-2-yl]amino}benzamide;
4-Chloro-N-[2-(4-morpholinyl)ethyl]-3-{[(5Z)-4-oxo-5-(6-quinolinylmethylidene)-4,5-dihydro-1,3-thiazol-2-yl]amino}benzamide;
4-chloro-N-[2-(4-morpholinyl)ethyl]-3-[(4-oxo-4,5-dihydro-1,3-thiazol-2-yl)amino]benzamide;
4-Chloro-N-[3-(4-morpholinyl)propyl]-3-{[(5Z)-4-oxo-5-(6-quinolinylmethylidene)-4,5-dihydro-1,3-thiazol-2-yl]amino}benzamide;
4-Chloro-N-(2-hydroxy-1,1-dimethylethyl)-3-{[(5Z)-4-oxo-5-(6-quinolinylmethylidene)-4,5-dihydro-1,3-thiazol-2-yl]amino}benzamide;
4-Chloro-N-(1-methyl-4-piperidinyl)-3-{[(5Z)-4-oxo-5-(6-quinolinylmethylidene)-4,5-dihydro-1,3-thiazol-2-yl]amino}benzamide;
(5Z)-2-[(2-Chloro-5-{[(3S)-3-hydroxy-1-pyrrolidinyl]carbonyl}phenyl)amino]-5-(6-quinolinylmethylidene)-1,3-thiazol-4(5H)-one;
4-Chloro-3-{[(5Z)-4-oxo-5-(6-quinolinylmethylidene)-4,5-dihydro-1,3-thiazol-2-yl ]amino}-N-[2-(2-thienyl)ethyl]benzamide;
(5Z)-2-{[2-Chloro-5-({4-[2-oxo-2-(1-pyrrolidinyl)ethyl]-1-piperazinyl}carbonyl)phenyl]amino}-5-(6-quinolinylmethylidene)-1,3-thiazol-4(5H)-one;
4-Chloro-N-(cyclopropylmethyl)-3-{[(5Z)-4-oxo-5-(6-quinolinylmethylidene)-4,5-dihydro-1,3-thiazol-2-yl]amino}benzamide;
4-Chloro-N-(trans-4-hydroxycyclohexyl)-3-{[(5Z)-4-oxo-5-(6-quinolinylmethylidene)-4,5-dihydro-1,3-thiazol-2-yl]amino}benzamide;
4-Chloro-N-(3-hydroxypropyl)-3-{[(5Z)-4-oxo-5-(6-quinolinylmethylidene)-4,5-dihydro-1,3-thiazol-2-yl]amino}benzamide;
4-Chloro-3-{[(5Z)-4-oxo-5-(6-quinolinylmethylidene)-4,5-dihydro-1,3-thiazol-2-yl]amino}-N-[2-(1-pyrrolidinyl)ethyl]benzamide;
4-Chloro-3-{[(5Z)-4-oxo-5-(6-quinolinylmethylidene)-4,5-dihydro-1,3-thiazol-2-yl]amino}-N-[2-(1-piperidinyl)ethyl]benzamide;
4-Chloro-N-[3-(4-methyl-1-piperazinyl)propyl]-3-{[(5Z)-4-oxo-5-(6-quinolinylmethylidene)-4,5-dihydro-1,3-thiazol-2-yl]amino}benzamide;
(5Z)-2-({2-Chloro-5-[(4-methyl-1-piperazinyl)carbonyl]phenyl}amino)-5-(6-quinolinylmethylidene)-1,3-thiazol-4(5H)-one;
4-Chloro-N-ethyl-3-{[(5Z)-4-oxo-5-(6-quinolinylmethylidene)-4,5-dihydro-1,3-thiazol-2-yl]amino}benzamide;
4-Chloro-N-[2-(dimethylamino)ethyl]-N-methyl-3-{[(5Z)-4-oxo-5-(6-quinolinylmethylidene)-4,5-dihydro-1,3-thiazol-2-yl]amino}benzamide; and
(5Z)-2-{[2-Chloro-5-(4-morpholinylcarbonyl)phenyl]amino}-5-(6-quinolinylmethylidene)-1,3-thiazol-4(5H)-one.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Compounds of Formula (I) are included in the pharmaceutical compositions of the invention and used in the methods of the invention.

By the term “aryl” as used herein, unless otherwise defined, is meant a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

By the term “C₁-C₁₂aryl” as used herein, unless otherwise defined, is meant a group selected from: phenyl, naphthalene, 3,4-methylenedioxyphenyl, pyridine, biphenyl, quinoline, pyrimidine, quinazoline, thiophene, thiazole, furan, pyrrole, pyrazole, imidazole, indole, oxazole, quinoxaline, 1,3-benzothiazole, indene, pyrazine, 1,3-dihydro-2H-benzimidazole, benzimidazole, benzothiophene, tetrahydrobenzothiophene and tetrazole.

Suitably, the term “C₁-C₁₂aryl” means a group selected from: phenyl, naphthalene, 3,4-methylenedioxyphenyl, pyridine, biphenyl, quinoline, pyrimidine, quinazoline, thiophene, thiazole, furan, pyrrole, pyrazole, imidazole, indole, indene, pyrazine, 1,3-dihydro-2H-benzimidazole, benzimidazole, benzothiophene, tetrahydrobenzothiophene and tetrazole.

The term “substituted” as used herein, unless otherwise defined, is meant that the subject chemical moiety has one or more substituents selected from the group consisting of:

aryl,
aryl substituted with one or more substituents selected from alkyl, hydroxy, alkoxy, amino, alkylamino, alkylamino substituted by oxo, dialkylamino, dialkylamino substituted by one or more oxo groups, oxo, C₁-C₁₂aryl optionally substituted with one or more substituents selected from hydroxy, alkoxy oxo, cyano, amino, alkylamino, dialkylamino, alkyl and alkoxy, cyano, trifluoromethyl, —SO₂NR²¹R²², N-acylamino, —CO₂R²⁰, and halogen,
cycloalkyl substituted with one or more substituents selected from alkyl, hydroxy, alkoxy, trifluoromethyl, —SO₂NR²¹R²², amino, —CO₂R²⁰, N-acylamino and halogen,
cycloalkyl containing from 1 to 4 heteroatoms substituted with one or more subsitituents selected from alkyl, hydroxy, alkoxy, —SO₂NR²¹R²², amino, —CO₂R²⁰, trifluoromethyl, N-acylamino and halogen,
alkoxy substituted with one or more substituents selected form alkyl, —CO₂H, hydroxy, C₁-C₁₂aryl, alkoxy, amino and halogen,
cycloalkyl, cycloalkyl containing from 1 to 4 heteroatoms, C₁-C₄alkylcycloalkyl containing from 1 to 3 heteroatomsC₁-C₄alkyl, —C(O)NHS(O)₂R²⁰, (CH₂)_{g}NR²³S(O)₂R²⁰, hydroxyalkyl, alkoxy, —(CH₂)_{g}NR²¹R²², —S(O)₂NR²¹R²², (CH₂)_{g}N(R²⁰)C(O)ₘR²⁰, —(CH₂)_{g}N═C(H)R⁵⁰ where R⁵⁰ is selected from amine, alkylamine and dialkylamine, —(CH₂)_{g}C(O)ₘR²⁰, acyloxy, alkyl, —OCF₃, amino, hydroxy, alkylamino, acetamide, aminoalkyl, aminoalkoxy, alkylaminoalkoxy, dialkylaminoalkoxy, alkoxyalkylamide, alkoxyC₁-C₁₂aryl, C₁-C₁₂aryl, C₁-C₁₂arylalkyl, dialkylamino, N-acylamino, aminoalkylN-acylamino, —(CH₂)_{g}S(O)ₙR²³, nitro, cyano, oxo, halogen, trifluoromethyloxy and trifluoromethyl;
where
g is 0 to 6,
n is 0 to 2,
m is 1 or 2;
R²³ is selected from hydrogen, C₁-C₁₂aryl, C₁-C₁₂aryl substituted with one or more substituents selected from C₁-C₆alkyl and halogen, alkylamino substituted by oxo, dialkylamino substituted by one or more oxo groups, C₁-C₁₂arylC₁-C₆alkyl, C₁-C₁₂arylC₁-C₆alkyl substituted with one or more substituents selected from C₁-C₆alkyl and halogen, or alkyl,
each R²⁰ is independently selected form hydrogen, hydroxy, alkyl optionally substituted with one or more substituents selected from hydroxy and halogen, C₁-C₆alkyloxyC₁-C₆alkyl, C₁-C₆alkyloxyC₁-C₆alkylamine, C₁-C₄alkylC(O)OC₁-C₄alkyl, —C₁-C₆alkylC(O)OH, amino, alkylamino where the alkyl is optionally substituted with one or more substituents selected from hydroxy, oxo, cycloalkyl containing from 1 to 4 heteroatoms where cycloalkyl containing from 1 to 4 heteroatoms is optionally substituted by one or more substituents selected form C₁-C₆alkyl, halogen and oxo, and C₁-C₆alkyl, dialkylamino where each alkyl is independently and optionally substituted with one or more substituents selected from hydroxyl, oxo, C₁-C₆alkyl, amino, alkylamino and dialkylamino, aminoC₁-C₆alkyl, alkylaminoC₁-C₆alkyl where the alkyl is optionally substituted by one or more substituents selected from oxo, alkoxy and halogen, dialkylaminoC₁-C₆alkyl where each alkyl is independently and optionally substituted with one or more substituents selected from hydroxy, oxo, C₁-C₆alkyl, amino, alkylamino and dialkylamino, —C(O)OH, alkoxy, aryloxy, arylamino, diarylamino, arylalkylamino where the aryl is optionally substituted with one or more substituents selected from hydroxyl, alkoxy, hydroxyalkyl, oxo, C₁-C₆alkyl, amino, alkylamino and dialkylamino, cycloalkylalkylamino, aryl, aryl substituted with one or more substituents selected from oxo, hydroxyl, —N(H)C(O)C₁-C₆alkyl, alkoxy, nitro, amine and alkyl, arylC₁-C₄alkyl optionally substituted with one or more substituents selected from oxo, amino, alkylamino, alkylamino substituted by oxo, dialkylamino, dialkylamino substituted by one or more oxo groups, hydroxy, halogen, alkoxy and alkyl, —CH₂C(O)cycloalkyl containing from 1 to 4 heteroatoms, cycloalkylC₁-C₆alkyl where the cycloalkyl is optionally substituted with one or more substituents selected from C₁-C₆alkyl, oxo, halogen, —C(O)OC₁-C₆alkyl and —C₁-C₆alkylC(O)OH, C₁-C₄alkyl substituted with cycloalkyl containing from 1 to 4 heteroatoms where the cycloalkyl containing from 1 to 4 heteroatoms is optionally substituted with one or more substituents selected from C₁-C₆alkyl, oxo, halogen, —C(O)OC₁-C₆alkyl and —C₁-C₆alkylC(O)OH, cycloalkyl, —N(H)cycloalkyl, cycloalkyl substituted with one or more substituents selected from oxo, hydroxy, halogen, amino, alkylamino, dialkylamino, —C(O)OH, —C(O)NR⁸⁰R⁹⁰ where R⁸⁰ and R⁹⁰ are each independently selected form hydrogen and C₁-C₈alkyl, and alkyl, —N(H)cycloalkyl substituted with one or more substituents selected from oxo, hydroxy and alkyl, cycloalkyl containing from 1 to 4 heteroatoms, cycloalkyl containing from 1 to 4 heteroatoms substituted with one or more substituents selected from oxo, alkoxy, hydroxyl and alkyl where alkyl is optionally substituted with one or more substituents selected from halogen, hydroxy, alkoxy, oxo and cycloalkyl containing from 1 to 4 heteroatoms, —N(H)cycloalkyl containing from 1 to 4 heteroatoms substituted with one or more substituents selected from oxo, hydroxy and alkyl, and trifluoromethyl, and R²¹ and R²² are independently selected form hydrogen, alkyl, C₁-C₆alkyl substituted with one of more substituents selected from hydroxy, cycloalkyl containing from 1 to 4 heteroatoms optionally substituted with one or more substituents selected from C₁-C₆alkyl, hydroxy, oxo and halogen, ═NH, and ≡N, —S(O)₂aryl where aryl is optionally substituted with one or more substituents selected from: halogen, alkylamino and dialkylamino, C₁-C₁₂aryl, cycloalkyl containing from 1 to 4 heteroatoms, cycloalkyl containing from 1 to 4 heteroatoms substituted with one or more substituents selected from oxo, hydroxy, and alkyl, cycloalkyl, cycloalkyl substituted with one or more substituents selected from oxo, hydroxy, and alkyl, arylC₁-C₆alkyl optionally substituted with one or more substituents selected from oxo, hydroxy, and alkyl, cycloalkyl containing from 1 to 4 heteroatoms optionally substituted with one or more substituents selected from oxo, hydroxyl and alkyl, C₁-C₆alkoxy, C₁-C₄alkyloxyC₁-C₄alkyl, aryl and trifluoromethyl.

Suitably, the term “substituted” whenever used herein means that the subject chemical moiety has from one to five of the indicated substituents. Suitably, the term “substituted” whenever used herein means that the subject chemical moiety has from one to four of the indicated substituents. Suitably, the term “substituted” whenever used herein means that the subject chemical moiety has from one to three of the indicated substituents. Suitably, the term “substituted” whenever used herein means that the subject chemical moiety has one or two of the indicated substituents.

By the term “alkoxy” as used herein is meant -Oalkyl where alkyl is as described herein including —OCH₃ and —OC(CH₃)₂CH₃.

The term “cycloalkyl” as used herein unless otherwise defined, is meant a nonaromatic, unsaturated or saturated, cyclic or polycyclic C₃-C₁₂.

Examples of cycloalkyl and substituted cycloalkyl substituents as used herein include: cyclohexyl, aminocyclohexyl, cyclobutyl, aminocyclobutyl, 4-hydroxy-cyclohexyl, 2-ethylcyclohexyl, propyl4-methoxycyclohexyl, 4-methoxycyclohexyl, 4-carboxycyclohexyl, cyclopropyl, aminocyclopentyl, and cyclopentyl.

The term “cycloalkyl containing from 1 to 4 heteroatoms” and the term “cycloalkyl containing from 1 to 3 heteroatoms” as used herein unless otherwise defined, is meant a nonaromatic, unsaturated or saturated, cyclic or polycyclic ring containing from 1 to 12 carbons and containing from one to four heteroatoms or from one to three heteroatoms (respectively), provided that when the number of carbon atoms is 1 the aromatic ring contains at least four heteroatoms (applicable only where “cycloalkyl containing from 1 to 4 heteroatoms” is indicated), when the number of carbon atoms is 2 the aromatic ring contains at least three heteroatoms, when the number of carbon atoms is 3 the nonaromatic ring contains at least two heteroatoms and when the number of carbon atoms is 4 the nonaromatic ring contains at least one heteroatom.

Examples of cycloalkyl containing from 1 to 4 heteroatoms, cycloalkyl containing from 1 to 3 heteroatoms, substituted cycloalkyl containing from 1 to 4 heteroatoms and substituted cycloalkyl containing from 1 to 3 heteroatoms as used herein include: piperidine, piperazine, pyrrolidine, 3-methylaminopyrrolidine, piperazinly, tetrazole, hexahydrodiazepine and morpholine.

By the term “acyloxy” as used herein is meant —OC(O)alkyl where alkyl is as described herein. Examples of acyloxy substituents as used herein include: —OC(O)CH₃, —OC(O)CH(CH₃)₂ and —OC(O)(CH₂)₃CH₃.

By the term “N-acylamino” as used herein is meant —N(H)C(O)alkyl, where alkyl is as described herein. Examples of N-acylamino substituents as used herein include: —N(H)C(O)CH₃, —N(H)C(O)CH(CH₃)₂ and —N(H)C(O)(CH₂)₃CH₃.

By the term “aryloxy” as used herein is meant -Oaryl where aryl is phenyl, naphthyl, 3,4-methylenedioxyphenyl, pyridyl or biphenyl optionally substituted with one or more substituents selected from the group consisting of: alkyl, hydroxyalkyl, alkoxy, trifluoromethyl, acyloxy, amino, N-acylamino, hydroxy, —(CH₂)_{g}C(O)OR²⁵, —S(O)ₙR²⁵, nitro, cyano, halogen and protected —OH, where g is 0-6, R²⁵ is hydrogen or alkyl, and n is 0-2. Examples of aryloxy substituents as used herein include: phenoxy, 4-fluorophenyloxy and biphenyloxy.

By the term “heteroatom” as used herein is meant oxygen, nitrogen or sulfur.

By the term “halogen” as used herein is meant a substituent selected from bromide, iodide, chloride and fluoride.

By the term “alkyl” and derivatives thereof and in all carbon chains as used herein, including alkyl chains defined by the term “—(CH₂)ₙ”, “—(CH₂)ₘ” and the like, is meant a linear or branched, saturated or unsaturated hydrocarbon chain, and unless otherwise defined, the carbon chain will contain from 1 to 12 carbon atoms. Examples of alkyl and substituted alkyl substituents as used herein include: —CH₃, —CH₂—CH₃, —CH₂—CH₂—CH₃, —CH(CH₃)₂, —CH₂—CH₂—C(CH₃)₃, —CH₂—CF₃, —C≡C—C(CH₃)₃, —C≡C—CH₂—OH, cyclopropylmethyl, —CH₂—C(CH₃)₂—CH₂—NH₂, —C≡C—C₆H₅, —C≡C—C(CH₃)₂—OH, —CH₂—CH(OH)—CH(OH)—CH(OH)—CH(OH)—CH₂—OH, piperidinylmethyl, methoxyphenylethyl, —C(CH₃)₃, —(CH₂)₃—CH₃, —CH₂—CH(CH₃)₂, —CH(CH₃)—CH₂—CH₃, —CH═CH₂, and —C═C—CH₃.

By the term “treating” and derivatives thereof as used herein, is meant prophylatic and therapeutic therapy.

As used herein, the crisscrossed double bond indicated by the symbol [symbol] denotes Z and/or E stereochemistry around the double bond. In other words a compound of formula I or II can be either in the Z or E stereochemistry around this double bond, or a compound of formula I or II can also be in a mixture of Z and E stereochemistry around the double bond. However, in formulas I and II, the preferred compounds have Z stereochemistry around the double bond to which radical Q is attached.

The compounds of Formulas I and II naturally may exist in one tautomeric form or in a mixture of tautomeric forms. For example, for sake simplicity, compounds of formula I and II are expressed in one tautomeric form, usually as an exo form, i.e.

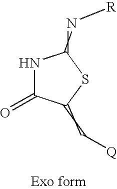

However, a person of ordinary skill can readily appreciate, the compounds of formulas I and II can also exist in endo forms.

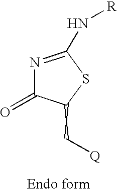

The present invention contemplates all possible tautomeric forms.

Certain compounds described herein may contain one or more chiral atoms, or may otherwise be capable of existing as two enantiomers, or two or more diastereoisomers. Accordingly, the compounds of this invention include mixtures of enantiomers/diastereoisomers as well as purified enantiomers/diastereoisomers or enantiomerically/diastereoisomerically enriched mixtures. Also included within the scope of the invention are the individual isomers of the compounds represented by formula I or II above as well as any wholly or partially equilibrated mixtures thereof. The present invention also covers the individual isomers of the compounds represented by the formulas above as mixtures with isomers thereof in which one or more chiral centers are inverted. Further, an example of a possible tautomer is an oxo substituent in place of a hydroxy substituent. Also, as stated above, it is understood that all tautomers and mixtures of tautomers are included within the scope of the compounds of Formula I or II.

Compounds of Formula (I) are included in the pharmaceutical compositions of the invention and used in the methods of the invention. Where a —COOH or —OH group is present, pharmaceutically acceptable esters can be employed, for example methyl, ethyl, pivaloyloxymethyl, and the like for —COOH, and acetate maleate and the like for —OH, and those esters known in the art for modifying solubility or hydrolysis characteristics, for use as sustained release or prodrug formulations.

The novel compounds of Formulas I and II are prepared as shown in Schemes I and II below, or by analogous methods, wherein the ‘Q’ and ‘R’ substituents are as defined in Formulas I and II respectively and provided that the ‘Q’ and ‘R’ substituents do not include any such substituents that render inoperative the processes of Schemes I to II. All of the starting materials are commercially available or are readily made from commercially available starting materials by those of skill in the art.

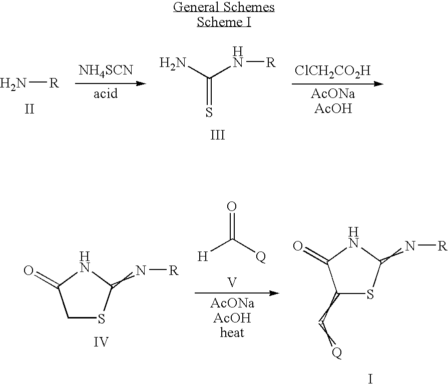

Briefly in Scheme 1, a mixture of aniline derivative of formula II (1 equivalent) and NH4SCN (about 1.3 equivalent) in an acid (typically 4N—HCl) is heated to reflux at about 110 C. ° for 6 hours. After cooling, the mixture is treated with H₂O, which process usually forms a solid, followed by desiccation in vacuo to give a compound of formula III.

A mixture of formula III compound, ClCH₂CO₂H (1 equivalent), and AcONa (1 equivalent) in AcOH is heated to reflux at around 110 C. ° for about 4 h. The mixture is poured onto water thereby a solid is typically formed, which is isolated by filtration. The solid is washed with a solvent such as MeOH to afford a compound of formula IV.

A mixture of formula IV compound, an aldehyde of formula V (1 equivalent), a catalyst such as AcONa (3 equivalent) in AcOH or piperidine with or without AcOH is heated to reflux at about 110 C. ° for about 10 to 48 hours or heated in a microwave reactor. After cooling, the reaction mixture is purified to afford a target product of Formula I.

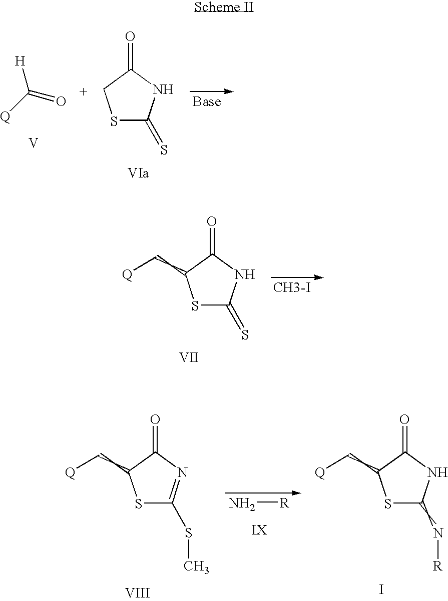

Briefly in Scheme 2, a mixture of an aldehyde of formula V (1 equivalent), Rhodanine (1 equivalent), sodium acetate (about 3 equivalents), and acetic acid is heated at around 110 C. ° for about 48 h. The reaction mixture is cooled to room temperature to afford a product of formula VII.

Then, to a room temperature suspension of VII (1 equivalent) in a suitable solvent such as ethanol is added Hunig's base (about 2 equivalents) followed by iodomethane (about 5 equivalents). The resultant suspension is stirred at room temperature for 3.5 h to yield a compound of VII.

A mixture of VII (1 equivalent) and an amine of formula IX (1˜2 equivalent) in a suitable solvent such as ethanol or valeric acid with or without a base such as 1,8-diazabicyclo[5.4.0]undec-7-ene is heated at a suitable temperature in a microwave reactor or otherwise until the reaction is judged complete. The desired product of Formula I is obtained after purification.

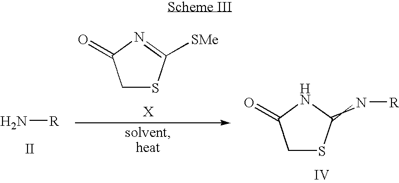

Briefly in Scheme 3, a compound of formula IV can also be prepared by heating a thiazolidinone of formula X with an amine of formula I in a suitable solvent, such as ethanol under reflux.

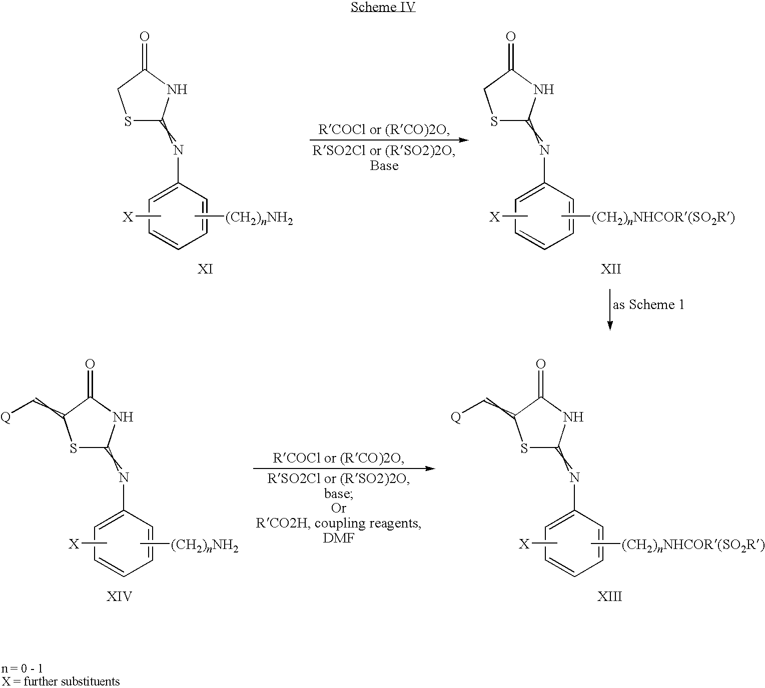

Briefly in Scheme 4, amino groups compounds of formula XI and XIV may be acylated or sulfonylated using acid or sulfonyl chlorides or anhydrides with or without a suitable base, such as pyridine; or coupled with an acid using standard coupling reagents in a suitable solvent such as DMF to give the compounds XII after purification.

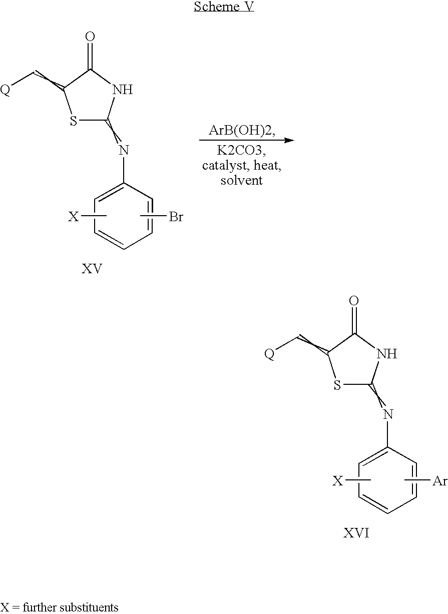

Briefly in Scheme 5, a bromide of formula XV may be converted under Suzuki conditions using a boronic acid to give the compounds XVI after purification.

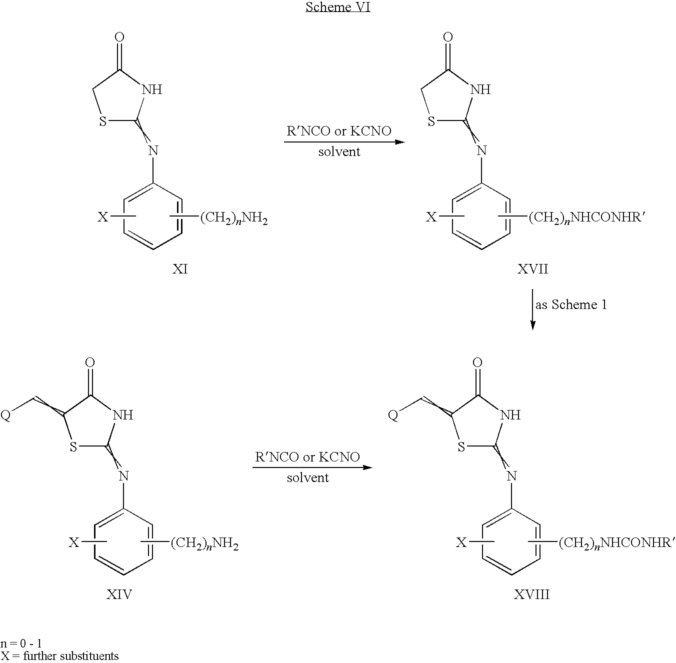

Briefly in Scheme 6, amino groups compounds of formula XI and XIV may be converted into substituted or unsubstituted ureas of formula XVII and XVIII using potassium cyanate or an organic isocyanate in a suitable solvent.

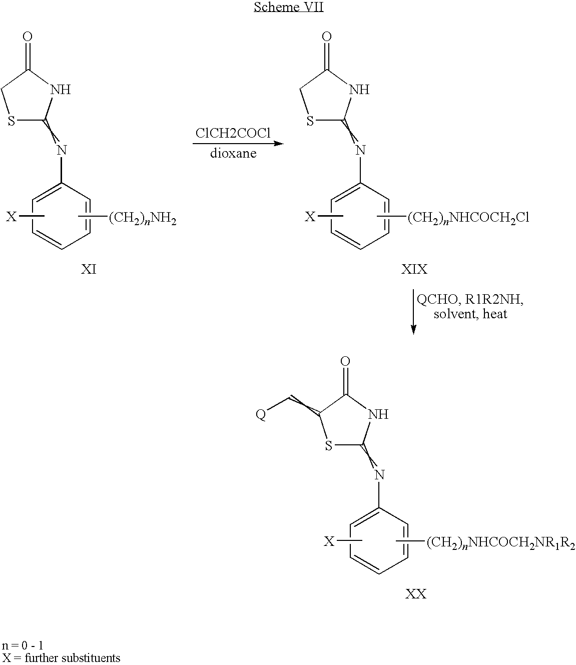

Briefly in Scheme 7, amines of formula XI may be acylated with chloroacetyl chloride in dioxane to produce intermediates of formula XIX. These compounds may be heated with an aldehyde QCHO and an amine R1R2NH in a suitable solvent in a microwave reactor to give the compounds of formula XX after purification.

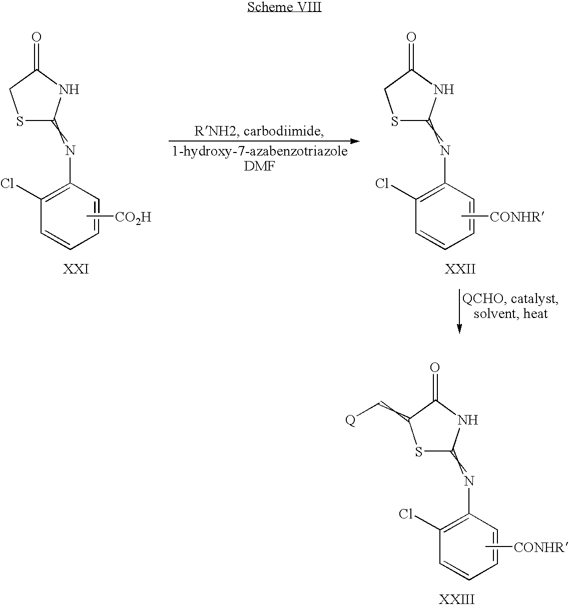

Briefly in Scheme 8, acids of formula XXI may be coupled with amines RNH2 using a carbodiimide and 1-hydroxy-7-azabenzotriazole in DMF to give the compounds of formula XXII. These can be converted using the methods of scheme 1 to the compounds of formula XXIII after purification.

In Schemes 1 and 8, the meaning of R and Q are as defined in Formula I.

In other embodiments, additional compounds of the invention can also be synthesized whereby a compound of Formula I is first made by a process of Scheme 1 or 2 (or a variant thereof), and Q and R radicals in compounds of Formula I thus made are further converted by routine organic reaction techniques into different Q and R groups.

By the term “co-administering” and derivatives thereof as used herein is meant either simultaneous administration or any manner of separate sequential administration of a hYAK3 inhibiting compound, as described herein, and a further active ingredient or ingredients, known to be useful in treating diseases of the hematopoietic system, particularly anemias, including EPO or a derivative thereof. The term further active ingredient or ingredients, as used herein, includes any compound or therapeutic agent known to or that demonstrates advantageous properties when administered to a patient in need of treatment for diseases of the hematopoietic system, particularly anemias, and any compound or therapeutic agent known to or that demonstrates advantageous properties when administered in combination with a hYAK3 inhibiting compound. Preferably, if the administration is not simultaneous, the compounds are administered in a close time proximity to each other. Furthermore, it does not matter if the compounds are administered in the same dosage form, e.g. one compound may be administered topically and another compound may be administered orally.

Because the pharmaceutically active compounds of the present invention are active as hYAK3 inhibitors they exhibit therapeutic utility in treating diseases of the hematopoietic system, particularly anemias.

The pharmaceutically active compounds within the scope of this invention are useful as hYAK inhibitors in mammals, particularly humans, in need thereof.

The present invention therefore provides a method of treating diseases of the hematopoietic system, particularly anemias and other conditions requiring hYAK inhibition, which comprises administering an effective compound of Formula (I) or a pharmaceutically acceptable salt, hydrate, solvate or pro-drug thereof. The compounds of Formula (I) also provide for a method of treating the above indicated disease states because of their ability to act as hYAK inhibitors. The drug may be administered to a patient in need thereof by any conventional route of administration, including, but not limited to, intravenous, intramuscular, oral, subcutaneous, intradermal, and parenteral.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-100 mg/kg of active compound, preferably 0.001-50 mg/kg. When treating a human patient in need of a hYAK inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages is preferred. Parenteral administration, at high dosages, however, also can be used when safe and convenient for the patient.

Optimal dosages to be administered may be readily determined by those skilled in the art, and will vary with the particular hYAK inhibitor in use, the strength of the preparation, the mode of administration, and the advancement of the disease condition. Additional factors depending on the particular patient being treated will result in a need to adjust dosages, including patient age, weight, diet, and time of administration.

The method of this invention of inducing hYAK inhibitory activity in mammals, including humans, comprises administering to a subject in need of such activity an effective hYAK inhibiting amount of a pharmaceutically active compound of the present invention.

The invention also provides for the use of a compound of Formula (I) in the manufacture of a medicament for use as a hYAK inhibitor.

The invention also provides for the use of a compound of Formula (I) in the manufacture of a medicament for use in therapy.

The invention also provides for the use of a compound of Formula (I) in the manufacture of a medicament for use in treating diseases of the hematopoietic system, particularly anemias.

The invention also provides for a pharmaceutical composition for use as a hYAK inhibitor which comprises a compound of Formula (I) and a pharmaceutically acceptable carrier.

The invention also provides for a pharmaceutical composition for use in the treatment of diseases of the hematopoietic system, particularly anemias which comprises a compound of Formula (I) and a pharmaceutically acceptable carrier.

No unacceptable toxicological effects are expected when compounds of the invention are administered in accordance with the present invention.

In addition, the pharmaceutically active compounds of the present invention can be co-administered with further active ingredients, such as other compounds known to treat diseases of the hematopoietic system, particularly anemias, or compounds known to have utility when used in combination with a hYAK inhibitor.

Without further elaboration, it is believed that one skilled in the art can, using the preceding description, utilize the present invention to its fullest extent. The following Examples are, therefore, to be construed as merely illustrative and not a limitation of the scope of the present invention in any way.

### EXPERIMENTAL DETAILS

The compounds of Examples 1 to 181 are readily made according to Schemes I and II or by analogous methods.

### Example 1

### (5Z)-2-[(2-Chloro-3-pyridinyl)amino]-5-(6-quinolinylmethylidene)-1,3-thiazol-4(5H)-one

A mixture of 3-amino-2-chloropyridine (68 mg, 0.53 mmol), (5Z)-2-(methylthio)-5-(6-quinolinylmethylidene)-1,3-thiazol-4(51)-one (T. Rueckle et al., PCT Int. Appl., 2005, WO2005011686A1; 100 mg, 0.35 mmol) and dioxan (1.0 mL) were sealed in a pressure bottle and heated at 160° C. for 3 hours. The mixture was cooled, some ethanol added and the product collected, washed with ethanol and dichloromethane to give the title compound (25 mg, 19%). 1H NMR (400 MHz, DMSO-d₆) δ ppm 7.50 (dd, J=7.6 and 4.4 Hz, 1H), 7.57 (dd, J=8.4 and 4.4 Hz, 1H), 7.68 (dd, J=8.0 and 1.6 Hz, 1H), 7.87 (d, J=7.2 Hz, 1H) 7.89 (s, 1H), 8.08 (d, J=8.8 Hz, 1H), 8.16 (d, J=1.6 Hz, 1H), 8.27 (dd, J=4.4 and 1.6 Hz, 1H), 8.46 (d, J=8.0 Hz, 1H), 8.94 (dd, J=4.4 and 1.6 Hz, 1H), 12.50 (br. s, 1H).

### Example 2

### (5Z)-2-{[2-Chloro-5-(2-pyridinyl)phenyl]amino}-5-(6-quinolinylmethylidene)-1,3-thiazol-4(5H)-one

a) 2-(4-Chloro-3-nitrophenyl)pyridine. A mixture of 4-chloro-3-nitrophenylboronic acid (500 mg, 2.58 mmoles), 2-bromopyridine (380 mg, 2.58 mmoles), tetrakistriphenylphosphine palladium(0) (100 mg, 0.086 mmoles), sodium carbonate (800 mg, 7.5 mmoles) in dimethylformamide (15 mL) and water (2.5 mL) was heated at 100° C. for 2 hours. The mixture was diluted with water and extracted into ethyl acetate (×2). The combined extracts were washed with water and brine, dried and evaporated. Flash chromatography (fine silica, dichloromethane) afforded the title compound (300 mg, 50%). 1H NMR (400 MHz, CHLOROFORM-d) δ ppm 7.36 (ddd, J=7.39, 4.86, 1.14 Hz, 1H) 7.67 (d, J=8.34 Hz, 1H) 7.78-7.89 (m, 2H) 8.21 (dd, J=8.34, 2.02 Hz, 1H) 8.58 (d, J=2.02 Hz, 1H) 8.75 (d, J=4.80 Hz, 1H)

b) [2-Chloro-5-(2-pyridinyl)phenyl]amine. A mixture of 2-(4-chloro-3-nitrophenyl)pyridine (300 mg,) and tin II chloride (3.0 g,) was heated in ethanol (15 mL) at 60° C. for 2 hours. Water was added and the mixture basified with 1N sodium hydroxide solution. The mixture was filtered through celite and washed through with water and ethyl acetate. The layers were separated; the ethyl acetate was washed with brine, dried and evaporated to a yellow oil (300 mg, quant.). 1H NMR (400 MHz, CHLOROFORM-d) δ ppm 4.25 (s, 2H, NH) 7.26-7.38 (m, 3H) 7.57 (d, J=2.02 Hz, 1H) 7.75 (d, J=8.08 Hz, 1H) 7.85 (td, J=7.77, 1.89 Hz, 1H) 8.72 (dd, J=4.04, 1.01 Hz, 1H).

c) (5Z)-2-{[2-Chloro-5-(2-pyridinyl)phenyl]amino}-5-(6-quinolinylmethylidene)-1,3-thiazol-4(5H)-one. A mixture of [2-chloro-5-(2-pyridinyl)phenyl]amine (206 mg, 1.0 mmol) and (5Z)-2-(methylthio)-5-(6-quinolinylmethylidene)-1,3-thiazol-4(5H)-one (180 mg, 0.63 mmol) in dimethylformamide (0.5 mL) were heated in a pressure bottle at 160° C. for 2 hours. The mixture was cooled, some ethanol added, the product collected, slurried in hot ethanol, washed with ethanol and dichloromethane to give the title compound (50 mg, 18%) 1H NMR (400 MHz, DMSO-d₆) δ ppm 7.38 (dd, J=6.57, 4.80 Hz, 1H) 7.53 (dd, J=8.34, 4.29 Hz, 1H) 7.68 (d, J=8.34 Hz, 1H) 7.82-7.92 (m, 4H) 7.97 (dd, J=8.46, 2.15 Hz, 1H) 8.05 (dd, J=8.84, 1.77 Hz, 2H) 8.14 (d, J=1.52 Hz, 1H) 8.42 (d, J=7.83 Hz, 1H) 8.66 (d, J=3.79 Hz, 1H) 8.91 (dd, J=4.17, 1.64 Hz, 1H) 12.85 (br. s, 1H)

### Example 3

### N-(4-Chloro-3-{[(5Z)-4-oxo-5-(6-quinolinylmethylidene)-4,5-dihydro-1,3-thiazol-2-yl]amino}phenyl)cyclobutanecarboxamide

a) 2-[(2-Chloro-5-nitrophenyl)amino]-1,3-thiazol-4(5H)-one. N-(2-chloro-5-nitrophenyl)thiourea (M. Sedlak et. al., J. Phys. Org. Chem., 2001, 14(3), 187; 5.46 g, 23.6 mmol) in acetic acid (38.0 mL) was treated with sodium acetate (1.95 g, 23.7 mmole) and chloroacetic acid (2.30 g, 24.3 mmol.). The reaction was heated to 130° C. for four hours and then cooled and poured into ice (300 g.). The crude mixture was filtered and dried to provide a pale yellow solid (5.43 g, 84%). 1H NMR (400 MHz, DMSO-d₆) δ ppm 12.21 (s, 1H) 7.98 (dd, J=8.72, 2.65 Hz, 1H) 7.90 (s, 1H) 7.82 (d, J=8.84 Hz, 1H) 4.08 (s, 2H)

b) 2-[(5-Amino-2-chlorophenyl)amino]-1,3-thiazol-4(5H)-one. 2-[(2-chloro-5-nitrophenyl)amino]-1,3-thiazol-4(5H)-one (2.03 g, 7.491 mmole) and 10% Palladium on Carbon Degussa (2.10 g) were charged to a 500 mL round bottom flask. The flask was evacuated with nitrogen and then methanol (160.0 mL) was added. The flask was then purged with hydrogen three times. The reaction was allowed to stir for 12 hours at 1 atm hydrogen. The reaction mixture was filtered, solids washed with dioxane, and concentrated and triturated carefully with methanol to provide the title compound (1.35 g, 74%) as a white solid. ¹H NMR (400 MHz, DMSO) δ 11.87 (s, 1H) 7.06 (d, J=8.55, 1H) 6.32 (d, J=6.67, H) 6.18 (s, 1H) 5.29 (bs, 2H) 3.99 (s, 2H)

c) N-(4-Chloro-3-{[(5Z)-4-oxo-5-(6-quinolinylmethylidene)-4,5-dihydro-1,3-thiazol-2-yl]amino}phenyl)cyclobutanecarboxamide. 2-[(5-amino-2-chlorophenyl)amino]-1,3-thiazol-4(5H)-one (0.150 g, 0.622 mmol) in dioxane (2.0 mL) was treated with cyclobutylacetyl chloride (0.140 mL, 1.23 mmol). The reaction was stirred for 30 min. and then quenched with water, diluted with ethyl acetate and extracted twice. The organics were combined and dried over MgSO₄, filtered, and concentrated under reduced pressure to obtain a crude white solid (0.104 g, 0.320 mmol) which was dissolved in ethanol (3.0 mL) and sodium acetate (0.053 g, 0.640 mmol) and 6-quinolinecarbaldehyde (0.050 g, 0.320 mmol) added. The reaction was heated in a microwave to 150° C. for 40 min. The reaction mixture was diluted with water (3.0 mL) and filtered and washed with water and ethyl acetate to obtain a yellow solid (0.030 g, 10% for two steps). 1H NMR (400 MHz, DMSO-d₆) δ ppm 12.78 (s, 1H) 9.94 (s, 1H) 8.94 (dd, J=4.17, 1.64 Hz, 1H) 8.47 (d, J=7.83 Hz, 1H) 8.17 (s, 1H) 8.09 (d, J=8.84 Hz, 1H) 7.83-7.90 (m, 2H) 7.57 (dd, J=8.34, 4.29 Hz, 1H) 7.40-7.50 (m, 3H) 3.16-3.26 (m, 1H) 2.15-2.27 (m, 2H) 2.04-2.14 (m, J=8.08 Hz, 2H) 1.87-1.98 (m, 1H) 1.74-1.84 (m, J=9.35 Hz, 1H)

### Example 4

### N-(4-chloro-3-{[(5Z)-4-oxo-5-(6-quinoxalinylmethylidene)-4,5-dihydro-1,3-thiazol-2-yl]amino}phenyl)-2-methylpropanamide

a) N-{4-Chloro-3-[(4-oxo-4,5-dihydro-1,3-thiazol-2-yl)amino]phenyl}-2-methylpropanamide. 2-[(5-amino-2-chlorophenyl)amino]-1,3-thiazol-4(5H)-one (example 3b), 0.200 g, 0.8264 mmole) in dioxane (4.0 mL) was treated with isobutyryl chloride (0.433 mL, 5 eq.) and allowed to stir for 1 hour. The reaction was diluted with methylene chloride and filtered and then resubmitted to the above reaction conditions and stirred for an additional 12 hours. The reaction was then diluted with ethyl acetate and extracted twice with 1N HCl, and one time with brine, dried over MgSO₄, filtered and concentrated. The product was purified via Isco (CH₂Cl₂ to 5% MeOH/CH₂Cl₂) to obtain a white solid (0.165 g, 64%) 1H NMR (400 MHz, DMSO-d₆) δ ppm 12.00 (s, 1H) 9.97 (s, 1H) 7.45 (s, 1H) 7.39 (d, J=8.70, 1H) 7.31 (d, J=2.11, 1H) 4.03 (s, 2H) 2.57 (m, 1H) 1.10 (s, 3H) 1.08 (s, 3H)

b) N-(4-Chloro-3-{[(5Z)-4-oxo-5-(6-quinoxalinylmethylidene)-4,5-dihydro-1,3-thiazol-2-yl]amino}phenyl)-2-methylpropanamide. N-{4-chloro-3-[(4-oxo-4,5-dihydro-1,3-thiazol-2-yl)amino]phenyl}-2-methylpropanamide (0.165 g, 0.529 mmole) and 6-quinoxalinecarbaldehyde (0.083 g, 0.532 mmole) in ethanol (2.0 mL) was treated with piperidine (0.052 mL, 0.525 mmol.) The reaction was heated in a microwave to 150° C. for 40 min. The reaction mixture was diluted with water (3.0 mL) and washed with 1N Hydrochloric acid and then water and dried. This was reprecipitated by dissolving in basic ethanol and re-acidfied with acetic acid. The yellow solid was filtered and dried to provide (0.0482 g, 20%.) 1H NMR (400 MHz, DMSO-d₆) δ ppm 12.36 (bs, 1H) 10.01 (s, 1H) 8.95 (s, 1H) 8.18 (s, 1H) 8.16 (d, J=8.76, 1H) 7.99 (d, J=8.61, 1H) 7.83 (s, 1H) 7.43 (s, 3H) 2.80 (m, 1H) 1.09 (s, 3H) 1.08 (s, 3H)

### Example 5

### N-(4-chloro-3-{[(5Z)-4-oxo-5-(6-quinolinylmethylidene)-4,5-dihydro-1,3-thiazol-2-yl]amino}phenyl)methanesulfonamide

2-[(5-Amino-2-chlorophenyl)amino]-1,3-thiazol-4(5H)-one (example 3b), 0.100 g, 0.415 mmol) in dichloromethane (2.0 mL) was treated with pyridine (2.0 mL, 24.73 mmol) and methanesulfonic anhydride (0.15 g, 0.861 mmol.) The reaction was stirred for 30 min. and then quenched with water, diluted with ethyl acetate and extracted twice. The organics were combined and dried over MgSO₄, filtered, and concentrated under reduced pressure to provide (0.116 g, 0.363 mmol.) of solid. This was then dissolved in ethanol (4.0 mL) and 6-quinolinecarbaldehyde (0.057 g, 0.363 mmol) and piperidine (0.036 mL, 0.363 mmol.) were added. The reaction was heated in a microwave to 150° C. for 20 min. The reaction mixture was diluted with water (3.0 mL) and washed with 1M hydrochloric acid and then water and dried. HPLC purification gave a brown solid (0.004 g, 1.7% for two steps). 1H NMR (400 MHz, DMSO-d₆) δ ppm 12.81 (s, 1H) 10.03 (s, 1H) 8.96 (dd, J=1.6, 2.58, 1H) 8.47 (d, J=7.86, 1H) 8.17 (d, J=1.64, 1H) 8.10 (d, J=8.75, 1H) 7.90 (d, J=1.97, 1H) 7.87 (s, 1H) 7.60 (dd, J=4.29, 4.21, 1H) 7.53 (d, J=8.67, 1H) 7.05 (dd, J=2.65, 6.10, 1H) 6.98 (s, 1H) 3.08 (s, 3H)

### Example 6

### N-[(4-Chloro-3-{[(5Z)-4-oxo-5-(6-quinolinylmethylidene)-4,5-dihydro-1,3-thiazol-2-yl]amino}phenyl)methyl]-2-(methyloxy)acetamide

a) 1,1-Dimethylethyl ({4-chloro-3-[(4-oxo-4,5-dihydro-1,3-thiazol-2-yl)amino]phenyl}methyl)carbamate. A solution of 1,1-dimethylethyl [(3-amino-4-chlorophenyl)methyl]carbamate (G. Zhao et. al., Bioorg. Med. Chem. Lett., 2004, 14(2), 309; 4.62 g, 18.0 mmol) and 2-(methylthio)-1,3-thiazol-4(5H)-one (A. I. Khodair, J. Heterocyclic Chem., 2002, 39, 1153; 3.18 g, 21.6 mmol) in ethanol (60 mL) was heated under reflux for 40 h, then cooled. The solvent was evaporated under reduced pressure and the residue chromatographed (silica gel, 30-70% ethyl acetate/hexanes) to give the title compound (4.99 g, 78%) as a solid. ¹H NMR (400 MHz, DMSO-d₆) δ ppm 12.03 (br s, 1H), 7.46-7.42 (m, 2H), 6.99 (d, J=8.4 Hz, 1H), 6.88 (s, 1H), 4.09 (d, J=6.4 Hz, 2H), 4.02 (s, 2H), 1.39 (s, 9H).

b) 1,1-Dimethylethyl [(4-chloro-3-{[(5Z)-4-oxo-5-(6-quinolinylmethylidene)-4,5-dihydro-1,3-thiazol-2-yl]amino}phenyl)methyl]carbamate. A mixture of 1,1-dimethylethyl ({4-chloro-3-[(4-oxo-4,5-dihydro-1,3-thiazol-2-yl)amino]phenyl}methyl)carbamate (1.67 g, 4.69 mmol), quinoline-6-carbaldehyde (0.884 g, 5.62 mmol), piperidinium acetate in ethanol (0.2M, 5 mL, 1.00 mmol), and toluene (15 mL) was heated under reflux for 18 h, then cooled and the solid filtered, washed (10% ethanol/toluene, then toluene) and dried to give the title compound (1.30 g, 56%) as a solid. ¹H NMR (400 MHz, DMSO-d₆) δ ppm 12.76 (br s, 1H), 8.95 (s, 1H), 8.45 (d, J=8.1 Hz, 1H), 8.15 (s, 1H), 8.07 (d, J=8.8 Hz, 1H), 7.86 (m, 2H), 7.58 (dd, J=8.3, 4.3 Hz, 1H), 7.50 (d, J=8.4 Hz, 1H), 7.44 (br t, J=6.2 Hz, 1H), 7.09 (m, 1H), 7.01 (s, 1H), 4.14 (d, J=6.1 Hz, 2H), 1.30 (s, 9H).

c) (5Z)-2-{[5-(Aminomethyl)-2-chlorophenyl]amino}-5-(6-quinolinylmethylidene)-1,3-thiazol-4(5H)-one. A solution of 1,1-dimethylethyl [(4-chloro-3-{[(5Z)-4-oxo-5-(6-quinolinylmethylidene)-4,5-dihydro-1,3-thiazol-2-yl]amino}phenyl)methyl]carbamate (1.30 g, 2.67 mmol) in trifluoroacetic acid (20 mL) was stirred 20 min, then the solvent removed under reduced pressure and the residue dissolved in 0.5M aqueous potassium carbonate (75 mL). The solution was filtered and the pH adjusted to 8 with acetic acid. The solid was filtered, washed (water) and dried to give the title compound (1.00 g, 96%) as a pale orange powder. 1H NMR (400 MHz, DMSO-d₆+1% TFA) δ ppm 4.08 (q, J=5.56 Hz, 2H) 7.26 (s, 1H), 7.31 (dd, J=8.2, 1.7 Hz, 1H), 7.65 (d, J=8.1 Hz, 1H), 7.73 (dd, J=8.3, 4.6 Hz, 1H), 7.91 (s, 1H), 7.95 (dd, J=8.8, 1.5 Hz, 1H), 8.15 (d, J=8.9 Hz, 1H), 8.21 (br s, 3H), 8.25 (s, 1H), 8.63 (d, J=8.1 Hz, 1H), 9.05 (dd, J=4.3, 1.1 Hz, 1H).

d) N-[(4-Chloro-3-{[(5Z)-4-oxo-5-(6-quinolinylmethylidene)-4,5-dihydro-1,3-thiazol-2-yl]amino}phenyl)methyl]-2-(methyloxy)acetamide. A mixture of (5Z)-2-{[5-(aminomethyl)-2-chlorophenyl]amino}-5-(6-quinolinylmethylidene)-1,3-thiazol-4(5H)-one (0.060 g, 0.152 mmol), 1-hydroxy-7-azabenzotriazole (0.023 g, 0.167 mmol), methoxyacetic acid (0.015 g, 0.167 mmol), 1-[3-(dimethylamino)propyl]-3-ethylcarbodiimide hydrochloride (0.032 g, 0.167 mmol) and dimethylformamide (1 mL) was stirred 18 h, then diluted with water (2 mL) and the solid filtered, washed (water) and dried to give the title compound (0.046 g, 65%) as an orange powder. 1H NMR (400 MHz, DMSO-d₆) δ ppm 3.25 (s, 3H), 3.84 (s, 2H), 4.31 (d, J=6.0 Hz, 2H), 7.06 (s, 1H), 7.11 (dd, J=8.4, 1.6 Hz, 1H), 7.50 (d, J=8.4 Hz, 1H), 7.58 (dd, J=8.0, 4.0 Hz, 1H), 7.86-7.88 (m, 2H), 8.08 (d, J=8.8 Hz, 1H), 8.16 (d, J=1.6 Hz, 1H), 8.46-8.49 (m, 2H), 8.95 (dd, J=4.4, 2.0 Hz, 1H), 12.78 (br s, 1H).

### Example 7

### (5Z)-2-[(2-Chloro-5-{[(1-methylethyl)amino]methyl}phenyl)amino]-5-(6-quinolinylmethylidene)-1,3-thiazol-4(5H)-one

Sodium triacetoxyborohydride (0.135 g, 0.637 mmol) was added to a stirred mixture of (5Z)-2-{[5-(aminomethyl)-2-chlorophenyl]amino}-5-(6-quinolinylmethylidene)-1,3-thiazol-4(5H)-one (example 6c), 0.050 g, 0.127 mmol), acetone (3 mL) and dimethylformamide (3 mL). After stirring 18 h, water (40 mL) was added and the pH adjusted to 8 with sodium bicarbonate. The solid was filtered, washed with water, and dried to give the title compound (0.026 g, 47%) as a yellow powder. 1H NMR (400 MHz, DMSO-d₆+1% TFA) δ 1.26 (d, J=6.6 Hz, 6H), 3.32 (m, 1H), 4.19 (t, J=6.0 Hz, 2H), 7.33 (s, 1H), 7.36 (d, J=8.4 Hz, 1H), 7.67 (d, J=8.1 Hz, 1H), 7.71 (dd, J=8.3, 4.6 Hz, 1H), 7.91 (s, 1H), 7.94 (dd, J=8.8, 1.5 Hz, 1H), 8.14 (d, J=8.8 Hz, 1H), 8.25 (s, 1H), 8.62 (d, J=8.3 Hz, 1H), 8.75 (s, 2H), 9.05 (dd, J=4.3, 1.3 Hz, 1H), 12.83 (br s, 1H).

### Example 8

### (2Z,5Z)-2-[(5-Chloro-1H-benzimidazol-6-yl)imino]-5-(quinolin-6-ylmethylene)-1,3-thiazolidin-4-one

a) 5-Chloro-6-nitro-1H-benzimidazole. A mixture of 4-chloro-5-nitro-1,2-phenylenediamine (R. Nasielski-Hinkens et al., Heterocycles, 1987, 26(9), 2433; 0.187 g, 1.00 mmol) and formic acid (2 mL) was stirred in a microwave reactor at 140° C. for 30 min, then cooled and diluted with water (5 mL). The pH was adjusted to 9 with concentrated aqueous ammonia solution and the solid filtered after cooling, washed (water) and dried to give the title compound (0.208 g, ˜100%) as a solid. 1H NMR (400 MHz, DMSO-d₆) δ ppm 7.95 (s, 1H), 8.39 (s, 1H), 8.56 (s, 1H), 12.89 (br s, 1H).

b) 5-Chloro-1H-benzimidazol-6-amine. A mixture of 5-chloro-6-nitro-1H-benzimidazole (0.206 g, 1.04 mmol), indium (0.603 g, 5.25 mmol), ammonium chloride (0.563 g, 10.5 mmol) and ethanol/water (2:1, 9 mL) was heated under reflux for 2 h, then cooled and diluted with brine (150 mL). The mixture was extracted with ethyl acetate and the extracts dried (Na₂SO₄), then evaporated under reduced pressure to give the title compound (0.151 g, 87%) as a waxy solid. LCMS (ES) m/e 168 (M+H)⁺.

c) 2-[(5-Chloro-1H-benzimidazol-6-yl)amino]-1,3-thiazol-4(5H)-one. The method of example 6a) was followed here, using 5-chloro-1H-benzimidazol-6-amine in place of 1,1-dimethylethyl [(3-amino-4-chlorophenyl)methyl]carbamate, to give the title compound (49%) as a solid. LCMS (ES) m/e 267 (M+H)⁺.

d) (2Z,5Z)-2-[(5-Chloro-1H-benzimidazol-6-yl)imino]-5-(quinolin-6-ylmethylene)-1,3-thiazolidin-4-one. The method of example 6b) was followed here, using 2-[(5-chloro-1H-benzimidazol-6-yl)amino]-1,3-thiazol-4(5H)-one in place of 1,1-dimethylethyl ({4-chloro-3-[(4-oxo-4,5-dihydro-1,3-thiazol-2-yl)amino]phenyl}methyl)carbamate, to give the title compound (55%) as a solid. 1H NMR (400 MHz, DMSO-d₆+1% TFA) δ 7.66 (s, 1H), 7.70 (dd, J=8.3, 4.5 Hz, 1H), 7.91 (s, 1H), 7.94 (dd, J=9.1, 2.0 Hz, 1H), 8.07-8.17 (m, 2H), 8.23 (d, J=1.8 Hz, 1H), 8.63 (d, J=8.1 Hz, 1H), 9.04 (dd, J=4.4, 1.6 Hz, 1H), 9.43 (s, 1H).

### Example 9

### 4-Chloro-N-cyclobutyl-3-{[(5Z)-4-oxo-5-(6-quinolinylmethylidene)-4,5-dihydro-1,3-thiazol-2-yl]amino}benzenesulfonamide

a) 4-Chloro-N-cyclobutyl-3-nitrobenzenesulfonamide. A solution of cyclobutylamine (0.552 mL, 6.44 mmol) and pyridine (0.709 mL, 8.79 mmol) in dichloromethane (15 mL) was cooled at 0° C. After the addition of 4-chloro-3-nitrobenzenesulfonyl chloride (1.5 g, 5.86 mmol), the mixture was warmed to room temperature and stirring was continued for 2.5 h. The solvent was evaporated at reduced pressure and the residue was partitioned between 1N HCl and ethyl acetate. The organic was washed with water, brine, dried over Na₂SO₄ and evaporated to give the desired product as a yellow solid (1.21 g, 71%). ¹H NMR (400 MHz, CDCl₃) δ ppm 8.35 (d, J=2.3 Hz, 1H), 7.99 (dd, J=8.5, 2.2, 1H), 7.72 (d, J=8.3 Hz, 1H), 4.96 (d, J=8.5 Hz, 1H), 3.87 (sext, J=8.2 Hz, 1H), 2.26-2.19 (m, 2H), 1.90-1.80 (m, 2H), 1.73-1.62 (m, 2H).

b) 3-Amino-4-chloro-N-cyclobutylbenzenesulfonamide. A mixture of the compound from Example 9a) (500 mg, 1.72 mmol) and SnCl₂ (1.96 g, 10.32 mmol) in EtOH (10 mL) was stirred at 70° C. for 4 h, then cooled to room temperature, poured into 1N NaOH and extracted with ethyl acetate. The organics were washed with water, brine, dried over Na₂SO₄ and evaporated to give the title compound as a yellow oil (410 mg, 91%). ¹H NMR (400 MHz, CDCl₃) δ ppm 7.33 (d, J=8.3 Hz, 1H), 7.30 (d, J=2.0, 1H), 7.12 (dd, J=8.3, 2.0 Hz, 1H), 5.20 (d, J=8.6 Hz, 1H), 3.76 (sext, J=8.1 Hz, 1H), 2.14-2.07 (m, 2H), 1.84-1.74 (m, 2H), 1.64-1.50 (m, 2H).

c) 3-[(Aminocarbonothioyl)amino]-4-chloro-N-cyclobutylbenzenesulfonamide. Benzoyl isothiocyanate (0.278 mL, 2.07 mmol) was added dropwise to a solution of 3-amino-4-chloro-N-cyclobutylbenzenesulfonamide (450 mg, 1.73 mmol) in acetone (5 mL). The mixture was stirred overnight and poured onto ice. The precipitate was collected, filtered, washed with water and dried. The solid was suspended in MeOH and a solution of NaOMe (25% wt. in MeOH, 0.747 mL) was added dropwise. The mixture was stirred overnight, then treated with 1N HCl to pH neutral, concentrated and extracted with ethyl acetate. The organics were washed with water, then brine, dried over Na₂SO₄ and evaporated at reduced pressure to afford the title compound as yellow oil (440 mg, 79%). MS (ES+) m/e 320 [M+H]⁺.

d) 4-Chloro-N-cyclobutyl-3-[(4-oxo-4,5-dihydro-1,3-thiazol-2-yl)amino]benzenesulfonamide. A solution of 3-[(aminocarbonothioyl)amino]-4-chloro-N-cyclobutylbenzenesulfonamide (430 mg, 1.34 mmol), chloroacetic acid (133.5 mg, 1.41 mmol) and sodium acetate (115.7 mg, 1.41 mg) in acetic acid (10 mL) was stirred at reflux temperature for 5 h. Water (10 mL) was then added while cooling and the mixture was allowed to stand at room temperature overnight. The solid obtained was collected by filtration, washed with water and dried to give the title compound as tan solid (315 mg, 65%) which was used directly in the following step without further purification.

4-Chloro-N-cyclobutyl-3-{[(5Z)-4-oxo-5-(6-quinolinylmethylidene)-4,5-dihydro-1,3-thiazol-2-yl]amino}benzenesulfonamide. A solution of the 4-chloro-N-cyclobutyl-3-[(4-oxo-4,5-dihydro-1,3-thiazol-2-yl)amino]benzenesulfonamide (300 mg, 0.834 mmol), 6-quinolinecarbaldehyde (131 mg, 0.834 mmol) and piperidine (0.082 mL, 0.834 mmol) in ethanol (2.0 mL) was stirred and heated at 150° C. for 30 min. in a Biotage Initiator microwave synthesizer. The reaction mixture was then cooled, poured onto water, and the pH of the mixture was adjusted to about 6 by the addition of 1N aqueous HCl. The solid was collected, washed with water and dried to give 320 mg of crude material, which was triturated form hot EtOH to give the title compound as a yellow powder (105 mg, 25%). ¹H NMR (400 MHz, DMSO-d₆) δ ppm 12.93 (br s, 1H), 8.94 (dd, J=4.2, 1.6 Hz, 1H), 8.40 (d, J=8.1 Hz, 1H), 8.16 (d, J=1.3 Hz, 1H), 8.13 (d, J=8.8 Hz, 1H), 8.05 (d, J=8.8 Hz, 1H), 7.90 (s, 1H), 7.86 (dd, J=8.9, 1.6 Hz, 1H), 7.80 (d, J=8.6 Hz, 1H), 7.61-7.55 (m, 2H), 7.52 (d, J=2.0 Hz, 1H), 3.65 (sext, J=8.0 Hz, 1H), 1.97-1.89 (m, 2H), 1.81-1.71 (m, 2H), 1.40-1.31 (m, 2H).

### Example 10

### (5Z)-2-(4-Pyrimidinylamino)-5-(6-quinolinylmethylidene)-1,3-thiazol-4(5H)-one

A mixture of 4-aminopyrimidine (70 mg, 0.74 mmoles) and (5Z)-2-(methylthio)-5-(6-quinolinylmethylidene)-1,3-thiazol-4(5H)-one (106 mg, 0.37 mmoles) in dioxan (0.5 mL) was sealed in a pressure flask and heated at 180° C. for 2 hours. When cool, the mixture was slurried in dichloromethane, the solid collected and washed with dichloromethane and slurried in boiling ethanol to afford the title compound (37 mg, 30%). 1H NMR (400 MHz, DMSO-d₆) δ ppm 7.28 (s, 1H) 7.59-7.67 (m, 1H) 7.89-7.95 (m, 1H) 8.04 (dd, J=8.84, 2.02 Hz, 1H) 8.13-8.21 (m, 1H) 8.33 (d, J=1.77 Hz, 1H) 8.51 (dd, J=8.8, 1.01 Hz, 1H) 8.76 (d, J=5.56 Hz, 1H) 8.94-9.02 (m, 1H) 9.14 (s, 1H) 12.88 (s, 1H).

### Example 11

### (5Z)-2-(1H-Pyrazol-3-ylamino)-5-(6-quinolinylmethylidene)-1,3-thiazol-4(5H)-one

A mixture of 3-aminopyrazole (80 mg, 1.0 mmoles) and (5Z)-2-(methylthio)-5-(6-quinolinylmethylidene)-1,3-thiazol-4(5H)-one (58 mg, 0.2 mmoles) in dioxan (1.0 mL) was sealed in a pressure flask and heated at 150° C. for 15 minutes. When cool, the mixture was slurried in dimethylformamide, the solid collected and washed with dimethylformamide and diethyl ether to afford the title compound (50 mg, 78%). 1H NMR (400 MHz, DMSO-d₆) δ ppm 6.17 (t, J=2.02 Hz, 1H) 7.62 (dd, J=8.34, 4.29 Hz, 1H) 7.75-7.83 (m, 2H) 7.98 (dd, J=8.84, 2.02 Hz, 1H) 8.19 (d, J=8.59 Hz, 1H) 8.26 (d, J=1.77 Hz, 1H) 8.46 (d, J=8.08 Hz, 1H) 8.98 (dd, J=4.29, 1.77 Hz, 1H) 12.32 (s, 1H) 12.82 (s, 1H)

### Example 12

### (5Z)-2-(4-Pyridinylamino)-5-(6-quinolinylmethylidene)-1,3-thiazol-4(5H)-one

A mixture of 4-aminopyridine (80 mg, 0.85 mmoles) and (5Z)-2-(methylthio)-5-(6-quinolinylmethylidene)-1,3-thiazol-4(5H)-one (95 mg, 0.34 mmoles) in dioxan (0.5 mL) was sealed in a pressure flask and heated at 180° C. for 2.5 hours. When cool, the mixture was slurried in ethanol, the solid collected and washed with ethanol and dichloromethane to afford the title compound (22 mg, 20%). 1H NMR (400 MHz, DMSO-d₆) δ ppm 7.34 (s, 1H) 7.59 (dd, J=8.21, 4.17 Hz, 1H) 7.88 (s, 1H) 7.93 (d, J=8.34 Hz, 1H) 8.12 (d, J=8.59 Hz, 1H) 8.20 (s, 1H) 8.47 (dd, J=8.59, 1.01 Hz, 1H) 8.56 (dd, J=4.80, 1.52 Hz, 2H) 8.96 (dd, J=4.17, 1.64 Hz, 1H) 12.55 (s, 1H)

### Example 13

### (5Z)-2-(2-Quinolinylamino)-5-(6-quinolinylmethylidene)-1,3-thiazol-4(5H)-one

Prepared by the method of example 12 using 2-aminoquinoline (68 mg, 0.47 mmoles) instead of 4-aminopyridine to give the title compound (92 mg, 72%). 1H NMR (400 MHz, DMSO-d₆) δ ppm 7.39 (d, J=8.59 Hz, 1H) 7.53-7.62 (m, 1H) 7.65 (dd, J=8.34, 4.29 Hz, 1H) 7.76-7.86 (m, 1H) 7.89 (s, 1H) 7.98 (d, J=7.83 Hz, 1H) 8.12 (d, J=8.59 Hz, 2H) 8.22 (d, J=8.84 Hz, 1H) 8.36 (s, 1H) 8.42 (d, J=8.59 Hz, 1H) 8.53 (d, J=7.83 Hz, 1H) 8.99 (dd, J=4.17, 1.64 Hz, 1H) 12.73 (d, J=4.29 Hz, 1H)

### Example 14

### (5Z)-2-[(4-Methyl-2-pyridinyl)amino]-5-(6-quinolinylmethylidene)-1,3-thiazol-4(5H)-one

Prepared by the method of example 12 using 2-amino-4-picoline (66 mg, 0.61 mmoles) instead of 4-aminopyridine to give the title compound (85 mg, 74%). 1H NMR (400 MHz, DMSO-d₆) δ ppm 2.36 (s, 3H) 7.04-7.11 (m, 2H) 7.63 (dd, J=8.21, 4.17 Hz, 1H) 7.82 (s, 1H) 8.03 (dd, J=8.84, 1.77 Hz, 1H) 8.17 (d, J=8.84 Hz, 1H) 8.30 (s, 1H) 8.42 (d, J=5.05 Hz, 1H) 8.50 (d, J=7.83 Hz, 1H) 8.98 (dd, J=4.17, 1.64 Hz, 1H) 12.53 (s, 1H)

### Example 15

### (5Z)-2-[(3-Methyl-2-pyridinyl)amino]-5-(6-quinolinylmethylidene)-1,3-thiazol-4(5H)-one

Prepared by the method of example 12 using 2-amino-3-picoline (68 mg, 0.63 mmoles) instead of 4-aminopyridine to give the title compound (71 mg, 62%). 1H NMR (400 MHz, DMSO-d₆) δ ppm 2.32-2.39 (m, 3H) 7.14 (dd, J=7.45, 4.93 Hz, 1H) 7.61 (dd, J=8.21, 4.17 Hz, 1H) 7.71 (d, J=6.57 Hz, 1H) 7.79 (s, 1H) 8.02 (dd, J=8.84, 2.02 Hz, 1H) 8.09-8.19 (m, 1H) 8.27 (d, J=1.77 Hz, 1H) 8.39 (dd, J=4.80, 1.26 Hz, 1H) 8.48 (d, J=7.58 Hz, 1H) 8.94-9.00 (m, 1H) 12.47 (s, 1H)

### Example 16

### (5Z)-2-[(6-Methyl-2-pyridinyl)amino]-5-(6-quinolinylmethylidene)-1,3-thiazol-4(5H)-one

Prepared by the method of example 12 using 2-amino-6-picoline (56 mg, 0.58 mmoles) instead of 4-aminopyridine to give the title compound (60 mg, 52%). 1H NMR (400 MHz, DMSO-d₆) δ ppm 2.60 (s, 3H) 7.02-7.12 (m, 2H) 7.64 (dd, J=8.34, 4.29 Hz, 1H) 7.77 (t, J=7.71 Hz, 1H) 7.81 (s, 1H) 8.05 (dd, J=8.97, 1.64 Hz, 1H) 8.16 (d, J=8.84 Hz, 1H) 8.30 (d, J=1.26 Hz, 1H) 8.45 (d, J=7.83 Hz, 1H) 8.98 (dd, J=4.17, 1.64 Hz, 1H) 12.50 (s, 1H)

### Example 17

### (5Z)-2-[(5-Iodo-2-pyridinyl)amino]-5-(6-quinolinylmethylidene)-1,3-thiazol-4(5H)-one

Prepared by the method of example 12 using 2-amino-5-iodopyridine (118 mg, 0.53 mmoles) instead of 4-aminopyridine to give the title compound (103 mg, 68%). 1H NMR (400 MHz, DMSO-d₆) δ ppm 7.07 (d, J=8.34 Hz, 1H) 7.62 (dd, J=8.21, 4.17 Hz, 1H) 7.79-7.89 (m, 1H) 8.02 (dd, J=8.84, 1.77 Hz, 1H) 8.12-8.23 (m, 2H) 8.28 (d, J=1.52 Hz, 1H) 8.52 (d, J=8.08 Hz, 1H) 8.79 (d, J=1.77 Hz, 1H) 8.98 (dd, J=4.17, 1.64 Hz, 1H) 12.62 (s, 1H)

### Example 18

### (5Z)-2-(1H-Benzimidazol-2-ylamino)-5-(6-quinolinylmethylidene)-1,3-thiazol-4(5H)-one

Prepared by the method of example 12 using 2-aminobenzimidazole (69 mg, 0.52 mmoles) instead of 4-aminopyridine to give the title compound (84 mg, 68%). 1H NMR (400 MHz, DMSO-d₆) δ ppm 7.08 (dd, J=5.81, 3.03 Hz, 1H) 7.26 (dd, J=5.81, 3.28 Hz, 2H) 7.62 (dd, J=8.34, 4.04 Hz, 2H) 7.86 (s, 1H) 8.02 (d, J=8.34 Hz, 1H) 8.16 (d, J=8.84 Hz, 1H) 8.27 (s, 1H) 8.50 (d, J=8.08 Hz, 1H) 8.97 (d, J=2.78 Hz, 1H) 12.52 (br. s, 2H).

### Example 19

### N-(3-{[(5Z)-4-Oxo-5-(6-quinolinylmethylidene)-4,5-dihydro-1,3-thiazol-2-yl]amino}phenyl)acetamide

A microwave vial was charged with (5Z)-2-(methylthio)-5-(6-quinolinylmethylidene)-1,3-thiazol-4(5H)-one (0.100 g, 0.349 mmole) and N-(3-aminophenyl)acetamide (0.105 g, 0.699 mmole) in ethylene glycol (2.0 mL). The contents were sealed and irradiated at 120° C. for 30 min in a microwave reactor. The mixture was allowed to cool to room temperature and taken up in water. The resulting precipitate was filtered off, washed with water and ethyl acetate and dried under vacuum to afford the title compound (0.106 g, 78%). 1H NMR (400 MHz, DMSO-d₆) δ ppm 2.05 (d, 3H), 6.75 (s, 1H), 7.27-7.46 (m, 2H), 7.49-7.66 (m, 1H), 7.78-8.02 (m, 2H), 8.02-8.28 (m, 2H), 8.45 (t, J=8.72 Hz, 1H), 8.95 (dd, J=16.17, 3.28 Hz, 1H), 10.08 (d, J=41.68 Hz, 1H), 11.68 (s, 1H), 12.46 (s, 1H).

### Example 20

### 1,1-Dimethylethyl (2-{[(5Z)-4-oxo-5-(6-quinolinylmethylidene)-4,5-dihydro-1,3-thiazol-2-yl]amino}phenyl)carbamate

The procedure of example 19 was followed here, using 1,1-dimethylethyl (2-aminophenyl)carbamate in place of N-(3-aminophenyl)acetamide. The product was recrystallised (ethyl acetate) to afford the title compound (0.078 g, 46%) as an orange solid. 1H NMR (400 MHz, DMSO-d₆) δ ppm 1.35-1.53 (m, 9H), 7.01-7.22 (m, 3H), 7.57 (dd, J=8.34, 4.04 Hz, 1H), 7.80 (d, J=7.83 Hz, 1H), 7.83 (s, 1H), 7.88 (dd, J=8.84, 2.02 Hz, 1H), 8.10 (d, J=8.84 Hz, 1H), 8.13-8.22 (m, J=2.02 Hz, 2H), 8.45 (d, J=7.58 Hz, 1H), 8.94 (dd, J=4.17, 1.64 Hz, 1H), 12.56 (s, 1H).

### Example 21

### N-(2-{[(5Z)-4-Oxo-5-(6-quinolinylmethylidene)-4,5-dihydro-1,3-thiazol-2-yl]amino}phenyl)acetamide

The procedure of example 19 was followed here, using N-(2-aminophenyl)acetamide in place of N-(3-aminophenyl)acetamide. The product was recrystallised (ethyl acetate) to afford the title compound (0.015 g, 21%) as a solid. 1H NMR (400 MHz, DMSO-d₆) δ ppm 1.87 (s, 3H), 6.85-6.91 (m, 1H), 6.92-7.00 (m, 2H), 7.38 (dd, J=8.34, 4.04 Hz, 1H), 7.66 (s, 1H), 7.69 (dd, J=8.84, 2.02 Hz, 1H), 7.74-7.80 (m, 1H), 7.91 (d, J=8.84 Hz, 1H), 7.96 (d, J=1.77 Hz, 1H), 8.26 (d, J=7.58 Hz, 1H), 8.75 (dd, J=4.17, 1.64 Hz, 1H), 8.83 (s, 1H), 12.30 (s, 1H).

### Example 22

### (5Z)-2-(2-Pyrimidinylamino)-5-(6-quinolinylmethylidene)-1,3-thiazol-4(5H)-one

Prepared by the method of example 12 using 2-aminopyrimidine (60 mg, 0.55 mmoles) instead of 4-aminopyridine gave the title compound (8 mg, 4%) after flash chromatography (silica gel, 0-10% methanol in dichloromethane) 1H NMR (400 MHz, DMSO-d₆) δ ppm 7.32 (s, 1H) 7.64 (dd, J=8.34, 4.29 Hz, 1H) 7.90 (s, 1H) 8.05 (d, J=6.82 Hz, 1H) 8.18 (d, J=8.84 Hz, 1H) 8.33 (s, 1H) 8.50 (s, 1H) 8.87 (d, J=4.80 Hz, 2H) 8.99 (dd, J=4.17, 1.64 Hz, 1H) 12.88 (s, 1H).

### Example 23

### (5Z)-2-(3-Quinolinylamino)-5-(6-quinolinylmethylidene)-1,3-thiazol-4(5H)-one

Prepared by the method of example 12 using 3-aminoquinoline (77 mg, 0.50 mmoles) instead of 4-aminopyridine to give the title compound (10 mg, 7%) after flash chromatography (silica gel, 0-10% methanol in dichloromethane) 1H NMR (400 MHz, DMSO-d₆) δ ppm 7.53 (dd, J=8.34, 4.29 Hz, 1H) 7.65 (t, J=7.20 Hz, 1H) 7.75 (t, J=7.58 Hz, 1H) 7.86 (d, J=2.02 Hz, 1H) 7.88 (s, 1H) 7.97-8.08 (m, 4H) 8.16 (d, J=1.26 Hz, 1H) 8.43 (d, J=8.08 Hz, 1H) 8.72 (d, J=2.27 Hz, 1H) 8.92 (dd, J=4.04, 1.52 Hz, 1H) 12.78 (s, 1H).

### Example 24

### (5Z)-2-[(1-Methyl-1H-indol-2-yl)amino]-5-(6-quinolinylmethylidene)-1,3-thiazol-4(5H)-one trifluoroacetate

Prepared by the method of example 12 using 2-amino-1-methylbenzimidazole (95 mg, 0.65 mmoles) instead of 4-aminopyridine to give the title compound (28 mg, 19%) after preparative HPLC (water-acetonitrile-0.10% TFA). 1H NMR (400 MHz, DMSO-d₆) δ ppm 3.78 (s, 3H) 7.24 (s, 1H) 7.37 (s, 1H) 7.46-7.55 (m, 1H) 7.63 (s, 1H) 7.65 (d, J=2.27 Hz, 1H) 7.76 (s, 1H) 7.85 (s, 1H) 7.91 (s, 1H) 8.04 (s, 1H) 8.13 (d, J=8.08 Hz, 1H) 8.24 (s, 1H) 8.32 (s, 1H) 8.52 (s, 1H) 8.96 (s, 1H) 12.73 (s, 1H) 12.88 (s, 1H)

### Example 25

### (5Z)-2-[(5-Chloro-2-pyridinyl)amino]-5-(6-quinolinylmethylidene)-1,3-thiazol-4(5H)-one

Prepared by the method of example 12 using 2-amino-5-chloropyridine (68 mg, 0.53 mmoles) instead of 4-aminopyridine to give the title compound (35 mg, 20%) from ethanol. 1H NMR (400 MHz, DMSO-d₆) δ ppm 7.26 (d, J=8.59 Hz, 1H) 7.63 (dd, J=8.34, 4.29 Hz, 1H) 7.84 (s, 1H) 8.00 (ddd, J=17.18, 8.72, 2.15 Hz, 2H) 8.16 (d, J=8.84 Hz, 1H) 8.29 (d, J=1.77 Hz, 1H) 8.51 (d, J=7.83 Hz, 1H) 8.63 (d, J=2.27 Hz, 1H) 8.98 (dd, J=4.29, 1.77 Hz, 1H) 12.63 (s, 1H)

### Example 26

### 1,1-Dimethylethyl [(2-{[(5Z)-4-oxo-5-(6-quinolinylmethylidene)-4,5-dihydro-1,3-thiazol-2-yl]amino}phenyl)methyl]carbamate

a) 1,1-Dimethylethyl ({2-[(4-oxo-4,5-dihydro-1,3-thiazol-2-yl)amino]phenyl}methyl)carbamate. The method of example 6(a) was followed, using 1,1-dimethylethyl [(2-aminophenyl)methyl]carbamate (J-M. Hah et. al., J. Med. Chem., 2003, 46(9), 1661) in place of 1,1-dimethylethyl [(3-amino-4-chlorophenyl)methyl]carbamate, to give the title compound in an impure form, used directly in the next step.

b) 1,1-Dimethylethyl [(2-{[(5Z)-4-oxo-5-(6-quinolinylmethylidene)-4,5-dihydro-1,3-thiazol-2-yl]amino}phenyl)methyl]carbamate. A mixture of the compound from example 26(a) (0.500 g, 1.56 mmol), quinoline-6-carbaldehyde (0.250 g, 1.59 mmol), morpholine (0.024 g, 0.275 mmol) and toluene (8 mL) was heated under reflux for 12 h, then cooled and evaporated to dryness under reduced pressure. The residue was slurried in methanol (20 mL) and water (2 mL) added. The precipitate was filtered, washed (10% aq methanol) and dried to give the title compound (0.522 g, 73%) as a solid. 1H NMR (400 MHz, DMSO-d₆) δ 1.33 (s, 9H), 4.08 (d, J=6.0 Hz, 2H), 6.97 (br s, 1H), 7.17-7.32 (m, 4H), 7.56 (dd, J=8.4, 4.0 Hz, 1H), 7.81 (s, 1H), 7.84 (dd, J=8.8, 2.0 Hz, 1H), 8.08 (d, J=8.8 Hz, 1H), 8.13 (d, J=1.2 Hz, 1H), 8.43 (d, J=8.4 Hz, 1H), 8.93 (dd, J=4.0, 1.6 Hz, 1H), 12.50 (br s, 1H).

### Example 27

### (5Z)-2-{[2-Chloro-6-(trifluoromethyl)-3-pyridinyl]amino}-5-(6-quinolinylmethylidene)-1,3-thiazol-4(5H)-one trifluoroacetate

A mixture of 3-amino-2-chloro-6-trifluoromethylpyridine (80 mg, 0.40 mmoles) and (5Z)-2-(methylthio)-5-(6-quinolinylmethylidene)-1,3-thiazol-4(5H)-one (70 mg, 0.24 mmoles) in acetic acid (1.0 mL) was sealed in a pressure flask and heated at 180° C. for 3.0 hours. When cool, the mixture was purified by preparative HPLC, then crystallized from methanol to afford the title compound (10 mg, 10%). 1H NMR (400 MHz, DMSO-d₆) δ ppm 7.59 (dd, J=8.21, 4.17 Hz, 1H) 7.93 (s, 3H) 8.02 (s, 1H) 8.09 (d, J=8.84 Hz, 1H) 8.20 (s, 1H) 8.48 (s, 1H) 8.95 (dd, J=4.04, 1.52 Hz, 1H) 13.07 (s, 1H)

### Example 28

### N-(4-{[(5Z)-4-Oxo-5-(6-quinolinylmethylidene)-4,5-dihydro-1,3-thiazol-2-yl]amino}phenyl)acetamide

The procedure of example 19 was followed here, using N-(4-aminophenyl)acetamide in place of N-(3-aminophenyl)acetamide, to afford the title compound (0.122 g, 90%). 1H NMR (400 MHz, DMSO-d₆) δ ppm 2.06 (d, J=4.55 Hz, 3H), 7.04 (s, 1H), 7.57 (dd, J=8.34, 4.04 Hz, 1H), 7.60-7.68 (m, 3H), 7.69-7.77 (m, 1H), 7.85-7.93 (m, 1H), 8.16 (dd, J=5.18, 3.16 Hz, 1H), 8.42-8.51 (m, 1H), 8.86-9.01 (m, 1H), 9.95-10.08 (m, 1H), 11.66 (s, 1H), 12.44 (s, 1H).

### Example 29

### (5Z)-2-{[6-(Methyloxy)-4-pyrimidinyl]amino}-5-(6-quinolinylmethylidene)-1,3-thiazol-4(5H)-one

A mixture of 4-amino-6-methoxypyrimidine (68 mg, 0.54 mmoles) and (5Z)-2-(methylthio)-5-(6-quinolinylmethylidene)-1,3-thiazol-4(5H)-one (111 mg, 0.39 mmoles) in acetic acid (1.0 mL) was sealed in a pressure flask and heated at 180° C. for 4.0 hours. When cool, the mixture was diluted with ethanol, the solid collected, slurried in boiling ethanol to give the title compound (13 mg, 9%). 1H NMR (400 MHz, DMSO-d₆) δ ppm 3.96 (s, 3H) 7.64 (dd, J=8.34, 4.04 Hz, 1H) 7.88 (s, 1H) 8.02 (dd, J=8.84, 2.02 Hz, 1H) 8.18 (d, J=8.84 Hz, 1H) 8.31 (d, J=1.77 Hz, 1H) 8.50 (d, J=7.58 Hz, 1H) 8.80 (s, 1H) 8.99 (dd, J=4.29, 1.77 Hz, 1H) 12.56 (s, 1H)

### Example 30

### 3-Methyl-N-(2-{[(5Z)-4-oxo-5-(6-quinolinylmethylidene)-4,5-dihydro-1,3-thiazol-2-yl]amino}phenyl)butanamide

1,1-Dimethylethyl (2-{[(5Z)-4-oxo-5-(6-quinolinylmethylidene)-4,5-dihydro-1,3-thiazol-2-yl]amino}phenyl)carbamate (example 20, 0.030 g, 0.0672 mmole) was treated with trifluoroacetic acid (1.0 mL) and allowed to stir for 15 minutes. This was then diluted with dichloromethane and concentrated, then dried. The crude amine salt in methyl ethyl ketone (0.50 mL) was treated with potassium carbonate (0.029 g, 0.209 mmol) and isovaleryl chloride (0.008 mL, 0.0656 mmol) and allowed to stir for 2 hours at 21° C. to 80° C. The reaction mixture was diluted with ethyl acetate and water and extracted. The organic was concentrated and purified via column chromatography (1:1 ethyl acetate:hexane to 10% methanol in dichloromethane) to provide the title compound (0.010 g, 33%) as a yellow solid. 1H NMR (400 MHz, DMSO-d₆) δ ppm 0.91 (d, J=6.57 Hz, 6H), 2.05 (m, 1H), 2.22 (d, J=7.07 Hz, 2H), 7.06 (s, 1H), 7.15 (s, 1H), 7.57 (dd, J=8.34, 4.04 Hz, 1H), 7.83 (s, 1H), 7.85-7.90 (m, 1H), 7.93 (d, J=8.34 Hz, 1H), 8.09 (d, J=8.59 Hz, 1H), 8.15 (s, 1H), 8.45 (d, J=8.59 Hz, 1H), 8.94 (dd, J=4.17, 1.64 Hz, 1H).

### Example 31

### N-(4-Chloro-3-{[(5Z)-4-oxo-5-(6-quinolinylmethylidene)-4,5-dihydro-1,3-thiazol-2-yl]amino}phenyl)-3-methylbutanamide, trifluoroacetate salt

a) 1,1-Dimethylethyl (3-amino-4-chlorophenyl)carbamate. A solution of di-t-butyl dicarbonate (3.37 g, 15.4 mmol) in dichloromethane (8 mL) was added over 5 min to a stirred suspension of 2-chloro-1,3-benzenediamine (2.00 g, 14.0 mmol) in dichloromethane (22 mL) at room temperature. After stirring 48 h, the solvent was removed under reduced pressure and the residue slurried in dichloromethane (15 mL), then filtered, washed with 20% dichloromethane/hexane and dried. The filtrate was evaporated to dryness under reduced pressure and a second crop of product obtained by recrystallisation from 2:1 hexane:ether. Both crops were combined to give the title compound (2.83 g, 83%) as a solid. 1H NMR (400 MHz, DMSO-d₆) δ 1.46 (s, 9H), 5.25 (br s, 2H), 6.56 (dd, J=8.7, 2.4 Hz, 1H), 7.01 (d, J=8.6 Hz, 1H), 7.08 (d, J=2.0 Hz, 1H), 9.18 (s, 1H).

b) 1,1-Dimethylethyl {4-chloro-3-[(4-oxo-4,5-dihydro-1,3-thiazol-2-yl)amino]phenyl}carbamate. A mixture of the compound from example 31(a) (1.75 g, 7.21 mmol), 2-(methylthio)-1,3-thiazol-4(5H)-one (1.27 g, 8.63 mmol) and ethanol (25 mL) was heated under reflux for 24 h, then cooled. The solid was filtered, washed with ethanol and dried to give the title compound (1.38 g, 56%) as a solid. 1H NMR (400 MHz, DMSO-d₆) δ1.47 (s, 9H), 4.03 (s, 2H), 7.14-7.25 (m, 2H), 7.33 (d, J=8.6, 1H), 9.51 (s, 1H), 11.97 (br s, 1H).

c) 2-[(5-Amino-2-chlorophenyl)amino]-1,3-thiazol-4(5H)-one. A solution of the compound from example 31(b) (0.200 g, 0.585 mmol) in trifluoroacetic acid (3 mL) was stirred at room temperature for 20 min, then evaporated under reduced pressure. The residue was dissolved in water (20 mL), filtered, then adjusted to pH 7-8 with aq NaOH. The precipitate was filtered after 2 h, washed with water and dried to leave the title compound (0.105 g, 74%) as a pale orange powder. 1H NMR (400 MHz, DMSO-d₆) δ ppm 3.99 (s, 2H) 5.27 (s, 2H) 6.19 (s, 1H) 6.33 (d, J=6.57 Hz, 1H) 7.05 (d, J=8.59 Hz, 1H) 11.84 (s, 1H)

d) N-(4-Chloro-3-{[(5Z)-4-oxo-5-(6-quinolinylmethylidene)-4,5-dihydro-1,3-thiazol-2-yl]amino}phenyl)-3-methylbutanamide, trifluoroacetate salt. Potassium carbonate (0.066 g, 0.478 mmol) was added to a slurry of the compound from example 31(c) (0.058 g, 0.238 mmol) in 2-butanone (4 mL), followed by isovaleryl chloride (0.029 mL, 0.238 mmol). The mixture was stirred at room temperature for 18 h, then evaporated under reduced pressure and the residue partitioned between brine and ethyl acetate. The extracts were dried (MgSO₄) and evaporated under reduced pressure to give the crude amide product. A mixture of the amide, quinoline-6-carbaldehyde (0.038 g, 0.239 mmol), sodium acetate (0.040 g, 0.488 mmol) and ethanol (2 mL) was irradiated at 150° C. for 40 min in a microwave reactor. The mixture was allowed to cool to room temperature, then evaporated under reduced pressure and purified by rp-HPLC (ODS, 10-90% acetonitrile/water+0.1% trifluoroacetic acid) to afford the title compound (0.006 g, 4%) as a solid. 1H NMR (400 MHz, DMSO-d₆) δ ppm 0.92 (d, J=6.57 Hz, 6H), 2.02-2.11 (m, 1H), 2.19 (d, J=7.07 Hz, 2H), 7.39-7.49 (m, 3H), 7.60 (dd, J=8.21, 4.17 Hz, 1H), 7.86-7.91 (m, 2H), 8.10 (d, J=8.59 Hz, 1H), 8.19 (d, J=1.52 Hz, 1H), 8.50 (d, J=8.84 Hz, 1H), 8.96 (dd, J=4.29, 1.52 Hz, 1H), 10.07 (s, 1H), 12.77 (brs, 1H).

### Example 32

### (5Z)-2-[(4-Methyl-1,3-thiazol-2-yl)amino]-5-(6-quinolinylmethylidene)-1,3-thiazol-4(5H)-one trifluoroacetate

A mixture of 2-amino-4-methylthiazole (34 mg, 0.30 mmoles) and (5Z)-2-(methylthio)-5-(6-quinolinylmethylidene)-1,3-thiazol-4(5H)-one (56 mg, 0.20 mmoles) in acetic acid (1.0 mL) was sealed in a pressure flask and heated at 180° C. for 4.0 hours. Purification by preparative HPLC gave the title compound (4.6 mg, 6.5%). 1H NMR (400 MHz, DMSO-d₆) δ ppm 2.41 (s, 3H) 6.98 (s, 0.5H) 7.08 (s, 0.5H) (rotamers), 7.64 (dd, J=8.21, 4.17 Hz, 1H) 7.82-7.92 (m, 1H) 7.97-8.06 (m, 1H) 8.11-8.22 (m, 1H) 8.23-8.31 (m, 1H) 8.49 (d, J=7.58 Hz, 1H) 8.94-9.01 (m, 1H) 12.75 (s, 1H)

### Example 33

### Ethyl 2-{[(5Z)-4-oxo-5-(6-quinolinylmethylidene)-4,5-dihydro-1,3-thiazol-2-yl]amino}-4,5,6,7-tetrahydro-1-benzothiophene-3-carboxylate trifluoroacetate

A mixture of ethyl 2-amino-4,5,6,7-tetrahydro-1-benzothiophene-3-carboxylate (73 mg, 0.32 mmoles) and (5Z)-2-(methylthio)-5-(6-quinolinylmethylidene)-1,3-thiazol-4(5H)-one (56 mg, 0.20 mmoles) in acetic acid (1.0 mL) was sealed in a pressure flask and heated at 180° C. for 4.0 hours. Purification by preparative HPLC gave the title compound (13 mg, 14%). 1H NMR (400 MHz, DMSO-d₆) δ ppm 1.16 (t, J=7.07 Hz, 3H) 1.77 (m, 4H) 2.68 (d, J=5.56 Hz, 4H) 4.11 (q, J=7.07 Hz, 2H) 7.63 (dd, J=8.21, 4.17 Hz, 1H) 7.89-7.98 (m, 2H) 8.14 (d, J=8.84 Hz, 1H) 8.23 (s, 1H) 8.54 (d, J=8.08 Hz, 1H) 8.98 (d, J=2.78 Hz, 1H) 12.88 (s, 1H)

### Example 34

### (5Z)-2-(2-Biphenylylamino)-5-(6-quinolinylmethylidene)-1,3-thiazol-4(5H)-one

A microwave vial was charged with (5Z)-2-[(2-bromophenyl)amino]-5-(6-quinolinylmethylidene)-1,3-thiazol-4(5H)-one (0.10 g, 0.244 mmole) was treated with phenyl boronic acid (0.030 g, 0.246 mmole) potassium carbonate (0.067 g, 0.0485 mmol) and tetrakis(triphenylphosphine)palladium(0) 0.017 g, 0.06 mole %) in dioxane (1.0 mL). The vial was flushed with nitrogen and the contents sealed and irradiated at 130° C. for 60 min in a microwave reactor. The mixture was allowed to cool to room temperature and diluted with water and extracted with ethyl acetate. Column chromatography purification (100% Ethyl acetate to 10% MeOH/CH₂Cl₂) provided the title compound (0.024 g, 27%) as a solid. 1H NMR (400 MHz, DMSO-d₆) δ ppm 7.13 (s, 1H), 7.26-7.36 (m, 2H), 7.37-7.49 (m, 6H), 7.57 (dd, J=8.34, 4.04 Hz, 1H), 7.81 (s, 1H), 7.88 (dd, J=8.84, 2.02 Hz, 1H), 8.10 (d, J=8.84 Hz, 1H), 8.16 (d, J=1.77 Hz, 1H), 8.47 (d, J=7.33 Hz, 1H), 8.94 (dd, J=4.17, 1.64 Hz, 1H), 12.51 (s, 1H).

### Example 35

### (5Z)-2-{[4′-(Methyloxy)-2-biphenylyl]amino}-5-(6-quinolinylmethylidene)-1,3-thiazol-4(5H)-one

The procedure of example 34 was followed here, using 4-methoxyphenyl boronic acid in place of phenyl boronic acid, to afford the title compound (0.047 g, 44%). 1H NMR (400 MHz, DMSO-d₆) δ ppm 3.77 (s, 3H), 6.98 (d, J=8.59 Hz, 2H), 7.09 (s, 1H), 7.27-7.48 (m, 5H), 7.57 (dd, J=8.21, 4.17 Hz, 1H), 7.82 (s, 1H), 7.84-7.90 (m, 1H), 8.10 (d, J=8.59 Hz, 1H), 8.15 (d, J=2.02 Hz, 1H), 8.46 (d, J=7.58 Hz, 1H), 8.94 (dd, J=4.29, 1.77 Hz, 1H), 12.51 (s, 1H)

### Example 36

### (5Z)-2-{[4′-(Dimethylamino)-2-biphenylyl]amino}-5-(6-quinolinylmethylidene)-1,3-thiazol-4(5H)-one

The procedure of example 34 was followed here, using 4-dimethylaminophenyl boronic acid in place of phenyl boronic acid, to afford the title compound (0.024 g, 22%). [MS (ES+) m/e 451.4 [M+H]⁺.

### Example 37

### 2′-{[(5Z)-4-Oxo-5-(6-quinolinylmethylidene)-4,5-dihydro-1,3-thiazol-2-yl]amino}-3-biphenylcarbonitrile

The procedure of example 34 was followed here, using 3-cyanophenyl boronic acid in place of phenyl boronic acid, to afford the title compound (0.004 g, 5%). 1H NMR (400 MHz, DMSO-d₆) δ ppm 7.18 (d, J=6.82 Hz, 1H), 7.35 (t, J=6.95 Hz, 1H), 7.45-7.54 (m, 2H), 7.57 (d, J=8.34, 4.04 Hz, 1H), 7.63 (t, J=7.83 Hz, 1H), 7.75 (d, J=8.08 Hz, 1H), 7.78-7.83 (m, 2H), 7.84-7.90 (m, 2H), 8.09 (d, J=8.84 Hz, 1H), 8.15 (d, J=1.77 Hz, 1H), 8.46 (d, J=7.83 Hz, 1H), 8.94 (dd, J=4.17, 1.64 Hz, 1H), 12.53 (s, 1H)

### Example 38

### (5Z)-2-{[2-(1,3-Benzodioxol-5-yl)phenyl]amino}-5-(6-quinolinylmethylidene)-1,3-thiazol-4(5H)-one

The procedure of example 34 was followed here, using 3,4-methenedioxyphenyl boronic acid in place of phenyl boronic acid, to afford the title compound (0.021 g, 19%). 1H NMR (400 MHz, DMSO-d₆) δ ppm 6.04 (s, 2H), 6.86 (dd, J=7.96, 1.89 Hz, 1H), 6.92-6.99 (m, 2H), 7.10 (s, 1H), 7.23-7.33 (m, 1H), 7.35-7.46 (m, 2H), 7.57 (dd, J=8.34, 4.29 Hz, 1H), 7.81 (s, 1H), 7.87 (dd, J=8.84, 2.02 Hz, 1H), 8.09 (d, J=8.84 Hz, 1H), 8.15 (d, J=1.77 Hz, 1H), 8.46 (d, J=8.59 Hz, 1H), 8.94 (dd, J=4.17, 1.64 Hz, 1H), 12.53 (s, 1H)

### Example 39

### Ethyl 1-methyl-5-{[(5Z)-4-oxo-5-(6-quinolinylmethylidene)-4,5-dihydro-1,3-thiazol-2-yl]amino}-1H-pyrazole-4-carboxylate trifluoroacetate

A mixture of ethyl 2-amino-4,5,6,7-tetrahydro-1-benzothiophene-3-carboxylate (50 mg, 0.30 mmoles) and (5Z)-2-(methylthio)-5-(6-quinolinylmethylidene)-1,3-thiazol-4(5H)-one (56 mg, 0.20 mmoles) in isobutyric acid (1.0 mL) was heated under reflux overnight. Purification by preparative HPLC gave the title compound (4.5 mg, 5.5%). 1H NMR (400 MHz, DMSO-d₆) δ ppm 1.13 (t, J=7.20 Hz, 3H) 3.65 (s, 3H) 4.10 (q, J=6.99 Hz, 2H) 7.60 (dd, J=8.34, 4.04 Hz, 1H) 7.82-7.91 (m, 2H) 7.93 (s, 1H) 8.11 (d, J=8.59 Hz, 1H) 8.18 (s, 1H) 8.50 (d, J=7.58 Hz, 1H) 8.96 (dd, J=4.29, 1.52 Hz, 1H) 13.13 (s, 1H)

### Example 40

### (5Z)-2-{[2-(4-Pyridinyl)phenyl]amino}-5-(6-quinolinylmethylidene)-1,3-thiazol-4(5H)-one

The procedure of example 34 was followed here, using pyridine-4-boronic acid in place of phenyl boronic acid, to afford the title compound (0.019 g, 19%). 1H NMR (400 MHz, DMSO-d₆) δ ppm 7.18 (d, J=7.83 Hz, 1H), 7.36 (t, J=7.07 Hz, 1H), 7.40-7.47 (m, 2H), 7.47-7.55 (m, 2H), 7.57 (dd, J=8.34, 4.29 Hz, 1H), 7.82 (s, 1H), 7.87 (dd, J=8.84, 2.02 Hz, 1H), 8.09 (d, J=9.09 Hz, 1H), 8.16 (d, J=1.77 Hz, 1H), 8.46 (d, J=7.83 Hz, 1H), 8.56-8.62 (m, 2H), 8.94 (dd, J=4.17, 1.64 Hz, 1H), 12.54 (s, 1H)

### Example 41

### (5Z)-2-{[2-Chloro-5-(hydroxymethyl)phenyl]amino}-5-(6-quinolinylmethylidene)-1,3-thiazol-4(5H)-one

a) (3-Amino-4-chlorophenyl)methanol. A solution of borane in tetrahydrofuran (1M, 6.00 mL, 6.00 mmol) was injected into a stirred, ice-cooled solution of 3-amino-4-chlorobenzoic acid (0.350 g, 2.04 mmol) in tetrahydrofuran (10 mL) under nitrogen. The mixture was stirred 4 h at room temperature, then cooled in ice and 1M aqueous sodium hydroxide (18 mL) added. Water (150 mL) was added and the pH adjusted to 10 with aqueous hydrochloric acid, before extracting with ethyl acetate. The extracts were washed (brine), dried (MgSO₄), evaporated under reduced pressure and the residue chromatographed (silica gel, 30-80% ethyl acetate/hexane) to give the title compound (0.270 g, 84%) as a white solid. 1H NMR (400 MHz, DMSO-d₆) δ 4.35 (d, J=5.6 Hz, 2H), 5.12 (t, J=5.6 Hz, 1H), 5.29 (br s, 2H), 6.47 (dd, J=8.0, 2.0 Hz, 1H), 6.77 (d, J=2.0 Hz, 1H), 7.10 (d, J=8.0 Hz, 1H).

b) 2-{[2-Chloro-5-(hydroxymethyl)phenyl]amino}-1,3-thiazol-4(5H)-one. The method of example 6(a) was followed, using the compound of example 41(a) in place of 1,1-dimethylethyl [(3-amino-4-chlorophenyl)methyl]carbamate, to give the title compound (34%) as an oil. 1H NMR (400 MHz, DMSO-d₆) δ 4.02 (s, 2H), 4.47 (d, J=5.6 Hz, 2H), 5.31 (t, J=5.8 Hz, 1H), 6.97 (s, 1H), 7.07 (d, J=7.6 Hz, 1H), 7.43 (d, J=8.4 Hz, 1H), 11.98 (br s, 1H).

c) (5Z)-2-{[2-Chloro-5-(hydroxymethyl)phenyl]amino}-5-(6-quinolinylmethylidene)-1,3-thiazol-4(5H)-one. A mixture of the compound from example 41(b) (0.060 g, 0.234 mmol), quinoline-6-carbaldehyde (0.037 g, 0.235 mmol), morpholine (0.020 g, 0.230 mmol) and toluene (2 mL) was heated under reflux for 2 h, then acetic acid (0.020 g, 0.333 mmol) added and the reflux continued for 2 h. Acetic acid (0.1 g, 1.7 mmol) was added and the mixture cooled, then saturated aqueous ammonium chloride (˜0.1 mL) added. The liquor was decanted off and residue washed (toluene, water, ether) and dried to give the title compound (0.043 g, 50%) as a yellow solid. 1H NMR (400 MHz, DMSO-d₆) δ 4.51 (d, J=6.0 Hz, 2H), 5.34 (t, J=5.8 Hz, 1H), 7.08 (s, 1H), 7.17 (dd, J=8.0, 1.6 Hz, 1H), 7.50 (d, J=8.4 Hz, 1H), 7.57 (dd, J=8.4, 4.0 Hz, 1H), 7.84-7.87 (m, 2H), 8.09 (d, J=8.8 Hz, 1H), 8.15 (d, J=1.2 Hz, 1H), 8.45 (d, J=8.0 Hz, 1H), 8.94 (dd, J=4.0, 1.6 Hz, 1H), 12.71 (brs, 1H).

### Example 42

### 1,1-Dimethylethyl [(4-chloro-3-{[(5Z)-4-oxo-5-(6-quinolinylmethylidene)-4,5-dihydro-1,3-thiazol-2-yl]amino}phenyl)methyl]carbamate

Preparation described in example 6(b).

### Example 43

### (5Z)-2-{[5-(Aminomethyl)-2-chlorophenyl]amino}-5-(6-quinolinylmethylidene)-1,3-thiazol-4(5H)-one

Preparation described in example 6(c).

### Example 44

### N-(4-Chloro-3-{[(5Z)-4-oxo-5-(6-quinolinylmethylidene)-4,5-dihydro-1,3-thiazol-2-yl]amino}phenyl)propanamide

Propionyl chloride (0.072 mL, 0.830 mmol) was injected into a stirred mixture of the compound from example 31(c) (0.200 g, 0.830 mmol), potassium carbonate (0.02 g, 0.145 mmol) and dioxane (2 mL) and stirring continued for 0.5 h. The mixture was diluted with water (20 mL) and extracted with ethyl acetate. The extracts were dried (MgSO₄) and evaporated under reduced pressure. The residue was triturated with a small amount of methanol to give the crude amide as a white solid (0.177 g, ˜0.593 mmol). A mixture of the amide, quinoline-6-carbaldehyde (0.093 g, 0.593 mmol), sodium acetate (0.097 g, 1.18 mmol) and ethanol (2 mL) was irradiated at 150° C. for 40 min in a microwave reactor. The mixture was allowed to cool to room temperature, diluted with water (2 mL) and the solid filtered, washed with ethyl acetate and dried to afford the title compound (0.052 g, 14%) as a yellow solid. 1H NMR (400 MHz, DMSO-d₆) δ ppm 1.06 (t, J=7.5 Hz, 3H) 2.33 (q, J=7.49 Hz, 2H) 7.39-7.49 (m, 3H) 7.57 (dd, J=8.34, 4.29 Hz, 1H) 7.83-7.91 (m, 2H) 8.09 (d, J=8.84 Hz, 1H) 8.17 (d, J=1.52 Hz, 1H) 8.47 (d, J=7.83 Hz, 1H) 8.94 (dd, J=4.29, 1.77 Hz, 1H) 10.08 (s, 1H) 12.77 (s, 1H)

### Example 45

### N-(4-Chloro-3-{[(5Z)-4-oxo-5-(6-quinolinylmethylidene)-4,5-dihydro-1,3-thiazol-2-yl]amino}phenyl)-2-(methyloxy)acetamide

The procedure of example 44 was followed here, using methoxyacetyl chloride in place of propionyl chloride, to give the title compound (17%) as a solid. 1H NMR (400 MHz, DMSO-d₆) δ ppm 3.36 (s, 3H) 4.01 (s, 2H) 7.49-7.58 (m, 4H) 7.84-7.88 (m, 2H) 8.09 (d, J=8.84 Hz, 1H) 8.17 (s, 1H) 8.47 (d, J=8.10 Hz, 1H) 8.94 (d, J=2.80 Hz, 1H) 9.99 (s, 1H) 12.78 (s, 1H)

### Example 46

### N-(4-Chloro-3-{[(5Z)-4-oxo-5-(6-quinolinylmethylidene)-4,5-dihydro-1,3-thiazol-2-yl]amino}phenyl)-2-(2-thienyl)acetamide

Thiophene-2-acetyl chloride (0.250 mL, 2.03 mmol) was injected into a stirred mixture of the compound from example 31(c) (0.190 g, 0.788 mmol), pyridine (0.03 g, 0.379 mmol) and dioxane (4 mL) and stirring continued for 0.5 h. The mixture was diluted with water (40 mL) and extracted with ethyl acetate. The extracts were washed with 1M aqueous hydrochloric acid and brine, then dried (MgSO₄) and evaporated under reduced pressure. The residue was triturated with a small amount of methanol to give the crude amide. A mixture of the amide, quinoline-6-carbaldehyde (0.064 g, 0.407 mmol), sodium acetate (0.067 g, 0.817 mmol) and ethanol (2 mL) was irradiated at 150° C. for 40 min in a microwave reactor. The mixture was allowed to cool to room temperature, diluted with water (2 mL) and the solid filtered, washed with ethyl acetate and dried to afford the title compound (0.046 g, 12%) as a solid. 1H NMR (400 MHz, DMSO-d₆) δ ppm 3.88 (m, 2H) 6.95-7.00 (m, 2H) 7.38-7.50 (m, 4H) 7.56 (dd, J=8.34, 4.29 Hz, 1H) 7.83-7.88 (m, 2H) 8.08 (d, J=8.59 Hz, 1H) 8.16 (s, 1H) 8.45 (d, J=8.08 Hz, 1H) 8.94 (dd, J=4.17, 1.64 Hz, 1H) 10.42 (s, 1H) 12.77 (s, 1H).

### Example 47

### N-[(4-Chloro-3-{[(5Z)-4-oxo-5-(6-quinolinylmethylidene)-4,5-dihydro-1,3-thiazol-2-yl]amino}phenyl)methyl]acetamide

The method of example 6(d) was followed, using acetic acid in place of methoxyacetic acid, to give the title compound (52%) as a solid. 1H NMR (400 MHz, DMSO-d₆) δ 1.86 (s, 3H), 4.26 (d, J=6.4 Hz, 2H), 7.05 (s, 1H), 7.10 (dd, J=8.4, 1.6 Hz, 1H), 7.50 (d, J=8.0 Hz, 1H), 7.58 (dd, J=8.4, 4.4 Hz, 1H), 7.86 (s, 1H), 7.88 (dd, J=9.2, 2.0 Hz, 1H), 8.08 (d, J=8.8 Hz, 1H), 8.17 (d, J=2.0 Hz, 1H), 8.44 (t, J=6.0 Hz, 1H), 8.49 (dd, J=8.8, 0.8 Hz, 1H), 8.94 (dd, J=4.4, 1.6 Hz, 1H), 12.78 (brs, 1H).

### Example 48

### N-[(4-Chloro-3-{[(5Z)-4-oxo-5-(6-quinolinylmethylidene)-4,5-dihydro-1,3-thiazol-2-yl]amino}phenyl)methyl]-2-methylpropanamide

The method of example 6(d) was followed, using 2-methylpropanoic acid in place of methoxyacetic acid, to give the title compound (73%) as a solid. 1H NMR (400 MHz, DMSO-d₆) δ 0.96 (d, J=6.8 Hz, 6H), 2.40 (m, 1H), 4.27 (d, J=5.6 Hz, 2H), 7.02 (s, 1H), 7.08 (dd, J=8.4, 1.6 Hz, 1H), 7.50 (d, J=8.4 Hz, 1H), 7.58 (dd, J=8.4, 4.0 Hz, 1H), 7.86 (s, 1H), 7.87 (dd, J=9.2, 2.0 Hz, 1H), 8.07 (d, J=8.8 Hz, 1H), 8.16 (d, J=1.6 Hz, 1H), 8.35 (t, J=6.2 Hz, 1H), 8.49 (dd, J=8.4, 0.8 Hz, 1H), 8.95 (dd, J=4.4, 1.6 Hz, 1H), 12.77 (br s, 1H).

### Example 49

### N˜1˜(4-Chloro-3-{[(5Z)-4-oxo-5-(6-quinolinylmethylidene)-4,5-dihydro-1,3-thiazol-2-yl]amino}phenyl)-N˜2˜,N˜2˜-dimethylglycinamide hydrochloride

The procedure of example 46 was followed here, using 2-(dimethylamino)acetyl chloride hydrochloride in place of thiophene-2-acetyl chloride. In addition, the intermediate amide was purified by chromatography (silica gel) and the final compound was precipitated from the reaction solution with aq HCl to give the title compound. 1H NMR (400 MHz, DMSO-d₆) δ ppm 2.83 (s, 6H) 4.11 (s, 2H) 7.42-7.51 (m, 2H) 7.55-7.64 (m, 2H) 7.86 (dd, J=8.97, 1.89 Hz, 1H) 7.88 (s, 1H) 8.09 (d, J=8.84 Hz, 1H) 8.18 (d, J=1.77 Hz, 1H) 8.46 (s, 1H) 8.95 (dd, J=4.17, 1.64 Hz, 1H) 9.90 (br s, 1H) 10.95 (s, 1H) 12.78 (br s, 1H).

### Example 50

### N-[(4-Chloro-3-{[(5Z)-4-oxo-5-(6-quinolinylmethylidene)-4,5-dihydro-1,3-thiazol-2-yl]amino}phenyl)methyl]urea

Potassium cyanate (0.040 g, 0.493 mmol) was added to a stirred suspension of the compound from example 6(c) (0.050 g, 0.127 mmol) in ethanol (2 mL) and 0.1M aqueous hydrochloric acid (1.3 mL, 0.13 mmol). After stirring 18 h at room temperature, the precipitate was filtered, washed with water and dissolved again in methanol (4 mL) and 0.25M aqueous potassium carbonate (4 mL). Acetic acid was added to precipitate the product, which was filtered, washed with water and dried to give the title compound (0.038 g, 68%) as an orange powder. 1H NMR (400 MHz, DMSO-d₆) δ 4.19 (d, J=6.0 Hz, 2H), 5.59 (s, 2H), 6.51 (t, J=6.0 Hz, 1H), 7.02 (s, 1H), 7.09 (dd, J=8.8, 1.6 Hz, 1H), 7.49 (d, J=8.4 Hz, 1H), 7.57 (dd, J=8.4, 4.4 Hz, 1H), 7.85-7.88 (m, 2H), 8.09 (d, J=9.2 Hz, 1H), 8.16 (d, J=1.6 Hz, 1H), 8.47 (dd, J=8.4, 0.8 Hz, 1H), 8.94 (dd, J=4.4, 1.6 Hz, 1H), 12.75 (brs, 1H).

### Example 51

### N-[(4-Chloro-3-{[(5Z)-4-oxo-5-(6-quinolinylmethylidene)-4,5-dihydro-1,3-thiazol-2-yl]amino}phenyl)methyl]-2-hydroxyacetamide

The method of example 6(d) was followed, using hydroxyacetic acid in place of methoxyacetic acid, to give the title compound (70%) as a solid. 1H NMR (400 MHz, DMSO-d₆) δ 3.86 (d, J=5.6 Hz, 2H), 4.32 (d, J=6.0 Hz, 2H), 5.53 (t, J=6.0 Hz, 1H), 7.07 (s, 1H), 7.12 (dd, J=8.0, 1.2 Hz, 1H), 7.49 (d, J=8.4 Hz, 1H), 7.57 (dd, J=8.4, 4.4 Hz, 1H), 7.86-7.89 (m, 2H), 8.09 (d, J=8.8 Hz, 1H), 8.16 (d, J=1.6 Hz, 1H), 8.39 (t, J=6.2 Hz, 1H), 8.49 (d, J=8.0 Hz, 1H), 8.94 (dd, J=4.0, 1.2 Hz, 1H), 12.75 (br s, 1H).

### Example 52

### N˜1˜-[(4-Chloro-3-{[(5Z)-4-oxo-5-(6-quinolinylmethylidene)-4,5-dihydro-1,3-thiazol-2-yl ]amino}phenyl)methyl]-N˜2˜,N˜2˜-dimethylglycinamide

A mixture of (5Z)-2-{[5-(aminomethyl)-2-chlorophenyl]amino}-5-(6-quinolinylmethylidene)-1,3-thiazol-4(5H)-one (example 6(c), 0.060 g, 0.152 mmol), 1-hydroxy-7-azabenzotriazole (0.023 g, 0.167 mmol), dimethylglycine hydrochloride (0.023 g, 0.167 mmol), 1-[3-(dimethylamino)propyl]-3-ethylcarbodiimide hydrochloride (0.032 g, 0.167 mmol) and dimethylformamide (1 mL) was stirred 18 h, then diluted with water (2 mL). The pH was adjusted to 7 with aqueous sodium hydroxide and the mixture extracted with ethyl acetate. The extracts were washed with brine, dried (Na₂SO₄) and evaporated under reduced pressure. The residue was chromatographed (silica gel, 10% methanol/dichloromethane) then triturated with ether to give the title compound (55%) as a solid. 1H NMR (400 MHz, DMSO-d₆) δ 2.17 (s, 6H), 2.98 (s, 2H), 4.29 (d, J=6.4 Hz, 2H), 7.04 (s, 1H), 7.10 (d, J=8.0 Hz, 1H), 7.49 (d, J=8.0 Hz, 1H), 7.58 (dd, J=8.0, 4.0 Hz, 1H), 7.83 (s, 1H), 7.86 (dd, J=8.8, 1.6 Hz, 1H), 8.08 (d, 8.8 Hz, 1H), 8.15 (d, J=2.0 Hz, 1H), 8.44-8.49 (m, 2H), 8.94 (dd, J=4.0, 1.6 Hz, 1H), 12.39 (br s, 1H).

### Example 53

### 1,1-Dimethylethyl (2-{[(4-chloro-3-{[(5Z)-4-oxo-5-(6-quinolinylmethylidene)-4,5-dihydro-1,3-thiazol-2-yl]amino}phenyl)methyl]amino}-2-oxoethyl)carbamate

The method of example 6(d) was followed, using N-(t-butoxycarbonyl)glycine in place of methoxyacetic acid, to give the title compound (79%) as a solid. 1H NMR (400 MHz, DMSO-d₆) δ 1.34 (s, 9H), 3.56 (d, J=6.0 Hz, 2H), 4.30 (d, J=6.0 Hz, 2H), 7.02-7.11 (m, 3H), 7.49 (d, J=8.4 Hz, 1H), 7.58 (dd, J=8.0, 4.0 Hz, 1H), 7.86-7.88 (m, 2H), 8.09 (d, J=8.8 Hz, 1H), 8.17 (d, J=1.2 Hz, 1H), 8.39 (t, J=6.0 Hz, 1H), 8.48 (dd, J=8.8, 0.8 Hz, 1H), 8.94 (dd, J=4.0, 1.6 Hz, 1H), 12.77 (br s, 1H).

### Example 54

### 4-{[(4-Chloro-3-{[(5Z)-4-oxo-5-(6-quinolinylmethylidene)-4,5-dihydro-1,3-thiazol-2-yl]amino}phenyl)methyl]amino}-4-oxobutanoic acid

A solution of (5Z)-2-{[5-(aminomethyl)-2-chlorophenyl]amino}-5-(6-quinolinylmethylidene)-1,3-thiazol-4(5H)-one (example 6(c), 0.060 g, 0.152 mmol), 1-hydroxy-7-azabenzotriazole (0.023 g, 0.167 mmol) and succinic anhydride (0.017 g, 0.167 mmol) in dimethylformamide (1 mL) was stirred 18 h, then diluted with water (1.5 mL). The solid was filtered, washed with water and dried to give the title compound (0.056 g, 74%) as an orange powder. 1H NMR (400 MHz, DMSO-d₆) δ 2.37-2.45 (m, 4H), 4.29 (d, J=6.0 Hz, 2H), 7.04 (s, 1H), 7.10 (dd, J=8.4, 1.6 Hz, 1H), 7.49 (d, J=8.4 Hz, 1H), 7.58 (dd, J=8.4, 4.4 Hz, 1H), 7.86-7.88 (m, 2H), 8.09 (d, J=8.8 Hz, 1H), 8.16 (d, J=1.6 Hz, 1H), 8.44-8.49 (m, 2H), 8.94 (dd, J=4.4, 2.0 Hz, 1H), 12.08 (br s, 1H), 12.77 (br s, 1H).

### Example 55

### (5Z)-2-{[3-(1,3-Oxazol-4-yl)phenyl]amino}-5-(6-quinolinylmethylidene)-1,3-thiazol-4(5H)-one trifluoroacetate

a) 2-{[3-(1,3-Oxazol-4-yl)phenyl]amino}-1,3-thiazol-4(5H)-one. 3-(1,3-Oxazol-4-yl)aniline (Dumas, J; et al. PCT nt. Appl. (2003), WO2003040141A1, 300 mg, 1.87 mmoles) and 2-(methylthio)-1,3-thiazol-4(5H)-one (250 mg, 1.7 mmoles) in ethanol (30 mL) were heated together under reflux for 18 hours. The mixture was diluted with ethyl acetate and washed with 1 molar hydrochloric acid and brine. Flash chromatography (dichloromethane to 4% methanol in dichloromethane) gave the title compound as an oil (80 mg, 18%). 1H NMR (400 MHz, DMSO-d₆) δ ppm 3.97-4.07 (m, 2H) 7.41 (s, 1H) 7.42-7.50 (m, 1H) 7.51-7.61 (m, 2H) 8.46-8.54 (m, 1H) 8.63-8.73 (m, 1H) 11.27 (s, 1H) 11.84 (s, 1H) 12.08 (s, 1H).

b) (5Z)-2-{[3-(1,3-Oxazol-4-yl)phenyl]amino}-5-(6-quinolinylmethylidene)-1,3-thiazol-4(5H)-one. 2-{[3-(1,3-oxazol-4-yl)phenyl]amino}-1,3-thiazol-4(5H)-one (78 mg, 0.26 mmoles), sodium acetate (82 mg, 1.0 mmoles), quinoline-6-carboxaldehyde (41 mg, 0.26 mmoles) and ethanol (3.0 mL) were sealed in a pressure flask and heated in a microwave reactor at 180° C. for 40 minutes. Water was added and the crude product extracted into ethyl acetate. Preparative HPLC afforded the title compound (5.0 mg, 4.8%). 1H NMR (400 MHz, DMSO-d₆) δ ppm 7.05 (s, 1H) 7.47-7.55 (m, 1H) 7.58-7.69 (m, 1H) 7.80-7.92 (m, 1H) 7.95 (s, 1H) 8.01-8.12 (m, 1H) 8.15-8.26 (m, 1H) 8.30 (s, 1H) 8.47-8.56 (m, 2H) 8.68-8.74 (m, 1H) 8.94-9.03 (m, 1H) 11.81 (s, 1H)

### Example 56

### (5Z)-2-[(1-Ethyl-1H-pyrazol-5-yl)amino]-5-(6-quinolinylmethylidene)-1,3-thiazol-4(5H)-one trifluoroacetate

A mixture of 1-ethyl-1H-pyrazol-5-amine (48 mg, 0.43 mmoles) and (5Z)-2-(methylthio)-5-(6-quinolinylmethylidene)-1,3-thiazol-4(5H)-one (65 mg, 0.22 mmoles) in isobutyric acid (0.5 mL) was heated under reflux for 4 hours. Purification by preparative HPLC gave the title compound (5.0 mg, 6.5%). 1H NMR (400 MHz, DMSO-d₆) δ ppm 1.30 (t, J=7.33 Hz, 3H) 4.10 (q, J=7.33 Hz, 2H) 6.24 (d, J=2.02 Hz, 1H) 7.50 (d, J=2.02 Hz, 1H) 7.66 (dd, J=8.34, 4.29 Hz, 1H) 7.89 (s, 1H) 8.04 (dd, J=8.84, 2.02 Hz, 1H) 8.16 (d, J=8.84 Hz, 1H) 8.31 (d, J=1.77 Hz, 1H) 8.60 (d, J=7.83 Hz, 1H) 9.01 (dd, J=4.29, 1.52 Hz, 1H) 12.72 (s, 1H)

### Example 57

### N˜1˜-[(4-Chloro-3-{[(5Z)-4-oxo-5-(6-quinolinylmethylidene)-4,5-dihydro-1,3-thiazol-2-yl ]amino}phenyl)methyl]glycinamide

A solution of the compound from example 53 (0.033 g, 0.060 mmol) in trifluoroacetic acid (2 mL) was allowed to stand at room temperature for 0.5 h, then evaporated under reduced pressure. The residue was dissolved in 0.5 M aqueous potassium carbonate (3 mL) and the pH lowered to 8 by adding acetic acid. The precipitate was filtered, washed with water and dried to give the title compound (0.024 g, 89%) as a brown powder. 1H NMR (400 MHz, DMSO-d₆+1% TFA) δ 3.63 (q, J=5.6 Hz, 2H), 4.38 (d, J=6.0 Hz, 2H), 7.07 (d, J=1.6 Hz, 1H), 7.15 (dd, J=8.4, 1.6 Hz, 1H), 7.54 (d, J=8.0 Hz, 1H), 7.72 (dd, J=8.8, 4.8 Hz, 1H), 7.90 (s, 1H), 7.95 (dd, J=8.8, 2.0 Hz, 1H), 8.01 (br s, 3H), 8.16 (d, J=8.8 Hz, 1H), 8.25 (d, J=1.6 Hz, 1H), 8.66 (d, J=8.4 Hz, 1H), 8.89 (t, J=5.2 Hz, 1H), 9.05 (dd, J=4.4, 1.2 Hz, 1H).

### Example 58

### Ethyl (4-chloro-3-{[(5Z)-4-oxo-5-(6-quinolinylmethylidene)-4,5-dihydro-1,3-thiazol-2-yl]amino}phenyl)carbamate

The compound from example 31(c) (0.103 g, 0.427 mmol) in dioxane (2.0 mL) was treated with ethyl isocyanate (0.070 mL, 0.884 mmol). The reaction was stirred for 30 min. and then quenched with water, diluted with ethyl acetate, and extracted twice. The organics were combined and dried over MgSO₄, filtered, and concentrated under reduced pressure. The resulting solid was triturated with methanol to obtain a crude white solid (0.110 g, 0.319 mmol) which was charged to a microwave vial dissolved in ethanol (3.0 mL) followed by addition of sodium acetate (0.052 g, 0.634 mmol) and 6-quinolinecarbaldehyde (0.050 g, 0.318 mmol). The contents were sealed and irradiated at 180° C. for 40 min in a microwave reactor. The mixture was allowed to cool to room temperature and taken up in water. The resulting precipitate was filtered off, washed with water and dried under vacuum to afford the title compound (0.010 g, 5%) as a side product. 1H NMR (400 MHz, DMSO-d₆) δ ppm 4.12 (q, 2H) 1.24 (q, 3H) 7.23-7.33 (m, 2H) 7.45 (d, J=8.59 Hz, 1H) 7.57 (dd, J=8.34, 4.04 Hz, 1H) 7.82-7.91 (m, 2H) 8.09 (d, J=8.84 Hz, 1H) 8.17 (s, 1H) 8.46 (m, 1H) 8.94 (dd, J=4.29, 1.52 Hz, 1H) 9.88 (s, 1H) 12.77 (s, 1H)

### Example 59

### N-(4-Chloro-3-{[(5Z)-4-oxo-5-(6-quinolinylmethylidene)-4,5-dihydro-1,3-thiazol-2-yl]amino}phenyl)-2-cyclopropylacetamide

A solution of cyclopropylacetyl chloride (Bouzoubaa, M et al; J. Med. Chem., 1984, 27(10), 1291-4., 0.236 g, 2.00 mmol) in dioxane (1 mL) was injected into a stirred mixture of the compound from example 31(c) (0.200 g, 0.830 mmol) and dioxane (2 mL) and stirring continued for 2 h. The mixture was diluted with water (20 mL) and extracted with ethyl acetate. The extracts were washed with brine, then dried (MgSO₄) and evaporated under reduced pressure. A mixture of the crude amide, quinoline-6-carbaldehyde (0.069 g, 0.436 mmol), sodium acetate (0.072 g, 0.872 mmol) and ethanol (2 mL) was irradiated at 180° C. for 40 min in a microwave reactor. The mixture was allowed to cool to room temperature and evaporated under reduced pressure. The residue was chromatographed (silica gel, 5-10% methanol/dichloromethane) to afford the title compound (0.021 g, 6%) as a solid. 1H NMR (400 MHz, DMSO-d₆) δ ppm 0.16-0.20 (m, 2H) 0.45-0.49 (m, 2H) 1.06 (m, 1H) 2.21 (d, J=7.1 Hz, 2H) 7.42 (dd, J=8.8, 2.3 Hz, 1H) 7.46-7.48 (m, 2H) 7.56 (dd, J=8.4, 4.3 Hz, 1H) 7.85-7.89 (m, 2H) 8.09 (d, J=8.6 Hz, 1H) 8.17 (s, 1H) 8.46 (d, J=8.1 Hz, 1H) 8.94 (dd, J=4.1, 1.5 Hz, 1H) 10.03 (s, 1H) 12.77 (s, 1H)

### Example 60

### N-(4-Chloro-3-{[(5Z)-4-oxo-5-(6-quinolinylmethylidene)-4,5-dihydro-1,3-thiazol-2-yl]amino}phenyl)-N′-(phenylmethyl)urea

The compound from example 31(c) (0.200 g, 0.829 mmol) in dioxane (4.0 mL) was treated with benzyl isocyanate (0.2 mL, 1.619 mmol) and allowed to stir at room temperature until reaction was complete by LC/MS. The reaction was diluted with ethyl acetate, washed with water then brine, dried over MgSO₄, filtered and concentrated. The crude product was then charged to a microwave vial and dissolved in xylene (2.0 mL) followed by the addition of and 6-quinolinecarbaldehyde (0.024 g, 0.152 mmol) and piperidine (0.030 mL, 0.303 mmol). The contents were sealed and irradiated at 150° C. for 20 min in a microwave reactor. The mixture was allowed to cool to room temperature, evaporated under reduced pressure and taken up in dilute acid. The resulting precipitate was filtered off, washed with acid, water and ethyl acetate, and dried under vacuum to afford the title compound (0.029 g, 7%) as a yellow solid. 1H NMR (400 MHz, DMSO-d₆) δ ppm 4.28 (d, J=5.81 Hz, 2H) 6.71 (t, J=6.06 Hz, 1H) 7.18-7.25 (m, 3H) 7.26-7.35 (m, 4H) 7.38 (d, J=8.84 Hz, 1H) 7.55 (dd, J=8.34, 4.29 Hz, 1H) 7.81-7.91 (m, 2H) 8.09 (d, J=8.84 Hz, 1H) 8.17 (d, J=1.77 Hz, 1H) 8.46 (d, J=7.58 Hz, 1H) 8.80 (s, 1H) 8.94 (dd, J=4.17, 1.64 Hz, 1H) 12.72 (s, 1H)

### Example 61

### Ethyl 4-[(4-chloro-3-{[(5Z)-4-oxo-5-(6-quinolinylmethylidene)-4,5-dihydro-1,3-thiazol-2-yl]amino}phenyl)amino]-4-oxobutanoate

The method of example 59 was followed here, using 3-carbomethoxypropionyl chloride, piperidine and xylene in place of cyclopropylacetyl chloride, sodium acetate and ethanol respectively, to give the title compound (4%) as a solid. 1H NMR (400 MHz, DMSO-d₆) δ ppm 1.14 (t, J=7.1 Hz, 3H) 2.55-2.62 (m, 4H) 4.02 (q, J=7.1 Hz, 2H) 7.38 (m, 1H) 7.43-7.50 (m, 2H) 7.57 (dd, J=8.10, 4.10 Hz, 1H) 7.85-7.89 (m, 2H) 8.09 (d, J=9.09 Hz, 1H) 8.17 (d, J=1.52 Hz, 1H) 8.47 (d, J=7.58 Hz, 1H) 8.94 (dd, J=4.10, 1.50 Hz, 1H) 10.21 (s, 1H) 12.78 (s, 1H)

### Example 62

### N-(4-Chloro-3-{[(5Z)-4-oxo-5-(6-quinolinylmethylidene)-4,5-dihydro-1,3-thiazol-2-yl]amino}phenyl)-N′-(1-methylethyl)urea trifluoroacetate

The method of example 60 was followed here, using isopropyl isocyanate in place of benzyl isocyanate. Additionally, the material was purified by rp-HPLC (ODS, 10-90% acetonitrile/water+0.1% trifluoroacetic acid) to give the title compound (9%) as a yellow solid. 1H NMR (400 MHz, DMSO-d₆) δ ppm 1.08 (d, J=6.57 Hz, 6H) 3.65-3.79 (m, 1H) 6.09 (d, J=7.58 Hz, 1H) 7.15-7.21 (m, 1H) 7.26 (d, J=2.27 Hz, 1H) 7.37 (d, J=8.84 Hz, 1H) 7.59 (dd, J=8.34, 4.04 Hz, 1H) 7.83-7.93 (m, 2H) 8.11 (d, J=9.09 Hz, 1H) 8.19 (d, J=1.77 Hz, 1H) 8.48-8.56 (m, 2H) 8.96 (dd, J=4.29, 1.77 Hz, 1H) 12.74 (s, 1H).

### Example 63

### N-(4-Chloro-3-{[(5Z)-4-oxo-5-(6-quinolinylmethylidene)-4,5-dihydro-1,3-thiazol-2-yl]amino}phenyl)-1-piperidinecarboxamide trifluoroacetate

The procedure of example 62 gave the title compound as a by-product. 1H NMR (400 MHz, DMSO-d₆) δ ppm 1.42-1.60 (m, 6H) 3.36-3.45 (m, 4H) 7.31-7.41 (m, 3H) 7.62 (dd, J=8.21, 4.17 Hz, 1H) 7.87 (s, 1H) 7.91 (dd, J=8.97, 1.89 Hz, 1H) 8.12 (d, J=8.84 Hz, 1H) 8.21 (d, J=11.77 Hz, 1H) 8.54 (d, J=8.34 Hz, 1H) 8.63 (s, 1H) 8.98 (dd, J=4.17, 1.64 Hz, 1H) 12.77 (s, 1H)

### Example 64

### (5Z)-2-{[2-Chloro-5-(1H-imidazol-4-yl)phenyl]amino}-5-(6-quinolinylmethylidene)-1,3-thiazol-4(5H)-one trifluoroacetate

a) 4-(4-Chloro-3-nitrophenyl)-1H-imidazole. A mixture of 2-bromo-1-(4-chloro-3-nitrophenyl)ethanone (1.34 g, 4.79 mmoles) and formamide (10.0 mL, 15.86 mmoles) were heated together at 132° C. for 2 hours, then diluted with water and extracted with ethyl acetate (×3). The extracts were filtered and washed with brine, dried and evaporated. The solid was triturated with ether and dried to give the title compound (320 mg, 30%) 1H NMR (400 MHz, DMSO-d₆) δ ppm 7.75 (d, J=8.59 Hz, 1H) 7.82 (d, J=1.01 Hz, 1H) 7.90 (d, J=1.01 Hz, 1H) 8.08 (dd, J=8.34, 2.02 Hz, 1H) 8.40 (d, J=2.02 Hz, 1H) 12.48 (s, 1H).

b) 2-Chloro-5-(1H-imidazol-4-yl)aniline. 4-(4-Chloro-3-nitrophenyl)-1H-imidazole (300 mg, 1.34 mmoles) and 10% Pd/C catalyst (500 mg) in ethanol (50 mL) was shaken in a hydrogen atmosphere at 50 p.s.i. for 2 hours. The mixture was filtered and evaporated to a crude product which was chromatographed (dichloromethane to 10% methanol in dichloromethane) to give the title compound (120 mg, 46%). 1H NMR (400 MHz, CHLOROFORM-d) δ ppm 6.78 (dd, J=8.21, 2.15 Hz, 1H) 6.97 (d, J=2.02 Hz, 2H) 7.01 (d, J=8.34 Hz, 1H) 7.07 (d, J=1.01 Hz, 1H) 7.44 (d, J=1.01 Hz, 1H) 7.87 (s, 1H).

c) (5Z)-2-{[2-chloro-5-(1H-imidazol-4-yl)phenyl]amino}-5-(6-quinolinylmethylidene)-1,3-thiazol-4(5H)-one. A mixture of 2-chloro-5-(1H-imidazol-4-yl)aniline and (5Z)-2-(methylthio)-5-(6-quinolinylmethylidene)-1,3-thiazol-4(5H)-one (100 mg, 0.35 mmoles) in dioxan (1.0 mL) was sealed in a pressure flask and heated at 160° C. for 30 minutes. The mixture was cooled, diluted with ethanol and the crude product collected. Purification by preparative HPLC gave the title compound (26 mg, 17%). 1H NMR (400 MHz, DMSO-d₆) δ ppm 7.56 (dd, J=8.34, 4.04 Hz, 1H) 7.62-7.69 (m, 2H) 7.69-7.75 (m, 1H) 7.85 (dd, J=8.97, 1.89 Hz, 1H) 7.91 (s, 1H) 8.07 (d, J=8.84 Hz, 1H) 8.16 (d, J=6.82 Hz, 2H) 8.45 (d, J=7.83 Hz, 1H) 8.94 (dd, J=4.04, 1.52 Hz, 2H) 12.92 (s, 1H), 14.49 (br s, 1H)

### Example 65

### (5Z)-2-{[2-Chloro-5-(2-methyl-1,3-thiazol-4-yl)phenyl]amino}-5-(6-quinolinylmethylidene)-1,3-thiazol-4(5H)-one

a) 2-Chloro-5-(2-methyl-1,3-thiazol-4-yl)aniline. A mixture of 2-bromo-1-(4-chloro-3-nitrophenyl)ethanone (1.40 g, 5.02 mmoles) and thioacetamide (370 mg, 4.92 mmoles) in ethanol (10 mL) were heated together under reflux for 3 hours, cooled and diluted with ethanol. 10% Pd/C catalyst was added and the mixture was shaken in a hydrogen atmosphere (35 psi) for 40 minutes. The mixture was filtered through celite and evaporated to give the title compound as a solid from diethyl ether, (550 mg, 50%). 1H NMR (400 MHz, DMSO-d₆) δ ppm 2.71 (s, 3H) 7.14 (br s, 2H) 7.23 (d, J=8.34 Hz, 1H) 7.27-7.32 (m, 1H) 7.56 (s, 1H) 7.84 (s, 1H).

b) 2-{[2-Chloro-5-(2-methyl-1,3-thiazol-4-yl)phenyl]amino}-1,3-thiazol-4(5H)-one. 2-Chloro-5-(2-methyl-1,3-thiazol-4-yl)aniline (546 mg, 2.43 mmoles) and 2-(methylthio)-1,3-thiazol-4(5H)-one (360 mg, 2.44 mmoles) in ethanol (10 mL) were heated together under reflux for 18 hours. Flash Chromatography (hexane-ethyl acetate) returned starting material and desired product (80 mg, 10%). 1H NMR (400 MHz, DMSO-d₆) δ ppm 2.71 (s, 3H) 4.05 (s, 2H), 7.54 (d, J=8.34 Hz, 1H) 7.61 (s, 1H) 7.70 (s, 1H) 8.02 (s, 1H).

c) (5Z)-2-{[2-Chloro-5-(2-methyl-1,3-thiazol-4-yl)phenyl]amino}-5-(6-quinolinylmethylidene)-1,3-thiazol-4(5H)-one. A mixture of 2-{[2-chloro-5-(2-methyl-1,3-thiazol-4-yl)phenyl]amino}-1,3-thiazol-4(5H)-one (70 mg, 0.21 mmoles), quinoline-6-carboxaldehyde (34 mg, 0.21 mmoles) and sodium acetate (36 mg, 0.42 mmoles) in ethanol (2.0 mL) was sealed in a pressure flask and heated at 180° C. for 30 minutes in a microwave reactor. Piperidine (0.5 mL) was then added and the mixture was heated at 180° C. for a further 2 hours in the microwave reactor. Flash chromatography (0-10% methanol-dichloromethane) afforded the title compound which was recrystallized from dichloromethane (5.0 mg, 5.2%) 1H NMR (400 MHz, DMSO-d₆) δ ppm 2.70 (s, 3H) 7.54 (dd, J=8.34, 4.04 Hz, 1H) 7.62 (d, J=8.59 Hz, 1H) 7.72 (s, 1H) 7.80 (dd, J=8.72, 1.64 Hz, 1H) 7.85 (dd, J=8.84, 2.02 Hz, 1H) 7.88 (s, 1H) 8.03-8.09 (m, 2H) 8.15 (d, J=1.77 Hz, 1H) 8.44 (d, J=7.83 Hz, 1H) 8.92 (dd, J=4.17, 1.64 Hz, 1H) 12.84 (s, 1H)

### Example 66

### 5-Chloro-6-{[(5Z)-4-oxo-5-(6-quinolinylmethylidene)-4,5-dihydro-1,3-thiazol-2-yl]amino}-1,3-dihydro-2H-benzimidazol-2-one

a) 5-Chloro-6-[(4-oxo-4,5-dihydro-1,3-thiazol-2-yl)amino]-1,3-dihydro-2H-benzimidazol-2-one. A solution of 5-amino-6-chloro-1,3-dihydro-2H-benzimidazol-2-one (B. Lamatsch, PCT Int. Appl. (1999), WO9936402A1, 0.184 g, 1.00 mmol) and 2-(methylthio)-1,3-thiazol-4(5H)-one (0.175 g, 1.19 mmol) in ethanol (5 mL) was heated under reflux for 18 h, then cooled. The solvent was evaporated under reduced pressure and the residue boiled in ethyl acetate (20 mL), then cooled, filtered, washed with ethyl acetate and dried to give the title compound (0.243 g, 86%) as a light brown powder. ¹H NMR (400 MHz, DMSO-d₆) δ 3.99 (s, 2H), 6.60 (br s, 1H), 7.00 (s, 1H), 10.69 (br s, 1H), 10.74 (br s, 1H), 11.87 (br s, 1H).

b) 5-Chloro-6-{[(5Z)-4-oxo-5-(6-quinolinylmethylidene)-4,5-dihydro-1,3-thiazol-2-yl]amino}-1,3-dihydro-2H-benzimidazol-2-one. A mixture of the compound from example 66(a) (0.100 g, 0.354 mmol), quinoline-6-carbaldehyde (0.120 g, 0.763 mmol), piperidinium acetate in ethanol (1M, 0.763 mL, 0.763 mmol) and ethanol (2.5 mL) was heated in a microwave reactor at 150° C. for 0.5 h, then cooled. Acetic acid (˜0.1 mL) was added, followed by water (1 mL). The precipitate was filtered, washed with 50% aqueous ethanol and dried. The solid was dissolved in 0.5M aqueous potassium carbonate (2 mL) and methanol (20 mL) and the pH adjusted to 7 with acetic acid. The solid was filtered, washed with 50% aqueous methanol and water, then dried to give the title compound (0.074 g, 50%) as a brown powder. 1H NMR (400 MHz, DMSO-d₆) δ 6.74 (br s, 1H), 7.06 (s, 1H), 7.56 (dd, J=8.4, 4.4 Hz, 1H), 7.83 (s, 1H), 7.86 (dd, J=9.2, 2.4 Hz, 1H), 8.08 (d, J=8.4 Hz, 1H), 8.14 (d, J=2.0 Hz, 1H), 8.46 (dd, J=8.4, 0.8 Hz, 1H), 8.93 (dd, J=4.0, 1.6 Hz, 1H), 10.80 (br s, 1H), 10.84 (brs, 1H), 12.68 (brs, 1H).

### Example 67

### Methyl [(4-chloro-3-{[(5Z)-4-oxo-5-(6-quinolinylmethylidene)-4,5-dihydro-1,3-thiazol-2-yl]amino}phenyl)methyl]carbamate

Methyl chloroformate (0.028 mL, 0.354 mmol) was injected dropwise into a stirred, ice-cooled suspension of the compound from example 6(c) (0.127 g, 0.322 mmol) in pyridine (2 mL) under nitrogen. After 5 min, the mixture was warmed to room temperature and stirred 2 h, then diluted with water (20 mL). The solid was filtered, washed with water and dried, then chromatographed (silica gel, 5-10% methanol/dichloromethane) to give the title compound (0.082 g, 56%) as a solid. 1H NMR (400 MHz, DMSO-d₆) δ 3.50 (s, 3H), 4.20 (d, J=6.0 Hz, 2H), 7.04 (s, 1H), 7.10 (d, J=8.0 Hz, 1H), 7.51 (d, J=8.4 Hz, 1H), 7.58 (dd, J=8.4, 4.4 Hz, 1H), 7.77 (t, J=6.0 Hz, 1H), 7.86-7.88 (m, 2H), 8.08 (d, J=8.8 Hz, 1H), 8.16 (d, J=1.2 Hz, 1H), 8.47 (d, J=8.0 Hz, 1H), 8.94 (dd, J=4.0, 1.2 Hz, 1H), 12.78 (br s, 1H).

### Example 68

### N-[(4-Chloro-3-{[(5Z)-4-oxo-5-(6-quinolinylmethylidene)-4,5-dihydro-1,3-thiazol-2-yl]amino}phenyl)methyl]-N′-(1-methylethyl)urea

The method of example 67 was followed, using 2-isocyanatopropane in place of methyl chloroformate, and omitting the chomatography step to give the title compound (73%) as an orange-brown solid. 1H NMR (400 MHz, DMSO-d₆) δ 0.94 (d, J=6.4 Hz, 6H), 3.64 (m, 1H), 4.21 (d, J=6.0 Hz, 2H), 5.79 (d, J=7.6 Hz, 1H), 6.26 (t, J=6.0 Hz, 1H), 7.02 (s, 1H), 7.08 (dd, J=8.0, 0.8 Hz, 1H), 7.49 (d, J=8.4 Hz, 1H), 7.57 (dd, J=8.4, 4.4 Hz, 1H), 7.85-7.88 (m, 2H), 8.08 (d, J=8.8 Hz, 1H), 8.16 (d, J=0.8 Hz, 1H), 8.47 (d, J=8.0 Hz, 1H), 8.94 (dd, J=4.0, 1.6 Hz, 1H), 12.74 (brs, 1H).

### Example 69

### 2-[3,4-Bis(methyloxy)phenyl]-N-(4-chloro-3-{[(5Z)-4-oxo-5-(6-quinolinylmethylidene)-4,5-dihydro-1,3-thiazol-2-yl]amino}phenyl)acetamide

A solution of 3,4-dimethoxyphenylacetyl chloride in dioxane (1 mL) was injected into a stirred mixture of the compound from example 31(c) (0.130 g, 0.539 mmol) and dioxane (1 mL) and stirring continued for 2 h. The mixture was diluted with water (20 mL) and extracted with ethyl acetate. The extracts were washed with brine, then dried (MgSO₄) and evaporated under reduced pressure. A mixture of the crude amide, quinoline-6-carbaldehyde (0.085 g, 0.538 mmol), piperidine (0.053 mL, 0.535 mmol) and ethanol (4 mL) was irradiated at 150° C. for 20 min in a microwave reactor. The mixture was allowed to cool to room temperature, diluted with water and acidified with 1M aqueous hydrochloric acid. The precipitate was filtered, washed with water and dried to afford the title compound (0.038 g, 13%) as a solid. 1H NMR (400 MHz, DMSO-d₆) δ ppm 3.70 (s, 3H) 3.71 (s, 3H) 3.73 (s, 2H) 6.81-7.00 (m, 3H) 7.42-7.52 (m, 3H) 7.70 (dd, J=8.34, 4.55 Hz, 1H) 7.86 (s, 1H) 7.95 (dd, J=8.60, 1.60 Hz, 1H) 8.16 (d, J=8.84 Hz, 1H) 8.23 (d, J=1.52 Hz, 1H) 8.66 (d, J=8.08 Hz, 1H) 9.04 (dd, J=4.55, 1.52 Hz, 1H) 10.45 (s, 1H) 12.78 (s, 1H)

### Example 70

### (5Z)-2-{[2-Chloro-5-(1,3-oxazol-4-yl)phenyl]amino}-5-(6-quinolinylmethylidene)-1,3-thiazol-4(5H)-one

a) 4-(4-Chloro-3-nitrophenyl)-1,3-oxazole. A mixture of 2-bromo-1-(4-chloro-3-nitrophenyl)ethanone (4.0 g, 14.31 mmoles) and formamide (10.0 mL, 15.86 mmoles) were heated together at 130° C. for 3 hours, poured into water, the solid collected and washed with water. Flash chromatography (dichloromethane) gave the title compound (1.0 g, 31%). 1H NMR (400 MHz, CHLOROFORM-d) δ ppm 7.52 (d, J=8.34 Hz, 1H) 7.84 (dd, J=8.34, 2.02 Hz, 1H) 7.95 (m, 1H) 8.09 (s, 1H) 8.20 (d, J=2.02 Hz, 1H).

b) 2-Chloro-5-(1,3-oxazol-4-yl)aniline. A mixture of 4-(4-chloro-3-nitrophenyl)-1,3-oxazole (1.0 g, 4.45 mmoles) and 10% Pd/C catalyst (300 mg) was shaken in a hydrogen atmosphere (60 psi) for 4 hours. The mixture was filtered through celite and evaporated. The crude product was extracted with hot ethyl acetate; evaporation of the ethyl acetate extract gave the title compound as an oil (650 mg, 75%). 1H NMR (400 MHz, DMSO-d₆) δ ppm 7.37 (s, 2H, NH) 7.57 (dd, J=8.59, 2.27 Hz, 1H) 7.69 (d, J=8.84 Hz, 1H) 7.91 (d, J=2.27 Hz, 1H) 8.24 (d, J=1.52 Hz, 1H) 9.42 (s, 1H).

c) (5Z)-2-{[2-Chloro-5-(1,3-oxazol-4-yl)phenyl]amino}-5-(6-quinolinylmethylidene)-1,3-thiazol-4(5H)-one. A mixture of 2-chloro-5-(1,3-oxazol-4-yl)aniline (94 mg, 0.48 mmoles) and (5Z)-2-(methylthio)-5-(6-quinolinylmethylidene)-1,3-thiazol-4(5H)-one (106 mg, 0.38 mmoles) in dimethylformamide (1.0 mL) was heated at 200° C. in a sealed pressure flask for 1 hour. The mixture was cooled and diluted with ethanol to give a solid which was collected, washed with ethanol and dichloromethane to afford the title compound (75 mg, 46%). 1H NMR (400 MHz, DMSO-d₆) δ ppm 7.55 (dd, J=8.34, 4.29 Hz, 1H) 7.59 (s, 1H) 7.61-7.69 (m, 2H) 7.85 (dd, J=8.84, 2.02 Hz, 1H) 7.88 (s, 1H) 8.07 (d, J=8.84 Hz, 1H) 8.15 (d, J=1.77 Hz, 1H) 8.44 (d, J=7.83 Hz, 1H) 8.50 (d, J=1.01 Hz, 1H) 8.73 (s, 1H) 8.92 (dd, J=4.04, 1.52 Hz, 1H) 12.85 (s, 1H)

### Example 71

### (5Z)-2-{[5-(1,3-Benzothiazol-2-yl)-2-chlorophenyl]amino}-5-(6-quinolinylmethylidene)-1,3-thiazol-4(5H)-one

A mixture of 5-benzothiazol-2-yl-2-chlorophenylamine (110 mg, 0.42 mmoles) and (5Z)-2-(methylthio)-5-(6-quinolinylmethylidene)-1,3-thiazol-4(5H)-one (120 mg, 0.42 mmoles) in dimethylformamide (3.0 mL) was heated at 180° C. for 1 hour, allowing the DMF to distil. The mixture was cooled and ethanol added to give a solid. The crude product was slurried in boiling ethanol, collected, washed with ethanol and dichloromethane to give the title compound (26 mg, 12.5%). 1H NMR (400 MHz, DMSO-d₆) δ ppm 7.46-7.58 (m, 3H) 7.78 (d, J=8.34 Hz, 1H) 7.84-7.95 (m, 4H) 8.06 (dd, J=8.21, 6.19 Hz, 2H) 8.13-8.21 (m, 2H) 8.43 (d, J=7.58 Hz, 1H) 8.90 (dd, J=4.04, 1.52 Hz, 1H) 12.90 (s, 1H)

### Example 72

### N-(4-Chloro-3-{[(5Z)-4-oxo-5-(6-quinolinylmethylidene)-4,5-dihydro-1,3-thiazol-2-yl]amino}phenyl)-3-phenylpropanamide trifluoroacetate

The method of example 69 was followed here, using 3-phenylpropionyl chloride in place of 3,4-dimethoxyphenylacetyl chloride. Additionally, the material was purified by rp-HPLC (ODS, 10-90% acetonitrile/water+0.1% trifluoroacetic acid) to give the title compound (2%) as a solid. 1H NMR (400 MHz, DMSO-d₆) δ ppm 2.60-2.71 (m, 2H) 2.86-2.94 (m, 2H) 7.18-7.30 (m, 5H) 7.36-7.52 (m, 3H) 7.73 (dd, J=8.59, 4.55 Hz, 1H) 7.89 (s, 1H) 7.98 (dd, J=8.90, 1.80 Hz, 1H) 8.18 (d, J=8.84 Hz, 1H) 8.26 (d, J=1.77 Hz, 1H) 8.70 (d, J=8.8 Hz, 1H) 9.06 (dd, J=4.42, 1.39 Hz, 1H) 10.19 (s, 1H) 12.78 (s, 1H)

### Example 73

### N-(4-Chloro-3-{[(5Z)-4-oxo-5-(6-quinolinylmethylidene)-4,5-dihydro-1,3-thiazol-2-yl]amino}phenyl)-2-(4-fluorophenyl)acetamide trifluoroacetate

The method of example 59 was followed here, using (4-fluorophenyl)acetyl chloride in place of cyclopropylacetyl chloride. Additionally, the material was purified by rp-HPLC (ODS, 10-90% acetonitrile/water+0.1% trifluoroacetic acid) to give the title compound (17%) as a solid. 1H NMR (400 MHz, DMSO-d₆) δ ppm 3.65 (s, 2H) 7.11-7.16 (m, 2H) 7.33-7.50 (m, 5H) 7.58 (dd, J=8.30, 4.50 Hz, 1H), 7.85-7.88 (m, 2H), 8.09 (d, J=9.3 Hz, 1H) 8.16 (s, 1H) 8.46 (d, J=7.33 Hz, 1H) 8.95 (dd, J=4.29, 1.77 Hz, 1H) 10.36 (s, 1H) 12.76 (s, 1H)

### Example 74

### (5Z)-2-[(2-Amino-5-chloro-1H-benzimidazol-6-yl)amino]-5-(6-quinolinylmethylidene)-1,3-thiazol-4(5H)-one

a) 5-Chloro-6-nitro-1H-benzimidazol-2-amine. A solution of 4-chloro-5-nitro-1,2-phenylenediamine (0.187 g, 1.00 mmol) and cyanogen bromide (0.115 g, 1.09 mmol) in ethanol (5 mL) was heated under reflux for 3 h, then cooled and adjusted to pH 9 with concentrated aqueous ammonia solution. Water (10 mL) was added, and the precipitate filtered, washed with water and dried to give the title compound (0.196 g, 90%) as a brown solid. LCMS (ESI) m/z 213 (MH⁺).

b) 1,1-Dimethylethyl 2-(bis{[(1,1-dimethylethyl)oxy]carbonyl}amino)-5-chloro-6-nitro-1H-benzimidazole-1-carboxylate. Di-t-butyl dicarbonate (0.776 g, 3.56 mmol) was added to a stirred suspension of the compound from example 74(a) (0.189 g, 0.889 mmol) in dioxane (5 mL), followed by 4-(dimethylamino)pyridine (0.010 g, 0.08 mmol). The mixture was stirred for 60 h, diluted with toluene (10 mL) and loaded on to a silica gel column. Elution with 10-50% ethyl acetate/hexane gave the title compound (0.400 g, 73%) as a mixture of isomers. 1H NMR (400 MHz, CDCl₃) δ 1.45 (s, 18H), 1.70 (s, 9H), 7.92 (s, 0.5H), 8.28 (s, 0.5H), 8.30 (s, 0.5H), 8.62 (s, 0.5H).

c) 1,1-Dimethylethyl 2-(bis{[(1,1-dimethylethyl)oxy]carbonyl}amino)-6-amino-5-chloro-1H-benzimidazole-1-carboxylate. A mixture of the compound from example 74(b) (0.400 g, 0.780 mmol), tin (II) chloride (1.48 g, 7.80 mmol), ethanol (3 mL) and dimethylformamide (3 mL) was stirred at room temperature for 1 h, then at 50° C. for 3 h. After cooling, the mixture was poured into saturated aqueous sodium bicarbonate solution and extracted with ethyl acetate. The extracts were washed with water and brine, dried (MgSO₄) and evaporated to dryness under reduced pressure to give the title compound (0.322 g, 85%) as a mixture of isomers. LCMS (ESI) m/z 483 (MH⁺).

d) 1,1-Dimethylethyl {5-chloro-6-[(4-oxo-4,5-dihydro-1,3-thiazol-2-yl)amino]-1H-benzimidazol-2-yl}carbamate. The method of example 66(a) was followed, using the compound from example 74(c) in place of 5-amino-6-chloro-1,3-dihydro-2H-benzimidazol-2-one, to give the title compound (51%) as a cream solid. 1H NMR (400 MHz, DMSO-d₆) δ 1.53 (s, 9H), 3.98 (s, 2H), 7.07 (br s, 1H), 7.48 (br s, 1H), 11.10 (br s, 1H), 11.89 (br s, 2H).

e) (5Z)-2-[(2-Amino-5-chloro-1H-benzimidazol-6-yl)amino]-5-(6-quinolinylmethylidene)-1,3-thiazol-4(5H)-one. A mixture of the compound from example 74(d) (0.050 g, 0.131 mmol), quinoline-6-carbaldehyde (0.023 g, 0.144 mmol), 0.5M piperidinium acetate solution in ethanol (0.260 mL, 0.130 mmol) and ethanol 1 mL was heated in a microwave reactor at 150° C. for 0.5 h, then cooled. After adding water (1 mL), the precipitate was filtered. LCMS showed that this material was predominantly an ethyl carbamate derivative of the title compound. A solution of this intermediate in 1M aqueous sodium hydroxide (3 mL) was heated in the microwave reactor at 120° C. for 0.5 h, then cooled. After adding acetic acid to pH 5-6, the precipitate was filtered, then re-dissolved in 1:1 methanol/1M aqueous sodium hydroxide and reprecipitated with acetic acid. The solid was filtered, washed with water and dried to give the title compound (0.027 g, 49%) as an orange solid. 1H NMR (400 MHz, DMSO-d₆) δ 6.49 (br s, 2H), 6.94 (s, 1H), 7.24 (s, 1H), 7.54 (dd, J=8.4, 4.4 Hz, 1H), 7.83 (s, 1H), 7.85 (dd, J=8.8, 2.0 Hz, 1H), 8.07 (d, J=8.8 Hz, 1H), 8.13 (d, J=2.0 Hz, 1H), 8.44 (d, J=8.8 Hz, 1H), 8.92 (dd, J=4.4, 1.6 Hz, 1H), 11.38 (br s, 1H), 11.74 (br s, 1H).

### Example 75

### N-(4-Chloro-3-{[(5Z)-4-oxo-5-(6-quinolinylmethylidene)-4,5-dihydro-1,3-thiazol-2-yl]amino}phenyl)-1-phenylmethanesulfonamide

A solution of phenylmethanesulfonyl chloride (0.240 g, 1.26 mmol) in dioxane (1 mL) was injected into a stirred mixture of the compound from example 31(c) (0.200 g, 0.830 mmol), 1,4-diazabicyclo[2.2.2]octane (0.062 g, 0.553 mmol) and dioxane (2 mL) and stirring continued for 1 h. The mixture was diluted with water (20 mL) and extracted with ethyl acetate. The extracts were dried (MgSO₄) and evaporated under reduced pressure. The residue was chromatographed (silica gel, 0-10% methanol/chloroform) to give the partially purified sulfonamide intermediate (0.090 g). A mixture of the crude sulfonamide, quinoline-6-carbaldehyde (0.040 g, 0.255 mmol), piperidine (0.045 mL, 0.454 mmol) and ethanol (2 mL) was irradiated at 150° C. for 20 min in a microwave reactor. The mixture was allowed to cool to room temperature and diluted with water (2 mL), acidified with 1M aq HCl and the precipitate filtered, washed with water and dried to give the title compound (0.074 g, 17%) as a solid. 1H NMR (400 MHz, DMSO-d₆) δ ppm 4.57 (s, 2H) 6.96 (d, J=2.53 Hz, 1H) 7.03 (dd, J=8.84, 2.53 Hz, 1H) 7.22-7.32 (m, 5H) 7.47 (dd, J=8.34, 4.29 Hz, 1H) 7.51 (d, J=8.84 Hz, 1H) 7.85-7.92 (m, 2H) 7.99 (d, J=8.84 Hz, 1H) 8.14 (d, J=1.52 Hz, 1H) 8.27 (d, J=7.83 Hz, 1H) 8.90 (dd, J=4.17, 1.64 Hz, 1H) 10.13 (s, 1H) 12.83 (s, 1H)

### Example 76

### N′-(4-Chloro-3-{[(5Z)-4-oxo-5-(6-quinolinylmethylidene)-4,5-dihydro-1,3-thiazol-2-yl]amino}phenyl)-N,N-dimethylsulfamide trifluoroacetate

The method of example 60 was followed here, using dimethylsulfamoyl chloride in place of benzyl isocyanate and ethanol in place of xylene. Additionally, the material was purified by rp-HPLC (ODS, 10-90% acetonitrile/water+0.1% trifluoroacetic acid) to give the title compound (1%) as a solid. LC/MS MS (ES+) m/e 488 [M+H]⁺.

### Example 77

### (5Z)-2-[(7-Chloro-6-quinoxalinyl)amino]-5-(6-quinolinylmethylidene)-1,3-thiazol-4(5H)-one

a) 2-[(4-Amino-2-chloro-5-nitrophenyl)amino]-1,3-thiazol-4(5H)-one. A mixture of 2-chloro-5-nitro-1,4-benzenediamine (2.10 g, 11.2 mmol), 2-(methylthio)-1,3-thiazol-4(5H)-one (2.10 g, 14.3 mmol) and ethanol (50 mL) was heated under reflux for 18 h, then cooled. The solid was filtered, washed with ethanol and dried to give the title compound (2.69 g, 84%) as a brown powder. 1H NMR (400 MHz, DMSO-d₆) δ 4.02 (s, 2H), 7.23 (s, 1H), 7.44 (br s, 2H), 7.65 (s, 1H), 11.98 (br s, 1H).

b) 2-[(7-Chloro-6-quinoxalinyl)amino]-1,3-thiazol-4(5H)-one. A mixture of the compound from example 77(a) (0.143 g, 0.500 mmol), tin (II) chloride (0.950 g, 5.00 mmol), ethanol (3 mL) and dimethylformamide (3 mL) was stirred at 50° C. for 3 h, then cooled and partitioned between saturated aqueous sodium bicarbonate and ethyl acetate. The extracts were dried (Na₂SO₄) and evaporated to dryness under reduced pressure. A solution of the residue in water (2 mL) and acetonitrile (0.5 mL) was heated under reflux with glyoxal (0.158 mL of a 40% aqueous solution, 1.09 mmol) for 1 h. The mixture was cooled, diluted with water (10 mL) and the solid filtered, washed with water and dried to give the title compound (0.075 g, 54%) as a solid. 1H NMR (400 MHz, DMSO-d₆) δ 4.09 (s, 2H), 7.67 (s, 1H), 8.30 (s, 1H), 8.90 (d, J=1.6 Hz, 1H), 8.93 (d, J=1.6 Hz, 1H), 12.24 (brs, 1H).

c) (5Z)-2-[(7-Chloro-6-quinoxalinyl)amino]-5-(6-quinolinylmethylidene)-1,3-thiazol-4(5H)-one. The method of example 66(b) was followed, using the compound from example 77(b) in place of the compound from example 66(a), to give the title compound (76%) as a brown powder. 1H NMR (400 MHz, DMSO-d₆) δ 7.53 (dd, J=8.4, 4.4 Hz, 1H), 7.83-7.87 (m, 3H), 8.04 (d, J=8.8 Hz, 1H), 8.15 (d, J=1.6 Hz, 1H), 8.35 (s, 1H), 8.42 (d, J=8.0 Hz, 1H), 8.91-8.96 (m, 3H), 12.96 (br s, 1H).

### Example 78

### N-(4-{[(4-Chloro-3-{([(5Z)-4-oxo-5-(6-quinolinylmethylidene)-4,5-dihydro-1,3-thiazol-2-yl]amino}phenyl)amino]sulfonyl}-5-methyl-1,3-thiazol-2-yl)acetamide

The method of example 75 was followed here, using

2-acetamido-4-methyl-5-thiazolesulfonyl chloride in place of phenylmethanesulfonyl chloride, to give the title compound (14%) as a solid. 1H NMR (400 MHz, DMSO-d₆) δ ppm 2.04 (s, 3H) 2.38 (s, 3H) 6.89-6.98 (m, 2H) 7.48 (d, J=8.34 Hz, 1H) 7.59 (dd, J=8.34, 4.04 Hz, 1H) 7.84-7.87 (m, 2H) 8.10 (d, J=8.84 Hz, 1H) 8.17 (s, 1H) 8.45 (d, J=8.08 Hz, 1H) 8.97 (dd, J=4.0, 1.5 Hz, 1H) 10.70 (s, 1H) 12.47 (s, 1H) 12.77 (s, 1H)

### Example 79

### N-(4-Chloro-3-{[(5Z)-4-oxo-5-(6-quinolinylmethylidene)-4,5-dihydro-1,3-thiazol-2-yl]amino}phenyl)-2-thiophenesulfonamide trifluoroacetate

The method of example 75 was followed here, using 2-thiophenesulfonyl chloride in place of phenylmethanesulfonyl chloride. Additionally, the material was purified by rp-HPLC (ODS, 10-90% acetonitrile/water+0.1% trifluoroacetic acid) to give the title compound (6%) as a solid. 1H NMR (400 MHz, DMSO-d₆) δ ppm 6.91-6.98 (m, 2H) 7.03 (dd, J=4.93, 3.92 Hz, 1H) 7.46 (d, J=8.84 Hz, 1H) 7.58-7.63 (m, 2H) 7.82 (dd, J=5.05, 1.26 Hz, 1H) 7.84-7.91 (m, 2H) 8.13 (d, J=8.84 Hz, 1H) 8.19 (d, J=1.52 Hz, 1H) 8.47 (d, J=7.83 Hz, 1H) 8.98 (dd, J=4.29, 1.77 Hz, 1H) 10.65 (s, 1H) 12.76 (s, 1H)

### Example 80

### (5Z)-2-{[2-Chloro-5-(1H-tetrazol-5-yl)phenyl]amino}-5-(6-quinolinylmethylidene)-1,3-thiazol-4(5H)-one

a) 5-(4-Chloro-3-nitrophenyl)-1H-tetrazole. 5-(4-Chlorophenyl)-1H-tetrazole (1.9 g, 10.5 mmoles) was heated in fuming nitric acid (15 mL) at 65° C. for 6 hours. The cooled mixture was poured onto ice, the solid collected, well washed with water and dried. Product (1.8 g, 38%) about 50% pure, used without further purification. 1H NMR (400 MHz, DMSO-d₆) δ ppm 7.93 (d, J=8.34 Hz, 1H) 8.18 (dd, J=8.46, 2.15 Hz, 1H) 8.50 (d, J=2.02 Hz, 1H) 13.84 (s, 1H).

b) 2-Chloro-5-(1H-tetrazol-5-yl)aniline. 5-(4-Chloro-3-nitrophenyl)-1H-tetrazole (1.8 g, 4.0 mmoles) and tin II chloride (3.0 g, 15.8 mmoles) were heated together in ethanol (15 mL) at 65° C. for 2 hours. 1N sodium hydroxide solution was added dropwise to the cooled mixture until a thick solid precipitate formed. The mixture was diluted with water, collected and well washed with ethyl acetate. The layers were separated and the aqueous solution was extracted with ethyl acetate. The combined organic solutions were dried and evaporated to a solid (500 mg, contains about 33% desired product, used without purification). 1H NMR (400 MHz, DMSO-d₆) δ ppm 5.62 (s, 2H) 7.08 (dd, J=8.21, 2.15 Hz, 1H) 7.29 (d, J=8.34 Hz, 1H) 7.40 (d, J=2.02 Hz, 1H) 12.83 (s, 1H).

c) (5Z)-2-{[2-Chloro-5-(1H-tetrazol-5-yl)phenyl]amino}-5-(6-quinolinylmethylidene)-1,3-thiazol-4(5H)-one. A mixture of 2-chloro-5-(1H-tetrazol-5-yl)aniline (480 mg, 33% pure, 0.817 mmoles) and (5Z)-2-(methylthio)-5-(6-quinolinylmethylidene)-1,3-thiazol-4(5H)-one (120 mg, 0.42 mmoles) in dimethylformamide (3.0 mL) was heated at 160° C. for 2 hours, then at 180° C. for a further 1 hour. The mixture was cooled and ethanol added to give a solid. The crude product was slurried in boiling ethanol, collected, washed with ethanol and dichloromethane to give the title compound (60 mg, 17%). 1H NMR (400 MHz, DMSO-d₆) δ ppm 7.56 (dd, J=8.34, 4.29 Hz, 1H) 7.66 (d, J=1.77 Hz, 1H) 7.68-7.73 (m, 1H) 7.77 (dd, J=8.84, 2.02 Hz, 1H) 7.86 (dd, J=8.84, 2.02 Hz, 1H) 7.88 (s, 1H) 8.08 (d, J=8.84 Hz, 1H) 8.16 (d, J=1.52 Hz, 1H) 8.46 (d, J=7.58 Hz, 1H) 8.94 (dd, J=4.17, 1.64 Hz, 1H) 12.86 (s, 1H) 13.30 (s, 1H)

### Example 81

N˜1˜-(4-Chloro-3-{[(5Z)-4-oxo-5-(6-quinolinylmethylidene)-4,5-dihydro-1,3-thiazol-2-yl]amino}phenyl)glycinamide trifluoroacetate

a) 1,1-Dimethylethyl (4-chloro-3-{[(5Z)-4-oxo-5-(6-quinolinylmethylidene)-4,5-dihydro-1,3-thiazol-2-yl ]amino}phenyl)carbamate. A mixture of the compound from example 31(b) (0.100 g, 0.293 mmol), quinoline-6-carbaldehyde (0.060 g, 0.380 mmol), piperidinium acetate in ethanol (1M, 0.293 mL, 0.293 mmol) and ethanol (2 mL) was heated in a microwave reactor at 150° C. for 0.5 h, then cooled. The solid was filtered, washed with ethanol, then dried and chromatographed (silica gel, 1-8% methanol/dichloromethane) to give the title compound (0.086 g, 61%) as a solid. LC/MS MS (ES+) m/e 481 [M+H]⁺

b) (5Z)-2-[(5-Amino-2-chlorophenyl)amino]-5-(6-quinolinylmethylidene)-1,3-thiazol-4(5H)-one. A solution of the compound from example 81(a) (0.086 g, 0.179 mmol) in trifluoroacetic acid (3 mL) was stirred at room temperature for 25 min, then evaporated under reduced pressure. The residue azeotroped with methanol twice, then dissolved in methanol (10 mL). 1M aqueous NaOH was added to adjust the pH to 13, then 1M aq hydrochloric acid was added to neutralise. The precipitate was filtered, washed with water and dried to leave the title compound (0.063 g, 93%) as a yellow powder. 1H NMR (400 MHz, DMSO-d₆) δ ppm 5.37 (s, 2H) 6.29 (s, 1H) 6.41 (dd, J=8.59, 2.53 Hz, 1H) 7.12 (d, J=8.59 Hz, 1H) 7.57 (dd, J=8.34, 4.29 Hz, 1H) 7.84 (s, 1H) 7.87 (dd, J=8.84, 2.02 Hz, 1H) 8.11 (d, J=8.84 Hz, 1H) 8.16 (d, J=1.52 Hz, 1H) 8.47 (d, J=7.58 Hz, 1H) 8.94 (dd, J=4.17, 1.64 Hz, 1H) 12.61 (brs, 1H)

c) N˜1˜-(4-Chloro-3-{[(5Z)-4-oxo-5-(6-quinolinylmethylidene)-4,5-dihydro-1,3-thiazol-2-yl]amino}phenyl)glycinamide. FMOC-aminoacetyl chloride (0.150 g, 0.427 mmol) was added to a stirred mixture of the product from example 81(b) (0.050 g, 0.131 mmol), 2,6-lutidine (0.076 mL, 0.652 mmol) and dioxane (2 mL) and the resulting mixture stirred at 70° C. for 1 h, then cooled. The mixture was purified by rp-HPLC (ODS, 10-90% acetonitrile/water+0.1% trifluoroacetic acid) to give the FMOC intermediate, which was dissolved in 20% piperidine/dimethylformamide solution. After 1 h, the solution was neutralised with acetic acid and purified by rp-HPLC (ODS, 10-90% acetonitrile/water+0.1% trifluoroacetic acid) to give the title compound (0.015 g, 21%) as a solid. 1H NMR (400 MHz, DMSO-d₆) δ 3.78 (d, J=5.81 Hz, 2H) 7.37-7.46 (m, 2H) 7.55 (d, J=8.84 Hz, 1H) 7.58 (m, 1H) 7.86 (dd, J=8.72, 1.89 Hz, 1H) 7.89 (s, 1H) 8.05 (s, 3H) 8.10 (d, J=8.84 Hz, 1H) 8.18 (s, 1H) 8.46 (d, J=8.59 Hz, 1H) 8.95 (dd, J=4.04, 1.52 Hz, 1H) 10.58 (s, 1H) 12.80 (s, 1H).

### Example 82

### N-(4-Chloro-3-{[(5Z)-4-oxo-5-(6-quinolinylmethylidene)-4,5-dihydro-1,3-thiazol-2-yl]amino}phenyl)-2-(1-piperidinyl)acetamide trifluoroacetate

a) 2-Chloro-N-{4-chloro-3-[(4-oxo-4,5-dihydro-1,3-thiazol-2-yl)amino]phenyl}acetamide. Chloroacetyl chloride (0.500 mL, 6.28 mmol) was injected into a stirred mixture of the compound from example 31(c) (0.619 g, 2.56 mmol) and dioxane (5 mL) and stirring continued for 18 h. The mixture was diluted with water (10 mL) and ethyl acetate (10 mL) and the precipitate filtered, washed with water and dried to afford the title compound (0.810 g, 100%) as a solid. 1H NMR (400 MHz, DMSO-d₆) δ ppm 4.04 (s, 2H) 4.26 (s, 2H) 7.29 (dd, J=8.72, 1.89 Hz, 1H) 7.40 (s, 1H) 7.44 (d, J=8.59 Hz, 1H) 10.46 (s, 1H) 12.04 (s, 1H).

b) N-(4-Chloro-3-{[(5Z)-4-oxo-5-(6-quinolinylmethylidene)-4,5-dihydro-1,3-thiazol-2-yl]amino}phenyl)-2-(1-piperidinyl)acetamide. A mixture the compound from example 82(a) (0.100 g, 0.315 mmol), quinoline-6-carbaldehyde (0.050 g, 0.315 mmol), piperidine (0.063 mL, 0.636 mmol) and ethanol (2 mL) was irradiated at 150° C. for 20 min in a microwave reactor. The mixture was allowed to cool to room temperature, diluted with water and acidified with 1M aqueous hydrochloric acid. The mixture was readjusted to pH 10 with aqueous potassium carbonate and the precipitate filtered, washed with water and dried. The solid was purified by rp-HPLC (ODS, 10-90% acetonitrile/water+0.1% trifluoroacetic acid) to afford the title compound (0.028 g, 12%) as a solid. 1H NMR (400 MHz, DMSO-d₆) δ ppm 1.37 (s, 1H) 1.63-1.80 (m, 5H) 2.98-3.09 (m, 2H) 3.41-3.47 (m, 2H) 4.10 (d, J=4.04 Hz, 2H) 7.40 (dd, J=8.59, 2.27 Hz, 1H) 7.46 (d, J=2.02 Hz, 1H) 7.57 (d, J=8.84 Hz, 1H) 7.59 (dd, J=8.34, 4.29 Hz, 1H) 7.86 (dd, J=8.84, 1.77 Hz, 1H) 7.89 (s, 1H) 8.10 (d, J=8.84 Hz, 1H) 8.18 (d, J=1.52 Hz, 1H) 8.47 (d, J=8.08 Hz, 1H) 8.96 (dd, J=4.17, 1.64 Hz, 1H) 9.63 (s, 1H) 10.74 (s, 1H) 12.80 (s, 1H)

### Example 83

### ((5Z)-2-{[2-Chloro-5-(2-pyrimidinyl)phenyl]amino}-5-(6-quinolinylmethylidene)-1,3-thiazol-4(5H)-one

a) 2-(4-Chloro-3-nitrophenyl)pyrimidine. A mixture of 4-chloro-3-nitrophenylboronic acid (520 mg, 2.58 mmoles), 2-bromopyrimidine (410 mg, 2.58 mmoles), tetrakis(triphenylphosphine)palladium(0) (100 mg, 0.08 mmoles) and sodium carbonate (800 mg, 7.5 mmoles) in water (2.5 mL) and dimethylformamide (15 mL) was heated at 100° C. for 1 hour. Water was added and the mixture extracted with ethyl acetate (×2). The combined extracts were dried and evaporated then chromatographed (dichloromethane-methanol) to give the title compound (390 mg, 64%). 1H NMR (400 MHz, CHLOROFORM-d) δ ppm 7.32 (t, J=4.93 Hz, 1H) 7.69 (d, J=8.59 Hz, 1H) 8.64 (dd, J=8.34, 2.02 Hz, 1H) 8.87 (d, J=5.05 Hz, 2H) 9.02 (d, J=2.02 Hz, 1H).

b) 2-Chloro-5-(2-pyrimidinyl)aniline. A mixture of 2-(4-chloro-3-nitrophenyl)pyrimidine (380 mg, 1.6 mmoles) and tin II chloride (3.2 g, 16.8 mmoles) was heated under reflux in ethanol (30 mL) for 2 hours. The mixture was cooled and basified with 1N sodium hydroxide solution and ethyl acetate added. The resultant emulsion was filtered through celite, and the organic layer dried and evaporated to a brown solid (300 mg, 91%). 1H NMR (400 MHz, CHLOROFORM-d) δ ppm 4.17 (s, 2H) 7.26 (t, J=4.80 Hz, 1H) 7.40 (d, J=8.34 Hz, 1H) 7.83 (dd, J=8.34, 2.02 Hz, 1H) 7.96 (d, J=2.02 Hz, 1H) 8.84 (d, J=5.05 Hz, 2H).

c) (5Z)-2-{[2-Chloro-5-(2-pyrimidinyl)phenyl]amino}-5-(6-quinolinylmethylidene)-1,3-thiazol-4(5H)-one. A mixture of 2-chloro-5-(2-pyrimidyl)aniline (120 mg, 0.58 mmoles) and (5Z)-2-(methylthio)-5-(6-quinolinylmethylidene)-1,3-thiazol-4(5H)-one (110 mg, 0.38 mmoles) in dimethylformamide (0.5 mL) was heated at 180° C. for 2 hours. The mixture was cooled and ethanol added to give a solid. The crude product was slurried in boiling ethanol, collected, washed with ethanol and dichloromethane to give the title compound (79 mg, 47%). 1H NMR (400 MHz, DMSO-d₆) δ ppm 7.49 (t, J=4.80 Hz, 1H) 7.53 (dd, J=8.34, 4.29 Hz, 1H) 7.73 (d, J=8.59 Hz, 1H) 7.84 (dd, J=8.84, 2.02 Hz, 1H) 7.88 (s, 1H) 8.05 (d, J=8.84 Hz, 1H) 8.13 (dd, J=8.72, 1.64 Hz, 2H) 8.23 (dd, J=8.34, 2.02 Hz, 1H) 8.42 (d, J=7.58 Hz, 1H) 8.92 (d, J=4.80 Hz, 3H) 12.85 (s, 1H)

### Example 84

### N-(4-Chloro-3-{[(5Z)-4-oxo-5-(6-quinolinylmethylidene)-4,5-dihydro-1,3-thiazol-2-yl]amino}phenyl)-2-cyclopentylacetamide trifluoroacetate

Cyclopentylacetyl chloride (0.028 mL, 0.191 mmol) was added to a mixture of the compound from example 81(b) (0.037 g, 0.097 mmol) and pyridine (0.780 mL, 9.64 mmol) and the mixture stirred at room temperature for 18 h and at 50° C. for 4 h then cooled. The mixture was filtered through a short silica gel column (ethyl acetate) and the residue purified by rp-HPLC (ODS, 10-90% acetonitrile/water+0.1% trifluoroacetic acid) to afford the title compound (0.003 g, 5%) as a solid. LC/MS MS (ES+) m/e 491 [M+H]⁺

### Example 85

### N-(4-Chloro-3-{[(5Z)-4-oxo-5-(6-quinolinylmethylidene)-4,5-dihydro-1,3-thiazol-2-yl]amino}phenyl)-2-(1-pyrrolidinyl)acetamide trifluoroacetate

The method of example 82(b) was followed here, using pyrrolidine in place of piperidine, to give the title compound (8%) as a solid. 1H NMR (400 MHz, DMSO-d₆) δ ppm 1.89 (m, 2H) 1.99 (m, 2H) 3.12 (m, 2H) 3.58 (m 2H) 4.24 (d, J=4.29 Hz, 2H) 7.38-7.48 (m, 2H) 7.56 (d, J=8.84, 1H) 7.60 (dd, J=8.34, 4.29 Hz, 1H) 7.83-7.91 (m, 2H) 8.10 (d, J=8.84 Hz, 1H) 8.19 (d, J=1.77 Hz, 1H) 8.49 (d, J=7.83 Hz, 1H) 8.97 (dd, J=4.17, 1.64 Hz, 1H) 10.03 (s, 1H) 10.76 (s, 1H) 12.83 (s, 1H)

### Example 86

N˜1˜-(4-Chloro-3-{[(5Z)-4-oxo-5-(6-quinolinylmethylidene)-4,5-dihydro-1,3-thiazol-2-yl]amino}phenyl)-N˜2˜-ethyl-N˜2˜-methylglycinamide trifluoroacetate

The method of example 82(b) was followed here, using N-ethylmethylamine in place of piperidine, to give the title compound (7%) as a solid. 1H NMR (400 MHz, DMSO-d₆) δ ppm 1.21 (t, J=7.20 Hz, 3H) 2.83 (d, J=4.29 Hz, 3H) 3.14 (m, 1H) 3.24 (m, 1H) 4.05 (dd, J=15.41, 3.79 Hz, 1H) 4.18 (dd, J=15.41, 5.31 Hz, 1H) 7.40-7.46 (m, 2H) 7.57 (d, J=9.09 Hz, 1H) 7.59 (dd, J=8.34, 4.29 Hz, 1H) 7.86 (dd, J=8.97, 1.89 Hz, 1H) 7.89 (s, 1H) 8.10 (d, J=8.84 Hz, 1H) 8.18 (d, J=1.26 Hz, 1H) 8.46 (d, J=8.34 Hz, 1H) 8.96 (dd, J=4.17, 1.64 Hz, 1H) 9.63 (s, 1H) 10.77 (s, 1H) 12.83 (s, 1H)

### Example 87

### N-(4-Chloro-3-{[(5Z)-4-oxo-5-(6-quinolinylmethylidene)-4,5-dihydro-1,3-thiazol-2-yl]amino}phenyl)-2-(4-morpholinyl)acetamide trifluoroacetate

The method of example 82(b) was followed here, using morpholine in place of piperidine, to give the title compound (16%) as a solid. 1H NMR (400 MHz, DMSO-d₆) δ ppm 3.31 (m, 4H) 3.84 (m, 4H) 4.18 (s, 2H) 7.36-7.47 (m, 2H) 7.57 (d, J=8.59 Hz, 1H) 7.59 (dd, J=8.34, 4.29 Hz, 1H) 7.86 (dd, J=8.97, 1.89 Hz, 1H) 7.89 (s, 1H) 8.10 (d, J=8.59 Hz, 1H) 8.18 (d, J=1.52 Hz, 1H) 8.48 (d, J=7.83 Hz, 1H) 8.96 (dd, J=4.04, 1.52 Hz, 1H) 10.42 (s, 1H) 10.85 (s, 1H) 12.83 (s, 1H)

### Example 88

### 1,1-Dimethylethyl 4-{2-[(4-chloro-3-{[(5Z)-4-oxo-5-(6-quinolinylmethylidene)-4,5-dihydro-1,3-thiazol-2-yl]amino}phenyl)amino]-2-oxoethyl}-1-piperazinecarboxylate

A mixture the compound from example 82(a) (0.150 g, 0.473 mmol), quinoline-6-carbaldehyde (0.075 g, 0.478 mmol), N-(t-butoxycarbonyl)piperazine (0.220 g, 1.18 mmol) and ethanol (2 mL) was irradiated at 150° C. for 20 min in a microwave reactor. The mixture was allowed to cool to room temperature, diluted with water and the precipitate filtered, washed with water and dried. The solid was chromatographed (silica gel, 0-10% methanol/dichloromethane) to afford the title compound (0.092 g, 12%) as a yellow solid. 1H NMR (400 MHz, DMSO-d₆) δ ppm 1.37 (s, 9H) 2.43-2.49 (m, 4H) 3.17 (s, 2H) 3.32-3.40 (m, 4H) 7.47-7.52 (m, 3H) 7.57 (dd, J=8.34, 4.29 Hz, 1H) 7.82-7.90 (m, 2H) 8.09 (d, J=8.84 Hz, 1H) 8.17 (d, J=1.52 Hz, 1H) 8.46 (d, J=8.08 Hz, 1H) 8.94 (dd, J=4.29, 1.77 Hz, 1H) 9.95 (s, 1H) 12.77 (s, 1H)

### Example 89

N′-[(4-Chloro-3-{[(5Z)-4-oxo-5-(6-quinolinylmethylidene)-4,5-dihydro-1,3-thiazol-2-yl]amino}phenyl)methyl]-N,N-dimethylimidoformamide

A solution of (5Z)-2-{[5-(aminomethyl)-2-chlorophenyl]amino}-5-(6-quinolinylmethylidene)-1,3-thiazol-4(5H)-one (example 6(c), 0.094 g, 0.238 mmol), dimethylformamide dimethyl acetal (0.064 mL, 0.482 mmol) and p-toluenesulfonic acid (˜0.002 g) in dimethylformamide (1.5 mL) was heated in a microwave reactor at 100° C. for 0.5 h, then cooled. The solid was filtered, washed with dimethylformamide and ethyl acetate, then dried to give the title compound (0.073 g, 68%) as a pale orange solid. 1H NMR (400 MHz, DMSO-d₆+1% TFA) δ 2.98 (s, 3H), 3.15 (s, 3H), 4.56 (d, J=5.6 Hz, 2H), 7.20 (d, J=1.6 Hz, 1H), 7.24 (dd, J=8.4, 2.0 Hz, 1H), 7.60 (d, J=8.0 Hz, 1H), 7.70 (dd, J=8.4, 4.4 Hz, 1H), 7.90-7.96 (m, 2H), 8.14 (d, J=8.8 Hz, 1H), 8.24 (d, J=1.6 Hz, 1H), 8.28 (d, J=13.2 Hz, 1H), 8.61 (d, J=8.4 Hz, 1H), 9.04 (dd, J=8.8, 2.0 Hz, 1H), 9.51 (dt, J=13.2, 6.4 Hz, 1H), 12.86 (1H).

### Example 90

### N-(4-Chloro-3-{[(5Z)-4-oxo-5-(6-quinolinylmethylidene)-4,5-dihydro-1,3-thiazol-2-yl]amino}phenyl)-2-(1-piperazinyl)acetamide trifluoroacetate

Trifluoroacetic acid (0.5 mL) was added to a solution of the compound from example 88 (0.082 g, 0.135 mmol) in dichloromethane (4 mL) and the mixture allowed to stand for 0.5 h, then evaporated to dryness under reduced pressure. The residue was purified by rp-HPLC (ODS, 10-90% acetonitrile/water+0.1% trifluoroacetic acid) to afford the title compound (0.012 g, 18%) as a solid. LC/MS MS (ES+) m/e 507 [M+H]⁺

### Example 91

### N-(4-Chloro-3-{[(5Z)-4-oxo-5-(6-quinolinylmethylidene)-4,5-dihydro-1,3-thiazol-2-yl]amino}phenyl)-2-pyridinecarboxamide

Nicotinoyl chloride hydrochloride (0.070 mL, 0.393 mmol) was added to a mixture of the compound from example 81(b) (0.100 g, 0.263 mmol) and pyridine (1 mL) and the mixture stirred at 50° C. for 18 h then cooled. Water (5 mL) was added and the precipitate filtered, washed with 1M aqueous hydrochloric acid and ethyl acetate, then dried to afford the title compound (0.020 g, 14%) as a solid. 1H NMR (400 MHz, DMSO-d₆) δ ppm 7.55-7.67 (m, 5H) 7.89 (s, 1H) 7.91 (dd, J=8.97, 1.89 Hz, 1H) 8.12 (d, J=8.84 Hz, 1H) 8.22 (d, J=1.26 Hz, 1H) 8.34 (dt, J=8.08, 1.89 Hz, 1H) 8.55 (d, J=8.34 Hz, 1H) 8.79 (dd, J=4.67, 1.39 Hz, 1H) 8.98 (dd, J=4.29, 1.52 Hz, 1H) 9.13 (d, J=1.77 Hz, 1H) 10.69 (s, 1H) 12.83 (s, 1H)

### Example 92

### N-(4-Chloro-3-{[(5Z)-4-oxo-5-(6-quinolinylmethylidene)-4,5-dihydro-1,3-thiazol-2-yl]amino}phenyl)cyclohexanecarboxamide

a) N-(4-Chloro-3-nitrophenyl)cyclohexanecarboxamide. Cyclohexanecarbonyl chloride (1.80 mL, 11.3 mmol) was injected slowly into a stirred solution of 4-chloro-3-nitroaniline (2.00 g, 11.59 mmol) and pyridine (1.00 mL, 12.5 mmol) in dichloromethane (15 mL) at room temperature and the mixture stirred for 18 h. 1M aqueous hydrochloric acid (50 mL) was added and the mixture extracted with dichloromethane. The extracts were dried (MgSO₄) and evaporated under reduced pressure to leave the title compound (3.30 g, 100%) as a brown solid. 1H NMR (400 MHz, CHLOROFORM-d) δ 1.25-1.36 (m, 3H) 1.51-1.61 (m, 2H) 1.74 (dd, J=9.09, 3.28 Hz, 1H) 1.85-1.91 (m, 2H) 1.94-2.01 (m, 2H) 2.28 (tt, J=11.65, 3.51 Hz, 1H) 7.40 (br s, 1H) 7.49 (d, J=8.84 Hz, 1H) 7.73 (dd, J=8.84, 2.78 Hz, 1H) 8.21 (d, J=2.53 Hz, 1H)

b) N-(3-Amino-4-chlorophenyl)cyclohexanecarboxamide. Zinc powder (1.16 g, 17.7 mmol) was added in portions to a stirred solution of the compound from example 92(a) (0.501 g, 1.77 mmol) in acetic acid (15 mL). The mixture was stirred 0.5 h then filtered and poured into water (100 mL) and made basic with aqueous sodium hydroxide. The solid was filtered, washed with water and dried to leave the title compound (0.38 g, 85%) as a white solid. 1H NMR (400 MHz, CHLOROFORM-d) δ ppm 1.24-1.34 (m, 3H) 1.48-1.55 (m, 2H) 1.71 (m, 1H) 1.79-1.88 (m, 2H) 1.91-1.98 (m, 2H) 2.20 (tt, J=11.62, 3.41 Hz, 1H) 4.07 (s, 2H) 6.56 (dd, J=8.59, 2.53 Hz, 1H) 7.02 (s, 1H) 7.14 (d, J=8.34 Hz, 1H) 7.39 (d, J=2.27 Hz, 1H)

c) N-(4-Chloro-3-{[(5Z)-4-oxo-5-(6-quinolinylmethylidene)-4,5-dihydro-1,3-thiazol-2-yl]amino}phenyl)cyclohexanecarboxamide. A mixture of the compound from example 92(b) (0.052 g, 0.206 mmol), (5Z)-2-(methylthio)-5-(6-quinolinylmethylidene)-1,3-thiazol-4(5H)-one (0.050 g, 0.175 mmol) and pentanoic acid (0.5 mL) was stirred at 180° C. for 0.5 h, then cooled and diluted with ethanol (1 mL). The solid was filtered, washed with dichloromethane and dried to give the title compound (0.045 g, 44%) as a pale yellow solid. 1H NMR (400 MHz, DMSO-d₆) δ ppm 1.15-1.44 (m, 5H) 1.63 (m, 1H) 1.72-1.83 (m, 4H) 2.31 (tt, J=11.62, 3.03 Hz, 1H) 7.40-7.50 (m, 3H) 7.56 (dd, J=8.34, 4.04 Hz, 1H) 7.83-7.88 (m, 2H) 8.09 (d, J=8.84 Hz, 1H) 8.17 (d, J=1.52 Hz, 1H) 8.47 (d, J=7.58 Hz, 1H) 8.94 (dd, J=4.04, 1.52 Hz, 1H) 10.03 (s, 1H) 12.76 (s, 1H)

### Example 93

### N-(4-Chloro-3-{[(5Z)-4-oxo-5-(6-quinolinylmethylidene)-4,5-dihydro-1,3-thiazol-2-yl]amino}phenyl)benzamide trifluoroacetate

The method of example 69 was followed here, using benzoyl chloride in place of 3,4-dimethoxyphenylacetyl chloride. Additionally, the material was purified by rp-HPLC (ODS, 10-90% acetonitrile/water+0.1% trifluoroacetic acid) to give the title compound (1%) as a solid. 1H NMR (400 MHz, DMSO-d₆) δ ppm 7.51-7.66 (m, 7H) 7.83 (s, 1H) 7.87 (dd, J=8.84, 1.52 Hz, 1H) 7.93-7.96 (m, 2H) 8.08 (d, J=8.84 Hz, 1H) 8.16 (d, J=1.77 Hz, 1H) 8.45 (d, J=7.58 Hz, 1H) 8.92 (dd, J=4.17, 1.64 Hz, 1H) 10.44 (s, 1H) 12.75 (s, 1H)

### Example 94

### N-(4-Chloro-3-{[(5Z)-4-oxo-5-(6-quinolinylmethylidene)-4,5-dihydro-1,3-thiazol-2-yl]amino}phenyl)cyclopentanecarboxamide

a) N-{4-Chloro-3-[(4-oxo-4,5-dihydro-1,3-thiazol-2-yl)amino]phenyl}cyclopentanecarboxamide. A solution of the compound from example 31(b) (0.100 g, 0.293 mmol) in trifluoroacetic acid (1 mL) was stirred at room temperature for 20 min, then evaporated under reduced pressure. The residue was dissolved in pyridine (2 mL) and cyclopentanecarbonyl chloride (0.071 mL, 0.585 mmol) injected dropwise with stirring. After 18 h, added water (10 mL) and heated under reflux for 1 h, then cooled, acidified with 1M aqueous hydrochloric acid and extracted with ethyl acetate. The extracts were washed (aqueous sodium bicarbonate, water, brine), dried (MgSO₄) and evaporated under reduced pressure. The residue was chromatographed (silica gel, 2-8% methanol/dichloromethane) to give the title compound (0.042 g, 42%) as a yellow solid. 1H NMR (400 MHz, DMSO-d₆) δ 1.54-1.88 (m, 8H), 2.75 (m, 1H), 4.03 (s, 2H), 7.29 (d, J=8.6 Hz, 1H), 7.38 (d, J=8.9 Hz, 1H), 7.45 (s, 1H), 9.99 (s, 1H), 11.98 (br s, 1H).

b) N-(4-Chloro-3-{[(5Z)-4-oxo-5-(6-quinolinylmethylidene)-4,5-dihydro-1,3-thiazol-2-yl]amino}phenyl)cyclopentanecarboxamide. The method of example 66(b) was followed, using the compound from example 94(c) in place of the compound from example 66(a), to give the title compound (53%) as a yellow powder. 1H NMR (400 MHz, DMSO-d₆) δ 1.52-1.88 (m, 8H), 2.76 (m, 1H), 7.43-7.45 (m, 3H), 7.56 (dd, J=8.4, 4.3 Hz, 1H), 7.82 (s, 1H), 7.87 (d, J=8.6 Hz, 1H), 8.08 (d, J=8.6 Hz, 1H), 8.15 (d, J=1.5 Hz, 1H), 8.45 (d, J=7.6 Hz, 1H), 8.93 (dd, J=4.3, 1.5 Hz, 1H), 10.04 (s, 1H), 12.69 (br s, 1H).

### Example 95

### (5Z)-2-{[2-Chloro-5-(3-thienyl)phenyl]amino}-5-(6-quinolinylmethylidene)-1,3-thiazol-4(5H)-one

a) (5Z)-2-[(5-Bromo-2-chlorophenyl)amino]-5-(2-naphthalenylmethylidene)-1,3-thiazol-4(5H)-one. A mixture of 5-bromo-2-chloroaniline (Suthers et. al., JOC, 1962, 27; 447, 1.06 g, 5.13 mmoles) and (5Z)-2-(methylthio)-5-(6-quinolinylmethylidene)-1,3-thiazol-4(5H)-one (1.0 g, 3.49 mmoles) in valeric acid (4.0 mL) was heated at 180° C. for 30 minutes. The mixture was cooled and ethanol added to give a solid. The crude product was collected, washed with ethanol and dichloromethane to give the title compound (1.26 g, 81%). 1H NMR (400 MHz, DMSO-d₆) δ ppm 7.39-7.47 (m, 2H) 7.52-7.58 (m, 2H) 7.86 (d, J=2.02 Hz, 1H) 7.89 (s, 2H) 8.10 (d, J=8.59 Hz, 1H) 8.18 (d, J=1.52 Hz, 1H) 8.48 (d, J=7.83 Hz, 1H) 8.95 (dd, J=4.17, 1.64 Hz, 1H) 12.87 (s, 1H).

b) (5Z)-2-{[2-Chloro-5-(3-thienyl)phenyl]amino}-5-(6-quinolinylmethylidene)-1,3-thiazol-4(5H)-one. A mixture of 3-thiopheneboronic acid (30 mg, 0.23 mmoles), (5Z)-2-[(5-bromo-2-chlorophenyl)amino]-5-(2-naphthalenylmethylidene)-1,3-thiazol-4(5H)-one (100 mg, 0.22 mmoles), sodium carbonate (100 mg, 0.94 mmoles) and tetrakis(triphenylphosphine)palladium(0) (15 mg, 0.013 mmoles) in water (1.0 mL) and DMF (2.0 mL) was heated at 100° C. for 18 hours. The mixture was diluted with water and the resulting solid was collected, washed with water, ethanol and dichloromethane. Dissolved in ethanol dil. NaOH solution and reprecipitated with acetic acid to give the title compound as a yellow solid (55 mg, 56%). 1H NMR (400 MHz, DMSO-d₆) δ ppm 7.52-7.66 (m, 6H) 7.84-7.86 (m, 2H) 8.00 (s, 1H) 8.06 (d, J=8.84 Hz, 1H) 8.14 (s, 1H) 8.43 (d, J=7.83 Hz, 1H) 8.91 (dd, J=4.29, 1.52 Hz, 1H) 12.80 (s, 1H)

### Example 96

### (5Z)-2-({2-Chloro-5-[6-(methyloxy)-2-pyridinyl]phenyl}amino)-5-(6-quinolinylmethylidene)-1,3-thiazol-4(5H)-one

A mixture of [6-(methyloxy)-2-pyridinyl]boronic acid (100 mg, 0.65 mmoles), (5Z)-2-[(5-bromo-2-chlorophenyl)amino]-5-(2-naphthalenylmethylidene)-1,3-thiazol-4(5H)-one (130 mg, 0.29 mmoles), sodium carbonate (150 mg, 1.41 mmoles) and tetrakis(triphenylphosphine)palladium(0) (30 mg, 0.026 mmoles) in water (1.0 mL) and DMF (2.0 mL) was heated at 150° C. for 3 minutes in a microwave reactor. The mixture was diluted with water and extracted with ethyl acetate (×2), the combined extracts dried and evaporated. Recrystallization from ethanol gave the title compound (10 mg, 7.3%). 1H NMR (400 MHz, DMSO-d₆) δ ppm 3.88 (s, 3H) 6.91 (d, J=8.59 Hz, 1H) 7.51 (s, 1H) 7.55 (dd, J=8.21, 4.17 Hz, 2H) 7.64 (d, J=8.34 Hz, 1H) 7.83-7.93 (m, 2H) 8.07 (td, J=5.75, 2.65 Hz, 2H) 8.16 (s, 1H) 8.44 (d, J=8.08 Hz, 1H) 8.56 (d, J=2.27 Hz, 1H) 8.93 (d, J=2.78 Hz, 1H) 12.85 (s, 1H)

### Example 97

### (5Z)-2-[(3,5-Dichloro-2,6-dimethyl-4-pyridinyl)amino]-5-(6-quinoxalinylmethylidene)-1,3-thiazol-4(5H)-one

a) 3,5-Dichloro-2,6-dimethyl-4-pyridinamine. 3,4,5-Trichloro-2,6-dimethylpyridine (5.0 g, 23.75 mmoles) in 7.0 molar ammonia in methanol (100 mL) was heated in a steel bomb at 180° C. for 20 hours. The cooled mixture was evaporated, the solid extracted into dichloromethane and chromatographed (0-10% methanol in dichloromethane) to give the title compound (2.38 g, 52%). 1H NMR (400 MHz, CHLOROFORM-d) δ ppm 2.53 (s, 6H) 4.99 (s, 2H).

b) (5Z)-2-[(3,5-Dichloro-2,6-dimethyl-4-pyridinyl)amino]-5-(6-quinoxalinylmethylidene)-1,3-thiazol-4(5H)-one. Sodium hydride (60% in mineral oil, 238 mg, 5.9 mmoles) was added to a solution of 3,5-dichloro-2,6-dimethyl-4-pyridinamine (338 mg, 1.77 mmoles) in N-methylpyrrolidinone (12 mL) under argon atmosphere. After stirring for 10 minutes, (5Z)-2-(methylthio)-5-(6-quinoxalinylmethylidene)-1,3-thiazol-4(5H)-one (363 mg, 1.26 mmoles) was added and stirring was continued for 60 minutes. The mixture was poured onto ice, water added, and the solid collected onto celite and washed with water. The collection flask was changed and the product extracted by washing through with dichloromethane-methanol. Flash chromatography (0-5% methanol in dichloromethane gave crude material that was purified by preparative HPLC (0-70% acetonitrile-water-0.1% TFA) gave the title compound (47 mg, 8.7%) 1H NMR (400 MHz, DMSO-d₆) δ ppm 2.55 (s, 6H) 7.99 (dd, J=8.84, 2.02 Hz, 1H) 8.03 (s, 1H) 8.17 (d, J=8.84 Hz, 1H) 8.23 (d, J=2.02 Hz, 1H) 8.99 (s, 2H) 13.18 (s, 1H)

### Example 98

### (5Z)-2-{[2-Chloro-5-(4-morpholinyl)phenyl]amino}-5-(6-quinolinylmethylidene)-1,3-thiazol-4(5H)-one

a) 4-(4-Chloro-3-nitrophenyl)morpholine. A mixture of 4-chloro-3-nitroaniline (6.24 g, 36.1 mmoles), bis(2-bromoethyl)ether (5.4 mL, 43.3 mmoles) and potassium carbonate (10.0 g, 72 mmoles) were heated together under reflux in dimethoxyethane (20 mL) for 48 hours. The title compound was obtained as a solid after separation by preparative HPLC (2.0 g, 23%). 1H NMR (400 MHz, CHLOROFORM-d) δ ppm 3.21-3.27 (m, 4H) 3.86-3.94 (m, 4H) 7.06 (dd, J=9.09, 3.03 Hz, 1H) 7.37 (d, J=3.03 Hz, 1H) 7.41 (d, J=8.84 Hz, 1H).

b) 2-Chloro-5-(4-morpholinyl)aniline. A mixture of 4-(4-chloro-3-nitrophenyl)morpholine (1.3 g, 5.35 mmoles) and tin II chloride (9.0 g, 47.4 mmoles) in ethanol (50 mL) was heated under reflux for 1.5 hours. The mixture was poured into ice-water, basified with dilute sodium hydroxide solution and filtered through celite. The filter bed was well washed with water and ethyl acetate, the layers separated and the aqueous extracted with ethyl acetate. The organic solution was dried and evaporated to give the title compound (1.0 g, 88%). 1H NMR (400 MHz, CHLOROFORM-d) δ ppm 3.09-3.18 (m, 4H) 3.84-3.94 (m, 4H) 6.33 (dd, J=8.72, 2.65 Hz, 1H) 6.38 (d, J=2.53 Hz, 1H) 7.14 (d, J=8.59 Hz, 1H).

c) (5Z)-2-{[2-Chloro-5-(4-morpholinyl)phenyl]amino}-5-(6-quinolinylmethylidene)-1,3-thiazol-4(5H)-one. A mixture of 2-chloro-5-(4-morpholinyl)aniline (145 mg, 0.68 mmoles) and (5Z)-2-(methylthio)-5-(6-quinolinylmethylidene)-1,3-thiazol-4(5H)-one (128 mg, 0.45 mmoles) in valeric acid (0.5 mL) was heated at 150° C. for 30 minutes. The cooled mixture was diluted with ethanol and the solid collected. The solid was dissolved in hot ethanol and 1N sodium hydroxide and re-precipitated with acetic acid to give the title compound (90 mg, 44%). 1H NMR (400 MHz, DMSO-d₆) δ ppm 3.10-3.17 (m, 4H) 3.68-3.76 (m, 4H) 6.70 (s, 1H) 6.80 (dd, J=8.97, 2.65 Hz, 1H) 7.35 (d, J=9.09 Hz, 1H) 7.57 (dd, J=8.34, 4.04 Hz, 1H) 7.84-7.89 (m, 2H) 8.09 (d, J=8.84 Hz, 1H) 8.15 (d, J=1.77 Hz, 1H) 8.46 (d, J=7.58 Hz, 1H) 8.94 (dd, J=4.17, 1.64 Hz, 1H) 12.73 (s, 1H)

### Example 99

N˜1˜-(4-Chloro-3-{[(5Z)-4-oxo-5-(6-quinolinylmethylidene)-4,5-dihydro-1,3-thiazol-2-yl]amino}phenyl)leucinamide trifluoroacetate

FMOC-Leu chloride (0.290 g, 0.780 mmol) was added to a stirred mixture of the product from example 81(b) (0.100 g, 0.262 mmol), 2,6-lutidine (0.152 mL, 1.30 mmol) and dioxane (2 mL) and the resulting mixture stirred at room temperature for 60 h. Dichloromethane was added and the resulting precipitate filtered and dried. The FMOC intermediate was dissolved in 20% piperidine/dimethylformamide (2 mL) solution. After 1 h, the solution was neutralised with acetic acid and purified by rp-HPLC (ODS, 10-90% acetonitrile/water+0.1% trifluoroacetic acid) to give the title compound (0.004 g, 5%) as a solid. 1H NMR (400 MHz, DMSO-d₆) δ ppm 0.91 (d, J=3.28 Hz, 3H) 0.92 (d, J=3.54 Hz, 3H) 1.66 (m, 3H) 3.90 (m, 1H) 7.42 (dd, J=8.84, 2.53 Hz, 1H) 7.47 (s, 1H) 7.57 (d, J=8.34 Hz, 1H) 7.59 (dd, J=8.34, 4.04 Hz, 1H) 7.86 (dd, J=8.84, 2.02 Hz, 1H) 7.89 (s, 1H) 8.10 (d, J=8.84 Hz, 1H) 8.18-8.22 (m, 4H) 8.46 (d, J=7.33 Hz, 1H) 8.95 (dd, J=4.17, 1.64 Hz, 1H) 10.66 (s, 1H) 12.83 (s, 1H)

### Example 100

### N-(4-Chloro-3-{[(5Z)-4-oxo-5-(6-quinolinylmethylidene)-4,5-dihydro-1,3-thiazol-2-yl]amino}phenyl)-2-methylpropanamide trifluoroacetate

The method of example 69 was followed here, using 2-methylpropionyl chloride in place of 3,4-dimethoxyphenylacetyl chloride. Additionally, the material was purified by rp-HPLC (ODS, 10-90% acetonitrile/water+0.1% trifluoroacetic acid) to give the title compound (6%) as a solid. 1H NMR (400 MHz, DMSO-d₆) δ ppm 1.09 (d, J=6.82 Hz, 6H) 2.58 (septet, J=6.82, 1H) 7.41-7.51 (m, 3H) 7.57 (dd, J=8.21, 4.17 Hz, 1H) 7.85 (dd, J=8.84, 2.02 Hz, 1H) 7.87 (s, 1H) 8.09 (d, J=8.84 Hz, 1H) 8.17 (d, J=1.77 Hz, 1H) 8.47 (d, J=7.83 Hz, 1H) 8.94 (dd, J=4.17, 1.64 Hz, 1H) 10.05 (s, 1H) 12.77 (s, 1H)

### Example 101

### (5Z)-2-{[5-(2-Amino-5-pyrimidinyl)-2-chlorophenyl]amino}-5-(6-quinolinylmethylidene)-1,3-thiazol-4(5H)-one trifluoroacetate

a) 5-(4-Chloro-3-nitrophenyl)-2-pyrimidinamine. A mixture of 4-chloro-3-nitrophenylboronic acid (600 mg, 3.0 mmoles), 2-amino-5-iodopyrimidine (680 mg, 3.0 mmoles), sodium carbonate (900 mg, 8.5 mmoles) and tetrakis(triphenylphosphine)palladium(0) (80 mg, 0.07 mmoles) in 3:1 dimethylformamide-water (10 mL) was sealed in a pressure tube and heated at 150° C. for 10 minutes in a microwave reactor. The mixture was diluted with water and extracted with ethyl acetate (×2). Combined extracts were washed with brine, dried and evaporated. Flash chromatography (0-5% methanol-dichloromethane) gave the title compound (525 mg, 70%). 1H NMR (400 MHz, DMSO-d₆) δ ppm 7.04 (s, 2H) 7.81 (d, J=8.59 Hz, 1H) 8.00 (dd, J=8.59, 2.27 Hz, 1H) 8.36 (d, J=2.27 Hz, 1H) 8.70 (s, 2H).

b) 5-(3-Amino-4-chlorophenyl)-2-pyrimidinamine. A mixture of 5-(4-chloro-3-nitrophenyl)-2-pyrimidinamine (520 mg, 2.08 mmoles) and tin II chloride (6.0 g, 32 mmoles) was heated under reflux in ethanol (100 mL) for 2 hours. The mixture was diluted with water and basified with 1N sodium hydroxide solution. The suspension was filtered through celite and the filter bed well washed with ethyl acetate. The organic solution was separated, dried and evaporated to a solid that was slurried in ether, collected, washed with ether and hexane to give the title compound (300 mg, 65%). 1H NMR (400 MHz, DMSO-d₆) δ ppm 6.80 (dd, J=8.21, 2.15 Hz, 1H) 7.00 (d, J=2.27 Hz, 1H) 7.25 (d, J=8.08 Hz, 1H) 8.54 (s, 2H).

c) (5Z)-2-{[5-(2-Amino-5-pyrimidinyl)-2-chlorophenyl]amino}-5-(6-quinolinylmethylidene)-1,3-thiazol-4(5H)-one. A mixture of 5-(3-amino-4-chlorophenyl)-2-pyrimidinamine (108 mg, 0.49 mmoles) and (5Z)-2-(methylthio)-5-(6-quinolinylmethylidene)-1,3-thiazol-4(5H)-one (100 mg, 0.40 mmoles) in valeric acid (2.0 mL) was heated at 180° C. for 20 minutes. The liquid decanted and diluted with ethanol and the solid collected. The solid was purified by preparative HPLC (10-80% acetonitrile-water-0.1% TFA) to give the title compound (15 mg, 9%). 1H NMR (400 MHz, DMSO-d₆) δ ppm 6.88 (s, 2H) 7.47-7.52 (m, 2H) 7.54-7.61 (m, 2H) 7.83-7.92 (m, 2H) 8.07 (d, J=8.59 Hz, 1H) 8.16 (s, 1H) 8.45 (d, J=7.58 Hz, 1H) 8.64 (s, 2H) 8.93 (d, J=2.78 Hz, 1H) 12.83 (s, 1H)

### Example 102

### (5Z)-2-({2-Chloro-5-[(dimethylamino)methyl]phenyl}amino)-5-(6-quinolinylmethylidene)-1,3-thiazol-4(5H)-one

A mixture of the compound from example 6(c) (0.050 g, 0.127 mmol), formaldehyde (0.094 mL of a 37% aqueous solution, 1.27 mmol) and formic acid (1 mL) was heated under reflux for 5 h, then cooled, diluted with water (10 mL) and extracted with ethyl acetate. The extracts were dried (Na₂SO₄) and evaporated under reduced pressure. The residue was boiled in ethyl acetate (10 mL) and, after cooling, the solid filtered. The material was further purified by chromatography (silica gel, 8-10% methanol/dichloromethane) to give the title compound (0.034 g, 63%) as a yellow solid. 1H NMR (400 MHz, DMSO-d₆+1% TFA) δ 2.77 (d, J=4 Hz, 6H), 4.31 (d, J=4.0 Hz, 2H), 7.33 (s, 1H), 7.36 (dd, J=8.4, 2.1 Hz, 1H), 7.68-7.76 (m, 2H), 7.91 (s, 1H), 7.95 (dd, J=8.9, 1.5 Hz, 1H), 8.14 (d, J=8.9 Hz, 1H), 8.23 (s, 1H), 8.61 (d, J=7.8 Hz, 1H), 9.04 (dd, J=4.3, 1.3 Hz, 1H), 9.84 (brs, 1H), 12.91 (brs, 1H).

### Example 103

### N-[(4-Chloro-3-{[(5Z)-4-oxo-5-(6-quinolinylmethylidene)-4,5-dihydro-1,3-thiazol-2-yl]amino}phenyl)methyl]methanesulfonamide

Methanesulfonyl chloride was added to a stirred suspension of the compound from example 6(c) (0.050 g, 0.127 mmol) in pyridine (1 mL). After stirring 5 h at room temperature, the mixture was diluted with water (10 mL). Aqueous sodium hydroxide was added to pH 13, and the solid re-precipitated by adding acetic acid. After filtering, the material was chromatographed (silica gel, 1-9% methanol/dichloromethane), then triturated with ether and dried to give the title compound (0.016 g, 27%) as a pale yellow powder. 1H NMR (400 MHz, DMSO-d₆) δ 2.89 (s, 3H), 4.19 (d, J=6.3 Hz, 2H), 7.16 (s, 1H), 7.20 (d, J=8.4 Hz, 1H), 7.54 (d, J=8.4 Hz, 1H), 7.58 (dd, J=8.3, 4.3 Hz, 1H), 7.66 (t, J=6.3 Hz, 1H), 7.82-7.91 (m, 2H), 8.08 (d, J=8.8 Hz, 1H), 8.15 (d, J=1.3 Hz, 1H), 8.44 (d, J=7.8 Hz, 1H), 8.94 (dd, J=4.3, 1.5 Hz, 1H), 12.77 (br s, 1H).

### Example 104

### 4-Chloro-N,N-dimethyl-3-{[(5Z)-4-oxo-5-(6-quinolinylmethylidene)-4,5-dihydro-1,3-thiazol-2-yl]amino}benzenesulfonamide

The title compound was obtained as a yellow powder (102 mg, 45%) by following the procedure of Examples 9, except substituting N,N-dimethylamine for cyclobutylamine. ¹H NMR (400 MHz, DMSO-d₆) δ ppm 12.92 (br s, 1H), 8.94 (dd, J=4.2, 1.6 Hz, 1H), 8.41 (d, J=7.8 Hz, 1H), 8.14 (d, J=1.3 Hz, 1H), 8.08 (d, J=8.8 Hz, 1H), 7.89-7.84 (m, 3H), 7.59-7.56 (m, 3H), 2.68 (s, 6H).

### Example 105

### N-(2,4-Dichloro-5-{[(5Z)-4-oxo-5-(6-quinolinylmethylidene)-4,5-dihydro-1,3-thiazol-2-yl]amino}phenyl)cyclobutanecarboxamide

a) 4,6-Dichloro-1,3-benzenediamine. A solution of 1,5-dichloro-2,4-dinitrobenzene (1.08 g, 4.56 mmol) in methanol (50 mL) was stirred with Raney® nickel (˜0.1 g) under hydrogen at room pressure for 18 h, then the hydrogen flushed out with nitrogen and the mixture filtered through a micropore filter. The solvent was removed under reduced pressure to give the title compound (0.810 g, 100%). 1H NMR (400 MHz, DMSO-d₆) δ 5.13 (br s, 4H), 6.21 (s, 1H), 6.99 (s, 1H).

b) 2-[(5-Amino-2,4-dichlorophenyl)amino]-1,3-thiazol-4(5H)-one. A mixture of 4,6-dichloro-1,3-benzenediamine (0.684 g, 3.86 mmol) and 2-(methylthio)-1,3-thiazol-4(5H)-one (0.294 g, 2.00 mmol) in ethanol (10 mL) was heated under reflux for 5 h, then cooled. The solvent was evaporated under reduced pressure and the residue chromatographed (silica gel, 1-7% methanol/dichloromethane) to give the title compound (0.380 g, 69%) as a solid. ¹H NMR (400 MHz, DMSO-d₆) δ 4.02 (s, 2H), 5.53 (br s, 2H), 6.41 (s, 1H), 7.31 (s, 1H), 11.93 (br s, 1H).

c) N-{2,4-Dichloro-5-[(4-oxo-4,5-dihydro-1,3-thiazol-2-yl)amino]phenyl}cyclobutanecarboxamide. Cyclobutanecarbonyl chloride (0.068 mL, 0.596 mmol) was injected dropwise into a stirred solution of the compound from example 105(b) (0.110 g, 0.398 mmol) in pyridine (1 mL). After 18 h, added 1M aqueous potassium hydroxide (5 mL) and methanol (5 mL) and stirred 0.5 h, then acidified with 1M aqueous hydrochloric acid and extracted with ethyl acetate. The extracts were washed (aqueous hydrochloric acid, aqueous sodium bicarbonate, water, brine), dried (MgSO₄) and evaporated under reduced pressure, then the residue chromatographed (silica gel, 30-70% ethyl acetate/hexane) to give the title compound (0.134 g, 94%) as a solid. 1H NMR (400 MHz, DMSO-d₆) δ 1.80-2.24 (m, 6H), 3.37 (m, 1H), 4.05 (s, 2H), 7.49 (s, 1H), 7.69 (s, 1H), 9.33 (s, 1H), 12.07 (brs, 1H).

d) N-(2,4-Dichloro-5-{[(5Z)-4-oxo-5-(6-quinolinylmethylidene)-4,5-dihydro-1,3-thiazol-2-yl]amino}phenyl)cyclobutanecarboxamide. A mixture of the compound from example 105(c) (0.060 g, 0.167 mmol), quinoline-6-carbaldehyde (0.031 g, 0.197 mmol), piperidinium acetate in ethanol (1M, 0.167 mL, 0.167 mmol) and ethanol (0.5 mL) was heated in a microwave reactor at 150° C. for 0.5 h, then cooled, The solid was filtered, washed with ethanol and dried to give the title compound (0.061 g, 73%) as a pale yellow powder. 1H NMR (400 MHz, DMSO-d₆) δ 1.81 (m, 1H), 1.91 (m, 1H), 2.11-2.24 (m, 4H), 3.38 (m, 1H), 7.56-7.60 (m, 2H), 7.76 (s, 1H), 7.86 (s, 1H), 7.89 (dd, J=9.1, 1.7 Hz, 1H), 8.09 (d, J=8.9 Hz, 1H), 8.20 (d, J=2.0 Hz, 1H), 8.48 (d, J=7.6 Hz, 1H), 8.95 (dd, J=4.0, 1.5 Hz, 1H), 9.40 (s, 1H), 12.79 (br s, 1H).

### Example 106

### (5Z)-2-{[4-Chloro-3′-(methyloxy)-3-biphenylyl]amino}-5-(6-quinolinylmethylidene)-1,3-thiazol-4(5H)-one

a) 4-Chloro-3′-(methyloxy)-3-nitrobiphenyl. A mixture of 4-chloro-3-nitrophenylboronic acid (427 mg, 2.13 mmoles), 3-iodoanisole (500 mg, 2.13 mmoles), sodium carbonate (600 mg, 5.66 mmoles) and tetrakis(triphenylphosphine)palladium(0) (50 mg, 0.04 mmoles) in 3:1 dimethylformamide-water (5 mL) was sealed in a pressure tube and heated at 150° C. for 10 minutes in a microwave reactor. The mixture was diluted with water and extracted with ethyl acetate (×2). Combined extracts were washed with brine, dried and evaporated. Flash chromatography (0-30% ethyl acetate-hexane) gave the title compound (250 mg, 44.5%). 1H NMR (400 MHz, CHLOROFORM-d) δ ppm 3.89 (s, 3H) 6.98 (ddd, J=8.34, 2.53, 0.76 Hz, 1H) 7.06-7.12 (m, 1H) 7.16 (ddd, J=7.71, 1.64, 0.76 Hz, 1H) 7.41 (t, J=7.96 Hz, 1H) 7.61 (d, J=8.59 Hz, 1H) 7.73 (dd, J=8.34, 2.27 Hz, 1H) 8.08 (d, J=2.02 Hz, 1H).

b) 4-Chloro-3′-(methyloxy)-3-biphenylamine. A mixture of 4-chloro-3′-(methyloxy)-3-nitrobiphenyl (250 mg, 0.95 mmoles) and tin II chloride (1.0 g, 5.29 mmoles) was heated under reflux in ethanol (50 mL) for 2 hours. The mixture was diluted with water and basified with 1N sodium hydroxide solution. The suspension was filtered through celite and the filter bed well washed with ethyl acetate. The organic solution was separated, dried and evaporated to give the title compound (220 mg, quant.). 1H NMR (400 MHz, CHLOROFORM-d) δ ppm 3.88 (s, 3H) 6.91-6.95 (m, 2H) 7.00 (d, J=2.27 Hz, 1H) 7.08-7.11 (m, 1H) 7.14 (ddd, J=7.64, 1.58, 0.88 Hz, 1H) 7.32 (d, J=8.08 Hz, 1H) 7.36 (t, J=7.96 Hz, 1H).

c) (5Z)-2-{[4-Chloro-3′-(methyloxy)-3-biphenylyl]amino}-5-(6-quinolinylmethylidene)-1,3-thiazol-4(5H)-one. A mixture of 4-chloro-3′-(methyloxy)-3-biphenylamine (220 mg, 0.94 mmoles) and (5Z)-2-(methylthio)-5-(6-quinolinylmethylidene)-1,3-thiazol-4(5H)-one (183 mg, 0.65 mmoles) in valeric acid (3.0 mL) was heated at 180° C. for 40 minutes. The mixture was cooled and diluted with ethanol and the solid collected. The solid was purified by dissolving in ethanol-NaOH solution and reprecipitating with acetic acid to give the title compound (130 mg, 42%). 1H NMR (400 MHz, DMSO-d₆) δ ppm 3.80 (s, 3H) 6.95 (dd, J=7.96, 1.89 Hz, 1H) 7.24 (s, 1H) 7.28 (d, J=7.83 Hz, 1H) 7.38 (t, J=7.83 Hz, 1H) 7.51 (s, 1H) 7.55 (dd, J=8.34, 4.04 Hz, 2H) 7.59-7.66 (m, 1H) 7.85 (dd, J=8.84, 2.02 Hz, 1H) 7.88 (s, 1H) 8.07 (d, J=8.84 Hz, 1H) 8.16 (d, J=1.52 Hz, 1H) 8.44 (d, J=7.33 Hz, 1H) 8.92 (dd, J=4.29, 1.52 Hz, 1H) 12.84 (s, 1H)

### Example 107

### (5Z)-2-({2-Chloro-5-[(cyclopentylamino)methyl]phenyl}amino)-5-(6-quinolinylmethylidene)-1,3-thiazol-4(5H)-one

a) 2-({2-Chloro-5-[(cyclopentylamino)methyl]phenyl}amino)-1,3-thiazol-4(5H)-one.

A solution of the compound from example 6(a) (0.120 g, 0.337 mmol) in trifluoroacetic acid (1 mL) was allowed to stand at room temperature for 20 min. The volatiles removed under reduced pressure and the residue azeotroped with methanol twice before re-dissolving in methanol (2 mL). Cyclopentanone (0.300 mL, 3.39 mmol) was added, the mixture stirred 15 min, then sodium triacetoxyborohydride (0.358 g, 1.69 mmol) added and stirring continued for 18 h. A further portion of sodium triacetoxyborohydride (0.358 g, 1.69 mmol) was added over 15 min and the mixture stirred an additional 1 h. 0.5M aqueous hydrochloric acid (20 mL) was added and after stirring 15 min, the pH was adjusted to 9-10 with 1M aqueous sodium hydroxide. The precipitate was filtered, washed with water and dried to give the title compound (0.092 g, 84%) as a white powder. 1H NMR (400 MHz, DMSO-d₆+1% TFA) δ 1.51-1.58 (m, 2H), 1.61-1.72 (m, 4H), 1.91-2.02 (m, 2H), 3.47 (m, 1H), 4.07 (s, 2H), 4.15 (t, J=6.0 Hz, 2H), 7.19 (s, 1H), 7.27 (dd, J=8.3, 1.3 Hz, 1H), 7.59 (d, J=8.3 Hz, 1H), 8.86 (br s, 2H).

b) (5Z)-2-({2-Chloro-5-[(cyclopentylamino)methyl]phenyl}amino)-5-(6-quinolinylmethylidene)-1,3-thiazol-4(5H)-one. The method of example 105(d) was followed, using the compound from example 107(a) in place of the compound from example 105(c), to give the title compound (32%) as a solid. 1H NMR (400 MHz, DMSO-d₆+1% TFA) δ 1.46-1.48 (m, 2H), 1.58-1.69 (m, 4H), 1.92-2.03 (m, 2H), 3.47 (m, 1H), 4.19 (t, J=5.7 Hz, 2H), 7.32 (d, J=1.5 Hz, 1H), 7.37 (dd, J=8.4, 1.8 Hz, 1H), 7.67 (d, J=8.4 Hz, 1H), 7.72 (dd, J=8.4, 4.3 Hz, 1H), 7.91 (s, 1H), 7.95 (dd, J=8.8, 1.7 Hz, 1H), 8.14 (d, J=8.9 Hz, 1H), 8.25 (s, 1H), 8.63 (d, J=8.3 Hz, 1H), 8.90 (br s, 2H), 9.05 (dd, J=4.3, 1.3 Hz, 1H), 12.85 (br s, 1H).

### Example 108

### N-(4-Chloro-3-{[(5Z)-4-oxo-5-(6-quinolinylmethylidene)-4,5-dihydro-1,3-thiazol-2-yl]amino}phenyl)-N-methylcyclobutanecarboxamide

a) N-(4-Chloro-3-nitrophenyl)-N-methylcyclobutanecarboxamide.

Cyclobutanecarbonyl chloride (0.364 mL, 3.19 mmol) was added dropwise to an ice-cooled, stirred solution of 4-chloro-3-nitroaniline (0.500 g, 2.90 mmol) in pyridine (0.5 mL)/dichloromethane (3 mL) under nitrogen. The mixture was stirred 1 h at room temperature, then the volatiles removed under reduced pressure. 4M aqueous potassium carbonate (2 mL) and methanol (10 mL) were added and the mixture stirred 0.5 h, then diluted with water (100 mL) and extracted with ethyl acetate. The extracts were washed (1M aqueous hydrochloric acid, water, brine), dried (MgSO₄) and evaporated under reduced pressure. The residue was azeotroped three times with toluene, then dissolved in tetrahydrofuran (8 mL) and cooled in ice under nitrogen. Sodium hydride (0.139 g of a 60% oil suspension, 3.48 mmol) was added with stirring, followed, after 10 min, by iodomethane (0.217 mL, 3.48 mmol). The mixture was stirred for 1 h at room temperature, then poured into 0.1M aqueous hydrochloric acid (100 mL) and extracted with ethyl acetate. The extracts were washed (water, brine), dried (MgSO₄) and evaporated under reduced pressure. The residue was triturated with hexane and dried to give the title compound (0.548 g, 70%) as a brown oil. 1H NMR (400 MHz, CDCl₃) δ 1.76-2.05 (m, 4H), 2.32-2.44 (m, 2H), 3.05 (m, 1H), 3.30 (s, 3H), 7.38 (br s, 1H), 7.61 (d, J=8.6 Hz, 1H), 7.73 (br s, 1H).

b) N-{4-Chloro-3-[(4-oxo-4,5-dihydro-1,3-thiazol-2-yl)amino]phenyl}-N-methylcyclobutanecarboxamide. The method of example 105(a) was followed, using the compound from example 108(a) in place of 1,5-dichloro-2,4-dinitrobenzene, to give the crude intermediate aniline as a gum. This aniline was converted, using the method of example 6(a), to the title compound (27%, 2 steps) as a solid. 1H NMR (400 MHz, DMSO-d₆) δ1.60-1.80 (m, 4H), 2.03-2.20 (m, 2H), 3.10 (m, 1H), 3.13 (s, 3H), 4.05 (s, 2H), 6.96 (s, 1H), 7.05 (dd, J=8.3, 2.0 Hz, 1H), 7.53 (d, J=8.3 Hz, 1H), 12.06 (brs, 1H).

c) N-(4-Chloro-3-{[(5Z)-4-oxo-5-(6-quinolinylmethylidene)-4,5-dihydro-1,3-thiazol-2-yl]amino}phenyl)-N-methylcyclobutanecarboxamide. The method of example 105(d) was followed, using the compound from example 108(b) in place of the compound from example 105(c), to give the title compound (73%) as a solid. 1H NMR (400 MHz, DMSO-d₆+1% TFA) δ 1.50-1.80 (m, 4H), 2.03-2.20 (m, 2H), 3.15 (s, 3H), 3.18 (m, 1H), 7.14 (d, J=2.0 Hz, 1H), 7.17 (dd, J=8.3, 2.5 Hz, 1H), 7.58 (dd, J=8.4, 4.1 Hz, 1H), 7.62 (d, J=8.6 Hz, 1H), 7.85-7.88 (m, 2H), 8.05 (d, J=8.8 Hz, 1H), 8.17 (d, J=1.5 Hz, 1H), 8.42 (d, J=7.9 Hz, 1H), 8.94 (dd, J=4.3, 1.8 Hz, 1H), 12.85 (brs, 1H).

### Example 109

### N-(4-Chloro-3-{[(5Z)-4-oxo-5-(6-quinolinylmethylidene)-4,5-dihydro-1,3-thiazol-2-yl]amino}phenyl)-N-(phenylsulfonyl)benzenesulfonamide

a) N-(3-Amino-4-chlorophenyl)-N-(phenylsulfonyl)benzenesulfonamide. Benzenesulfonyl chloride (1.10 mL, 8.62 mmol) was added to a stirred solution of 4-chloro-3-nitroaniline (0.500 g, 2.90 mmol) and a small quantity of 1,4-diazabicyclo[2.2.2]octane. After 18 h stirring, the mixture was diluted with dichloromethane and the solid filtered, washed with dichloromethane and dried. The crude sulfonamide was dissolved in acetic acid (3 mL) and zinc (0.760 g, 11.6 mmol) added. The mixture was stirred 2 h, filtered and neutralised with aqueous sodium hydroxide. Ice was added and the precipitated solid filtered and dried to give the title compound (0.065 g, 5%) as a solid. LC/MS MS (ES+) m/e 423 [M+H]⁺

b) N-(4-Chloro-3-{[(5Z)-4-oxo-5-(6-quinolinylmethylidene)-4,5-dihydro-1,3-thiazol-2-yl]amino}phenyl)-N-(phenylsulfonyl)benzenesulfonamide. The method of example 92(c) was followed, using the compound from example 109(a) in place of the compound from example 92(b), to give the title compound (60%) as a solid. 1H NMR (400 MHz, DMSO-d₆) δ ppm 6.83 (dd, J=8.59, 2.53 Hz, 1H) 6.95 (d, J=2.27 Hz, 1H) 7.50 (dd, J=8.34, 4.29 Hz, 1H) 7.57-7.67 (m, 5H) 7.74 (t, J=7.45 Hz, 2H) 7.85-7.90 (m, 5H) 7.93 (dd, J=8.84, 1.77 Hz, 1H) 8.07 (d, J=8.84 Hz, 1H) 8.19 (d, J=1.52 Hz, 1H) 8.31 (d, J=7.58 Hz, 1H) 8.90 (dd, J=4.04, 1.52 Hz, 1H) 12.85 (s, 1H)

### Example 110

### (5Z)-2-({2-Chloro-5-[(phenylmethyl)amino]phenyl}amino)-5-(6-quinolinylmethylidene)-1,3-thiazol-4(5H)-one

The method of example 107 was followed, using the compound from example 94(b) in place of the compound from example 6(a), and using benzaldehyde in place of cyclopentanone. Additionally, the final compound was purified by chromatography (silica gel, 1-7% methanol/dichloromethane) and trituration with ether to give the title compound (32%) as a solid. 1H NMR (400 MHz, DMSO-d₆) δ 4.26 (d, J=6.1 Hz, 2H), 6.27 (s, 1H), 6.46 (m, 1H), 6.57 (t, J=5.8 Hz, 1H), 7.03 (t, J=7.3 Hz, 1H), 7.16 (d, J=8.8 Hz, 1H), 7.24 (t, J=7.7 Hz, 2H), 7.34 (d, J=7.3 Hz, 2H), 7.59 (dd, J=8.3, 4.3 Hz, 1H), 7.81-7.89 (m, 2H), 8.09-8.18 (m, 2H), 8.46 (d, J=7.6 Hz, 1H), 8.96 (dd, J=4.2, 1.6 Hz, 1H), 12.61 (brs, 1H).

### Example 111

### (5Z)-2-({2-Chloro-5-[(1-methylethyl)amino]phenyl}amino)-5-(6-quinolinylmethylidene)-1,3-thiazol-4(5H)-one

a) 2-({2-Chloro-5-[(1-methylethyl)amino]phenyl}amino)-1,3-thiazol-4(5H)-one. The method of example 107(a) was followed, using acetone in place of benzaldehyde, to give the title compound (80%) as a light brown solid. 1H NMR (400 MHz, DMSO-d₆) δ ppm 1.11 (d, J=6.32 Hz, 6H) 3.46 (m, 1H) 3.99 (s, 2H) 5.62 (d, J=7.58 Hz, 1H) 6.15 (s, 1H) 6.34 (d, J=8.34 Hz, 1H) 7.09 (d, J=8.59 Hz, 1H) 11.85 (s, 1H)

b) (5Z)-2-({2-Chloro-5-[(1-methylethyl)amino]phenyl}amino)-5-(6-quinolinylmethylidene)-1,3-thiazol-4(5H)-one. The method of example 107(b) was followed here, using the compound from example 111(a) in place of the compound from example 107(a). Additionally, the compound was chromatographed (silica gel, 1-5% methanol/dichloromethane) to give the title compound (32%) as a yellow solid. 1H NMR (400 MHz, DMSO-d₆) δ 1.13 (d, J=6.1 Hz, 6H), 3.50 (m, 1H), 5.72 (d, J=7.8 Hz, 1H), 6.27 (s, 1H), 6.42 (dd, J=8.6, 2.3 Hz, 1H), 7.16 (d, J=8.8 Hz, 1H), 7.57 (dd, J=8.3, 4.3 Hz, 1H), 7.84 (s, 1H), 7.87 (dd, J=8.9, 1.8 Hz, 1H), 8.10 (d, J=8.9 Hz, 1H), 8.15 (d, J=1.5 Hz, 1H), 8.43 (d, J=7.8 Hz, 1H), 8.94 (dd, J=4.0, 1.5 Hz, 1H), 12.62 (br s, 1H).

### Example 112

### 1,1-Dimethylethyl (4-chloro-3-{[(5Z)-4-oxo-5-(6-quinolinylmethylidene)-4,5-dihydro-1,3-thiazol-2-yl]amino}phenyl)carbamate

The method of example 105(d) was followed, using the compound from example 94(b) in place of the compound from example 105(c). Additionally, the final compound was purified by chromatography (silica gel, 1-6% methanol/dichloromethane) to give the title compound (56%) as a solid. 1H NMR (400 MHz, DMSO-d₆) δ1.46 (s, 9H), 7.24-7.34 (m, 2H), 7.42 (d, J=8.8 Hz, 1H), 7.57 (dd, J=8.3, 4.3 Hz, 1H), 7.81-7.90 (m, 2H), 8.09 (d, J=8.8 Hz, 1H), 8.16 (d, J=1.5 Hz, 1H), 8.46 (d, J=7.8 Hz, 1H), 8.94 (dd, J=4.3, 1.5 Hz, 1H), 9.59 (s, 1H), 12.73 (br s, 1H).

### Example 113

### N-(4-Chloro-3-{[(5Z)-4-oxo-5-(6-quinolinylmethylidene)-4,5-dihydro-1,3-thiazol-2-yl]amino}phenyl)-L-prolinamide

a) 1,1-Dimethylethyl (2S)-2-{[(4-chloro-3-nitrophenyl)amino]carbonyl}-1-pyrrolidinecarboxylate. O-(7-Azabenzotriazol-1-yl)-N,N,N′,N′-tetramethyluronium hexafluorophosphate (2.42 g, 6.36 mmol) was added to a mixture of 4-chloro-3-nitroaniline (1.00 g, 5.79 mmol), BOC-L-proline (1.87 g, 8.69 mmol), di-isopropylethylamine (5.05 mL, 29.0 mmol) and dimethylformamide/dichloromethane (1:1, 10 mL) and the mixture stirred under nitrogen for 18 h. Ethyl acetate (100 mL) was added and the mixture was with 1M aqueous HCl, water, 1M aqueous NaOH, and brine, then dried (MgSO₄) and evaporated under reduced pressure. The residue was chromatographed (silica gel, 10-50% ethyl acetate/hexane) to give the title compound (1.21 g, 56%). 1H NMR (400 MHz, DMSO-d₆) δ ppm 1.27 (s) & 1.40 (s) (9H) 1.79-1.94 (m, 3H) 2.16-2.27 (m, 1H) 3.32-3.46 (m, 2H) 4.19 (dd J=8.08, 4.29 Hz) & 4.23 (dd J=8.34, 3.03 Hz) (1H) 7.70-7.76 (m, 1H) 7.80 (dd J=8.84, 2.27 Hz) & 7.86 (dd J=8.84, 2.53 Hz) (1H) 8.41 (d J=2.53 Hz) & 8.46 (d J=2.27 Hz) (1H) 10.56 (s) & 10.58 (s) (1H)

b) 1,1-Dimethylethyl (2S)-2-{[(3-amino-4-chlorophenyl)amino]carbonyl}-1-pyrrolidinecarboxylate.

The procedure of example 92(b) was followed here, using the compound from example 113(a) in place of the compound from example 92(a), to give the title compound (37%) as a white solid. 1H NMR (400 MHz, DMSO-d₆) δ ppm 1.27 (s) & 1.40 (s) (9H) 1.75-1.90 (m, 3H) 2.14-2.20 (m, 1H) 3.30-3.41 (m, 2H) 4.16 (dd J=8.08, 4.55 Hz) & 4.23 (dd J=8.59, 3.54 Hz) (1H) 5.35 (br s, 2H) 6.71-6.76 (m, 1H) 7.07 (d J=8.59 Hz) & 7.08 (d J=8.59 Hz) (1H) 7.17 (d J=2.53 Hz) & 7.19 (d J=2.53 Hz) (1H) 9.82 (s, 1H)

c) N-(4-Chloro-3-{[(5Z)-4-oxo-5-(6-quinolinylmethylidene)-4,5-dihydro-1,3-thiazol-2-yl]amino}phenyl)-L-prolinamide. A mixture of the compound from example 113(b) (0.553 g, 1.63 mmol), (5Z)-2-(methylthio)-5-(6-quinolinylmethylidene)-1,3-thiazol-4(5H)-one (0.120 g, 0.419 mmol) and pentanoic acid (0.5 mL) was stirred at 150° C. for 0.5 h, then cooled and diluted with water and extracted with ethyl acetate. The extracts were evaporated under reduced pressure and the residue triturated with aqueous methanol to give the BOC protected intermediate as a yellow solid. The solid was stirred in 30% trifluoroaectic acid/dichloromethane for 0.5 h, then the volatiles removed under reduced pressure and the residue chromatographed (silica gel, 10% methanol/dichloromethane) to give the title compound (0.020 g, 3%) as a solid. 1H NMR (400 MHz, DMSO-d₆) δ ppm 1.85-1.97 (m, 3H) 2.26-2.35 (m, 1H) 3.12-3.22 (m, 2H) 4.20 (m, 1H) 7.38-7.49 (m, 2H) 7.52 (m, 1H) 7.57 (dd, J=8.46, 4.17 Hz, 1H) 7.84-7.88 (m, 2H) 8.09 (d, J=8.84 Hz, 1H) 8.16 (d, J=1.52 Hz, 1H) 8.45 (d, J=7.58 Hz, 1H) 8.94 (dd, J=4.29, 1.52 Hz, 1H) 10.59 (s, 1H)

### Example 114

### N˜1˜-(4-Chloro-3-{[(5Z)-4-oxo-5-(6-quinolinylmethylidene)-4,5-dihydro-1,3-thiazol-2-yl]amino}phenyl)-L-alaninamide

The procedure of example 113 was followed here, using BOC-L-alanine in place of BOC-L-proline, to give the title compound (10%) as a solid. 1H NMR (400 MHz, DMSO-d₆) δ ppm 1.45 (d, J=7.07 Hz, 3H) 4.00 (m, 1H) 7.39-7.50 (m, 2H) 7.56 (d, J=8.84 Hz, 1H) 7.58 (dd, J=8.34, 4.29 Hz, 1H) 7.86 (dd, J=8.84, 1.77 Hz, 1H) 7.88 (s, 1H) 8.09 (d, J=8.84 Hz, 1H) 8.16-8.21 (m, 3H) 8.46 (d, J=7.83 Hz, 1H) 8.95 (dd, J=4.04, 1.26 Hz, 1H) 10.66 (s, 1H) 12.82 (s, 1H)

### Example 115

### 1,1-Dimethylethyl 4-{[(4-chloro-3-{[(5Z)-4-oxo-5-(6-quinolinylmethylidene)-4,5-dihydro-1,3-thiazol-2-yl]amino}phenyl)amino]carbonyl}-1-piperidinecarboxylate

The procedure of example 113 was followed here, using N—(BOC)-4-piperidinecarboxylic acid in place of BOC-L-proline. The BOC protected compound immediately preceding the final deprotection (the title compound) was isolated pure (31%) as a solid. 1H NMR (400 MHz, DMSO-d₆) δ ppm 1.39 (s, 9H) 1.41-1.48 (m, 2H) 1.75-1.80 (m, 2H) 2.48 (m, 1H) 2.76 (m, 2H) 3.95-4.01 (m, 2H) 7.40-7.50 (m, 3H) 7.56 (dd, J=8.34, 4.29 Hz, 1H) 7.86 (s, 1H) 7.86 (dd, J=8.59, 2.02 Hz, 1H) 8.09 (d, J=8.84 Hz, 1H) 8.17 (d, J=1.52 Hz, 1H) 8.46 (d, J=7.58 Hz, 1H) 8.94 (dd, J=4.17, 1.64 Hz, 1H) 10.14 (s, 1H) 12.77 (s, 1H)

### Example 116

### (5Z)-2-({2-Chloro-5-[6-(methylamino)-2-pyridinyl]phenyl}amino)-5-(6-quinolinylmethylidene)-1,3-thiazol-4(5H)-one

a) 1,1-Dimethylethyl (6-bromo-2-pyridinyl)methylcarbamate. Sodium hydride (60% in oil, 1.80 g, 45 mmoles) was added portionwise to a solution of 1,1-dimethylethyl (6-bromo-2-pyridinyl)carbamate (Patent WO 2004113331, 10.0 g, 36.6 mmoles) in dimethylformamide (60 mL) under argon. The mixture was stirred for 15 minutes then iodomethane (4.5 mL, 72.2 mmoles) was added dropwise. The mixture was stirred for 3 hours, partitioned between ethyl acetate-water and the aqueous extracted with ethyl acetate (×2). The combined extracts were washed with water, dried and evaporated to give the title compound (11 g, quant.). 1H NMR (400 MHz, DMSO-d₆) δ ppm 1.47 (s, 9H) 3.27 (s, 3H) 7.35 (dd, J=4.67, 3.66 Hz, 1H) 7.69 (d, J=1.26 Hz, 1H) 7.70 (s, 1H).

b) 1,1-Dimethylethyl [6-(4-chloro-3-nitrophenyl)-2-pyridinyl]methylcarbamate. A mixture of 4-chloro-3-nitrophenylboronic acid (245 mg, 1.225 mmoles), 1,1-dimethylethyl (6-bromo-2-pyridinyl)methylcarbamate (350 mg, 1.225 mmoles), sodium carbonate (400 mg, 3.68 mmoles) and tetrakis(triphenylphosphine)palladium(0) (50 mg, 0.04 mmoles) in 3:1 dimethylformamide-water (5 mL) was sealed in a pressure tube and heated at 150° C. for 20 minutes in a microwave reactor. The mixture was diluted with water and extracted with ethyl acetate (×2). Combined extracts were washed with brine, dried and evaporated. Flash chromatography (0-20% ethyl acetate-hexane) gave the title compound (300 mg, 68%). 1H NMR (400 MHz, CHLOROFORM-d) δ ppm 1.57 (s, 9H) 3.51 (s, 3H) 7.47 (dd, J=7.58, 0.76 Hz, 1H) 7.63 (d, J=8.59 Hz, 1H) 7.72-7.78 (m, 1H) 7.79-7.84 (m, 1H) 8.17 (dd, J=8.46, 2.15 Hz, 1H) 8.54 (d, J=2.02 Hz, 1H).

c) 1,1-Dimethylethyl [6-(3-amino-4-chlorophenyl)-2-pyridinyl]methylcarbamate. A mixture of 1,1-dimethylethyl [6-(4-chloro-3-nitrophenyl)-2-pyridinyl]methylcarbamate (300 mg, 0.82 mmoles) and zinc powder (540 mg, 8.2 mmoles) in acetic acid (30 mL) was stirred vigorously for 1.5 hr. The mixture was filtered, washed through with ethanol and evaporated to give the title compound (200 mg, 73%) 1H NMR (400 MHz, CHLOROFORM-d) δ ppm 1.56 (s, 9H) 3.52 (s, 3H) 7.32-7.34 (m, 2H) 7.41 (d, J=7.33 Hz, 1H) 7.50 (s, 1H) 7.62-7.66 (m, 1H) 7.66-7.72 (m, 1H).

d) (5Z)-2-({2-Chloro-5-[6-(methylamino)-2-pyridinyl]phenyl}amino)-5-(6-quinolinyl methylidene)-1,3-thiazol-4(5H)-one. A mixture of 1,1-dimethylethyl [6-(3-amino-4-chlorophenyl)-2-pyridinyl]methylcarbamate (175 mg, 0.525 mmoles) and (5Z)-2-(methylthio)-5-(6-quinolinylmethylidene)-1,3-thiazol-4(5H)-one (100 mg, 0.35 mmoles) in valeric acid (1.5 mL) was heated at 180° C. for 10 minutes. The mixture was cooled and diluted with ethanol and the solid collected. The solid was stirred in trifluoroacetic acid (3.0 mL) for 30 minutes, taken up in dichloromethane and washed with NaHCO₃ solution. The resulting solid was collected, washed with water, ethanol and hexane. Purified by dissolving in ethanol-NaOH solution and reprecipitating with acetic acid to give the title compound (60 mg, 36%). 1H NMR (400 MHz, DMSO-d₆) δ ppm 2.82 (d, J=4.80 Hz, 3H) 6.43 (d, J=8.08 Hz, 1H) 6.60 (q, J=4.55 Hz, 1H) 7.12 (d, J=7.07 Hz, 1H) 7.46 (t, J=7.83 Hz, 1H) 7.54 (dd, J=8.46, 4.17 Hz, 1H) 7.62 (d, J=8.34 Hz, 1H) 7.82-7.92 (m, 4H) 8.06 (d, J=8.84 Hz, 1H) 8.15 (d, J=1.77 Hz, 1H) 8.43 (d, J=8.08 Hz, 1H) 8.92 (d, J=2.78 Hz, 1H) 12.82 (s, 1H)

### Example 117

### (5Z)-2-[(3,5-Dichloro-4-pyridinyl)amino]-5-(6-quinolinylmethylidene)-1,3-thiazol-4(5H)-one trifluoroacetate

A mixture of 2,6-dichloro-4-pyridinamine (100 mg, 0.6 mmoles), (5Z)-2-(methylthio)-5-(6-quinolinylmethylidene)-1,3-thiazol-4(5H)-one (100 mg, 0.35 mmoles) and 1,8-diazabicyclo[5.4.0]undec-7-ene (0.25 mL) in dimethylformamide (1.5 mL) was heated at 140° C. for 10 minutes. The mixture was purified by preparative HPLC (10-90% acetonitrile-water-0.1% TFA) to give the title compound (18 mg, 13%). 1H NMR (400 MHz, DMSO-d₆) δ ppm 7.58 (dd, J=8.34, 4.04 Hz, 1H) 7.89 (d, J=8.84 Hz, 1H) 7.95 (s, 1H) 8.09 (d, J=8.59 Hz, 1H) 8.19 (s, 1H) 8.51 (d, J=8.08 Hz, 1H) 8.68 (s, 2H) 8.95 (d, J=3.03 Hz, 1H) 13.19 (s, 1H)

### Example 118

### 1,1-Dimethylethyl (3-chloro-4-{[(5Z)-4-oxo-5-(6-quinolinylmethylidene)-4,5-dihydro-1,3-thiazol-2-yl]amino}phenyl)carbamate

a) 1,1-Dimethylethyl (4-amino-3-chlorophenyl)carbamate. A solution of di-t-butyl dicarbonate (10.11 g, 46.3 mmol) in dichloromethane (20 mL) was added over 20 min to a stirred suspension of 2-chloro-1,4-benzenediamine (6.00 g, 42.1 mmol) in dichloromethane (80 mL) at room temperature. After stirring 18 h, the solution was loaded on to a silica gel pad and eluted with 20-30% ethyl acetate/hexane. The residue after removal of the solvent from the filtrate was chromatographed (silica gel, 10-30% ethyl acetate/hexane) to give the title compound (7.46 g, 73%). 1H NMR (400 MHz, DMSO-d₆) δ 1.45 (s, 9H), 4.98 (s, 2H), 6.69 (d, J=8.6 Hz, 1H), 7.00-7.09 (m, 1H), 7.36 (s, 1H), 9.02 (s, 1H).

b) 1,1-Dimethylethyl {3-chloro-4-[(4-oxo-4,5-dihydro-1,3-thiazol-2-yl)amino]phenyl}carbamate. The method of example 77(a) was followed, using the compound from example 118(a) in place of 2-chloro-5-nitro-1,4-benzenediamine, to give the title compound (93%) as a solid. 1H NMR (400 MHz, DMSO-d₆) δ 1.48 (s, 9H), 4.00 (s, 2H), 6.96 (m, 1H), 7.32 (dd, J=8.7, 2.2 Hz, 1H), 7.66 (s, 1H), 9.50 (s, 1H), 11.92 (br s, 1H).

c) 1,1-Dimethylethyl (3-chloro-4-{[(5Z)-4-oxo-5-(6-quinolinylmethylidene)-4,5-dihydro-1,3-thiazol-2-yl ]amino}phenyl)carbamate. The method of example 105(d) was followed, using the compound from example 118(b) in place of the compound from example 105(c). Additionally, the final compound was purified by chromatography (silica gel, 1-10% methanol/dichloromethane). The product was boiled in methanol, cooled, filtered, and dried to give the title compound (66%) as a solid. 1H NMR (400 MHz, DMSO-d₆) δ 1.50 (s, 9H), 7.09 (m, 1H), 7.40 (dd, J=8.6, 2.3 Hz, 1H), 7.56 (dd, J=8.3, 4.3 Hz, 1H), 7.73 (d, J=1.5 Hz, 1H), 7.81-7.90 (m, 2H), 8.09 (d, J=8.6 Hz, 1H), 8.15 (d, J=1.8 Hz, 1H), 8.47 (d, J=7.6 Hz, 1H), 8.94 (dd, J=4.3, 1.8 Hz, 1H), 9.59 (s, 1H), 12.70 (br s, 1H).

### Example 119

### N-(4-Chloro-3-{[(5Z)-4-oxo-5-(6-quinolinylmethylidene)-4,5-dihydro-1,3-thiazol-2-yl]amino}phenyl)-4-piperidinecarboxamide trifluoroacetate

The BOC deprotection of the compound from example 115 was carried out according to the procedure in example 113 to give the title compound (89%) as a yellow solid. 1H NMR (400 MHz, DMSO-d₆) δ ppm 1.73-1.84 (m, 2H) 1.91-1.97 (m, 2H) 2.62 (m, 1H) 2.86-2.96 (m, 2H) 3.30-3.36 (m, 2H) 7.42 (dd, J=8.59, 2.27 Hz, 1H) 7.46-7.51 (m, 2H) 7.58 (dd, J=8.34, 4.29 Hz, 1H) 7.85-7.88 (m, 2H) 8.09 (d, J=8.84 Hz, 1H) 8.18 (d, J=1.77 Hz, 1H) 8.29 (br s, 1H) 8.46 (d, J=8.45 Hz, 1H) 8.54 (br s, 1H) 8.95 (dd, J=4.04, 1.52 Hz, 1H) 10.26 (s, 1H) 12.79 (s, 1H)

### Example 120

### N-(4-Chloro-3-{[(5Z)-4-oxo-5-(6-quinolinylmethylidene)-4,5-dihydro-1,3-thiazol-2-yl]amino}phenyl)ethanesulfonamide

The method of example 75 was followed here, using ethanesulfonyl chloride in place of phenylmethanesulfonyl chloride and sodium acetate in place of piperidine. Additionally, the material was purified by rp-HPLC (ODS, 10-90% acetonitrile/water+0.1% trifluoroacetic acid) to give the title compound (7%) as a solid. 1H NMR (400 MHz, DMSO-d₆) δ ppm 1.19 (t, J=7.33 Hz, 3H) 3.18 (q, J=7.33 Hz, 2H) 6.99 (d, J=2.53 Hz, 1H) 7.06 (dd, J=8.59, 2.53 Hz, 1H) 7.52 (d, J=8.84 Hz, 1H) 7.62 (dd, J=8.34, 4.29 Hz, 1H) 7.88 (s, 1H) 7.91 (dd, J=8.84, 2.02 Hz, 1H) 8.11 (d, J=8.84 Hz, 1H) 8.19 (d, J=1.77 Hz, 1H) 8.51 (d, J=8.34 Hz, 1H) 8.98 (dd, J=4.17, 1.64 Hz, 1H) 10.06 (s, 1H) 12.81 (s, 1H)

### Example 121

### N-(4-Chloro-3-{[(5Z)-4-oxo-5-(6-quinolinylmethylidene)-4,5-dihydro-1,3-thiazol-2-yl]amino}phenyl)benzenesulfonamide

The method of example 75 was followed here, using benzenesulfonyl chloride in place of phenylmethanesulfonyl chloride and sodium acetate in place of piperidine. Additionally, the material was purified by rp-HPLC (ODS, 10-90% acetonitrile/water+0.1% trifluoroacetic acid) to give the title compound (10%) as a solid. 1H NMR (400 MHz, DMSO-d₆) δ ppm 6.86 (d, J=2.27 Hz, 1H) 6.90 (dd, J=8.59, 2.53 Hz, 1H) 7.35-7.49 (m, 4H) 7.63 (dd, J=8.34, 4.29 Hz, 1H) 7.77-7.79 (m, 2H) 7.83-7.90 (m, 2H) 8.13 (d, J=8.84 Hz, 1H) 8.18 (d, J=1.52 Hz, 1H) 8.48 (d, J=8.08 Hz, 1H) 8.99 (dd, J=4.29, 1.77 Hz, 1H) 10.57 (s, 1H) 12.78 (s, 1H)

### Example 122

### N-(4-Chloro-3-{[(5Z)-4-oxo-5-(6-quinolinylmethylidene)-4,5-dihydro-1,3-thiazol-2-yl]amino}phenyl)-1-propanesulfonamide

The method of example 75 was followed here, using propanesulfonyl chloride in place of phenylmethanesulfonyl chloride and sodium acetate in place of piperidine. Additionally, the material was purified by rp-HPLC (ODS, 10-90% acetonitrile/water+0.1% trifluoroacetic acid) to give the title compound (37%) as a solid. 1H NMR (400 MHz, DMSO-d₆) δ ppm 0.87 (t, J=7.45 Hz, 3H) 1.66 (sextet, J=7.53 Hz, 2H) 3.10-3.20 (m, 2H) 6.97 (d, J=2.53 Hz, 1H) 7.04 (dd, J=8.59, 2.53 Hz, 1H) 7.51 (d, J=8.84 Hz, 1H) 7.58 (dd, J=8.34, 4.29 Hz, 1H) 7.85-7.88 (m, 2H) 8.07 (d, J=8.84 Hz, 1H) 8.17 (d, J=1.77 Hz, 1H) 8.45 (d, J=8.34 Hz, 1H) 8.95 (dd, J=4.29, 1.77 Hz, 1H) 10.06 (s, 1H) 12.77 (s, 1H)

### Example 123

### N-(3-Chloro-4-{[(5Z)-4-oxo-5-(6-quinolinylmethylidene)-4,5-dihydro-1,3-thiazol-2-yl]amino}phenyl)acetamide

a) (5Z)-2-[(4-Amino-2-chlorophenyl)amino]-5-(6-quinolinylmethylidene)-1,3-thiazol-4(5H)-one, bis-trifluoroacetate salt. A solution of the compound from example 118(c) (0.780 g, 1.62 mmol) in trifluoroacetic acid (8 mL) was allowed to stand at room temperature for 0.5 h, then evaporated to dryness under reduced pressure and azeotroped four times with methanol to give the title compound (1.15 g) as an orange powder used without further purification. LCMS m/z 381 [M+H].

b) N-(3-Chloro-4-{[(5Z)-4-oxo-5-(6-quinolinylmethylidene)-4,5-dihydro-1,3-thiazol-2-yl]amino}phenyl)acetamide. Acetyl chloride (0.020 mL, 0.281 mmol) was injected into a stirred solution of the compound from example 123(a) (0.088 g, 0.145 mmol) in pyridine (1 mL) at room temperature under argon. After stirring 0.5 h, water (1 mL) was added followed by 1M aq NaOH (3 mL) and stirring continued an additional 0.25 h. The pH was adjusted to 7 with 6M aq HCl and water (20 mL) added. The solid was filtered, washed with water then redissolved in 1M aq NaOH and methanol (1:1, 10 mL). Acetic acid was added slowly to pH 7 and the precipitate filtered, washed with water and ether and dried to give the title compound (0.041 g, 67%) as a brown powder. 1H NMR (400 MHz, DMSO-d₆) δ 2.08 (s, 3H), 7.12 (d, J=8.3 Hz, 1H), 7.47 (dd, J=8.6, 2.0 Hz, 1H), 7.56 (dd, J=8.3, 4.0 Hz, 1H), 7.84-7.94 (m, 3H), 8.09 (d, J=8.8 Hz, 1H), 8.15 (d, J=1.3 Hz, 1H), 8.46 (d, J=8.1 Hz, 1H), 8.94 (d, J=2.5 Hz, 1H), 10.14 (s, 1H), 12.72 (br s, 1H).

### Example 124

### N-(3-Chloro-4-{[(5Z)-4-oxo-5-(6-quinolinylmethylidene)-4,5-dihydro-1,3-thiazol-2-yl]amino}phenyl)methanesulfonamide

The method of example 123(b) was followed, using methanesulfonyl chloride in place of acetyl chloride, to give the title compound (88%) as a tan solid. 1H NMR (400 MHz, DMSO-d₆) δ 3.10 (s, 3H), 7.15-7.25 (m, 2H), 7.37 (d, J=2.0 Hz, 1H), 7.57 (dd, J=8.3, 4.0 Hz, 1H), 7.81-7.91 (m, 2H), 8.09 (d, J=8.8 Hz, 1H), 8.16 (d, J=1.8 Hz, 1H), 8.47 (d, J=7.8 Hz, 1H), 8.94 (dd, J=4.2, 1.6 Hz, 1H), 9.97 (s, 1H), 12.76 (br s, 1H).

### Example 125

### N-(3-Chloro-4-{[(5Z)-4-oxo-5-(6-quinolinylmethylidene)-4,5-dihydro-1,3-thiazol-2-yl]amino}phenyl)benzamide

The method of example 123(b) was followed, using benzoyl chloride in place of acetyl chloride, to give the title compound (100%) as a yellow solid. 1H NMR (400 MHz, DMSO-d₆) δ 7.19 (d, J=8.8 Hz, 1H), 7.54-7.65 (m, 4H), 7.76 (dd, J=8.5, 1.2 Hz, 1H), 7.82-7.91 (m, 2H), 7.97 (d, J=7.3 Hz, 2H), 8.05-8.13 (m, 2H), 8.16 (s, 1H), 8.47 (d, J=8.1 Hz, 1H), 8.93 (d, J=2.8 Hz, 1H), 10.44 (s, 1H), 12.74 (brs, 1H).

### Example 126

### N-(3-Chloro-4-{[(5Z)-4-oxo-5-(6-quinolinylmethylidene)-4,5-dihydro-1,3-thiazol-2-yl]amino}phenyl)-2-methylpropanamide

The method of example 123(b) was followed, using isobutyryl chloride in place of acetyl chloride, to give the title compound (67%) as a yellow solid. 1H NMR (400 MHz, DMSO-d₆) δ 1.13 (d, J=6.8 Hz, 6H), 2.60 (m, 1H), 7.13 (m, 1H), 7.49-7.60 (m, 2H), 7.82-7.89 (m, 2H), 7.96 (s, 1H), 8.09 (d, J=8.8 Hz, 1H), 8.15 (s, 1H), 8.47 (d, J=8.1 Hz, 1H), 8.94 (d, J=3.0 Hz, 1H), 10.03 (s, 1H), 12.71 (br s, 1H).

### Example 127

### N-(4-Chloro-3-{[(5Z)-4-oxo-5-(6-quinolinylmethylidene)-4,5-dihydro-1,3-thiazol-2-yl]amino}phenyl)-4-methylbenzenesulfonamide

The method of example 75 was followed here, using p-toluenesulfonyl chloride in place of phenylmethanesulfonyl chloride and sodium acetate in place of piperidine. Additionally, the material was purified by rp-HPLC (ODS, 10-90% acetonitrile/water+0.1% trifluoroacetic acid) to give the title compound (6%) as a solid. 1H NMR (400 MHz, DMSO-d₆) δ ppm 2.12 (s, 3H) 6.86-6.91 (m, 2H) 7.27 (d, J=8.08 Hz, 2H) 7.42 (d, J=8.84 Hz, 1H) 7.62 (dd, J=8.34, 4.29 Hz, 1H) 7.66 (d, J=8.34 Hz, 2H) 7.88 (s, 1H) 7.89 (dd, J=8.84, 1.77 Hz, 1H) 8.13 (d, J=8.84 Hz, 1H) 8.20 (d, J=1.52 Hz, 1H) 8.51 (d, J=8.34 Hz, 1H) 8.99 (dd, J=4.29, 1.52 Hz, 1H) 10.47 (s, 1H) 12.78 (s, 1H)

### Example 128

### N-(3-Chloro-4-{[(5Z)-4-oxo-5-(6-quinolinylmethylidene)-4,5-dihydro-1,3-thiazol-2-yl]amino}phenyl)-2,2,2-trifluoroacetamide

The method of example 123(b) was followed, using trifluoromethanesulfonic anhydride in place of acetyl chloride. Additionally, the final compound was purified by chromatography (silica gel, 1-8% methanol/dichloromethane) and trituration with ether to give the title compound (11%) as a solid. 1H NMR (400 MHz, DMSO-d₆) δ 7.24 (d, J=8.6 Hz, 1H), 7.57 (dd, J=8.3, 4.3 Hz, 1H), 7.67 (dd, J=8.6, 2.3 Hz, 1H), 7.82-7.90 (m, 2H), 7.94 (d, J=2.3 Hz, 1H), 8.09 (d, J=8.8 Hz, 1H), 8.16 (d, J=1.5 Hz, 1H), 8.46 (d, J=8.1 Hz, 1H), 8.94 (dd, J=4.0, 1.5 Hz, 1H), 11.43 (s, 1H), 12.77 (br s, 1H).

### Example 129

### (3S)-3-Amino-N-(4-chloro-3-{[(5Z)-4-oxo-5-(6-quinolinylmethylidene)-4,5-dihydro-1,3-thiazol-2-yl]amino}phenyl)butanamide

The procedure of example 113 was followed here, using BOC-2(L)-aminobutyric acid in place of BOC-L-proline, to give the title compound (2%) as a yellow solid. 1H NMR (400 MHz, DMSO-d₆) δ ppm 0.94 (t, J=7.45 Hz, 3H) 1.85 (m, 2H) 3.89 (m, 1H) 7.42 (dd, J=8.59, 2.27 Hz, 1H) 7.48 (d, J=2.27 Hz, 1H) 7.56 (d, J=8.84 Hz, 1H) 7.58 (dd, J=8.08, 4.29 Hz, 1H) 7.86 (dd, J=8.84, 2.02 Hz, 1H) 7.88 (s, 1H) 8.09 (d, J=8.84 Hz, 1H) 8.18 (d, J=1.52 Hz, 1H) 8.22 (br s, 3H) 8.46 (d, J=7.83 Hz, 1H) 8.95 (dd, J=4.17, 1.64 Hz, 1 H) 10.70 (s, 1H) 12.82 (s, 1H)

### Example 130

### N-(4-Chloro-3-{[(5Z)-4-oxo-5-(6-quinolinylmethylidene)-4,5-dihydro-1,3-thiazol-2-yl]amino}phenyl)-L-phenylalaninamide

The method of example 81(c) was followed here, using FMOC-L-phenylalaminoyl chloride in place of FMOC-aminoacetyl chloride, to give the title compound (3%) as a solid. 1H NMR (400 MHz, DMSO-d₆) δ ppm 2.85 (m, 1H) 3.03 (m, 1H) 3.76 (m, 1H) 7.15-7.55 (m, 9H) 7.73 (s, 1H) 7.88 (d, J=8.84 Hz, 1H) 8.07 (d, J=8.84 Hz, 1H) 8.12 (d, J=1.52 Hz, 1H) 8.43 (d, J=8.08 Hz, 1H) 8.91 (dd, J=4.29, 1.52 Hz, 1H) 10.16 (br s, 1H)

### Example 131

### N-(4-Chloro-3-{[(5Z)-4-oxo-5-(6-quinolinylmethylidene)-4,5-dihydro-1,3-thiazol-2-yl]amino}phenyl)-4-nitrobenzenesulfonamide

The method of example 75 was followed here, using p-nitrobenzenesulfonyl chloride in place of phenylmethanesulfonyl chloride and sodium acetate in place of piperidine. Additionally, the material was purified by rp-HPLC (ODS, 10-90% acetonitrile/water+0.1% trifluoroacetic acid) to give the title compound (8%) as a solid. 1H NMR (400 MHz, DMSO-d₆) □ 6.88 (d, J=2.53 Hz, 1H) 6.92 (dd, J=8.84, 2.53 Hz, 1H) 7.46 (d, J=8.59 Hz, 1H) 7.60 (dd, J=8.34, 4.29 Hz, 1H) 7.84 (dd, J=8.97, 1.89 Hz, 1H) 7.87 (s, 1H) 8.04 (d, J=8.84 Hz, 2H) 8.10 (d, J=8.84 Hz, 1H) 8.17 (d, J=1.77 Hz, 1H) 8.35 (d, J=9.09 Hz, 2H) 8.47 (d, J=8.34 Hz, 1H) 8.97 (dd, J=4.29, 1.52 Hz, 1H) 10.89 (s, 1H) 12.78 (s, 1H)

### Example 132

### N-(4-Chloro-3-{[(5Z)-4-oxo-5-(6-quinolinylmethylidene)-4,5-dihydro-1,3-thiazol-2-yl]amino}phenyl)-2,2-dimethylpropanamide

a) N-(3-Amino-4-chlorophenyl)-2,2-dimethylpropanamide. Pivaloyl chloride (2.71 mL, 22.0 mmol) was injected dropwise into a stirred solution of 4-chloro-1,3-benzenediamine (2.85 g, 20.0 mmol) in pyridine (20 mL) at room temperature. After stirring 60 h, water (200 mL) was added and the mixture extracted with ethyl acetate. The extracts were washed with 0.1M aqueous hydrochloric acid, water and brine, dried (MgSO₄) and evaporated under reduced pressure. The residue was recrystallised (toluene) to give the title compound (3.11 g, 69%) as a solid. 1H NMR (400 MHz, DMSO-d₆) δ 1.20 (s, 9H), 5.26 (s, 2H), 6.77 (dd, J=8.6, 2.5 Hz, 1H), 7.06 (d, J=8.6 Hz, 1H), 7.22 (d, J=2.5 Hz, 1H), 9.01 (s, 1H).

b) N-{4-Chloro-3-[(4-oxo-4,5-dihydro-1,3-thiazol-2-yl)amino]phenyl}-2,2-dimethylpropanamide. The method of example 77(a) was followed here, using the compound from example 132(a) in place of 2-chloro-5-nitro-1,4-benzenediamine, to give the title compound (73%) as a cream solid. 1H NMR (400 MHz, DMSO-d₆) δ 1.22 (s, 9H), 4.03 (s, 2H), 7.38 (s, 2H), 7.49 (s, 1H), 9.29 (s, 1H), 11.98 (br s, 1H).

c) N-(4-Chloro-3-{[(5Z)-4-oxo-5-(6-quinolinylmethylidene)-4,5-dihydro-1,3-thiazol-2-yl]amino}phenyl)-2,2-dimethylpropanamide. The method of example 105(d) was followed, using the compound from example 132(b) in place of the compound from example 105(c) to give the title compound (83%) as a yellow solid. 1H NMR (400 MHz, DMSO-d₆) δ 1.22 (s, 9H), 7.44-7.64 (m, 4H), 7.82-7.92 (m, 2H), 8.09 (d, J=8.8 Hz, 1H), 8.17 (d, J=1.5 Hz, 1H), 8.46 (d, J=7.6 Hz, 1H), 8.94 (dd, J=4.2, 1.6 Hz, 1H), 9.36 (s, 1H), 12.75 (br s, 1H).

### Example 133

### 4-Chloro-N-(2-methylpropyl)-3-{[(5Z)-4-oxo-5-(6-quinoxalinylmethylidene)-4,5-dihydro-1,3-thiazol-2-yl]amino}benzamide

Potassium carbonate (0.066 g, 0.478 mmol) was added to a slurry of the compound from example 31(c) (0.058 g, 0.238 mmol) in 2-butanone (4 mL), followed by isovaleryl chloride (0.029 mL, 0.238 mmol). The mixture was stirred at room temperature for 18 h, then evaporated under reduced pressure and the residue partitioned between brine and ethyl acetate. The extracts were dried (MgSO₄) and evaporated under reduced pressure to give the crude amide product. A suspension of the crude amide (0.100 g; 0.307 mmol), 6-quinoxalinecarbaldehyde (0.049 g; 0.307 mmol), and piperidine (0.030 mL; 0.307 mmol) in ethanol (2.0 mL) was stirred and irradiated at 150° C. for 30 min. in a Biotage Initiator microwave synthesizer. Upon cooling, the reaction mixture was acidified with 1M aq. HCl and the resulting suspension was filtered, washed with water, and dried in vacuo to afford the title compound (0.111 g, 78%) as a yellow solid. ¹H NMR (400 MHz, DMSO-d₆) δ ppm 12.9 (s, 1H) 10.1 (s, 1H) 8.98 (s, 2H) 8.22 (d, J=1.5 Hz, 1H) 8.18 (d, J=8.6 Hz, 1H) 7.99 (dd, J=8.8, 1.8 Hz, 1H) 7.95 (s, 1H) 7.50 (d, J=1.5 Hz, 1H) 7.47 (d, J=9.3 Hz, 1H) 7.41 (dd, J=9.1, 2.0 Hz, 1H) 2.19 (d, J=7.1 Hz, 2H) 2.00-2.10 (m, 1H) 0.92 (d, J=6.6 Hz, 6H). MS (ES+) m/e 466 [M+H]⁺.

### Example 134

### N-(4-Chloro-3-{[(5Z)-4-oxo-5-(6-quinoxalinylmethylidene)-4,5-dihydro-1,3-thiazol-2-yl]amino}phenyl)propanamide

The procedure of example 44 was followed, using quinoxaline-6-carbaldehyde in place of quinoline-6-carbaldehyde and piperidine in place of sodium acetate, to give the title compound (16%) as a solid. 1H NMR (400 MHz, DMSO-d₆) δ ppm 1.06 (t, J=7.58 Hz, 3H) 2.33 (q, J=7.49 Hz, 2H) 7.40-7.51 (m, 3H) 7.94 (s, 1H) 7.97 (dd, J=8.84, 2.02 Hz, 1H) 8.16 (d, J=8.84 Hz, 1H) 8.19 (d, J=2.02 Hz, 1H) 8.96-8.98 (m, 2H) 10.14 (s, 1H) 12.86 (s, 1H)

### Example 135

### N-(4-Chloro-3-{[(5Z)-4-oxo-5-(6-quinoxalinylmethylidene)-4,5-dihydro-1,3-thiazol-2-yl]amino}phenyl)-2-(methyloxy)acetamide

The procedure of example 44 was followed, using methoxyacetyl chloride, quinoxaline-6-carbaldehyde and piperidine in place of propionyl chloride, quinoline-6-carbaldehyde and sodium acetate respectively, to give the title compound (28%) as a solid. 1H NMR (400 MHz, DMSO-d₆) δ ppm 3.36 (s, 3H) 4.01 (s, 2H) 7.49 (d, J=8.59 Hz, 1H) 7.53-7.57 (m, 2H) 7.96 (s, 1H) 7.99 (dd, J=8.84, 2.02 Hz, 1H) 8.18 (d, J=8.59 Hz, 1H) 8.21 (d, J=1.77 Hz, 1H) 8.97-8.99 (m, 2H) 10.01 (s, 1H) 12.87 (s, 1H)

### Example 136

### N-(4-Chloro-3-{[(5Z)-4-oxo-5-(6-quinoxalinylmethylidene)-4,5-dihydro-1,3-thiazol-2-yl]amino}phenyl)-2-(2-thienyl)acetamide trifluoroacetate

The procedure of example 46 was followed, using quinoxaline-6-carbaldehyde and piperidine in place of quinoline-6-carbaldehyde and sodium acetate respectively. Additionally, the material was purified by rp-HPLC (ODS, 10-90% acetonitrile/water+0.1% trifluoroacetic acid) to give the title compound (3%) as a solid. 1H NMR (400 MHz, DMSO-d₆) δ ppm 3.89 (s, 2H) 6.95-6.99 (m, 3H) 7.41 (dd, J=8.84, 2.02 Hz, 1H) 7.48-7.51 (m, 2H) 7.96 (s, 1H) 7.98 (dd, J=8.84, 2.02 Hz, 1H) 8.17 (d, J=8.59 Hz, 1H) 8.21 (d, J=2.02 Hz, 1H) 8.97-8.98 (m, 2H) 10.45 (s, 1H) 12.87 (s, 1H)

### Example 137

### N-(4-Chloro-3-{[(5Z)-4-oxo-5-(6-quinoxalinylmethylidene)-4,5-dihydro-1,3-thiazol-2-yl]amino}phenyl)-2-methylpropanamide, Sodium Salt

The compound of example 4 was dissolved in 1M aq NaOH and methanol with warming and the solution concentrated until solid appeared. After allowing to cool to room temperature, the solid was filtered, washed with a small amount of cold water and dried to give the title compound (24%) as the sodium salt. 1H NMR (400 MHz, DMSO-d₆) δ ppm 1.09 (d, J=6.82 Hz, 6H) 2.56 (m, 1H) 7.18-7.56 (m, 4H) 7.94-8.22 (m, 3H) 8.86-9.00 (m, 2H) 9.86 (s, 1H)

### Examples 138 and 139

### N-(4-Chloro-3-{[(5Z)-4-oxo-5-(6-quinoxalinylmethylidene)-4,5-dihydro-1,3-thiazol-2-yl]amino}phenyl)cyclobutanecarboxamide, sodium salt

a) N-(3-Amino-4-chlorophenyl)cyclobutanecarboxamide. Cyclobutylcarbonyl chloride (3.64 mL, 31.9 mmol) was added dropwise to an ice-cooled, stirred solution of 4-chloro-3-nitroaniline (5.00 g, 29.0 mmol) and pyridine (3.5 mL, 43.2 mmol) in dichloromethane (30 mL) under argon. The mixture was allowed to warm to room temperature and stirred 1 h, then the solvent removed under reduced pressure. 0.5 M aqueous potassium carbonate (10 mL) and methanol (30 mL) was added and the mixture stirred 0.5 h, then diluted with water (200 mL) and extracted with ethyl acetate. The extracts were washed (1M aq HCl, water, brine), dried (MgSO₄) and evaporated to dryness under reduced pressure. A solution of the crude amide in methanol (150 mL) was stirred with Raney® nickel (˜0.5 g) under 1 atm of hydrogen for 18 h. After removal of the hydrogen, the mixture was filtered through a PTFE micropore filter, then evaporated under reduced pressure to give the title compound (6.52 g, 100%) as an oil. 1H NMR (400 MHz, DMSO-d₆) δ 1.80 (m, 1H), 1.92 (m, 1H), 2.03-2.11 (m, 2H), 2.14-2.24 (m, 2H), 3.18 (m, 1H), 5.32 (s, 2H), 6.73 (dd, J=8.6, 2.5 Hz, 1H), 7.05 (d, J=8.6 Hz, 1H), 7.20 (d, J=2.5 Hz, 1H), 9.56 (s, 1H).

b) Quinoxaline-6-carbaldehyde. A suspension of 3,4-diaminotoluene (50.0 g; 0.409 mol.) and glyoxal (40% aq. soln.; 52.0 mL; 0.450 mol.) in water (150 mL) and CH₃CN (20.0 mL) was heated to 60° C. for 1 h. Heating was then discontinued and brine (100 mL) was added. The solution was extracted with EtOAc (3×150 mL) and the combined organic layers were dried over MgSO₄, filtered, and concentrated in vacuo. Purification via distillation under reduced pressure (120° C., 10 torr) provided 6-methylquinoxaline (48.0 g, 81%) as a clear, colorless oil. 1H NMR (400 MHz, CDCl₃) [symbol] ppm 2.61 (s, 3H) 7.61 (dd, J=8.59, 1.77 Hz, 1H) 7.88 (s, 1H) 8.00 (d, J=8.59 Hz, 1H) 8.79 (dd, J=9.85, 1.77 Hz, 2H) MS (ES+) m/e 145 [M+H]⁺. A suspension of 6-methylquinoxaline (8.0 g; 0.055 mol.) and selenium dioxide (6.77 g; 0.061 mol.) in 1,4-dioxane (5.0 mL) was irradiated at 200° C. for 30 min. in a Biotage Initiator microwave synthesizer. The above procedure was repeated five further times and the combined, cooled reaction mixtures were dissolved in CH₂Cl₂, filtered through a plug of celite, and concentrated in vacuo. Purification via flash column chromatography (silica gel, 20-50% ethyl acetate in hexanes) followed by crystallization from CH₂Cl₂ provided quinoxaline-6-carbaldehyde (40.0 g, 91%) as a white solid. ¹H NMR (400 MHz, CDCl₃) [symbol] ppm 10.25 (s, 1H) 8.95 (s, 2H) 8.57 (d, J=1.3 Hz, 1H) 8.24 (dd, J=8.6, 1.5 Hz, 1H) 8.20 (d, J=8.6 Hz, 1H). MS (ES+) m/e 159 [M+H]⁺.

c) N-{4-Chloro-3-[(4-oxo-4,5-dihydro-1,3-thiazol-2-yl)amino]phenyl}cyclobutane-carboxamide. The method of example 77(a) was followed here, using the compound from example 138(a) in place of 2-chloro-5-nitro-1,4-benzenediamine, to give the title compound (89%) as a brown solid. 1H NMR (400 MHz, DMSO-d₆) δ 1.82 (m, 1H), 1.95 (m, 1H), 2.10 (m, 2H), 2.21 (m, 2H), 3.21 (m, 1H), 4.03 (s, 2H), 7.30 (d, J=8.8 Hz, 1H), 7.38 (d, J=8.8 Hz, 1H), 7.45 (s, 1H), 9.85 (s, 1H), 11.99 (brs, 1H).

d) N-(4-Chloro-3-{[(5Z)-4-oxo-5-(6-quinoxalinylmethylidene)-4,5-dihydro-1,3-thiazol-2-yl]amino}phenyl)cyclobutanecarboxamide, sodium salt. The method of example 105(d) were followed here, using quinoxaline-6-carbaldehyde (example 138b) in place of quinoline-6-carbaldehyde. Additionally, the final compound was purified by chromatography (silica gel, 1-10% methanol/dichloromethane) and trituration with ethanol to give the title compound (67%) as the free acid. 1H NMR (400 MHz, DMSO-d₆) δ1.80 (m, 1H), 1.91 (m, 1H), 2.10 (qd, J=8.6, 3.0 Hz, 2H), 2.16-2.26 (m, 2H), 3.22 (m, 1H), 7.40-7.51 (m, 3H), 7.93-8.02 (m, 2H), 8.15-8.25 (m, 2H), 8.97 (s, 2H), 9.92 (s, 1H), 12.83 (1H, br s). The free acid (0.476 g, 1.03 mmol) was dissolved in 1M aq NaOH (2 mL) and water (5 mL) with warming. After allowing to cool to room temperature, the solid was filtered, washed with a small amount of cold water and dried to give the title compound (0.526 g, 89%) as the sodium salt, pentahydrate. 1H NMR (400 MHz, DMSO-d₆) δ 1.80 (m, 1H), 1.90 (m, 1H), 2.04-2.13 (m, 2H), 2.17-2.27 (m, 2H), 3.21 (m, 1H), 7.20-7.53 (m, 4H), 7.97-8.19 (m, 3H), 8.70-9.00 (m, 2H), 9.73 (br s, 1H).

### Example 140

### N-(4-Chloro-3-{[(5Z)-4-oxo-5-(6-quinoxalinylmethylidene)-4,5-dihydro-1,3-thiazol-2-yl]amino}phenyl)cyclopentanecarboxamide

The method of example 105(d) was followed, using the compound from example 94(c) in place of the compound from example 105(c) and quinoxaline-6-carbaldehyde (example 138b) in place of quinoline-6-carbaldehyde. Additionally, the final compound was purified by chromatography (silica gel, 1-7% methanol/dichloromethane) and boiling in ethanol, cooling, filtering and drying to give the title compound (28%) as a solid. 1H NMR (400 MHz, DMSO-d₆) δ 1.49-1.59 (m, 2H), 1.63-1.73 (m, 4H), 1.79-1.89 (m, 2H), 2.76 (m, 1H), 7.40-7.51 (m, 3H), 7.95 (s, 1H), 7.99 (dd, J=8.8, 1.8 Hz, 1H), 8.15-8.24 (m, 2H), 8.97 (s, 2H), 10.07 (s, 1H), 12.83 (br s, 1H).

### Example 141

### (5Z)-2-[(3,5-Dichloro-4-pyridinyl)amino]-5-(6-quinoxalinylmethylidene)-1,3-thiazol-4(5H)-one trifluoroacetate

4-Amino-3,5-dichloropyridine (110 mg, 0.67 mmoles) was stirred in N-methylpyrrolidinone (5.0 mL) in an argon atmosphere and treated with sodium hydride (60% in mineral oil, 90 mg, 2.25 mmoles) and stirred for 10 minutes until a pink color persisted. (5Z)-2-(methylthio)-5-(6-quinoxalinylmethylidene)-1,3-thiazol-4(5H)-one (T. Rueckle; et. al., PCT Int. Appl. (2005), WO2005011686A1,140 mg, 0.48 mmoles) was added and stirring was continued for 30 minutes. The mixture was poured onto ice and acidified with 6N hydrochloric acid, the solid collected, washed with water, ethanol, dichloromethane and hexane. Purification by preparative HPLC (acetonitrile-water-0.1% TFA) gave the title compound (25 mg, 13%). 1H NMR (400 MHz, CHLOROFORM-d) δ ppm 7.81 (dd, J=8.84, 2.02 Hz, 1H) 7.93 (s, 1H) 8.10-8.18 (m, 2H) 8.52 (s, 2H) 8.87 (s, 2H)

### Example 142

### N-(4-Chloro-3-{[(5Z)-4-oxo-5-(6-quinoxalinylmethylidene)-4,5-dihydro-1,3-thiazol-2-yl]amino}phenyl)-4-piperidinecarboxamide trifluoroacetate

The procedures of example 113(a) and (b) were followed, using N—(BOC)-4-piperidinecarboxylic acid in place of BOC-L-proline. The intermediate was treated according to the procedures of example 31(b) and example 105(d) and the resulting BOC protected compound dissolved in 1:1 trifluoroacetic acid/dichloromethane for 0.5 h. The solvent was removed under reduced pressure and the residue chromatographed (silica gel, 10% methanol/dichloromethane) to give the title compound (4% overall) as a brown solid. 1H NMR (400 MHz, DMSO-d₆) δ ppm 1.72-1.85 (m, 2H) 1.92-2.00 (m, 2H) 2.62 (m, 1H) 2.87-2.97 (m, 2H) ˜3.30-3.40 (m, 2H under DHO peak) 7.41 (dd, J=8.59, 2.27 Hz, 1H) 7.45-7.50 (m, 2H) 7.92 (s, 1H) 7.99 (dd, J=8.72, 1.64 Hz, 1H) 8.17 (d, J=8.84 Hz, 1H) 8.20 (d, J=1.26 Hz, 1H) 8.37 (br s, 2H) 8.97 (s, 2H) 10.24 (s, 1H) 12.83 (br s, 1H).

### Example 143

### N-(2-Chloro-3-{[(5Z)-4-oxo-5-(6-quinoxalinylmethylidene)-4,5-dihydro-1,3-thiazol-2-yl]amino}phenyl)-2-methylpropanamide

a) 2-Chloro-1,3-benzenediamine. A solution of 2,6-dinitrochlorobenzene (5.0 g, 24.65 mmoles) was dissolved in a hot mixture of ethanol (30 mL) and water (30 mL) and heated to reflux. Powdered iron (7.0 g, 125 mmoles) was then added followed by the dropwise addition of 6N HCl (7.0 mL) in ethanol (20 mL). Initial additions of the acid were violent. The mixture was then heated under reflux for 2 hours, the iron removed and the liquors basified with sodium hydroxide solution. The mixture was filtered through celite and evaporated to low volume, taken up in ethyl acetate, dried and evaporated to a solid (2.8 g, 80%). 1H NMR (400 MHz, DMSO-d₆) δ ppm 5.01 (s, 4H) 6.02 (d, J=8.08 Hz, 2H) 6.68 (t, J=7.96 Hz, 1H).

b) N-(3-Amino-2-chlorophenyl)-2-methylpropanamide. A mixture of 2-chloro-1,3-benzenediamine (300 mg, 2.1 mmoles) and isobutyric anhydride (350 uL, 2.1 mmoles) was stirred in chloroform (10 mL) for 20 hours. The mixture was evaporated onto silica gel and chromatographed (0-5% methanol-dichloromethane) to give the title compound (300 mg, 67%). 1H NMR (400 MHz, CHLOROFORM-d) δ ppm 1.31 (d, J=7.07 Hz, 6H) 2.62 (ddd, J=114.02, 6.95, 6.82 Hz, 1H) 6.57 (dd, J=7.96, 1.39 Hz, 1H) 7.09 (t, J=8.08 Hz, 1H) 7.66 (s, 1H) 7.82 (d, J=8.08 Hz, 1H).

c) (5Z)-5-(6-Quinoxalinylmethylidene)-2-thioxo-1,3-thiazolidin-4-one. A mixture of 6-quinoxalinecarbaldehyde (6.75 g, 42.7 mmoles), rhodanine (5.69 g, 42.7 mmoles) and sodium acetate (10.5 g, 128 mmoles) in acetic acid (150 mL) was heated under reflux for 18 hours. The mixture solidified during the reaction, which was triturated with water, the solid collected and washed successively with water, methanol and dichloromethane to give the title compound as a dark red solid (11.5 g, 98%). 1H NMR (400 MHz, DMSO-d₆) δ ppm 7.90 (s, 1H) 8.03 (dd, J=8.84, 1.77 Hz, 1H) 8.21 (d, J=8.59 Hz, 1H) 8.31 (d, J=1.77 Hz, 1H) 9.02 (dd, J=6.32, 1.77 Hz, 2H) 14.01 (s, 1H).

d) (5Z)-2-(Methylthio)-5-(6-quinoxalinylmethylidene)-1,3-thiazol-4(5H)-one. A mixture of (5Z)-5-(6-quinoxalinylmethylidene)-2-thioxo-1,3-thiazolidin-4-one (11.5 g, 42 mmoles), iodomethane (18.3 mL, 294 mmoles) and triethylamine (17.6 mL, 126 mmoles) in ethanol (250 mL) was sealed in a flask and stirred at ambient temperature for 4 hours. The mixture was partially evaporated, slurried with water, the solid collected, washed with methanol and dichloromethane to give crude product (7.0 g, 58%). A 2 g portion of the crude product was further washed with dichloromethane, ether and hexane to give essentially pure material. Additional product was recovered from the mother liquors. 1H NMR (400 MHz, DMSO-d₆) δ ppm 2.88 (s, 3H) 8.11 (d, J=2.02 Hz, 1H) 8.13 (s, 1H) 8.24 (d, J=8.59 Hz, 1H) 8.41 (d, J=1.77 Hz, 1H) 8.98-9.06 (m, 2H).

e) N-(2-Chloro-3-{[(5Z)-4-oxo-5-(6-quinoxalinylmethylidene)-4,5-dihydro-1,3-thiazol-2-yl]amino}phenyl)-2-methylpropanamide. A mixture of (5Z)-2-(methylthio)-5-(6-quinoxalinylmethylidene)-1,3-thiazol-4(5H)-one (145 mg, 0.5 mmoles) and N-(3-amino-2-chlorophenyl)-2-methylpropanamide (145 mg, 0.68 mmoles) was heated in valeric acid (1.5 mL) at 140° C. for 1 hour. The mixture was cooled and diluted with ether to give a solid that was collected and chromatographed (hexane-ethyl acetate) to give the title compound (45 mg, 20%). 1H NMR (400 MHz, DMSO-d₆) δ ppm 1.13 (d, J=6.82 Hz, 6H) 2.75 (dt, J=13.58, 6.73 Hz, 1H) 6.99 (d, J=8.34 Hz, 1H) 7.35 (t, J=8.08 Hz, 1H) 7.53 (d, J=8.08 Hz, 1H) 7.95 (s, 1H) 7.98 (dd, J=8.72, 1.89 Hz, 1H) 8.18 (d, J=8.59 Hz, 1H) 8.21 (d, J=1.77 Hz, 1H) 8.98 (s, 2H) 9.45 (s, 1H) 12.87 (s, 1H)

### Example 144

### N-(4-Chloro-3-{[(5Z)-4-oxo-5-(6-quinoxalinylmethylidene)-4,5-dihydro-1,3-thiazol-2-yl]amino}phenyl)-2,2-dimethylpropanamide

The method of example 105(d) was followed, using the compound from example 132(b) in place of the compound from example 105(c) and quinoxaline-6-carbaldehyde (example 138b) in place of quinoline-6-carbaldehyde. Additionally, the final compound was purified by boiling in ethanol, cooling, filtering and drying to give the title compound (46%) as an orange solid. 1H NMR (400 MHz, DMSO-d₆) δ 1.22 (s, 9H), 7.45-7.55 (m, 3H), 7.95 (s, 1H), 7.99 (dd, J=8.8, 2.0 Hz, 1H), 8.16-8.24 (m, 2H), 8.97 (s, 2H), 9.37 (s, 1H), 12.83 (br s, 1H).

### Example 145

### 1,1-Dimethylethyl {2-[(4-chloro-3-{[(5Z)-4-oxo-5-(6-quinoxalinylmethylidene)-4,5-dihydro-1,3-thiazol-2-yl]amino}phenyl)amino]-1,1-dimethyl-2-oxoethyl}carbamate

a) 1,1-Dimethylethyl {2-[(3-amino-4-chlorophenyl)amino]-1,1-dimethyl-2-oxoethyl}carbamate.

Benzotriazol-1-yloxytripyrrolidinophosphonium hexafluorophosphate (4.00 g, 7.69 mmol) was added to a mixture of 4-chloro-1,3-phenylenediamine (1.00 g, 7.01 mmol), N-(tert-butoxycarbonyl)-2-aminoisobutyric acid (1.60 g, 7.87 mmol), N,N-diisopropylethylamine (3.00 mL, 17.2 mmol) and dichloromethane (7 mL) and the mixture stirred 18 h under nitrogen. The mixture was diluted with ethyl acetate, washed with 1M aqueous hydrochloric acid and brine, then dried (MgSO₄). The solvent was removed under reduced pressure and the residue chromatographed (silica gel, 10-50% ethyl acetate/hexane) to give the title compound (0.75 g, 33%) as a solid. 1H NMR (400 MHz, DMSO-d₆) δ ppm 1.36 (m, 15H) 5.29 (s, 2H) 6.73 (br s, 1H) 6.88 (br s, 1H) 7.05 (d, J=8.59 Hz, 1H) 7.20 (s, 1H) 9.21 (s, 1H)

b) 1,1-Dimethylethyl [2-({4-chloro-3-[(4-oxo-4,5-dihydro-1,3-thiazol-2-yl)amino]phenyl}amino)-1,1-dimethyl-2-oxoethyl]carbamate. The procedure of example 31(b) was followed, using the compound from example 145(a) in place of the compound from example 31(a), to give the title compound (71%) as a solid. 1H NMR (400 MHz, DMSO-d₆) δ ppm 1.36 (s, 15H) 4.03 (s, 2H) 6.96 (s, 1H) 7.34-7.51 (m, 3H) 9.56 (s, 1H) 11.99 (s, 1H)

c) 1,1-Dimethylethyl {2-[(4-chloro-3-{[(5Z)-4-oxo-5-(6-quinoxalinylmethylidene)-4,5-dihydro-1,3-thiazol-2-yl]amino}phenyl)amino]-1,1-dimethyl-2-oxoethyl}carbamate. A mixture of the compound from example 145(b) (0.300 g, 0.703 mmol), quinoxaline-6-carbaldehyde (0.122 g, 0.772 mmol), piperidine (0.030 mL, 0.707 mmol) and ethanol (1 mL) was heated in a microwave reactor at 150° C. for 20 min, then cooled. 1M aqueous HCl (1 mL) was added, followed by water (1 mL). The precipitate was filtered and dried, then a sample purified by rp-HPLC (ODS, 10-90% acetonitrile/water+0.1% trifluoroacetic acid) to give the title compound (64%) as a solid. 1H NMR (400 MHz, DMSO-d₆) δ ppm 1.27 (s, 6H) 1.35 (s, 9H) 6.72 (s, 1H) 7.00 (s, 1H) 7.45 (d, J=8.84 Hz, 1H) 7.53 (s, 1H) 7.93 (s, 1H) 7.98 (dd, J=8.84, 1.77 Hz, 1H) 8.16 (d, J=8.59 Hz, 1H) 8.21 (m, 1H) 8.97 (s, 2H) 9.63 (s, 1H) 12.84 (s, 1H)

### Example 146

### N-(2-Chloro-3-{[(5Z)-4-oxo-5-(6-quinoxalinylmethylidene)-4,5-dihydro-1,3-thiazol-2-yl]amino}phenyl)cyclopropanecarboxamide

a) N-(3-Amino-2-chlorophenyl)cyclopropanecarboxamide. A mixture of 2-chloro-1,3-benzenediamine (500 mg, 3.5 mmoles), cyclopropylcarbonyl chloride (272 uL, 3.0 mmoles) and triethylamine (400 uL, 3.0 mmoles) was stirred in chloroform (5 mL) for 20 hours. The mixture was evaporated onto silica gel and chromatographed (hexane-ethyl acetate) to give the title compound (150 mg, 47%). 1H NMR (400 MHz, DMSO-d₆) δ ppm 0.73-0.80 (m, 4H) 1.90-2.01 (m, 1H) 5.37 (br s, 2H) 6.59 (dd, J=8.08, 1.52 Hz, 1H) 6.84-6.91 (m, 1H) 6.95 (t, J=7.96 Hz, 1H) 9.48 (s, 1H).

b) N-(2-Chloro-3-{[(5Z)-4-oxo-5-(6-quinoxalinylmethylidene)-4,5-dihydro-1,3-thiazol-2-yl]amino}phenyl)cyclopropanecarboxamide. A mixture of (5Z)-2-(methylthio)-5-(6-quinoxalinylmethylidene)-1,3-thiazol-4(5H)-one (140 mg, 0.48 mmoles) and N-(3-amino-2-chlorophenyl)cyclopropanecarboxamide (130 mg, 0.61 mmoles) was heated in valeric acid (1.0 mL) at 140° C. for 40 minutes. The mixture was cooled and diluted with ethanol to give a solid that was collected and purified by preparative HPLC (acetonitrile-water-0.1% TFA) to give the title compound (10 mg, 4.6%). 1H NMR (400 MHz, DMSO-d₆) δ ppm 0.82 (d, J=5.05 Hz, 4H) 2.02 (s, 1H) 6.97 (d, J=7.58 Hz, 1H) 7.34 (t, J=8.08 Hz, 1H) 7.58 (d, J=8.08 Hz, 1H) 7.95 (s, 1H) 7.98 (d, J=9.35 Hz, 1H) 8.18 (d, J=8.84 Hz, 1H) 8.21 (s, 1H) 8.97 (s, 2H) 9.81 (s, 1H) 12.88 (s, 1H)

### Example 147

### N-(2-Chloro-3-{[(5Z)-4-oxo-5-(6-quinoxalinylmethylidene)-4,5-dihydro-1,3-thiazol-2-yl]amino}phenyl)cyclobutanecarboxamide

a) N-(3-Amino-2-chlorophenyl)cyclobutanecarboxamide. A mixture of 2-chloro-1,3-benzenediamine (500 mg, 3.5 mmoles), cyclobutylcarbonyl chloride (325 uL, 3.0 mmoles) and triethylamine (400 uL, 3.0 mmoles) was stirred in chloroform (5 mL) for 20 hours. The mixture was evaporated onto silica gel and chromatographed (hexane-ethyl acetate) to give the title compound (180 mg, 26%). 1H NMR (400 MHz, CHLOROFORM-d) δ ppm 1.93-1.99 (m, 1H) 2.00-2.08 (m, 1H) 2.30 (ddd, J=17.81, 8.84, 8.72 Hz, 3H) 2.37-2.48 (m, 2H) 3.18-3.30 (m, 1H) 6.56 (d, J=7.07 Hz, 1H) 7.09 (t, J=8.08 Hz, 1H) 7.53 (s, 1H) 7.84 (d, J=8.08 Hz, 1H).

b) N-(2-Chloro-3-{[(5Z)-4-oxo-5-(6-quinoxalinylmethylidene)-4,5-dihydro-1,3-thiazol-2-yl]amino}phenyl)cyclobutanecarboxamide. A mixture of (5Z)-2-(methylthio)-5-(6-quinoxalinylmethylidene)-1,3-thiazol-4(5H)-one (163 mg, 0.66 mmoles) and N-(3-amino-2-chlorophenyl)cyclobutanecarboxamide (170 mg, 0.75 mmoles) was heated in valeric acid (2.0 mL) at 140° C. for 40 minutes. The mixture was cooled and diluted with ethanol to give a solid that was collected, washed with ethanol and dichloromethane to give the title compound (21 mg, 7%). 1H NMR (400 MHz, DMSO-d₆) δ ppm 1.81 (d, J=9.85 Hz, 1H) 1.88-2.00 (m, 1H) 2.13 (d, J=9.09 Hz, 2H) 2.18-2.29 (m, 2H) 6.98 (d, J=7.58 Hz, 1H) 7.35 (t, J=7.96 Hz, 1H) 7.57 (d, J=8.59 Hz, 1H) 7.95 (s, 1H) 7.98 (dd, J=8.84, 2.02 Hz, 1H) 8.18 (d, J=8.59 Hz, 1H) 8.21 (d, J=1.52 Hz, 1H) 8.98 (s, 2H) 9.34 (s, 1H) 12.87 (s, 1H)

### Example 148

### N˜1˜-(4-Chloro-3-{[(5Z)-4-oxo-5-(6-quinoxalinylmethylidene)-4,5-dihydro-1,3-thiazol-2-yl]amino}phenyl)-2-methylalaninamide trifluoroacetate

A solution of the compound from example 145 (0.072 g, 0.128 mmol) in trifluoroacetic acid (1 mL) was stirred 5 min, diluted with dichloromethane (10 mL) then, after 10 min, evaporated under reduced pressure and triturated with 6% methanol/dichloromethane to give the title compound (0.031 g, 41%) as a solid. 1H NMR (400 MHz, DMSO-d₆) δ ppm 1.59 (s, 6H) 7.45-7.52 (m, 2H) 7.58 (d, J=9.35 Hz, 1H) 7.97 (s, 1H) 8.00 (dd, J=8.84, 2.02 Hz, 1H) 8.19 (d, J=8.84 Hz, 1H) 8.21 (d, J=1.77 Hz, 1H) 8.25 (br s, 3H) 8.97-9.00 (m, 2H) 10.10 (s, 1H) 12.91 (s, 1H)

### Example 149

### N-(4-Chloro-3-{[(5Z)-4-oxo-5-(6-quinoxalinylmethylidene)-4,5-dihydro-1,3-thiazol-2-yl]amino}phenyl)ethanesulfonamide

The method of example 75 was followed here, using ethanesulfonyl chloride and quinoxaline-6-carbaldehyde in place of phenylmethanesulfonyl chloride and quinoline-6-carbaldehyde respectively. Additionally, the material was purified by rp-HPLC (ODS, 10-90% acetonitrile/water+0.1% trifluoroacetic acid) to give the title compound (9%) as a solid. 1H NMR (400 MHz, DMSO-d₆) δ ppm 1.22 (t, J=7.33 Hz, 3H) 3.19 (q, J=7.33 Hz, 2H) 7.02 (d, J=2.27 Hz, 1H) 7.06 (dd, J=8.84, 2.53 Hz, 1H) 7.52 (d, J=8.59 Hz, 1H) 7.96 (s, 1H) 8.00 (dd, J=8.84, 2.02 Hz, 1H) 8.18 (d, J=8.84 Hz, 1H) 8.22 (d, J=2.02 Hz, 1H) 8.99 (s, 2H) 10.09 (s, 1H) 12.88 (s, 1H)

### Example 150

### N-(4-Chloro-3-{[(5Z)-4-oxo-5-(6-quinoxalinylmethylidene)-4,5-dihydro-1,3-thiazol-2-yl]amino}phenyl)-2-propanesulfonamide

a) N-(3-Amino-4-chlorophenyl)-2-propanesulfonamide. A mixture of 4-chloro-1,3-phenylenediamine (0.500 g, 3.51 mmol), isopropylsulfonyl chloride (0.39 mL, 3.49 mmol), imidazole (0.240 g, 3.53 mmol) and dichloromethane (2 mL) was stirred 1 h at room temperature, then the mixture chromatographed (silica gel, ethyl acetate/hexane) to give the title compound (0.269 g, 31%) as an oil. 1H NMR (400 MHz, CHLOROFORM-d) δ ppm 1.38 (d, J=6.82 Hz, 6H) 3.34 (septet, J=6.82 Hz, 1H) 4.22 (s, 2H) 6.54 (dd, J=8.59, 2.53 Hz, 1H) 6.78 (d, J=2.53 Hz, 1H) 7.14 (d, J=8.59 Hz, 1H) 7.34 (s, 1H)

b) N-(4-Chloro-3-{[(5Z)-4-oxo-5-(6-quinoxalinylmethylidene)-4,5-dihydro-1,3-thiazol-2-yl]amino}phenyl)-2-propanesulfonamide. The compound from example 150(a) was processed according to the procedures of example 31(b) and example 145(c). Instead of the HPLC purification, the crude product was precipitated from dimethylformamide with water, filtered, washed with water and dried to give the title compound (8%) as a solid. 1H NMR (400 MHz, DMSO-d₆) δ ppm 1.26 (d, J=6.82 Hz, 6H) 3.33 (septet, J=6.82 Hz, 1H) 7.04 (s, 1H) 7.07 (dd, J=8.59, 2.53 Hz, 1H) 7.51 (d, J=8.59 Hz, 1H) 7.94 (s, 1H) 8.00 (dd, J=8.84, 2.02 Hz, 1H) 8.18 (d, J=8.59 Hz, 1H) 8.20 (d, J=1.77 Hz, 1H) 8.98 (s, 2H) 10.04 (s, 1H) 12.88 (s, 1H)

### Example 151

### N-(4-Chloro-3-{[(5Z)-4-oxo-5-(6-quinolinylmethylidene)-4,5-dihydro-1,3-thiazol-2-yl]amino}phenyl)-N-(1-methylethyl)acetamide

a) N-{4-Chloro-3-[(4-oxo-4,5-dihydro-1,3-thiazol-2-yl)amino]phenyl}-N-(1-methylethyl)acetamide. Acetyl chloride (0.355 mL, 4.99 mmol) was injected into a stirred solution of the compound from example 111(a) (0.284 g, 1.00 mmol) in pyridine (5 mL) at room temperature. The mixture was stirred 2.5 h, then 1M aqueous NaOH (10 mL) added. After stirring 1 h, the mixture was acidified to pH 2 (6M aqueous HCl) and extracted with ethyl acetate. The extracts were washed with water, brine, dried (MgSO₄) and evaporated under reduced pressure. The residue was chromatographed (silica gel, 2-10% methanol/dichloromethane) to give the title compound (77%) as a foam. LC/MS (ES+) m/e 326 [M+H]⁺.

b) N-(4-Chloro-3-{[(5Z)-4-oxo-5-(6-quinolinylmethylidene)-4,5-dihydro-1,3-thiazol-2-yl]amino}phenyl)-N-(1-methylethyl)acetamide. The method of example 105(d) was followed, using the compound from example 151(a) in place of the compound from example 105(c), to give the title compound (63%) as a yellow powder. 1H NMR (400 MHz, DMSO-d₆) δ ppm 1.04 (d, J=6.57 Hz, 6H) 1.74 (s, 3H) 4.81 (m, 1H) 7.07-7.17 (m, 2H) 7.59 (dd, J=8.34, 4.04 Hz, 1H) 7.65 (d, J=8.08 Hz, 1H) 7.84-7.88 (m, 2H) 8.07 (d, J=8.84 Hz, 1H) 8.09 (d, J=1.52 Hz, 1H) 8.32 (d, J=8.34 Hz, 1H) 8.95 (dd, J=4.29, 1.52 Hz, 1H) 12.83 (s, 1H)

### Example 152

### N-(4-Chloro-3-{[(5Z)-4-oxo-5-(6-quinoxalinylmethylidene)-4,5-dihydro-1,3-thiazol-2-yl]amino}phenyl)-N-(1-methylethyl)acetamide

The method of example 151 was followed, using quinoxaline-6-carbaldehyde in place of quinoline-6-carbaldehyde, to give the title compound (42%) as an orange powder. 1H NMR (400 MHz, DMSO-d₆) δ ppm 1.04 (d, J=6.57 Hz, 6H) 1.74 (s, 3H) 4.82 (m, 1H) 7.11 (d, J=8.08 Hz, 1H) 7.15 (s, 1H) 7.65 (d, J=8.34 Hz, 1H) 7.94 (s, 1H) 7.98 (dd, J=8.84, 2.02 Hz, 1H) 8.13-8.18 (m, 2H) 8.97 (d, J=1.77 Hz, 1H) 8.99 (d, J=1.77 Hz, 1H) 12.91 (s, 1H)

### Example 153

### N-(4-Chloro-3-{[(5Z)-4-oxo-5-(6-quinolinylmethylidene)-4,5-dihydro-1,3-thiazol-2-yl]amino}phenyl)-N-(1-methylethyl)methanesulfonamide

The method of example 151 was followed, using methanesulfonyl chloride in place of acetyl chloride, to give the title compound (24%) as an orange powder. 1H NMR (400 MHz, DMSO-d₆) δ ppm 1.15 (d, J=6.82 Hz, 6H) 3.11 (s, 3H) 4.34 (m, 1H) 7.15 (dd, J=8.34, 2.27 Hz, 1H) 7.21 (d, J=1.77 Hz, 1H) 7.60 (dd, J=8.34, 4.29 Hz, 1H) 7.63 (d, J=8.59 Hz, 1H) 7.84-7.89 (m, 2H) 8.07 (d, J=8.84 Hz, 1H) 8.11 (s, 1H) 8.34 (d, J=8.08 Hz, 1H) 8.95 (dd, J=4.17, 1.64 Hz, 1H) 12.83 (s, 1H)

### Example 154

### N-(4-Chloro-3-{[(5Z)-4-oxo-5-(6-quinoxalinylmethylidene)-4,5-dihydro-1,3-thiazol-2-yl]amino}phenyl)-N-(1-methylethyl)methanesulfonamide

The method of example 153 was followed, using quinoxaline-6-carbaldehyde in place of quinoline-6-carbaldehyde, to give the title compound (18%) as an orange powder. 1H NMR (400 MHz, DMSO-d₆) δ ppm 1.16 (d, J=6.82 Hz, 6H) 3.11 (s, 3H) 4.35 (septet, J=6.63 Hz, 1H) 7.15 (dd, J=8.59, 2.27 Hz, 1H) 7.21 (s, 1H) 7.63 (d, J=8.59 Hz, 1H) 7.94 (s, 1H) 7.98 (dd, J=8.84, 1.77 Hz, 1H) 8.16 (d, J=8.84 Hz, 1H) 8.18 (d, J=1.52 Hz, 1H) 8.98 (m, 2H) 12.91 (s, 1H)

### Example 155

### (5Z)-2-({2-Chloro-5-[(1-methylethyl)amino]phenyl}amino)-5-(6-quinoxalinylmethylidene)-1,3-thiazol-4(5H)-one

The method of example 111 was followed, using quinoxaline-6-carbaldehyde in place of quinoline-6-carbaldehyde, to give the title compound (44%) as an orange powder. 1H NMR (400 MHz, DMSO-d₆) δ ppm 1.13 (d, J=6.06 Hz, 6H) 3.51 (m, 1H) 5.74 (d, J=7.58 Hz, 1H) 6.27 (s, 1H) 6.42 (dd, J=8.59, 2.27 Hz, 1H) 7.17 (d, J=8.84 Hz, 1H) 7.92 (s, 1H) 7.99 (dd, J=8.84, 2.02 Hz, 1H) 8.18 (d, J=8.84 Hz, 1H) 8.21 (d, J=1.77 Hz, 1H) 8.97 (s, 2H) 12.71 (s, 1H)

### Example 156

### N-(2-Chloro-3-{[(5Z)-4-oxo-5-(6-quinoxalinylmethylidene)-4,5-dihydro-1,3-thiazol-2-yl]amino}phenyl)methanesulfonamide

a) N-(3-Amino-2-chlorophenyl)methanesulfonamide. A mixture of 2-chloro-1,3-benzenediamine (500 mg, 3.5 mmoles), methanesulfonyl chloride (233 uL, 3.0 mmoles) and triethylamine (400 uL, 3.0 mmoles) was stirred in chloroform (5 mL) for 20 hours. The mixture was evaporated onto silica gel and chromatographed (hexane-ethyl acetate) to give the title compound (450 mg, 67%). 1H NMR (400 MHz, DMSO-d₆) δ ppm 2.99 (s, 3H) 5.47 (s, 2H) 6.63 (dd, J=7.83, 1.26 Hz, 1H) 6.67 (dd, J=8.08, 1.52 Hz, 1H) 6.99 (t, J=8.08 Hz, 1H) 9.11 (s, 1H).

b) N-(2-Chloro-3-{[(5Z)-4-oxo-5-(6-quinoxalinylmethylidene)-4,5-dihydro-1,3-thiazol-2-yl]amino}phenyl)methanesulfonamide. A mixture of (5Z)-2-(methylthio)-5-(6-quinoxalinylmethylidene)-1,3-thiazol-4(5H)-one (287 mg, 1.0 mmoles) and N-(3-amino-2-chlorophenyl)methanesulfonamide (220 mg, 1.0 mmoles) was heated in valeric acid (2.0 mL) at 160° C. for 40 minutes. Separation by preparative HPLC (acetonitrile-water-0.1% TFA) gave impure material which was combined with the product of a similar reaction that was purified by flash chromatography (0-5% methanol-dichloromethane) and purified by preparative HPLC (acetonitrile-water-0.1% TFA) to give the title compound (50 mg, 5.4%). 1H NMR (400 MHz, DMSO-d₆) δ ppm 3.10 (s, 3H) 7.07 (d, J=7.33 Hz, 1H) 7.28-7.36 (m, 1H) 7.39 (t, J=7.96 Hz, 1H) 7.96 (s, 1H) 7.98 (dd, J=8.84, 2.02 Hz, 1H) 8.18 (d, J=8.84 Hz, 1H) 8.21 (d, J=2.02 Hz, 1H) 8.98 (s, 2H) 9.50 (s, 1H) 12.89 (s, 1H)

### Example 157

### N-(2-Chloro-3-{[(5Z)-4-oxo-5-(6-quinoxalinylmethylidene)-4,5-dihydro-1,3-thiazol-2-yl]amino}phenyl)benzamide

a) N-(3-Amino-2-chlorophenyl)benzamide. A mixture of 2-chloro-1,3-benzenediamine (500 mg, 3.5 mmoles), triethylamine (400 uL, 3.0 mmoles) and benzoyl chloride (350 uL, 3.0 mmoles) was stirred in chloroform (5 mL) for 20 hours. The mixture was evaporated onto silica gel and chromatographed (0-5% methanol-dichloromethane) to give the title compound (300 mg, 41%). 1H NMR (400 MHz, DMSO-d₆ δ ppm 5.44 (s, 2H) 6.72 (dd, J=8.08, 1.52 Hz, 1H) 6.78 (dd, J=7.83, 1.01 Hz, 1H) 7.04 (t, J=7.96 Hz, 1H) 7.52-7.56 (m, 2H) 7.98 (d, J=7.07 Hz, 2H) 9.84 (s, 1H)

b) (5Z)-2-{[2-Chloro-3-(phenylcarbonyl)phenyl]amino}-5-(6-quinoxalinylmethylidene)-1,3-thiazol-4(5H)-one. A mixture of N-(3-amino-2-chlorophenyl)benzamide (300 mg, 1.2 mmoles) and (5Z)-2-(methylthio)-5-(6-quinoxalinylmethylidene)-1,3-thiazol-4(5H)-one (350 mg, 1.2 mmoles) in valeric acid (3.0 mL) was heated at 140° C. for 1 hr. Flash chromatography (silica gel, 0-5% methanol in dichloromethane) gave crude product which was purified by preparative HPLC acetonitrile-water-0.1% TFA) to give the title compound (30 mg, 5%). 1H NMR (400 MHz, DMSO-d₆) δ ppm 7.11 (d, J=7.33 Hz, 1H) 7.43 (t, J=7.96 Hz, 1H) 7.46-7.51 (m, 1H) 7.55 (t, J=7.45 Hz, 2H) 7.59-7.63 (m, 1H) 7.97 (s, 1H) 7.98-8.02 (m, 2H) 8.02 (s, 1H) 8.20 (d, J=8.59 Hz, 1H) 8.22 (d, J=2.02 Hz, 1H) 8.98 (s, 2H) 10.10 (s, 1H) 12.90 (s, 1H)

### Example 158

### 1,1-Dimethylethyl {2-[(4-chloro-3-{[(5Z)-4-oxo-5-(6-quinolinylmethylidene)-4,5-dihydro-1,3-thiazol-2-yl]amino}phenyl)amino]-1,1-dimethyl-2-oxoethyl}carbamate

The method of example 145 was followed, using quinoline-6-carbaldehyde in place of quinoxaline-6-carbaldehyde, to give the title compound (4%) as a solid. 1H NMR (400 MHz, DMSO-d₆) δ ppm 1.27 (s, 6H) 1.35 (s, 9H) 7.01 (s, 1H) 7.45 (d, J=8.84 Hz, 1H) 7.49-7.59 (m, 3H) 7.81-7.89 (m, 2H) 8.07 (d, J=8.84 Hz, 1H) 8.15 (s, 1H) 8.44 (d, J=8.08 Hz, 1H) 8.93 (dd, J=4.29, 1.52 Hz, 1H) 9.63 (s, 1H) 12.76 (s, 1H)

### Example 159

### N¹-(4-Chloro-3-{[(5Z)-4-oxo-5-(6-quinolinylmethylidene)-4,5-dihydro-1,3-thiazol-2-yl]amino}phenyl)-2-methylalaninamide

The method of example 148 was followed, using the compound from example 158 in place of the compound from example 145. Additionally, the compound was chromatographed (silica gel, 0-10% methanol/dichloromethane) to give the title compound (33%) as a yellow solid. 1H NMR (400 MHz, DMSO-d₆) δ ppm 1.57 (s, 6H) 7.45-7.50 (m, 2H) 7.54-7.60 (m, 2H) 7.84-7.88 (m, 2H) 8.10 (d, J=8.84 Hz, 1H) 8.17 (d, J=1.52 Hz, 1H) 8.46 (d, J=7.07 Hz, 1H) 8.94 (dd, J=4.29, 1.52 Hz, 1H) 10.04 (s, 1H)

### Example 160

### N-(4-Chloro-3-{[(5Z)-4-oxo-5-(6-quinolinylmethylidene)-4,5-dihydro-1,3-thiazol-2-yl]amino}phenyl)-2-propanesulfonamide

The method of example 150 was followed, using quinoline-6-carbaldehyde in place of quinoxaline-6-carbaldehyde. In this case, the material was purified by rp-HPLC (ODS, 10-90% acetonitrile/water+0.1% trifluoroacetic acid) to give the title compound (2%) as a solid. 1H NMR (400 MHz, DMSO-d₆) δ ppm 1.23 (d, J=6.82 Hz, 6H) 3.32 (m, 1H) 7.03 (d, J=2.27 Hz, 1H) 7.07 (dd, J=8.84, 2.53 Hz, 1H) 7.51 (d, J=8.59 Hz, 1H) 7.59 (dd, J=8.34, 4.29 Hz, 1H) 7.85-7.91 (m, 2H) 8.09 (d, J=8.84 Hz, 1H) 8.17 (d, J=1.77 Hz, 1H) 8.46 (d, J=7.83 Hz, 1H) 8.95 (dd, J=4.17, 1.64 Hz, 1H) 10.01 (s, 1H) 12.79 (s, 1H)

### Example 161

### 4-Chloro-N-[2-(methyloxy)ethyl]-3-{[(5Z)-4-oxo-5-(6-quinolinylmethylidene)-4,5-dihydro-1,3-thiazol-2-yl]amino}benzamide trifluoroacetate

a) 3-[(Aminocarbonothioyl)amino]-4-chlorobenzoic acid. To a solution of benzoyl isothiocyanate (475 mg, 3 mmol) and acetone (5 mL) was added a solution of 3-amino-4-chlorobenzoic acid (500 mg, 3 mmol) and acetone (5 mL). The combined solution was stirred for 10 minutes. The acetone was removed and the resulting solid was refluxed in a 2.5 M NaOH solution (50 mL) for an additional 10 minutes. The aqueous layer was removed to afford the title compound. No further purification was attempted. M/S M+1:231.5.

b) 4-Chloro-3-[(4-oxo-4,5-dihydro-1,3-thiazol-2-yl)amino]benzoic acid. 3-[(aminocarbonothioyl)amino]-4-chlorobenzoic acid (672 mg, 3 mmol), chloroacetic acid (275 mg, 3 mmol), and sodium acetate (239 mg, 3 mmol) were dissolved in acetic acid (20 mL) and refluxed at 130° C. for four hours. Diethyl ether (3×25 mL) was used to extract the compound. The organic layers were combined, dried over magnesium sulfate, filtered, and rotovapped to afford the title compound (100 mg, 16%). M/S M+1:271.2.

c) 4-Chloro-N-[2-(methyloxy)ethyl]-3-[(4-oxo-4,5-dihydro-1,3-thiazol-2-yl)amino]benzamide. 4-Chloro-3-[(4-oxo-4,5-dihydro-1,3-thiazol-2-yl)amino]benzoic acid (100 mg, 0.44 mmol), N-cyclohexyl-N′-beta-(N-methylmorpholino)-ethyl]carbodiimide p-toluenesulfonate (276 mg, 0.65 mmol), and 1-hydroxy-7-azabenzotriazole (89 mg, 0.65 mmol) were dissolved in dimethylformamide (5 mL) and stirred for 20 minutes. 2-Methoxyethylamine (49 mg, 0.87 mmol) was then added to the solution and stirred for 18 hours. The solution was diluted with ethyl acetate (20 mL) and washed with water (3×20 mL). The organic layers were combined, dried over magnesium sulfate, filtered, and rotovapped to afford the title compound (74 mg, 56%). M/S M+1:328.4.

d) 4-Chloro-N-[2-(methyloxy)ethyl]-3-{[(5Z)-4-oxo-5-(6-quinolinylmethylidene)-4,5-dihydro-1,3-thiazol-2-yl]amino}benzamide. 4-chloro-N-[2-(methyloxy)ethyl]-3-[(4-oxo-4,5-dihydro-1,3-thiazol-2-l)amino]benzamide (50 mg, 0.16 mmol) and 6-quinolinecarbaldehyde (25 mg, 0.16 mmol) were dissolved in ethanol (2 mL). The reaction was heated in a microwave to 170° C. for 10 minutes. The solution was cooled, and concentrated down. The mixture was injected onto an Agilent Prep HPLC system and purified to afford the title compound. (24 mg, 25%). M/S M+1:468.0.

### Example 162

### 4-Chloro-3-{[(5Z)-4-oxo-5-(6-quinolinylmethylidene)-4,5-dihydro-1,3-thiazol-2-yl]amino}-N-propylbenzamide

The methods of example 161(c) and 161(d) were followed here, using N-propylamine in place of 2-methoxyethylamine, to give the title compound (32%). M/S M+1:452.0.

### Example 163

### 4-Chloro-N-(2-hydroxyethyl)-3-{[(5Z)-4-oxo-5-(6-quinolinylmethylidene)-4,5-dihydro-1,3-thiazol-2-yl]amino}benzamide

The methods of example 161(c) and 161(d) were followed here, using ethanolamine in place of 2-methoxyethylamine, to give the title compound (21%). M/S M+1:453.9.

### Example 164

### 4-Chloro-N-[3-(2-oxo-1-pyrrolidinyl)propyl]-3-{[(5Z)-4-oxo-5-(6-quinolinylmethylidene)-4,5-dihydro-1,3-thiazol-2-yl]amino}benzamide

a) 4-Chloro-3-[(4-oxo-4,5-dihydro-1,3-thiazol-2-yl)amino]-N-[3-(2-oxo-1-pyrrolidinyl)propyl]benzamide. The method of example 161(c) was followed here, using 1-(3-aminopropyl)-2-pyrrolidinone in place of 2-methoxyethylamine, to give the title compound (43%). M/S M+1:394.4.

b) 4-Chloro-N-[3-(2-oxo-1-pyrrolidinyl)propyl]-3-{[(5Z)-4-oxo-5-(6-quinolinylmethylidene)-4,5-dihydro-1,3-thiazol-2-yl]amino}benzamide. 4-Chloro-3-[(4-oxo-4,5-dihydro-1,3-thiazol-2-yl)amino]-N-[3-(2-oxo-1-pyrrolidinyl)propyl]-benzamide (50 mg, 0.16 mmol), 6-quinolinecarbaldehyde (25 mg, 0.16 mmol), and sodium acetate (39 mg, 0.48 mmol) were dissolved in acetic acid (5 mL) and refluxed for 48 hours. The solution was cooled, water was added and the product was collected and washed with water to afford the title compound (49 mg, 59%). M/S M+1:535.0.

### Example 165

### 4-Chloro-N-[2-(4-morpholinyl)ethyl]-3-{[(5Z)-4-oxo-5-(6-quinolinylmethylidene)-4,5-dihydro-1,3-thiazol-2-yl]amino}benzamide

The methods of example 161(c) and 164(b) were followed here, using [2-(4-morpholinyl)ethyl]amine in place of 2-methoxyethylamine, to give the title compound (23%). M/S M+1:523.0.

### Example 166

### 4-Chloro-N-[3-(4-morpholinyl)propyl]-3-{[(5Z)-4-oxo-5-(6-quinolinylmethylidene)-4,5-dihydro-1,3-thiazol-2-yl]amino}benzamide

The methods of example 161(c) and 164(b) were followed here, using [3-(4-morpholinyl)propyl]amine in place of 2-methoxyethylamine, to give the title compound (26%). M/S M+1:537.0.

### Example 167

### 4-Chloro-N-(2-hydroxy-1,1-dimethylethyl)-3-{[(5Z)-4-oxo-5-(6-quinolinylmethylidene)-4,5-dihydro-1,3-thiazol-2-yl]amino}benzamide trifluoroacetate

The methods of example 161(c) and (d) were followed here, using 2-amino-2-methyl-1-propanol in place of 2-methoxyethylamine, to give the title compound (7%). M/S M+1:482.0

### Example 168

### 4-Chloro-N-(1-methyl-4-piperidinyl)-3-{[(5Z)-4-oxo-5-(6-quinolinylmethylidene)-4,5-dihydro-1,3-thiazol-2-yl]amino}benzamide trifluoroacetate

The methods of example 161(c) and (d) were followed here, using 4-methyl-1-piperadinamine in place of 2-methoxyethylamine, to give the title compound (22%). M/S M+1:507.0.

### Example 169

### (5Z)-2-[(2-Chloro-5-{[(3S)-3-hydroxy-1-pyrrolidinyl]carbonyl}phenyl)amino]-5-(6-quinolinylmethylidene)-1,3-thiazol-4(5H)-one trifluoroacetate

The methods of example 161(c) and (d) were followed here, using (3S)-3-pyrrolidinol in place of 2-methoxyethylamine, to give the title compound (15%). M/S M+1:480.0.

### Example 170

### 4-Chloro-3-{[(5Z)-4-oxo-5-(6-quinolinylmethylidene)-4,5-dihydro-1,3-thiazol-2-yl]amino}-N-[2-(2-thienyl)ethyl]benzamide trifluoroacetate

The methods of example 161(c) and (d) were followed here, using [2-(2-thienyl)ethyl]amine in place of 2-methoxyethylamine, to give the title compound (25%). M/S M+1:520.0

### Example 171

### (5Z)-2-{[2-Chloro-5-({4-[2-oxo-2-(1-pyrrolidinyl)ethyl]-1-piperazinyl}carbonyl)phenyl]amino}-5-(6-quinolinylmethylidene)-1,3-thiazol-4(5H)-one trifluoroacetate

The methods of example 161(c) and (d) were followed here, using 1-[2-oxo-2-(1-pyrrolidinyl)ethyl]piperazine in place of 2-methoxyethylamine, to give the title compound (11%). M/S M+1:590.1

### Example 172

### 4-Chloro-N-(cyclopropylmethyl)-3-{[(5Z)-4-oxo-5-(6-quinolinylmethylidene)-4,5-dihydro-1,3-thiazol-2-yl]amino}benzamide trifluoroacetate

The methods of example 161(c) and (d) were followed here, using (cyclopropylmethyl)amine in place of 2-methoxyethylamine, to give the title compound (15%). M/S M+1:464.0

### Example 173

### 4-Chloro-N-(trans-4-hydroxycyclohexyl)-3-{[(5Z)-4-oxo-5-(6-quinolinylmethylidene)-4,5-dihydro-1,3-thiazol-2-yl]amino}benzamide trifluoroacetate

The methods of example 161(c) and (d) were followed here, using trans-4-aminocyclohexanol in place of 2-methoxyethylamine, to give the title compound (16%). M/S M+1:507.0.

### Example 174

### 4-Chloro-N-(3-hydroxypropyl)-3-{[(5Z)-4-oxo-5-(6-quinolinylmethylidene)-4,5-dihydro-1,3-thiazol-2-yl]amino}benzamide trifluoroacetate

The methods of example 161(c) and (d) were followed here, using 3-amino-1-propanol in place of 2-methoxyethylamine, to give the title compound (13%). M/S M+1:468.0.

### Example 175

### 4-Chloro-3-{[(5Z)-4-oxo-5-(6-quinolinylmethylidene)-4,5-dihydro-1,3-thiazol-2-yl]amino}-N-[2-(1-pyrrolidinyl)ethyl]benzamide trifluoroacetate

The methods of example 161(c) and (d) were followed here, using [2-(1-pyrrolidinyl)ethyl]amine in place of 2-methoxyethylamine, to give the title compound (20%). M/S M+1:507.0.

### Example 176

### 4-Chloro-3-{[(5Z)-4-oxo-5-(6-quinolinylmethylidene)-4,5-dihydro-1,3-thiazol-2-yl]amino}-N-[2-(1-piperidinyl)ethyl]benzamide trifluoroacetate

The methods of example 161(c) and (d) were followed here using [2-(1-piperidinyl)ethyl]amine in place of 2-methoxyethylamine, to give the title compound (18%). M/S M+1:521.0.

### Example 177

### 4-Chloro-N-[3-(4-methyl-1-piperazinyl)propyl]-3-{[(5Z)-4-oxo-5-(6-quinolinylmethylidene)-4,5-dihydro-1,3-thiazol-2-yl]amino}benzamide trifluoroacetate

The methods of example 161(c) and (d) were followed here using [3-(4-methyl-1-piperazinyl)propyl]amine in place of 2-methoxyethylamine, to give the title compound (18%). M/S M+1:550.1.

### Example 178

### (5Z)-2-({2-Chloro-5-[(4-methyl-1-piperazinyl)carbonyl]phenyl}amino)-5-(6-quinolinylmethylidene)-1,3-thiazol-4(5H)-one trifluoroacetate

The methods of example 161(c) and (d) were followed here using 1-methylpiperazine in place of 2-methoxyethylamine, to give the title compound (18%). M/S M+1:493.0.

### Example 179

### 4-Chloro-N-ethyl-3-{[(5Z)-4-oxo-5-(6-quinolinylmethylidene)-4,5-dihydro-1,3-thiazol-2-yl]amino}benzamide trifluoroacetate

The methods of example 161(c) and (d) were followed here using ethylamine in place of 2-methoxyethylamine, to give the title compound (21%). M/S M+1:437.9.

### Example 180

### 4-Chloro-N-[2-(dimethylamino)ethyl]-N-methyl-3-{[(5Z)-4-oxo-5-(6-quinolinylmethylidene)-4,5-dihydro-1,3-thiazol-2-yl]amino}benzamide trifluoroacetate

The methods of example 161(c) and (d) were followed here using N,N,N′-trimethyl-1,2-ethanediamine in place of 2-methoxyethylamine, to give the title compound (18%). M/S M+1:495.0.

### Example 181

### (5Z)-2-{[2-Chloro-5-(4-morpholinylcarbonyl)phenyl]amino}-5-(6-quinolinylmethylidene)-1,3-thiazol-4(5H)-one trifluoroacetate

The methods of example 161(c) and (d) were followed here using morpholine in place of 2-methoxyethylamine, to give the title compound (16%). M/S M+1:480.0.

### Example 182

### Capsule Composition

An oral dosage form for administering the present invention is produced by filing a standard two piece hard gelatin capsule with the ingredients in the proportions shown in Table I, below.

TABLE I

| INGREDIENTS | AMOUNTS |
|---|---|
| (5Z)-2-[(2-Chloro-3-pyridinyl)amino]-5-(6- | 25 mg |
| quinolinylmethylidene)-1,3-thiazol-4(5H)-one | |
| (Compound of Example 1) | |
| Lactose | 55 mg |
| Talc | 16 mg |
| Magnesium Stearate | 4 mg |

### Example 183

### Injectable Parenteral Composition

An injectable form for administering the present invention is produced by stirring 1.5% by weight of (5Z)-2-{[2-Chloro-5-(2-pyridinyl)phenyl]amino}-5-(6-quinolinylmethylidene)-1,3-thiazol-4(5H)-one (Compound of Example 2) in 10% by volume propylene glycol in water.

### Example 184

### Tablet Composition

The sucrose, calcium sulfate dihydrate and an hYAK inhibitor as shown in Table II below, are mixed and granulated in the proportions shown with a 10% gelatin solution. The wet granules are screened, dried, mixed with the starch, talc and stearic acid; screened and compressed into a tablet.

TABLE II

| INGREDIENTS | AMOUNTS | |
|---|---|---|
| N-(4-Chloro-3-{[(5Z)-4-oxo-5-(6- | 20 | mg |
| quinolinylmethylidene)-4,5-dihydro-1,3-thiazol- | | |
| 2-yl]amino}phenyl)cyclobutanecarboxamide | | |
| (Compound of Example 3) | | |
| calcium sulfate dihydrate | 30 | mg |
| sucrose | 4 | mg |
| starch | 2 | mg |
| talc | 1 | mg |
| stearic acid | 0.5 | mg |

### Biological Methods and Data

Because the compounds of the present invention are active as inhibitors of hYAK3 they exhibit therapeutic utility in treating diseases associated with hYAK3 activity, including but not limited to, anemia, anemias due to renal insufficiency or to chronic disease, such as autoimmunity, HIV, or cancer, and drug-induced anemias, myelodysplastic syndrome, aplastic anemia and myelosuppression, and cytopenia.

Substrate phosphorylation assays were carried out as follows:

YAK3 Scintillation Proximity Assays Using Ser164 of Myelin Basic Protein as the Phosphoacceptor

The source of Ser164 substrate peptide The biotinylated Ser164, S164A peptide(LGGRDSRAGS*PMARRKK-ahx-Biotin-Amide), sequence derived from the C-terminus of bovine myelin basic protein (MBP) with Ser 62 substituted as Ala 162, was purchased from California Peptide Research Inc. (Napa, Calif.), and its purity was determined by HPLC. Phosphorylation occurs at position 164 (marked S* above). The calculated molecular mass of the peptide is 2166 dalton. Solid sample was dissolved at 10 mM in DMSO, aliquoted, and stored at −20 C until use.

The source of enzyme:

hYAK3: Glutathione-S-Transferase (GST)-hYak3-His6 containing amino acid residues 142-526 of human YAK3 (aa 142-526 of SEQ ID NO 2. in U.S. Pat. No. 6,323,318) was purified from baculovirus expression system in Sf9 cells using Glutathione Sepharose 4B column chromatography followed by Ni-NTA-Agarose column chromatography. Purity greater than 65% typically is achieved. Samples, in 50 mM Tris, 150 mM NaCl, 10% glycerol, 0.1% Triton, 250 mM imidazole, 10 mM-mercapto ethanol, pH 8.0 were stored at −80 C until use.

Kinase assay of purified hYAK3: Assays were performed in 96 well (Costar, Catalog No. 3789) or 384 well plates (Costar, Catalog No. 3705). Reaction (in 10, 20, 25, or 40 μl volume) mix contained in final concentrations 25 mM Hepes buffer, pH 7.4; 10 mM MgCl₂; 10 mM-mercapto ethanol; 0.0025% Tween-20; 1 μM ATP, 0.1 μCi of [γ-³³P]ATP; purified hYAK3 (3.6-14 ng/assay; 4 nM final); and 4 μM Ser164 peptide. Compounds, titrated in DMSO, were evaluated at concentrations ranging from 50 μM to 0.2 nM. Final assay concentrations of DMSO did not exceed 5%, resulting in less than 15% loss of YAK3 activity relative to controls without DMSO. Reactions were incubated for 2 hours at room temperature and were stopped by addition of Streptavidin Scintillation Proximity beads (Amersham Pharmacia Biotech, Catalog No. RPNQ 0007 or Amersham Biosciences Catalog no. RPQ0626) in PBS, pH 7.4, 150 mM EDTA, and 0.1% Triton X-100. Under the assay conditions defined above, the Km (apparent) for ATP is determined to be 7.2+/−2.4 μM.

The data for compound dose responses were plotted as % Inhibition, calculated with the data reduction formula 100*(1−[(U1−C2)/(C1−C2)]), versus concentration of compound, where U is the unknown value, C1 is the average control value obtained for DMSO, and C2 is the average control value obtained for 0.05M EDTA. Data were fitted to the curve described by: y=((Vmax*x)/(K+x)) where Vmax is the upper asymptote and K is the IC50. The results for each compound were recorded as pIC50 calculated as follows: pIC50=−Log 10(K).

The compound of Example 1 was tested in the above assay and has pIC₅₀=8.8. The compounds of Example 2-5 were tested in the above assays and have pIC₅₀>7.

Utility of the Present Invention

The compounds of Formula I or II are useful for treating or preventing disease states in which hYAK3 proteins are implicated, especially diseases of the erythroid and hematopoietic systems, including but not limited to, anemias due to renal insufficiency or to chronic disease, such as autoimmunity, HIV, or cancer, and drug-induced anemias, myelodysplastic syndrome, aplastic anemia, myelosuppression, and cytopenia.

The compounds of Formula I or II are useful in treating diseases of the hematopoietic system, particularly anemias. Such anemias include an anemia selected from the group comprising: aplastic anemia and myelodysplastic syndrome. Such anemias also include those wherein the anemia is a consequence of a primary disease selected from the group consisting of: cancer, leukemia and lymphoma. Such anemias also include those wherein the anemia is a consequence of a primary disease selected from the group consisting of: renal disease, failure or damage. Such anemias include those wherein the anemia is a consequence of chemotherapy or radiation therapy, in particular wherein the chemotherapy is chemotherapy for cancer or AZT treatment for HIV infection. Such anemias include those wherein the anemia is a consequence of a bone marrow transplant or a stem cell transplant. Such anemias also include anemia of newborn infants. Such anemias also include those which are a consequence of viral, fungal, microbial or parasitic infection. Such anemias also include those which are a consequence of medical treatment such as surgical or pharmaceutical intervention.

The compounds of Formula I or II are also useful for enhancing normal red blood cell numbers. Such enhancement is desirable for a variety of purposes, especially medical purposes such as preparation of a patient for transfusion and preparation of a patient for surgery.

What is claimed is:

1. A compound of Formula (I): 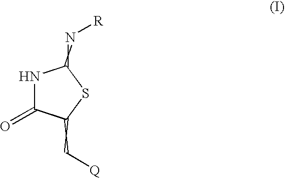
in which
R is selected form: aryl and substituted aryl; and
Q is 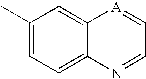
wherein
A is selected from CH and N;
provided that when R is 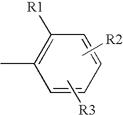
R¹ is not hydrogen, halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂C₁₋₆alkyl,
when R² and R³ are independently selected from: hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —CONH₂, —NH₂, —OCH₂(C═O)OH, —OCH₂CH₂OCH₃, —SO₂NH₂, —CH₂SO₂CH₃, —NH(C═NH)CH₃, 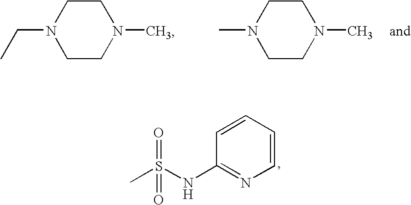
further provided that R is not naphthyl, and
further provided that R is not t-butylthiazol.

2. A compound of Formula (I), as descried in claim 1, in which A is nitrogen.

3. A pharmaceutically acceptable salt, hydrate, solvate or pro-drug of a compound of Formula (I), as described in claim 1.

4. A compound of claim 1 represented by the following Formula (II): 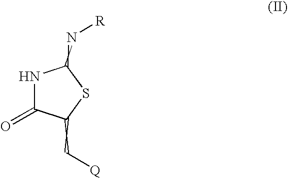
II
in which
R is selected form: C₁-C₁₂aryl and substituted C₁-C₁₂aryl; and
Q is 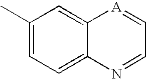
wherein
A is selected from CH and N;
provided that when R is 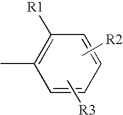
R¹ is not hydrogen, halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂C₁₋₆alkyl,
when R² and R³ are independently selected from: hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —CONH₂, —NH₂, —OCH₂(C═O)OH, —OCH₂CH₂OCH₃, —SO₂NH₂, —CH₂SO₂CH₃, —NH(C═NH)CH₃, 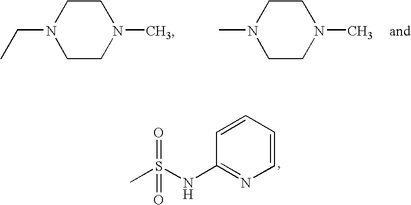
further provided that R is not naphthyl, and
further provided that R is not t-butylthiazol.

5. A compound of Formula (II), as descried in claim 4, in which A is nitrogen.

6. A pharmaceutically acceptable salt, hydrate, solvate or pro-drug of a compound of Formula (II), as described in claim 4.

7. A compound of Formula (II), as described in claim 4,
in which:
R is 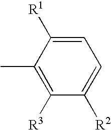
in which R¹ is selected from: halogen, C₁₋₆alkyl, substituted C₁₋₆alkyl, amine, C₁₋₆alkylamine and C₁₋₆dialkylamine;
R³ is selected from: hydrogen, halogen, C₁₋₆alkyl, substituted C₁₋₆alkyl, amine, C₁₋₆alkylamine and C₁₋₆dialkylamine; and
R² is selected from: —NR⁶⁰S(O)₂R⁷⁰ and —N(R⁷⁰)C(O)R⁷⁰,
where,
R⁶⁰ is selected from: hydrogen, C₁₋₆alkyl, C₁-C₁₂aryl and
C₁-C₁₂arylC₁-C₆alkyl, and
each R⁷⁰ is independently selected from: hydrogen, C₁₋₆alkyl, C₁-C₆alkyloxyC₁-C₆alkyl, C₁-C₆alkyloxyC₁-C₆alkylamine, C₁-C₄alkylC(O)OC₁-C₄alkyl, —C₁-C₆alkylC(O)OH, amino, alkylamino where the alkyl is optionally substituted with one or more substituents selected from hydroxy, oxo, cycloalkyl containing from 1 to 4 heteroatoms where cycloalkyl containing from 1 to 4 heteroatoms is optionally substituted by one or more substituents selected form C₁-C₆alkyl, halogen and oxo, and C₁-C₆alkyl, dialkylamino where each alkyl is independently and optionally substituted with one or more substituents selected from hydroxyl, oxo, C₁-C₆alkyl, amino, alkylamino and dialkylamino, aminoC₁-C₆alkyl, alkylaminoC₁-C₆alkyl where the alkyl is optionally substituted by one or more substituents selected from oxo, alkoxy and halogen, dialkylaminoC₁-C₆alkyl where each alkyl is independently and optionally substituted with one or more substituents selected from hydroxy, oxo, C₁-C₆alkyl, amino, alkylamino and dialkylamino, —C(O)OH, alkoxy, aryloxy, arylamino, diarylamino, arylalkylamino where the aryl is optionally substituted with one or more substituents selected from hydroxyl, alkoxy, hydroxyalkyl, oxo, C₁-C₆alkyl, amino, alkylamino and dialkylamino, cycloalkylalkylamino, aryl, aryl substituted with one or more substituents selected from oxo, hydroxyl, —N(H)C(O)C₁-C₆alkyl, alkoxy, nitro, amine and alkyl, arylC₁-C₄alkyl optionally substituted with one or more substituents selected from oxo, amino, alkylamino, alkylamino substituted by oxo, dialkylamino, dialkylamino substituted by one or more oxo groups, hydroxy, halogen, alkoxy and alkyl, —CH₂C(O)cycloalkyl containing from 1 to 4 heteroatoms, cycloalkylC₁-C₆alkyl where the cycloalkyl is optionally substituted with one or more substituents selected from C₁-C₆alkyl, halogen, —C(O)OC₁-C₆alkyl and —C₁-C₆alkylC(O)OH, C₁-C₄alkyl substituted with cycloalkyl containing from 1 to 4 heteroatoms where the cycloalkyl containing from 1 to 4 heteroatoms is optionally substituted with one or more substituents selected from C₁-C₆alkyl, oxo, halogen, —C(O)OC₁-C₆alkyl and —C₁-C₆alkylC(O)OH, cycloalkyl, —N(H)cycloalkyl, cycloalkyl substituted with one or more substituents selected from oxo, hydroxy, halogen, amino, alkylamino, dialkylamino, —C(O)OH, —C(O)NR⁸⁰R⁹⁰ where R⁸⁰ and R⁹⁰ are each independently selected form hydrogen and C₁-C₈alkyl, and alkyl, —N(H)cycloalkyl substituted with one or more substituents selected from oxo, hydroxy and alkyl, cycloalkyl containing from 1 to 4 heteroatoms, cycloalkyl containing from 1 to 4 heteroatoms substituted with one or more substituents selected from oxo, alkoxy, hydroxyl and alkyl where alkyl is optionally substituted with one or more substituents selected from halogen, hydroxy, alkoxy, oxo and cycloalkyl containing from 1 to 4 heteroatoms, —N(H)cycloalkyl containing from 1 to 4 heteroatoms substituted with one or more substituents selected from oxo, hydroxy and alkyl, and trifluoromethyl;
and
Q is 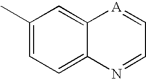
wherein
A is N.

8. A pharmaceutically acceptable salt, hydrate, solvate or pro-drug of a compound as described in claim 7.

9. A compound as described in claim 1,
in which:
R is 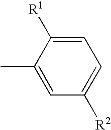
in which R¹ is halogen; and
R² is selected from: —NR⁶²S(O)₂R⁷² and —N(R⁷²)C(O)R⁷²,
where,
R⁶² is hydrogen, and
each R⁷² is independently selected from: hydrogen, C₁₋₆alkyl, C₁-C₆alkyloxyC₁-C₆alkyl, C₁-C₆alkyloxyC₁-C₆alkylamine, C₁-C₄alkylC(O)OC₁-C₄alkyl, —C₁-C₆alkylC(O)OH, amino, alkylamino where the alkyl is optionally substituted with one or more substituents selected from hydroxy, cycloalkyl containing from 1 to 4 heteroatoms where cycloalkyl containing from 1 to 4 heteroatoms is optionally substituted by one or more substituents selected form C₁-C₆alkyl, halogen and oxo, and C₁-C₆alkyl, dialkylamino where each alkyl is independently and optionally substituted with one or more substituents selected from hydroxy, oxo, C₁-C₆alkyl, amino, alkylamino and dialkylamino, aminoC₁-C₆alkyl, alkylaminoC₁-C₆alkyl where the alkyl is optionally substituted by one or more substituents selected from oxo, alkoxy and halogen, dialkylaminoC₁-C₆alkyl where each alkyl is independently and optionally substituted with one or more substituents selected from hydroxy, oxo, C₁-C₆alkyl, amino, alkylamino and dialkylamino, alkoxy, aryloxy, arylamino, diarylamino, arylalkylamino where the aryl is optionally substituted with one or more substituents selected from hydroxyl, alkoxy, hydroxyalkyl, oxo, C₁-C₆alkyl, amino, alkylamino and dialkylamino, cycloalkylalkylamino, aryl, aryl substituted with one or more substituents selected from oxo, hydroxyl, —N(H)C(O)C₁-C₆alkyl, alkoxy, nitro, amine and alkyl, arylC₁-C₄alkyl optionally substituted with one or more substituents selected from oxo, amino, alkylamino, alkylamino substituted by oxo, dialkylamino, dialkylamino substituted by one or more oxo groups, hydroxy, halogen, alkoxy and alkyl, —CH₂C(O)cycloalkyl containing from 1 to 4 heteroatoms, cycloalkylC₁-C₆alkyl where the cycloalkyl is optionally substituted with one or more substituents selected from C₁-C₆alkyl, halogen, —C(O)OC₁-C₆alkyl and —C₁-C₆alkylC(O)OH, C₁-C₄alkyl substituted with cycloalkyl containing from 1 to 4 heteroatoms where the cycloalkyl containing from 1 to 4 heteroatoms is optionally substituted with one or more substituents selected from C₁-C₆alkyl, oxo, halogen, —C(O)OC₁-C₆alkyl and —C₁-C₆alkylC(O)OH, cycloalkyl, —N(H)cycloalkyl, cycloalkyl substituted with one or more substituents selected from oxo, hydroxyl and alkyl, —N(H)cycloalkyl substituted with one or more substituents selected from oxo, hydroxy and alkyl, cycloalkyl containing from 1 to 4 heteroatoms, cycloalkyl containing from 1 to 4 heteroatoms substituted with one or more substituents selected from oxo, alkoxy, hydroxyl and alkyl where alkyl is optionally substituted with one or more substituents selected from halogen, hydroxy, alkoxy, oxo and cycloalkyl containing from 1 to 4 heteroatoms, —N(H)cycloalkyl containing from 1 to 4 heteroatoms substituted with one or more substituents selected from oxo, hydroxy and alkyl, and trifluoromethyl;
and
Q is 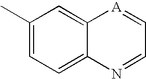
wherein
A is N.

10. A pharmaceutically acceptable salt, hydrate, solvate or pro-drug of a compound as described in claim 9.

11. A compound as described in claim 1,
in which:
R is 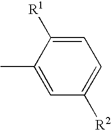
in which R¹ is chlorine; and
R² is —N(R⁷⁴)C(O)R⁷⁴,
where,
each R⁷⁴ is independently selected from: hydrogen, C₁₋₆alkyl, C₁-C₆alkyloxyC₁-C₆alkyl, C₁-C₆alkyloxyC₁-C₆alkylamine, C₁-C₄alkylC(O)OC₁-C₄alkyl, —C₁-C₆alkylC(O)OH, amino, alkylamino dialkylamino, aminoC₁-C₆alkyl, alkylaminoC₁-C₆alkyl, dialkylaminoC₁-C₆alkyl, alkoxy, C₁-C₁₂aryl, C₁-C₁₂arylC₁-C₄alkyl, cycloalkylC₁-C₆alkyl, C₁-C₄alkyl substituted with cycloalkyl containing from 1 to 4 heteroatoms, cycloalkyl, —N(H)cycloalkyl, cycloalkyl containing from 1 to 4 heteroatoms, —N(H)cycloalkyl containing from 1 to 4 heteroatoms, and trifluoromethyl;
and
Q is 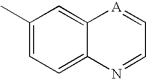
wherein
A is N.

12. A pharmaceutically acceptable salt, hydrate, solvate or pro-drug of a compound as described in claim 11.

13. A compound of claim 1 selected from:
(5Z)-2-[(2-Chloro-3-pyridinyl)amino]-5-(6-quinolinylmethylidene)-1,3-thiazol-4(5H)-one;
(5Z)-2-{[2-Chloro-5-(2-pyridinyl)phenyl]amino}-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)cyclobutanecarboxamide;
N-(4-chloro-3-{[(5Z)-4-oxo-5-(6-quinoxalinylmethylidene)-4,5-dihydro-1,3-thiazol-2-yl]amino}phenyl)-2-methylpropanamide;
N-(4-chloro-3-{[(5Z)-4-oxo-5-(6-quinolinylmethylidene)-4,5-dihydro-1,3-thiazol-2-yl]amino}phenyl)methanesulfonamide;
N-[(4-Chloro-3-{[(5Z)-4-oxo-5-(6-quinolinylmethylidene)-4,5-dihydro-1,3-thiazol-2-yl]amino}phenyl)methyl]-2-(methyloxy)acetamide;
(5Z)-2-[(2-Chloro-5-{[(1-methylethyl)amino]methyl}phenyl)amino]-5-(6-quinolinylmethylidene)-1,3-thiazol-4(5H)-one;
(2Z,5Z)-2-[(5-Chloro-1H-benzimidazol-6-yl)imino]-5-(quinolin-6-ylmethylene)-1,3-thiazolidin-4-one;
4-Chloro-N-cyclobutyl-3-{[(5Z)-4-oxo-5-(6-quinolinylmethylidene)-4,5-dihydro-1,3-thiazol-2-yl]amino}benzenesulfonamide;
(5Z)-2-(4-Pyrimidinylamino)-5-(6-quinolinylmethylidene)-1,3-thiazol-4(5H)-one;
(5Z)-2-(1H-Pyrazol-3-ylamino)-5-(6-quinolinylmethylidene)-1,3-thiazol-4(5H)-one;
(5Z)-2-(4-Pyridinylamino)-5-(6-quinolinylmethylidene)-1,3-thiazol-4(5H)-one;
(5Z)-2-(2-Quinolinylamino)-5-(6-quinolinylmethylidene)-1,3-thiazol-4(5H)-one;
(5Z)-2-[(4-Methyl-2-pyridinyl)amino]-5-(6-quinolinylmethylidene)-1,3-thiazol-4(5H)-one;
(5Z)-2-[(3-Methyl-2-pyridinyl)amino]-5-(6-quinolinylmethylidene)-1,3-thiazol-4(5H)-one;
(5Z)-2-[(6-Methyl-2-pyridinyl)amino]-5-(6-quinolinylmethylidene)-1,3-thiazol-4(5H)-one;
(5Z)-2-[(5-Iodo-2-pyridinyl)amino]-5-(6-quinolinylmethylidene)-1,3-thiazol-4(5H)-one;
(5Z)-2-(1H-Benzimidazol-2-ylamino)-5-(6-quinolinylmethylidene)-1,3-thiazol-4(5H)-one;
N-(3-{[(5Z)-4-Oxo-5-(6-quinolinylmethylidene)-4,5-dihydro-1,3-thiazol-2-yl]amino}phenyl)acetamide;
1,1-Dimethylethyl (2-{[(5Z)-4-oxo-5-(6-quinolinylmethylidene)-4,5-dihydro-1,3-thiazol-2-yl]amino}phenyl)carbamate;
N-(2-{[(5Z)-4-Oxo-5-(6-quinolinylmethylidene)-4,5-dihydro-1,3-thiazol-2-yl]amino}phenyl)acetamide;
(5Z)-2-(2-Pyrimidinylamino)-5-(6-quinolinylmethylidene)-1,3-thiazol-4(5H)-one;
(5Z)-2-(3-Quinolinylamino)-5-(6-quinolinylmethylidene)-1,3-thiazol-4(5H)-one;
(5Z)-2-[(1-Methyl-1H-indol-2-yl)amino]-5-(6-quinolinylmethylidene)-1,3-thiazol-4(5H)-one;
(5Z)-2-[(5-Chloro-2-pyridinyl)amino]-5-(6-quinolinylmethylidene)-1,3-thiazol-4(5H)-one;
1,1-Dimethylethyl [(2-{[(5Z)-4-oxo-5-(6-quinolinylmethylidene)-4,5-dihydro-1,3-thiazol-2-yl]amino}phenyl)methyl]carbamate;
(5Z)-2-{[2-Chloro-6-(trifluoromethyl)-3-pyridinyl]amino}-5-(6-quinolinylmethylidene)-1,3-thiazol-4(5H)-one;
N-(4-{[(5Z)-4-Oxo-5-(6-quinolinylmethylidene)-4,5-dihydro-1,3-thiazol-2-yl]amino}phenyl)acetamide;
(5Z)-2-{[6-(Methyloxy)-4-pyrimidinyl]amino}-5-(6-quinolinylmethylidene)-1,3-thiazol-4(5H)-one;
3-Methyl-N-(2-{[(5Z)-4-oxo-5-(6-quinolinylmethylidene)-4,5-dihydro-1,3-thiazol-2-yl]amino}phenyl)butanamide;
N-(4-Chloro-3-{[(5Z)-4-oxo-5-(6-quinolinylmethylidene)-4,5-dihydro-1,3-thiazol-2-yl]amino}phenyl)-3-methylbutanamide;
(5Z)-2-[(4-Methyl-1,3-thiazol-2-yl)amino]-5-(6-quinolinylmethylidene)-1,3-thiazol-4(5H)-one;
Ethyl 2-{[(5Z)-4-oxo-5-(6-quinolinylmethylidene)-4,5-dihydro-1,3-thiazol-2-yl]amino}-4,5,6,7-tetrahydro-1-benzothiophene-3-carboxylate;
(5Z)-2-(2-Biphenylylamino)-5-(6-quinolinylmethylidene)-1,3-thiazol-4(5H)-one;
(5Z)-2-{[4′-(Methyloxy)-2-biphenylyl]amino}-5-(6-quinolinylmethylidene)-1,3-thiazol-4(5H)-one;
(5Z)-2-{[4′-(Dimethylamino)-2-biphenylyl]amino}-5-(6-quinolinylmethylidene)-1,3-thiazol-4(5H)-one;
2′-{[(5Z)-4-Oxo-5-(6-quinolinylmethylidene)-4,5-dihydro-1,3-thiazol-2-yl]amino}-3-biphenylcarbonitrile;
(5Z)-2-{[2-(1,3-Benzodioxol-5-yl)phenyl]amino}-5-(6-quinolinylmethylidene)-1,3-thiazol-4(5H)-one;
Ethyl 1-methyl-5-{[(5Z)-4-oxo-5-(6-quinolinylmethylidene)-4,5-dihydro-1,3-thiazol-2-yl]amino}-1H-pyrazole-4-carboxylate;
(5Z)-2-{[2-(4-Pyridinyl)phenyl]amino}-5-(6-quinolinylmethylidene)-1,3-thiazol-4(5H)-one;
(5Z)-2-{[2-Chloro-5-(hydroxymethyl)phenyl]amino}-5-(6-quinolinylmethylidene)-1,3-thiazol-4(5H)-one;
1,1-Dimethylethyl [(4-chloro-3-{[(5Z)-4-oxo-5-(6-quinolinylmethylidene)-4,5-dihydro-1,3-thiazol-2-yl]amino}phenyl)methyl]carbamate;
(5Z)-2-{[5-(Aminomethyl)-2-chlorophenyl]amino}-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)propanamide;
N-(4-Chloro-3-{[(5Z)-4-oxo-5-(6-quinolinylmethylidene)-4,5-dihydro-1,3-thiazol-2-yl]amino}phenyl)-2-(methyloxy)acetamide;
N-(4-Chloro-3-{[(5Z)-4-oxo-5-(6-quinolinylmethylidene)-4,5-dihydro-1,3-thiazol-2-yl]amino}phenyl)-2-(2-thienyl)acetamide;
N-[(4-Chloro-3-{[(5Z)-4-oxo-5-(6-quinolinylmethylidene)-4,5-dihydro-1,3-thiazol-2-yl]amino}phenyl)methyl]acetamide;
N-[(4-Chloro-3-{[(5Z)-4-oxo-5-(6-quinolinylmethylidene)-4,5-dihydro-1,3-thiazol-2-yl]amino}phenyl)methyl]-2-methylpropanamide;
N˜1˜-(4-Chloro-3-{[(5Z)-4-oxo-5-(6-quinolinylmethylidene)-4,5-dihydro-1,3-thiazol-2-yl]amino}phenyl)-N˜2˜,N˜2˜-dimethylglycinamide;
N-[(4-Chloro-3-{[(5Z)-4-oxo-5-(6-quinolinylmethylidene)-4,5-dihydro-1,3-thiazol-2-yl]amino}phenyl)methyl]urea;
N-[(4-Chloro-3-{[(5Z)-4-oxo-5-(6-quinolinylmethylidene)-4,5-dihydro-1,3-thiazol-2-yl]amino}phenyl)methyl]-2-hydroxyacetamide;
N˜1˜-[(4-Chloro-3-{[(5Z)-4-oxo-5-(6-quinolinylmethylidene)-4,5-dihydro-1,3-thiazol-2-yl]amino}phenyl)methyl]-N˜2˜,N˜2˜-dimethylglycinamide;
1,1-Dimethylethyl (2-{[(4-chloro-3-{[(5Z)-4-oxo-5-(6-quinolinylmethylidene)-4,5-dihydro-1,3-thiazol-2-yl]amino}phenyl)methyl]amino}-2-oxoethyl)carbamate;
4-{[(4-Chloro-3-{[(5Z)-4-oxo-5-(6-quinolinylmethylidene)-4,5-dihydro-1,3-thiazol-2-yl]amino}phenyl)methyl]amino}-4-oxobutanoic acid;
(5Z)-2-{[3-(1,3-Oxazol-4-yl)phenyl]amino}-5-(6-quinolinylmethylidene)-1,3-thiazol-4(5H)-one;
(5Z)-2-[(1-Ethyl-1H-pyrazol-5-yl)amino]-5-(6-quinolinylmethylidene)-1,3-thiazol-4(5H)-one;
N˜1˜-[(4-Chloro-3-{[(5Z)-4-oxo-5-(6-quinolinylmethylidene)-4,5-dihydro-1,3-thiazol-2-yl]amino}phenyl)methyl]glycinamide;
Ethyl (4-chloro-3-{[(5Z)-4-oxo-5-(6-quinolinylmethylidene)-4,5-dihydro-1,3-thiazol-2-yl]amino}phenyl)carbamate;
N-(4-Chloro-3-{[(5Z)-4-oxo-5-(6-quinolinylmethylidene)-4,5-dihydro-1,3-thiazol-2-yl]amino}phenyl)-2-cyclopropylacetamide;
N-(4-Chloro-3-{[(5Z)-4-oxo-5-(6-quinolinylmethylidene)-4,5-dihydro-1,3-thiazol-2-yl]amino}phenyl)-N′-(phenylmethyl)urea;
Ethyl 4-[(4-chloro-3-{[(5Z)-4-oxo-5-(6-quinolinylmethylidene)-4,5-dihydro-1,3-thiazol-2-yl]amino}phenyl)amino]-4-oxobutanoate;
N-(4-Chloro-3-{[(5Z)-4-oxo-5-(6-quinolinylmethylidene)-4,5-dihydro-1,3-thiazol-2-yl]amino}phenyl)-N′-(1-methylethyl)urea;
N-(4-Chloro-3-{[(5Z)-4-oxo-5-(6-quinolinylmethylidene)-4,5-dihydro-1,3-thiazol-2-yl]amino}phenyl)-1-piperidinecarboxamide;
(5Z)-2-{[2-Chloro-5-(1H-imidazol-4-yl)phenyl]amino}-5-(6-quinolinylmethylidene)-1,3-thiazol-4(5H)-one;
(5Z)-2-{[2-Chloro-5-(2-methyl-1,3-thiazol-4-yl)phenyl]amino}-5-(6-quinolinylmethylidene)-1,3-thiazol-4(5H)-one;
5-Chloro-6-{[(5Z)-4-oxo-5-(6-quinolinylmethylidene)-4,5-dihydro-1,3-thiazol-2-yl]amino}-1,3-dihydro-2H-benzimidazol-2-one;
Methyl [(4-chloro-3-{[(5Z)-4-oxo-5-(6-quinolinylmethylidene)-4,5-dihydro-1,3-thiazol-2-yl]amino}phenyl)methyl]carbamate;
N-[(4-Chloro-3-{[(5Z)-4-oxo-5-(6-quinolinylmethylidene)-4,5-dihydro-1,3-thiazol-2-yl]amino}phenyl)methyl]-N′-(1-methylethyl)urea;
2-[3,4-Bis(methyloxy)phenyl]-N-(4-chloro-3-{[(5Z)-4-oxo-5-(6-quinolinylmethylidene)-4,5-dihydro-1,3-thiazol-2-yl]amino}phenyl)acetamide;
(5Z)-2-{[2-Chloro-5-(1,3-oxazol-4-yl)phenyl]amino}-5-(6-quinolinylmethylidene)-1,3-thiazol-4(5H)-one;
(5Z)-2-{[5-(1,3-Benzothiazol-2-yl)-2-chlorophenyl]amino}-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)-3-phenylpropanamide;
N-(4-Chloro-3-{[(5Z)-4-oxo-5-(6-quinolinylmethylidene)-4,5-dihydro-1,3-thiazol-2-yl]amino}phenyl)-2-(4-fluorophenyl)acetamide;
(5Z)-2-[(2-Amino-5-chloro-1H-benzimidazol-6-yl)amino]-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)-1-phenylmethanesulfonamide;
N′-(4-Chloro-3-{[(5Z)-4-oxo-5-(6-quinolinylmethylidene)-4,5-dihydro-1,3-thiazol-2-yl]amino}phenyl)-N,N-dimethylsulfamide;
(5Z)-2-[(7-Chloro-6-quinoxalinyl)amino]-5-(6-quinolinylmethylidene)-1,3-thiazol-4(5H)-one;
N-(4-{[(4-Chloro-3-{[(5Z)-4-oxo-5-(6-quinolinylmethylidene)-4,5-dihydro-1,3-thiazol-2-yl]amino}phenyl)amino]sulfonyl}-5-methyl-1,3-thiazol-2-yl)acetamide;
N-(4-Chloro-3-{[(5Z)-4-oxo-5-(6-quinolinylmethylidene)-4,5-dihydro-1,3-thiazol-2-yl]amino}phenyl)-2-thiophenesulfonamide;
(5Z)-2-{[2-Chloro-5-(1H-tetrazol-5-yl)phenyl]amino}-5-(6-quinolinylmethylidene)-1,3-thiazol-4(5H)-one;
N˜1˜-(4-Chloro-3-{[(5Z)-4-oxo-5-(6-quinolinylmethylidene)-4,5-dihydro-1,3-thiazol-2-yl]amino}phenyl)glycinamide;
N-(4-Chloro-3-{[(5Z)-4-oxo-5-(6-quinolinylmethylidene)-4,5-dihydro-1,3-thiazol-2-yl]amino}phenyl)-2-(1-piperidinyl)acetamide;
((5Z)-2-{[2-Chloro-5-(2-pyrimidinyl)phenyl]amino}-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)-2-cyclopentylacetamide;
N-(4-Chloro-3-{[(5Z)-4-oxo-5-(6-quinolinylmethylidene)-4,5-dihydro-1,3-thiazol-2-yl]amino}phenyl)-2-(1-pyrrolidinyl)acetamide;
N˜1˜-(4-Chloro-3-{[(5Z)-4-oxo-5-(6-quinolinylmethylidene)-4,5-dihydro-1,3-thiazol-2-yl]amino}phenyl)-N˜2˜-ethyl-N˜2˜-methylglycinamide;
N-(4-Chloro-3-{[(5Z)-4-oxo-5-(6-quinolinylmethylidene)-4,5-dihydro-1,3-thiazol-2-yl]amino}phenyl)-2-(4-morpholinyl)acetamide;
1,1-Dimethylethyl 4-{2-[(4-chloro-3-{[(5Z)-4-oxo-5-(6-quinolinylmethylidene)-4,5-dihydro-1,3-thiazol-2-yl]amino}phenyl)amino]-2-oxoethyl}-1-piperazinecarboxylate;
N′-[(4-Chloro-3-{[(5Z)-4-oxo-5-(6-quinolinylmethylidene)-4,5-dihydro-1,3-thiazol-2-yl]amino}phenyl)methyl]-N,N-dimethylimidoformamide;
N-(4-Chloro-3-{[(5Z)-4-oxo-5-(6-quinolinylmethylidene)-4,5-dihydro-1,3-thiazol-2-yl]amino}phenyl)-2-(1-piperazinyl)acetamide;
N-(4-Chloro-3-{[(5Z)-4-oxo-5-(6-quinolinylmethylidene)-4,5-dihydro-1,3-thiazol-2-yl]amino}phenyl)-2-pyridinecarboxamide;
N-(4-Chloro-3-{[(5Z)-4-oxo-5-(6-quinolinylmethylidene)-4,5-dihydro-1,3-thiazol-2-yl]amino}phenyl)cyclohexanecarboxamide;
N-(4-Chloro-3-{[(5Z)-4-oxo-5-(6-quinolinylmethylidene)-4,5-dihydro-1,3-thiazol-2-yl]amino}phenyl)benzamide;
N-(4-Chloro-3-{[(5Z)-4-oxo-5-(6-quinolinylmethylidene)-4,5-dihydro-1,3-thiazol-2-yl]amino}phenyl)cyclopentanecarboxamide;
(5Z)-2-{[2-Chloro-5-(3-thienyl)phenyl]amino}-5-(6-quinolinylmethylidene)-1,3-thiazol-4(5H)-one;
(5Z)-2-({2-Chloro-5-[6-(methyloxy)-2-pyridinyl]phenyl}amino)-5-(6-quinolinylmethylidene)-1,3-thiazol-4(5H)-one;
(5Z)-2-[(3,5-Dichloro-2,6-dimethyl-4-pyridinyl)amino]-5-(6-quinoxalinylmethylidene)-1,3-thiazol-4(5H)-one;
(5Z)-2-{[2-Chloro-5-(4-morpholinyl)phenyl]amino}-5-(6-quinolinylmethylidene)-1,3-thiazol-4(5H)-one;
N˜1˜-(4-Chloro-3-{[(5Z)-4-oxo-5-(6-quinolinylmethylidene)-4,5-dihydro-1,3-thiazol-2-yl]amino}phenyl)leucinamide;
N-(4-Chloro-3-{[(5Z)-4-oxo-5-(6-quinolinylmethylidene)-4,5-dihydro-1,3-thiazol-2-yl]amino}phenyl)-2-methylpropanamide;
(5Z)-2-{[5-(2-Amino-5-pyrimidinyl)-2-chlorophenyl]amino}-5-(6-quinolinylmethylidene)-1,3-thiazol-4(5H)-one;
(5Z)-2-({2-Chloro-5-[(dimethylamino)methyl]phenyl}amino)-5-(6-quinolinylmethylidene)-1,3-thiazol-4(5H)-one;
N-[(4-Chloro-3-{[(5Z)-4-oxo-5-(6-quinolinylmethylidene)-4,5-dihydro-1,3-thiazol-2-yl]amino}phenyl)methyl]methanesulfonamide;
4-Chloro-N,N-dimethyl-3-{[(5Z)-4-oxo-5-(6-quinolinylmethylidene)-4,5-dihydro-1,3-thiazol-2-yl]amino}benzenesulfonamide;
N-(2,4-Dichloro-5-{[(5Z)-4-oxo-5-(6-quinolinylmethylidene)-4,5-dihydro-1,3-thiazol-2-yl]amino}phenyl)cyclobutanecarboxamide;
(5Z)-2-{[4-Chloro-3′-(methyloxy)-3-biphenylyl]amino}-5-(6-quinolinylmethylidene)-1,3-thiazol-4(5H)-one;
(5Z)-2-({2-Chloro-5-[(cyclopentylamino)methyl]phenyl}amino)-5-(6-quinolinylmethylidene)-1,3-thiazol-4(5H)-one;
N-(4-Chloro-3-{[(5Z)-4-oxo-5-(6-quinolinylmethylidene)-4,5-dihydro-1,3-thiazol-2-yl]amino}phenyl)-N-methylcyclobutanecarboxamide;
N-(4-Chloro-3-{[(5Z)-4-oxo-5-(6-quinolinylmethylidene)-4,5-dihydro-1,3-thiazol-2-yl]amino}phenyl)-N-(phenylsulfonyl)benzenesulfonamide;
(5Z)-2-({2-Chloro-5-[(phenylmethyl)amino]phenyl}amino)-5-(6-quinolinylmethylidene)-1,3-thiazol-4(5H)-one;
(5Z)-2-({2-Chloro-5-[(1-methylethyl)amino]phenyl}amino)-5-(6-quinolinylmethylidene)-1,3-thiazol-4(5H)-one;
1,1-Dimethylethyl (4-chloro-3-{[(5Z)-4-oxo-5-(6-quinolinylmethylidene)-4,5-dihydro-1,3-thiazol-2-yl]amino}phenyl)carbamate;
N-(4-Chloro-3-{[(5Z)-4-oxo-5-(6-quinolinylmethylidene)-4,5-dihydro-1,3-thiazol-2-yl]amino}phenyl)-L-prolinamide;
N˜1˜-(4-Chloro-3-{[(5Z)-4-oxo-5-(6-quinolinylmethylidene)-4,5-dihydro-1,3-thiazol-2-yl]amino}phenyl)-L-alaninamide;
1,1-Dimethylethyl 4-{[(4-chloro-3-{[(5Z)-4-oxo-5-(6-quinolinylmethylidene)-4,5-dihydro-1,3-thiazol-2-yl]amino}phenyl)amino]carbonyl}-1-piperidinecarboxylate;
(5Z)-2-({2-Chloro-5-[6-(methylamino)-2-pyridinyl]phenyl}amino)-5-(6-quinolinylmethylidene)-1,3-thiazol-4(5H)-one;
(5Z)-2-[(3,5-Dichloro-4-pyridinyl)amino]-5-(6-quinolinylmethylidene)-1,3-thiazol-4(5H)-one;
1,1-Dimethylethyl (3-chloro-4-{[(5Z)-4-oxo-5-(6-quinolinylmethylidene)-4,5-dihydro-1,3-thiazol-2-yl]amino}phenyl)carbamate;
N-(4-Chloro-3-{[(5Z)-4-oxo-5-(6-quinolinylmethylidene)-4,5-dihydro-1,3-thiazol-2-yl]amino}phenyl)-4-piperidinecarboxamide;
N-(4-Chloro-3-{[(5Z)-4-oxo-5-(6-quinolinylmethylidene)-4,5-dihydro-1,3-thiazol-2-yl]amino}phenyl)ethanesulfonamide;
N-(4-Chloro-3-{[(5Z)-4-oxo-5-(6-quinolinylmethylidene)-4,5-dihydro-1,3-thiazol-2-yl]amino}phenyl)benzenesulfonamide;
N-(4-Chloro-3-{[(5Z)-4-oxo-5-(6-quinolinylmethylidene)-4,5-dihydro-1,3-thiazol-2-yl]amino}phenyl)-1-propanesulfonamide;
N-(3-Chloro-4-{[(5Z)-4-oxo-5-(6-quinolinylmethylidene)-4,5-dihydro-1,3-thiazol-2-yl]amino}phenyl)acetamide;
N-(3-Chloro-4-{[(5Z)-4-oxo-5-(6-quinolinylmethylidene)-4,5-dihydro-1,3-thiazol-2-yl]amino}phenyl)methanesulfonamide;
N-(3-Chloro-4-{[(5Z)-4-oxo-5-(6-quinolinylmethylidene)-4,5-dihydro-1,3-thiazol-2-yl]amino}phenyl)benzamide;
N-(3-Chloro-4-{[(5Z)-4-oxo-5-(6-quinolinylmethylidene)-4,5-dihydro-1,3-thiazol-2-yl]amino}phenyl)-2-methylpropanamide;
N-(4-Chloro-3-{[(5Z)-4-oxo-5-(6-quinolinylmethylidene)-4,5-dihydro-1,3-thiazol-2-yl]amino}phenyl)-4-methylbenzenesulfonamide;
N-(3-Chloro-4-{[(5Z)-4-oxo-5-(6-quinolinylmethylidene)-4,5-dihydro-1,3-thiazol-2-yl]amino}phenyl)-2,2,2-trifluoroacetamide;
(3S)-3-Amino-N-(4-chloro-3-{[(5Z)-4-oxo-5-(6-quinolinylmethylidene)-4,5-dihydro-1,3-thiazol-2-yl]amino}phenyl)butanamide;
N-(4-Chloro-3-{[(5Z)-4-oxo-5-(6-quinolinylmethylidene)-4,5-dihydro-1,3-thiazol-2-yl]amino}phenyl)-L-phenylalaninamide;
N-(4-Chloro-3-{[(5Z)-4-oxo-5-(6-quinolinylmethylidene)-4,5-dihydro-1,3-thiazol-2-yl]amino}phenyl)-4-nitrobenzenesulfonamide;
N-(4-Chloro-3-{[(5Z)-4-oxo-5-(6-quinolinylmethylidene)-4,5-dihydro-1,3-thiazol-2-yl]amino}phenyl)-2,2-dimethylpropanamide;
4-Chloro-N-(2-methylpropyl)-3-{[(5Z)-4-oxo-5-(6-quinoxalinylmethylidene)-4,5-dihydro-1,3-thiazol-2-yl]amino}benzamide;
N-(4-Chloro-3-{[(5Z)-4-oxo-5-(6-quinoxalinylmethylidene)-4,5-dihydro-1,3-thiazol-2-yl]amino}phenyl)propanamide;
N-(4-Chloro-3-{[(5Z)-4-oxo-5-(6-quinoxalinylmethylidene)-4,5-dihydro-1,3-thiazol-2-yl]amino}phenyl)-2-(methyloxy)acetamide;
N-(4-Chloro-3-{[(5Z)-4-oxo-5-(6-quinoxalinylmethylidene)-4,5-dihydro-1,3-thiazol-2-yl]amino}phenyl)-2-(2-thienyl)acetamide;
N-(4-Chloro-3-{[(5Z)-4-oxo-5-(6-quinoxalinylmethylidene)-4,5-dihydro-1,3-thiazol-2-yl]amino}phenyl)-2-methylpropanamide;
N-(4-Chloro-3-{[(5Z)-4-oxo-5-(6-quinoxalinylmethylidene)-4,5-dihydro-1,3-thiazol-2-yl]amino}phenyl)cyclobutanecarboxamide;
N-(4-Chloro-3-{[(5Z)-4-oxo-5-(6-quinoxalinylmethylidene)-4,5-dihydro-1,3-thiazol-2-yl]amino}phenyl)cyclopentanecarboxamide;
(5Z)-2-[(3,5-Dichloro-4-pyridinyl)amino]-5-(6-quinoxalinylmethylidene)-1,3-thiazol-4(5H)-one;
N-(4-Chloro-3-{[(5Z)-4-oxo-5-(6-quinoxalinylmethylidene)-4,5-dihydro-1,3-thiazol-2-yl]amino}phenyl)-4-piperidinecarboxamide;
N-(2-Chloro-3-{[(5Z)-4-oxo-5-(6-quinoxalinylmethylidene)-4,5-dihydro-1,3-thiazol-2-yl]amino}phenyl)-2-methylpropanamide;
N-(4-Chloro-3-{[(5Z)-4-oxo-5-(6-quinoxalinylmethylidene)-4,5-dihydro-1,3-thiazol-2-yl]amino}phenyl)-2,2-dimethylpropanamide;
1,1-Dimethylethyl {2-[(4-chloro-3-{[(5Z)-4-oxo-5-(6-quinoxalinylmethylidene)-4,5-dihydro-1,3-thiazol-2-yl]amino}phenyl)amino]-1,1-dimethyl-2-oxoethyl}carbamate;
N-(2-Chloro-3-{[(5Z)-4-oxo-5-(6-quinoxalinylmethylidene)-4,5-dihydro-1,3-thiazol-2-yl]amino}phenyl)cyclopropanecarboxamide;
N-(2-Chloro-3-{[(5Z)-4-oxo-5-(6-quinoxalinylmethylidene)-4,5-dihydro-1,3-thiazol-2-yl]amino}phenyl)cyclobutanecarboxamide;
N˜1˜-(4-Chloro-3-{[(5Z)-4-oxo-5-(6-quinoxalinylmethylidene)-4,5-dihydro-1,3-thiazol-2-yl]amino}phenyl)-2-methylalaninamide;
N-(4-Chloro-3-{[(5Z)-4-oxo-5-(6-quinoxalinylmethylidene)-4,5-dihydro-1,3-thiazol-2-yl]amino}phenyl)ethanesulfonamide;
N-(4-Chloro-3-{[(5Z)-4-oxo-5-(6-quinoxalinylmethylidene)-4,5-dihydro-1,3-thiazol-2-yl]amino}phenyl)-2-propanesulfonamide;
N-(4-Chloro-3-{[(5Z)-4-oxo-5-(6-quinolinylmethylidene)-4,5-dihydro-1,3-thiazol-2-yl]amino}phenyl)-N-(1-methylethyl)acetamide;
N-(4-Chloro-3-{[(5Z)-4-oxo-5-(6-quinoxalinylmethylidene)-4,5-dihydro-1,3-thiazol-2-yl]amino}phenyl)-N-(1-methylethyl)acetamide;
N-(4-Chloro-3-{[(5Z)-4-oxo-5-(6-quinolinylmethylidene)-4,5-dihydro-1,3-thiazol-2-yl]amino}phenyl)-N-(1-methylethyl)methanesulfonamide;
N-(4-Chloro-3-{[(5Z)-4-oxo-5-(6-quinoxalinylmethylidene)-4,5-dihydro-1,3-thiazol-2-yl]amino}phenyl)-N-(1-methylethyl)methanesulfonamide;
(5Z)-2-({2-Chloro-5-[(1-methylethyl)amino]phenyl}amino)-5-(6-quinoxalinylmethylidene)-1,3-thiazol-4(5H)-one;
N-(2-Chloro-3-{[(SZ)-4-oxo-5-(6-quinoxalinylmethylidene)-4,5-dihydro-1,3-thiazol-2-yl]amino}phenyl)methanesulfonamide;
N-(2-Chloro-3-{[(5Z)-4-oxo-5-(6-quinoxalinylmethylidene)-4,5-dihydro-1,3-thiazol-2-yl]amino}phenyl)benzamide;
1,1-Dimethylethyl {2-[(4-chloro-3-{[(5Z)-4-oxo-5-(6-quinolinylmethylidene)-4,5-dihydro-1,3-thiazol-2-yl]amino}phenyl)amino]-1,1-dimethyl-2-oxoethyl}carbamate;
N¹-(4-Chloro-3-{[(5Z)-4-oxo-5-(6-quinolinylmethylidene)-4,5-dihydro-1,3-thiazol-2-yl]amino}phenyl)-2-methylalaninamide;
N-(4-Chloro-3-{[(5Z)-4-oxo-5-(6-quinolinylmethylidene)-4,5-dihydro-1,3-thiazol-2-yl]amino}phenyl)-2-propanesulfonamide;
4-Chloro-N-[2-(methyloxy)ethyl]-3-{[(5Z)-4-oxo-5-(6-quinolinylmethylidene)-4,5-dihydro-1,3-thiazol-2-yl]amino}benzamide;
4-Chloro-3-{[(5Z)-4-oxo-5-(6-quinolinylmethylidene)-4,5-dihydro-1,3-thiazol-2-yl]amino}-N-propylbenzamide;
4-Chloro-N-(2-hydroxyethyl)-3-{[(5Z)-4-oxo-5-(6-quinolinylmethylidene)-4,5-dihydro-1,3-thiazol-2-yl]amino}benzamide;
4-Chloro-N-[3-(2-oxo-1-pyrrolidinyl)propyl]-3-{[(5Z)-4-oxo-5-(6-quinolinylmethylidene)-4,5-dihydro-1,3-thiazol-2-yl]amino}benzamide;
4-Chloro-N-[2-(4-morpholinyl)ethyl]-3-{[(5Z)-4-oxo-5-(6-quinolinylmethylidene)-4,5-dihydro-1,3-thiazol-2-yl]amino}benzamide;
4-chloro-N-[2-(4-morpholinyl)ethyl]-3-[(4-oxo-4,5-dihydro-1,3-thiazol-2-yl)amino]benzamide;
4-Chloro-N-[3-(4-morpholinyl)propyl]-3-{[(5Z)-4-oxo-5-(6-quinolinylmethylidene)-4,5-dihydro-1,3-thiazol-2-yl]amino}benzamide;
4-Chloro-N-(2-hydroxy-1,1-dimethylethyl)-3-{[(5Z)-4-oxo-5-(6-quinolinylmethylidene)-4,5-dihydro-1,3-thiazol-2-yl]amino}benzamide;
4-Chloro-N-(1-methyl-4-piperidinyl)-3-{[(5Z)-4-oxo-5-(6-quinolinylmethylidene)-4,5-dihydro-1,3-thiazol-2-yl]amino}benzamide;
(5Z)-2-[(2-Chloro-5-{[(3S)-3-hydroxy-1-pyrrolidinyl]carbonyl}phenyl)amino]-5-(6-quinolinylmethylidene)-1,3-thiazol-4(5H)-one;
4-Chloro-3-{[(5Z)-4-oxo-5-(6-quinolinylmethylidene)-4,5-dihydro-1,3-thiazol-2-yl]amino}-N-[2-(2-thienyl)ethyl]benzamide;
(5Z)-2-{[2-Chloro-5-({4-[2-oxo-2-(1-pyrrolidinyl)ethyl]-1-piperazinyl}carbonyl)phenyl]amino}-5-(6-quinolinylmethylidene)-1,3-thiazol-4(5H)-one;
4-Chloro-N-(cyclopropylmethyl)-3-{[(5Z)-4-oxo-5-(6-quinolinylmethylidene)-4,5-dihydro-1,3-thiazol-2-yl]amino}benzamide;
4-Chloro-N-(trans-4-hydroxycyclohexyl)-3-{[(5Z)-4-oxo-5-(6-quinolinylmethylidene)-4,5-dihydro-1,3-thiazol-2-yl]amino}benzamide;
4-Chloro-N-(3-hydroxypropyl)-3-{[(5Z)-4-oxo-5-(6-quinolinylmethylidene)-4,5-dihydro-1,3-thiazol-2-yl]amino}benzamide;
4-Chloro-3-{[(5Z)-4-oxo-5-(6-quinolinylmethylidene)-4,5-dihydro-1,3-thiazol-2-yl]amino}-N-[2-(1-pyrrolidinyl)ethyl]benzamide;
4-Chloro-3-{[(5Z)-4-oxo-5-(6-quinolinylmethylidene)-4,5-dihydro-1,3-thiazol-2-yl]amino}-N-[2-(1-piperidinyl)ethyl]benzamide;
4-Chloro-N-[3-(4-methyl-1-piperazinyl)propyl]-3-{[(5Z)-4-oxo-5-(6-quinolinylmethylidene)-4,5-dihydro-1,3-thiazol-2-yl]amino}benzamide;
(5Z)-2-({2-Chloro-5-[(4-methyl-1-piperazinyl)carbonyl]phenyl}amino)-5-(6-quinolinylmethylidene)-1,3-thiazol-4(5H)-one;
4-Chloro-N-ethyl-3-{[(5Z)-4-oxo-5-(6-quinolinylmethylidene)-4,5-dihydro-1,3-thiazol-2-yl]amino}benzamide;
4-Chloro-N-[2-(dimethylamino)ethyl]-N-methyl-3-{[(5Z)-4-oxo-5-(6-quinolinylmethylidene)-4,5-dihydro-1,3-thiazol-2-yl]amino}benzamide; and
(5Z)-2-{[2-Chloro-5-(4-morpholinylcarbonyl)phenyl}amino]-5-(6-quinolinylmethylidene)-1,3-thiazol-4(5H)-one.

14. A pharmaceutically acceptable salt, hydrate, solvate or pro-drug of a compound of claim 13.

15. A pharmaceutical composition comprising a compound according to claim 1, and/or a pharmaceutically acceptable salt, hydrate, solvate or pro-drug thereof and a pharmaceutically acceptable carrier.

16. A process for preparing a pharmaceutical composition containing a pharmaceutically acceptable carrier or diluent and an effective amount of a compound of Formula (I) as described in claim 1 and/or a pharmaceutically acceptable salt, hydrate, solvate or pro-drug thereof, which process comprises bringing the compound of Formula (I) and/or a pharmaceutically acceptable salt, hydrate, solvate or pro-drug thereof into association with a pharmaceutically acceptable carrier or diluent.

17. A method of inhibiting hYAK3 in a mammal; comprising, administering to the mammal a therapeutically effective amount of a compound of the Formula I, as described in claim 1, or a pharmaceutically acceptable salt, hydrate, solvate or pro-drug thereof.

18. A method of treating or preventing diseases of the erythroid and hematopoietic systems, caused by hYAK3 activity; comprising, administering to a mammal a therapeutically effective amount of a compound of claim 1, or a pharmaceutically acceptable salt, hydrate, solvate or pro-drug thereof and one or more of pharmaceutically acceptable carriers, diluents and excipients.

19. A method of claim 18 in which diseases of the erythroid and hematopoietic systems are selected from the group consisting of: anemia, aplastic anemia, myelodysplastic syndrome, myelosuppression, and cytopenia.

20. A method of treating or preventing diseases selected from the group consisting of: anemia, aplastic anemia, myelodysplastic syndrome, myelosuppression, and cytopenia; comprising, administering to a mammal a therapeutically effective amount of a compound of claim 1, or a pharmaceutically acceptable salt, hydrate, solvate or pro-drug thereof and one or more of pharmaceutically acceptable carriers, diluents and excipients.

21. The method of claim 18 wherein the mammal is a human.

22. A method of treating diseases of the hematopoietic system, in a mammal in need thereof, which comprises: administering to such mammal a therapeutically effective amount of
a) a compound of Formula (I), as described in claim 1 and/or a pharmaceutically acceptable salt, hydrate, solvate or pro-drug thereof; and
b) EPO or a derivative thereof.
